SEQUENCE LISTING (10 sequences)

SEQ ID NO: 1 - protein, 162 aa, homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 19 aa, homo sapiens
RHMIRMRQLIDIVDQLKNY

SEQ ID NO: 3 - protein, 11 aa, homo sapiens
EWSAFSCFQKA

SEQ ID NO: 4 - protein, 11 aa, homo sapiens
ERIINVSIKKL

SEQ ID NO: 5 - protein, 20 aa, homo sapiens
PKEFLERFKSLLQKMIHQHL

SEQ ID NO: 6 - protein, 214 aa, artificial
  mAb14 light chain
AIQLTQSPSSLSASVGDRVTITCRASQDIDSALAWYQQKPGKAPKILIHDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 447 aa, artificial
  mAb14 heavy chain
EVQLVESGGGLVKPGGSLRLSCAASGFIFSSYSMNWVRQAPGKGLEWVSSITSGSYYIHYADSVKGRFTISRDNAKNSLYLQMNSLRAEDTAVYYCVRERGWGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 369 aa, homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 214 aa, artificial; identical to SEQ ID NO: 6
  Fab35 light chain (Fab 35 is a Fab fragment of mAb14)

SEQ ID NO: 10 - protein, 221 aa, artificial
  Fab35 heavy chain (Fab35 is a Fab fragment of mAb14)
EVQLVESGGGLVKPGGSLRLSCAASGFIFSSYSMNWVRQAPGKGLEWVSSITSGSYYIHYADSVKGRFTISRDNAKNSLYLQMNSLRAEDTAVYYCVRERGWGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESK

(19) United States
(12) Patent Application Publication
(10) Pub. No.: US 2014/0170153 A1
(43) Pub. Date: Jun. 19, 2014

(54) IL-21 EPITOPE AND IL-21 LIGANDS

(71) Applicants: Anders Svensson, Malmoe (SE); Mette Dahl Andersen, Vaerloese (DK); Jens Breinholt, Dyssegaard (DK); Charlotte Wiberg, Bjaerred (SE); Hanne Benedicte Rasmussen, Hilleroed (DK); Berit Olsen Krogh, Roedovre (DK)

(72) Inventors: Anders Svensson, Malmoe (SE); Mette Dahl Andersen, Vaerloese (DK); Jens Breinholt, Dyssegaard (DK); Charlotte Wiberg, Bjaerred (SE); Hanne Benedicte Rasmussen, Hilleroed (DK); Berit Olsen Krogh, Roedovre (DK)

(73) Assignee: Novo Nordisk A/S, Bagsvaerd (DK)

(21) Appl. No.: 14/122,572

(22) PCT Filed: May 31, 2012
(86) PCT No.: PCT/EP2012/060248

§ 371 Date: Feb. 4, 2014

Related U.S. Application Data
(60) Provisional application No. 61/492,990, filed on Jun. 3, 2011.

(30) Foreign Application Priority Data
May 31, 2011 (EP) 11168327.2

(51) Int. Cl. C07K 16/24; G01N 33/68

(52) U.S. Cl. CPC C07K 16/244; G01N 33/6869
USPC 424/139.1; 530/387.9; 506/9

(57) ABSTRACT

The present invention relates to IL-21 ligands, such as e.g. antibodies, as well as use thereof.

The present invention is concerned with a discontinuous epitope present on IL-21, and ligands which bind to this epitope.

IL-21 is a type I cytokine, which exerts pleiotropic effects on both innate and adaptive immune responses. It is mainly produced by activated CD4+ T cells, follicular T cells and Natural killer cells (NKT). In addition, recent evidence suggests that Th17 cells can produce high amount of IL-21.

IL-21 increases the cytotoxicity of CD8+ T cells and can promote proliferation of CD8+ cells in the presence of antigens. IL-21 is induced by IL-6, a cytokine known to promote development of Th17 cells. IL-21 acts on T helper cells in an autocrine manner promoting its own production and supporting differentiation of T-helper cells into Th17 cells. In agreement with this, IL-21 deficient mice show an impaired Th17 response. IL-21 also acts on B-cells and increases antibody production; however, IL-21 is not essential for production of functional antibodies, whereas IL-21Rα negative mice exhibit both reduced proliferation as well as impaired cytotoxicity of CD8+ cells. A recent set of studies suggests that IL-21 produced by CD4+ cells is critical for the ability of CD8+ T cells to control viral infection.

Mature IL-21 is a 133 amino acid polypeptide (residues 30-162 of SEQ ID No. 1, FIG. 2) featured by four helical segments, arranged in an up-up-down-down topology. IL-21 signals through a heterodimeric receptor complex consisting of the private IL-21 receptor alpha chain (IL-21Rα and the common gamma chain (γC) (residues 23-369 of SEQ ID No. 8). IL-21 comprises two binding sites, binding site 1 (BS1) and 2 (BS2), via which it interacts with IL-21Rα and γC, respectively. IL-21 binds via BS1 to IL-21Rα with high affinity, but receptor activation and signaling requires constructive interaction between IL-21 and γC via BS2 as well, hereby forming a ternary complex. IL-21 variants which bind IL-21Rα with high affinity, but lack the ability to interact constructively with γC will occupy the IL21 receptor without inducing signaling, and, thus, function as IL-21 receptor antagonists.

The ability of IL-21 to augment immunity has spurred substantial interest in the therapeutic use of IL-21. It is currently evaluated in clinical trials against metastatic melanoma types and renal cancer. Animal studies have demonstrated a synergistic effect between IL-21 and tumor specific antibodies, which could suggest a future therapeutic use of IL-21 as a potentiator of anti-tumor antibodies. Furthermore, IL-21 plays a complex role in autoimmune diseases. The ability of IL-21 to downregulate IgE production suggests that it could be used therapeutically against asthma and allergy. Results from animal studies support this view. On the other hand, the ability of IL-21 to promote Th17 development makes it a pro-inflammatory cytokine and a number of different IL-21 and IL-21Rα antagonists/inhibitors are currently investigated for potential use in treatment of a range of different autoimmune diseases.

Monoclonal antibodies specific for IL-21 are known in the art, for example from WO2007111714 and WO2010055366 (Zymo-Genetics, Inc.). In particular, WO2010055366 describes an IL-21 antibody, designated by clone number 366.328.10.63 (herein referred to as “mAb14”) which has high affinity for its cognate antigen, and other desirable properties, showing specificity for human and cynomolgus monkey IL-21. This antibody was shown not to compete with neither IL-21Rα nor γC binding of IL-21 using either a homodimeric IL-21Rα-Fc construct or a heterodimeric IL-21Rα/γC-Fc construct.

### SUMMARY OF THE INVENTION

We herein define a novel epitope on IL-21. Binding of a IL-21 ligand, e.g. an antibody, to this epitope competes or interferes with binding of γC to IL-21 via BS2, but does not interfere with binding of IL-21Rα to IL-21 via BS1.

We also describe IL-21 ligands, such as antibodies, which bind specifically to the epitope according to the invention, provided that the ligand is not mAb14, and not γC, as well as methods for making and using such ligands. We also describe how binding of mAb14 to IL-21 interferes with the binding of γC to IL-21.

Distinctive features of IL-21 ligands according to the invention are their ability to compete or interfere with binding of γC to IL-21, while IL-21 complexed with the ligand will maintain an IL-21Rα binding competent BS1. Accordingly, ligands of the present invention will in the presence of IL-21 form ligand:IL-21 complexes having the ability to bind specifically, and with high affinity, to IL-21Rα present on cell surfaces.

IL-21 variants which retain the ability to bind to IL-21Rα with high affinity via BS1, but have a BS2 lacking the ability to interact with γC will occupy the IL-21Rα receptor and function as IL-21Rα receptor antagonists. One way of compromising BS2 binding is the introduction of one or more point mutations of IL-21 residues critically involved in the interaction with γC. Another way is to block BS2 by binding a BS2 ligand to IL-21. Thus, IL-21 ligands effectively blocking BS2, but leaving BS1 unaffected, essentially as described for ligands of the present invention, are in the presence of IL-21 expected to act as IL-21Rα receptor antagonists in vivo.

Commonly, monoclonal antibodies are used therapeutically to “neutralize” soluble targets, such as pro-inflammatory molecules in autoimmune and chronic inflammatory disease. Binding of a IL21 ligand interfering with BS2 on an IL-21 molecule in solution will result in “neutralization” of that particular IL-21 molecule. However, as the formed ligand:IL-21 complex acquires antagonistic properties, it will additionally be able to block and “neutralize” the function of one IL-21Rα molecule on a IL-21Rα bearing cell. This dual mode of action, i.e. neutralization of soluble IL-21 and blockade of membrane bound IL-21Rα, will potentially improve the potency of such BS2 blocking/interfering IL-21 ligands, as compared with ligands interfering with IL-21 BS1, where the ligand:IL-21 complex formed will not acquire IL-21Rα antagonistic properties.

Ligands of the invention may thus have improved potency due to the combined neutralizing and receptor blocking properties.

Generally, a ligand of the invention will bind to IL-21 and form a ligand:IL-21 complex which retains a competent BS1 and thereby the ability to bind with high affinity to IL-21Rα. Therefore, the ligand:IL-21 complex is capable of binding to soluble fragments of IL-21Rα (e.g. its extra cellular domain) or membrane bound IL-21Rα present on cell surfaces. In other words ligands according to the invention may in the presence of IL-21 have the ability to bind specifically to IL-21Rα bearing cells.

In case the ligand is an antibody comprising a Fc domain capable of inducing ADCC and/or CDC, such ligand may, by virtue of its high affinity and specific binding to IL-21Rαbearing cells, possess the ability to kill such IL-21Rα bearing cells.

Thus, in another aspect ligands of the invention, e.g. antibodies comprising an Fc domain with built in effector functions, may mediate specific depletion of cells carrying IL-21Rα on their surfaces.

Depletion of specific cellular sub-sets, e.g. T cells and macrophages in the gut of patients with Crohn's disease (CD), has been shown to be an important component in the mode of action in current anti-TNFα therapy in CD (MacDonald, Nature Medicine, 16 (2010), p. 1194-1195, and references therein). Thus, depletion of specific inflammatory cells may be advantageous in the treatment of some inflammatory diseases.

The effector functions of antibodies are dependent on the isotype and can be modulated by several methods known in the art, including introduction of mutations in the Fc domain which will alter the binding of the antibody to Fc receptors. Ligands of the present invention include such ligands with modified effector functions.

IL-21 ligands binding to the epitope of the invention competes or interferes with γC binding to IL-21. Using experimental and homology modelling methods we predicted the location of the binding interface between IL-21 and γC and the specific amino acid residues in IL-21 which are involved in the interaction, and, thus, are targets for IL-21 ligands designed to inhibit the activity of IL-21 through disruption of the interaction between IL-21 and γC.

The following IL-21 amino acids, or a sub set thereof (with reference to SEQ ID NO 1) are bound by antibodies having CDR sequences similar to those of mAb14 (referred to as antibody 366.328.10.63 in WO2010055366): Glu 65, Asp 66, Val 67, Glu 68, Thr 69, Asn 70, Glu 72, Trp 73, Lys 117, His 118, Arg 119, Leu 143, Lys 146, Met 147, His 149, Gln 150 and His 151 as shown herein by X-ray crystallographic data.

### DETAILED DESCRIPTION OF THE INVENTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

### BRIEF DESCRIPTION OF THE DRAWINGS

FIG. 1: The amino acid sequences referred to herein.

FIG. 2: The mature IL-21 amino acid sequence (residues 30-162 of SEQ ID NO 1) is shown with helix A, B, C and D (corresponding to amino acids 34-50 of SEQ ID NO 1 (SEQ ID NO 2), 72-82 of SEQ ID NO 1 (SEQ ID NO 3), 93-103 of SEQ ID NO 1 (SEQ ID NO 4) and 133-152 of SEQ ID NO 1 (SEQ ID NO 5), respectively) appearing bold and underlined. Residues belonging to BS1, BS2 and the epitopes of mAb14 and mAb5 (Epitope14 and Epitope5, respectively) are marked below the amino acid sequence by “X”. The Mab5 epitope is indicated as “epitope5” in the figure. The Mab14 epitope is indicated as “epitope14” in the figure.

FIG. 3: HX monitored by mass spectrometry identifies regions in hIL-21 involved in mAb binding. For all panels the upper spectrum shows the non-deuterated control, the lower panel shows the deuterated control, i.e. hIL-21 in the absence of mAbs after 30 sec incubation in D₂O. The middle panels show the peptide after 30 sec in-exchange in the presence of mAbs as indicated.

(A) Mass/charge spectra corresponding to the peptide fragment 29-44, MQGQDRHMIRMRQLID (m/z=676.68, z=3) situated in helix A. mAb5 result in exchange protection in this region.
(B) Mass/charge spectra corresponding to the peptide fragment 67-76, VETNCEWSAF (m/z=1185.49, z=1) situated in a loop and helix B. mAb14 result in exchange protection in this region.
(C) Mass/charge spectra corresponding to the peptide fragment 93-98, ERIINV (m/z=743.47, z=1) situated in helix C. mAb5 result in exchange protection in this region.
(D) Mass/charge spectra corresponding to the peptide fragment 138-162, ERFKSLLQKMIHQHLSSRTHGSEDS (m/z=738.63, z=4) situated in helix D. mAb14 result in exchange protection in this region.

FIG. 4: Hydrogen exchange time-plots of representative peptides of hIL-21 in the absence or presence of mAb5 or mAb14. Deuterium incorporation (Da) of hIL-21 peptides is plotted against time on a logarithmic scale in the absence (black diamonds, ♦) or presence of mAb5 (white triangles, Δ) or mAb14 (white circles, ◯).

FIG. 5: Sequence coverage of HX analyzed peptides of hIL-21 in the presence and absence of mAb14. The primary sequence is displayed above the HX analyzed peptides (shown as horizontal bars). Peptides showing similar exchange patterns both in the presence and absence of mAb14 are displayed in white whereas peptides showing reduced deuterium incorporation upon mAb14 binding are coloured black. Boxed sequence regions define the epitope.

FIG. 6: Modelled hIL-21 residues in the X-ray structures of the different hIL-21/Fab complexes. Fab35 (From Example 1) is added for comparison.

FIG. 7: Summary of the Fab56, Fab57, Fab59 and Fab60 hIL-21 epitopes on hIL-21 identified by running the CONTACT software of the CCP4 program suite (Bailey, 1994). ‘=’ denotes a 4.0 Å distance cut-off between the Fab fragment and the hIL-21 molecule. ‘-’ denotes distances between 4.0 and 5.0 Å between the Fab fragment and the hIL-21 molecule.

### DEFINITIONS

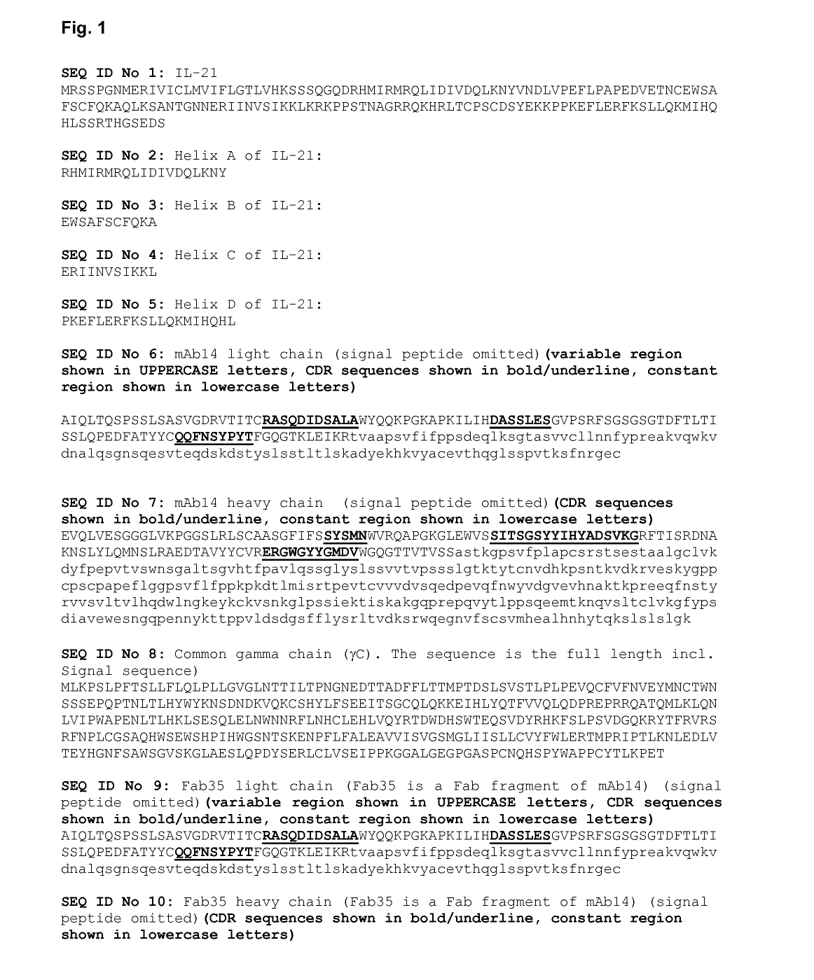

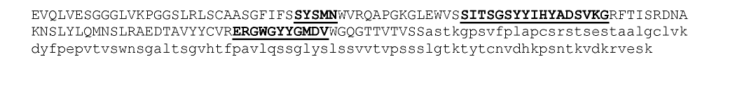

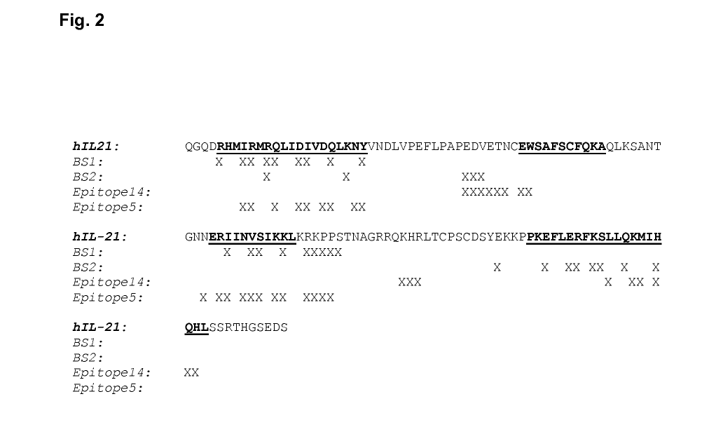

IL-21 refers, unless otherwise specifically stated, to human IL-21. The amino acid sequence of IL-21, including its signal sequence, is shown in FIG. 1 (SEQ ID NO 1). The mature IL-21 polypeptide corresponds to residues 30-162 of SEQ ID NO 1. IL-21 is featured by four helical segments, arranged in an up-up-down-down topology typical for the class I cytokines. IL-21 signals through a heterodimeric receptor complex consisting of the private chain IL-21Rα and γC the latter being shared by IL-2, IL-4, IL-7, IL-9, and IL-15. IL-21Rα binds IL-21 with high affinity via binding site 1 (BS1) on IL-21. The interaction between IL-21 and γC is, on the other hand, of a relatively low affinity. IL-21 binds to γC via its binding site 2 (BS2). IL-21 binding to both IL-21Rα and γC is required for signaling. Thus, IL-21 variants having high affinity for IL-21Rα and no or strongly reduced affinity for γC are expected to bind to IL-21Rα on the surface of IL-21R expressing cells and thereby block intracellular IL-21 induced signaling.

The structure of human IL-21 has previously been determined by NMR spectroscopy (Bondensgaard et. al J. Biol. Chem. (2007), 282, 23326-23336). The crystal structure of IL-21, free or in complex with receptor chains, has not yet been published but the structurally related IL-2 molecule in complex with its three receptor chains (IL-2:IL2Rα:IL-2Rβ:γC) determined by X-ray crystallography has been published and its coordinates have been deposited in a publicly available database (Protein Data Bank).

Ligands interfering with γC binding to IL-21: Ligands according to the invention that have the ability to interfere with binding of γC to IL-21 does in this context mean ligands that bind to IL-21 and in doing so either directly compete with γC for binding to IL-21 or reduce its ability to bind to/affinity for IL-21. Such ligands will furthermore not interfere with binding of IL-21Rα to IL-21. This means that ligands according to the invention may bind to an epitope that either overlaps with or is situated close enough to BS2 to provide sterical hindrance for γC-binding and thereby reducing its ability to bind to IL-21 by at least 25%, preferably at least 50%, preferably at least 60%, preferably at least 70%, preferably at least 75%, preferably at least 80%, preferably at least 90%, and most preferably at least 95%. It follows that the epitope on IL-21 of the ligand according to the invention is well separated from BS1 because binding of the ligands according to the invention does not significantly interfere with IL-21Rα binding to IL-21. Interference with γC binding can be detected by e.g. Surface Plasom Resonance (SPR) as shown in the examples.

The term “treatment”, as used herein, refers to the medical therapy of any human or other animal subject in need thereof. Said subject is expected to have undergone physical examination by a medical or veterinary medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health. Thus, said treatment may be prophylactic, palliative, symptomatic and/or curative.

In terms of the present invention, prophylactic, palliative, symptomatic and/or curative treatments may represent separate aspects of the invention.

The present invention concerns an epitope which has been discovered on human IL-21. Polypeptides having this epitope, therefore, are polypeptides which share at least part of the three-dimensional structure of human IL-21.

A fragment of a polypeptide is a polypeptide which is truncated at the C or N terminus, or which has had one or more amino acids removed from its sequence. In the context of the present invention, a fragment should retain sufficient three-dimensional structure to define the epitope or paratope of the invention.

Screening for binding activity (or any other desired activity) is conducted according to methods well known in the art, for instance SPR (Surface Plasmon Resonance), FACS, ELISA, etc. Screening allows selection of members of a repertoire according to desired characteristics.

As used herein, an “isolated” compound is a compound that has been removed from its natural environment.

IL-21 variants: IL-21 mimics/variants according to the present invention comprises the discontinuous epitope comprising at least one amino acid residue from at least two of the following IL-21 peptide segments: Glu 65 to Phe 73, Lys 117 to Arg 119, and Leu 143 to His 151, as set forth in SEQ ID No 1. Such mimics/variants may be produced in a number of ways, one of which is the mutation of native IL-21 by insertion, substitution or deletion of amino acids. The insertion, substitution or deletion may vary in size and extent, largely as a function of its position in the molecule. For example, large N or C-terminal insertions may be tolerated without modifying the epitope of the invention, as can C-terminal deletions. Elsewhere, smaller insertions, deletions or substitutions may be better tolerated.

Antibodies: The term “antibody” as referred to herein refers to a poly-peptide derived from a germline immunoglobulin sequence. The term includes full-length antibodies and any antigen binding fragment as e.g. Fab fragments, and other monovalent antibodies. The term “antibody”, “monoclonal antibody” and “mAb” as used herein, is intended to refer to immunoglobulin molecules and fragments thereof that have the ability to specifically bind to an antigen. A sub-class of the immunoglobulins of particular pharmaceutical interest are those belonging to the IgG family, which can be sub-divided into the iso-types IgG1, IgG2, IgG3 and IgG4. IgG molecules are composed of two heavy chains interlinked by two or several disulfide bonds and two light chains, one attached to each of the heavy chains by a disulfide bond. The IgG heavy chain is composed of four Ig-domains, including the variable domain (VH) and three constant domains (CH1, CH2, and CH3). Each light chain is comprised of a light chain variable region (VL) and a light chain constant region (CL). The variable regions of the heavy and light chains contain a binding domain that interacts with an antigen. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Examples of antigen-binding fragments include Fab, Fab′, F(ab)2, F(ab′)2, F(ab)S, Fv (typically the VL and VH domains of a single arm of an antibody), single-chain Fv (scFv; see e.g. Bird et al., Science 1988; 242:42 S-426; and Huston et al. PNAS 1988; 85:5879-5883), dsFv, Fd (typically the VH and CHI domain), and dAb (typically a VH domain) fragments; VH, VL, VhH, and V-NAR domains; monovalent molecules comprising a single VH and a single VL chain; minibodies, diabodies, triabodies, tetrabodies, and kappa bodies (see, e.g., Ill et al. Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 2S:1126-1136; WO2005040219, and published U.S. Patent Applications 20050238646 and 20020161201.

The Fc domain of an antibody according to the invention may be modified in order to modulate certain effector functions such as e.g. complement binding and/or binding to certain Fcγ receptors. The Fc domain may furthermore be modulated in order to increase affinity to the neonatal Fc receptor (FcRn). Mutations in positions 234, 235 and 237 (residue numbering according to the EU index) in an IgG1 Fc domain will generally result in reduced binding to the FcγRI receptor and possibly also the FcγRIIa and the FcγRIII receptors. These mutations do not alter binding to the FcRn receptor, which promotes a long circulatory half life by an endocytic recycling pathway. Preferably, a modified IgG1 Fc domain of an antibody according to the invention comprises one or more of the following mutations that will result in decreased affinity to certain Fcγ receptors (L234A, L235E, and G237A) and in reduced C1q-mediated complement fixation (A330S and P331S), respectively (residue numbering according to the EU index). Alternatively, the Fc domain may be an IgG4 Fc domain optionally comprising the S241P/S228P mutation (S241P denotes residue numbering according to Kabat, S228P denotes residue numbering according to the EU numbering system (Edelman G. M. et AL., Proc. Natl. Acad. USA 63, 78-85 (1969).

The term “human antibody”, as used herein, means antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences, e.g. the so-called “humanized antibodies” or human/mouse chimera antibodies.

The term “chimeric antibody” or “chimeric antibodies” refers to antibodies whose light and heavy chain genes have been constructed, typically by genetic engineering, from immunoglobulin variable and constant region genes belonging to different species. For example, the variable segments of genes from a mouse monoclonal antibody may be joined to human constant segments.

Half life extending moiety: The ligand according to the invention may be modified in order to increase its serum half-life, for example, by adding molecules—such as fatty acids or fatty acid derivates, PEG (poly ethylene glycol) or other water soluble polymers, including polysaccharide polymers to increase circulatory half-life. “Protractive groups”/“half life extending moiety” is herein understood as one or more chemical groups attached to one or more amino acid site chain functionalities such as —SH, —OH, —COOH, —CONH2, —NH2, or one or more N- and/or O-glycan structures and that can increase in vivo circulatory half life of a number of therapeutic proteins/peptides when conjugated to these proteins/peptides. Examples of protractive groups/half life extending moiety include but not limited to are: Biocompatible fatty acids and derivatives thereof, Hydroxy Alkyl Starch (HAS) e.g. Hydroxy Ethyl Starch (HES), Poly Ethylen Glycol (PEG), Poly (Glyx-Sery)n (HAP), Hyaluronic acid (HA), Heparosan polymers (HEP), Phosphorylcholine-based polymers (PC polymer), Fleximers, Dextran, Poly-sialic acids (PSA), an Fc domain, Transferrin, Albumin, Elastin like peptides, XTEN polymers, Albumin binding peptides, a CTP peptide, and any combination thereof.

Binning/competition binding: Antibodies binding to the same antigen can be characterized with respect to their ability to bind to their common antigen simultaneously. Antibodies may be subjected to “binning”, which term in the present context refers to a method of grouping antibodies that bind to the same antigen. “Binning” of antibodies may be based on competition binding of two antibodies to their common antigen in assays based on standard techniques such as surface plasmon resonance (SPR), ELISA or flow cytometry.

A “bin” is defined by a reference antibody. If a second antibody is unable to bind to the antigen at the same time as the reference antibody, the second antibody is said to belong to the same “bin” as the reference antibody, In this case the reference and the second antibody are competing for binding to the antigen, thus the pair of antibodies is termed “competing antibodies”. If a second antibody is capable of binding to the antigen at the same time as the reference antibody, the second antibody is said to belong to a separate “bin”. In this case the reference and the second antibody are not competing for binding to the antigen, thus the pair of antibodies is termed “non-competing antibodies”.

Antibody “binning” does not provide direct information about the epitope. Competing antibodies, i.e. antibodies belonging to the same “bin” may have identical epitopes, overlapping epitopes or even separate epitopes. The latter is the case if the reference antibody bound to its epitope on the antigen takes up the space required for the second antibody to contact its epitope on the antigen (“steric hindrance”). Non-competing antibodies have separate epitopes.

Epitope, paratope and antigen: The term “epitope”, as used herein, is defined in the context of a molecular interaction between an “antigen binding molecule”, such as an antibody (Ab), and its corresponding “antigen” (Ag). The term antigen (Ag) may refer to the molecular entity used for immunization of an immunocompetent vertebrate to produce the antibody (Ab) that recognizes the Ag. Herein, Ag is termed more broadly and is generally intended to include target molecules that are specifically recognized by the Ab, thus including fragments or mimics of the molecule used in the immunization process for raising the Ab. Generally, “epitope” refers to the area or region on an Ag to which an Ab specifically binds, i.e. the area or region in physical contact with the Ab. Physical contact may be defined through distance criteria (e.g. a distance cut-off of 4 Å) for atoms in the Ab and Ag molecules.

A “discontinuous epitope” is an epitope which is formed by two or more regions of a polypeptide which are not adjacent to each other in the linear peptide sequence, but which are arranged in the three-dimensional structure of the polypeptide to form a structural epitope. Other types of epitopes include: linear peptide epitopes, conformational epitopes which consist of two or more non-contiguous amino acids located near each other in the three-dimensional structure of the antigen; and post-translational epitopes which consist, either in whole or part, of molecular structures covalently attached to the antigen, such as carbohydrate groups.

The epitope for a given antibody (Ab)/antigen (Ag) pair can be defined and characterized at different levels of detail using a variety of experimental and computational epitope mapping methods. The experimental methods include mutagenesis, X-ray crystallography, Nuclear Magnetic Resonance (NMR) spectroscopy and Hydrogen deuterium eXchange Mass Spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

At its most detailed level, the epitope for the interaction between the Ag and the Ab can be described by the spatial coordinates defining the atomic contacts present in the Ag-Ab interaction, as well as information about their relative contributions to the binding thermodynamics. At a less detailed level, the epitope can be described by the spatial coordinates defining the atomic contacts between the Ag and Ab. At an even less detailed level the epitope can be described by the amino acid residues that it comprises as defined by a specific criteria such as the distance between atoms in the Ab and the Ag. At a further less detailed level the Ab-Ag interaction can be characterized through function, e.g. by competition binding with other Abs and “binning” although competition binding does not provide any structural information about the epitope.

In the context of an X-ray derived crystal structure defined by spatial coordinates of a complex between an Ab, e.g. a Fab fragment, and its Ag, the term epitope is herein, unless otherwise specified or contradicted by context, specifically defined as IL21 residues characterized by having a heavy atom (i.e. a non-hydrogen atom) within a distance of about 3.5 to about 5.0 Å, such as e.g. 4 Å from a heavy atom in the Ab.

From the fact that descriptions and definitions of epitopes, dependant on the epitope mapping method used, are obtained at different levels of detail, it follows that comparison of epitopes for different Abs on the same Ag can similarly be conducted at different levels of detail.

Epitopes described on the amino acid level, e.g. determined from an X-ray structure, are said to be identical if they contain the same set of amino acid residues. Epitopes are said to overlap if at least one amino acid is shared by the epitopes. Epitopes are said to be separate (unique) if no amino acid residue are shared by the epitopes.

The definition of the term “paratope” is derived from the above definition of “epitope” by reversing the perspective. Thus, the term “paratope” refers to the area or region on the Ab to which an Ag specifically binds, i.e. with which it makes physical contact to the Ag.

In the context of an X-ray derived crystal structure, defined by spatial coordinates of a complex between an Ab, such as a Fab fragment, and its Ag, the term paratope is herein, unless otherwise specified or contradicted by context, specifically defined as Ab residues characterized by having a heavy atom (i.e. a non-hydrogen atom) within a distance of about 4 Å (3.5 to 5.0 Å) from a heavy atom in IL21.

The epitope and paratope for a given antibody (Ab)/antigen (Ag) pair may be described by routine methods. For example, the overall location of an epitope may be determined by assessing the ability of an antibody to bind to different fragments or variants of IL21. The specific amino acids within IL21 that make contact with an antibody (epitope) and the specific amino acids in an antibody that make contact with IL21 (paratope) may also be determined using routine methods. For example, the Ab and Ag molecules may be combined and the Ab/Ag complex may be crystallised. The crystal structure of the complex may be determined and used to identify specific sites of interaction between the Ab and Ag.

Binding affinity between two molecules, e.g. an antibody, or fragment thereof, and an antigen, through a monovalent interaction may be quantified by determination of the equilibrium dissociation constant (KD). In turn, KD can be determined by measurement of the kinetics of complex formation and dissociation, e.g. by the SPR method. The rate constants corresponding to the association and the dissociation of a monovalent complex are referred to as the association rate constant ka (or kon) and dissociation rate constant kd (or koff), respectively. KD is related to ka and kd through the equation KD=kd/ka. Following the above definition, binding affinities associated with different molecular interactions, such as comparison of the binding affinity of different antibodies for a given antigen, may be compared by comparison of the KD values for the individual antibody/antigen complexes.

Non-Antibody Ligands: Ligands specific for the epitope according to the present invention can also encompass antibody mimics comprising one or more IL-21 binding portions built on a molecular scaffold (such as a protein or carbohydrate scaffold) specific for the epitope described herein. Proteins having relatively defined three-dimensional structures, commonly referred to as protein scaffolds, may be used as templates for the design of antibody mimics. These scaffolds typically contain one or more regions which are amenable to specific or random sequence variation, and such sequence randomization is often carried out to produce libraries of proteins from which desired products may be selected. For example, an antibody mimic can comprise a chimeric non-immunoglobulin binding polypeptide having an immunoglobulin-like domain containing scaffold having two or more solvent exposed loops containing a different CDR from a parent antibody inserted into each of the loops and exhibiting selective binding activity toward a ligand bound by the parent antibody. Non-immunoglobulin protein scaffolds have been proposed for obtaining proteins with novel binding properties.

Structure of ligands: As described above, a ligand as referred to herein may be an antibody (for example IgG, IgM, IgA, IgE) or fragment thereof (for example Fab, Fv, disulphide linked Fv, scFv, diabody) which comprises at least one heavy and a light chain variable domain which are complementary to one another and thus can associate with one another to form a VH/VL pair. It may be derived from any species naturally producing an antibody, or created by recombinant DNA technology; whether isolated from serum, B-cells, hybridomas, transfectomas, mammalian cells, yeast or bacteria.

Therapeutic Applications: IL-21 is involved in T-cell mediated immunity, and has been shown to promote a number of inflammatory cytokines. Accordingly, the ligands according to invention can be used in the treatment of diseases involving an inappropriate or undesired immune response (immunological disorders), such as inflammation, autoimmunity, conditions involving such mechanisms as well as graft vs. host disease. In one embodiment, such disease or disorder is an autoimmune and/or inflammatory disease. Examples of such autoimmune and/or inflammatory diseases are Systemic Lupus Erythematosus (SLE), Rheumatoid Arthritis (RA) and inflammatory bowel disease (IBD) (including ulcerative colitis (UC) and Crohn's disease (CD)), multiple sclerosis (MS), scleroderma and type 1 diabetes (T1 D), and other diseases and disorders, such as PV (pemphigus vulgaris), psoriasis, atopic dermatitis, celiac disease, kol, hashimoto's thyroiditis, graves' disease (thyroid), Sjogren's syndrome, guillain-barre syndrome, goodpasture's syndrome, additon's disease, Wegener's granulomatosis, primary biliary sclerosis, sclerosing cholangitis, autoimmune hepatitis, polymyalgia rheumatica, paynaud's phenomenon, temporal arteritis, giant cell arteritis, autoimmune hemolytic anemia, pernicious anemia, polyarteritis nodosa, behcet's disease, primary bilary cirrhosis, uveitis, myocarditis, rheumatic fever, ankylosing spondylitis, glomerulenephritis, sarcoidosis, dermatomyositis, myasthenia gravis, polymyositis, alopecia greata, type I diabetes, Colitis-Associated Tumorigenesis, and vitilgo.

In one embodiment, such disease or disorder is SLE, RA or IBD. In one embodiment, such disease or disorder is MS.

The IL-21 ligands of the present invention may be administered in combination with other medicaments as is known in the art.

The present invention further includes pharmaceutical compositions/formulations, comprising a pharmaceutically acceptable carrier and a polypeptide/ligand/antibody according to the invention as well as kits comprising such compositions. The pharmaceutical composition according to the invention may be in the form of an aqueous formulation or a dry formulation that is reconstituted in water/an aqueous buffer prior to administration.

Pharmaceutical compositions comprising ligands/antibodies/polypeptides according to the invention may be supplied as a kit comprising a container that comprises the compound according to the invention. Therapeutic polypeptides can be provided in the form of an injectable solution for single or multiple doses, or as a sterile powder that will be reconstituted before injection. Pharmaceutical compositions comprising compounds according to the invention are suitable for subcutaneous and/or IV administration.

Combination treatment: antibodies according to the invention may be co-administered with one or other more other therapeutic agents or formulations. The other agent may be intended to treat other symptoms or conditions of the patient. For example, the other agent may be an analgesic, an immunosuppressant or an anti-inflammatory agent.

Combined administration of two or more agents may be achieved in a number of different ways. In one embodiment, the antibody and the other agent may be administered together in a single composition. In another embodiment, the antibody and the other agent may be administered in separate compositions as part of a combined therapy. For example, the modulator may be administered before, after or concurrently with the other agent.

The antibodies/proteins according to the present invention may be administered along with other drugs (e.g. methotrexate, dexamethasone, and prednisone) and/or other biological drugs. Agents already in use in autoimmunity include immune modulators such as IFNbeta, Orencia (CTLA4-Ig), Humira (anti-TNF), Cimzia (anti-TNF, PEG Fab), Tysabri (a4-integrin mAb), Simponi, Rituxan/MabThera, Actemra/RoActemra, Kineret, Non-steroidal anti-inflammatory drugs (NSAIDS) like Asprin, Ibuprofen etc, Corticosteroids, disease-modifying antirheumatic drugs (DMARDS) like Plaquenil, Azulfidine, Methotrexate etc, Copaxone (glatirimer acetate), Gilneya (fingolimod), Antibiotics like Flagyl, Cipro, Topical (skin applied) medications including topical corticosteroids, vitamin D analogue creams (Dovonex), topical retinoids (Tazorac), moisturizers, topical immunomodulators (tacrolimus and pimecrolimus), coal tar, anthralin, and others, Raptiva, Ustekimumab, light therapy like PUVA, UVB, CellCept (mycophenolate mofetil).

### EMBODIMENTS

The following list of embodiments represents examples of embodiments of the present invention and should thus not be understood as limiting the invention.

1. An IL-21 mimic comprising an epitope comprising the following amino acids: Glu 65, Asp 66, Val 67, and His 149 as set forth in SEQ ID No. 1.
2. The mimic according to embodiment 1, wherein the epitope of said mimic further comprises one or more of the following amino acids: Arg 40, Lys 50, Glu 129, Glu 135, Glu 138, Arg 139, Lys 141, Ser 142, and Gln 145 as set forth in SEQ ID NO 1.
3. The mimic according to embodiment 1, wherein the epitope of said mimic further comprises one or more of the following amino acids: Glu 68, Thr 69, Asn 70, Glu 72, Trp 73, Lys 117, His 118, Arg 119, Leu 143, Lys 146, Met 147, Gln 150, and His 151.
4. The mimic according to any one of embodiments 1 to 3, wherein the epitope of said mimic further comprises the following amino acids: Glu 68, Thr 69, Asn 70, Glu 72, Trp 73, Lys 117, His 118, Arg 119, Leu 143, Lys 146, Met 147, Gln 150, and His 151.
5. A method for selecting a ligand which binds to IL-21, comprising screening one or more libraries of ligands with an IL-21 mimic according to any one of embodiments 1-4, and isolating one or more ligands which bind to said epitope.
6. Use of an IL-21 mimic according to any one of embodiments 1-4, for selecting a ligand which binds selectively to IL-21.
7. A ligand, wherein said ligand is preferably an antibody, which ligand binds specifically to the epitope of the IL-21 mimic according to any one of embodiments 1-4, provided that the ligand is not: (i) naturally occurring common γC (SEQ ID No. 8), and not (ii) the monoclonal antibody mAb14, the light and heavy chains of which are set forth in SEQ ID No. 6 and SEQ ID No. 7, respectively. If the ligand is an antibody, the antibody is not the monoclonal mAb14 antibody.
8. A ligand, wherein said ligand is preferably an antibody, which ligand binds to an epitope on IL-21, wherein said epitope comprises one or more of the Arg 40 to Val 67 amino acids as well as one or more of the Glu 129 to His 149 amino acids, as set forth in SEQ ID No. 1, provided that the ligand is not: (i) naturally occurring common gamma chain (SEQ ID No. 8), and not (ii) mAb14, the light and heavy chains of which are set forth in SEQ ID No. 6 and SEQ ID No. 7 respectively. Said ligand preferably comprises one or more of the Glu 65 to Val 67 amino acids and one or more of the Glu 129 to His 149 amino acids. If the ligand is an antibody, the antibody is not the monoclonal mAb14 antibody.
9. A ligand which binds to IL-21, wherein said ligand is preferably an antibody, wherein said ligand binds to at least one of the Arg 40, Lys 50, Glu 65, Asp 66, Val 67, Glu 129, Glu 135, Glu 138, Arg 139, Lys 141, Ser 142, Gln 145, and His 149 amino acids as set forth in SEQ ID NO 1, provided that the ligand is not: (i) naturally occurring common γC (SEQ ID No. 8), and not (ii) mAb14, the light and heavy chains of which are set forth in SEQ ID No. 6 and SEQ ID No. 7, respectively.
10. A ligand according to embodiment 9, wherein the said ligand binds to the Arg 40, Lys 50, Glu 65, Asp 66, Val 67, Glu 129, Glu 135, Glu 138, Arg 139, Lys 141, Ser 142, Gln 145, and His 149 amino acids as set forth in SEQ ID NO 1.
11. A ligand which binds to IL-21, wherein said ligand is preferably an antibody, wherein said ligand binds to at least one of the amino acids Glu 72 to Ala 82 in IL-21 (SEQ ID NO 1) provided that the ligand is not mAb14, the light and heavy chains of which are set forth in SEQ ID No. 6 and SEQ ID No. 7 respectively. Preferably, said ligand binds to at least one of the amino acids Glu 65 to Trp 73, provided that the ligand is not naturally occurring common γC (SEQ ID No. 8) and not mAb14, the light and heavy chains of which are set forth in SEQ ID No. 6 and SEQ ID No. 7, respectively. If the latter ligand is an antibody, the antibody is not the monoclonal mAb14 antibody.
12. A ligand according to any one of embodiments 7-11, wherein said ligand is preferably an antibody, wherein said ligand binds to amino acids Asn 70, Glu 72, and Trp 73 in IL-21 (SEQ ID NO 1).
13. A ligand according to any one of embodiments 7-12, wherein said ligand is preferably an antibody, wherein said ligand furthermore binds one or more of amino acids Glu 65, Asp 66, and Val 67 as set forth in SEQ ID NO 1.
14. A ligand according to any one of embodiments 7-13, wherein said ligand is preferably an antibody, wherein said ligand furthermore binds amino acid His 149 as set forth in SEQ ID NO 1.
15. A ligand according to any one of embodiments 7-14, wherein said ligand is preferably an antibody, wherein said ligand binds amino acids Glu 65, Asp 66, Val 67, and His 149 as set forth in SEQ ID NO 1.
16. A ligand which binds to IL-21, wherein said ligand is preferably an antibody, wherein said ligand binds to an epitope comprising 1, 2, 3, 4, 5, 6, 7, 8, 9, 10, 11, or 12 of the following amino acids: Arg 40, Lys 50, Glu 65, Asp 66, Val 67, Glu 129, Glu 135, Glu 138, Arg 139, Lys 141, Ser 142, Gln 145, and His 149 as set forth in SEQ ID No. 1, provided that the ligand is not: (i) naturally occurring common gamma chain (SEQ ID No. 8), and not (ii) mAb14, the light and heavy chains of which are set forth in SEQ ID No. 6 and SEQ ID No. 7, respectively. Preferably the ligand binds to the following amino acids: Arg 40, Lys 50, Glu 65, Asp 66, Val 67, Glu 129, Glu 135, Glu 138, Arg 139, Lys 141, Ser 142, Gln 145, and His 149 as set forth in SEQ ID No. 1.
17. A ligand according to embodiment 16, wherein said ligand is preferably an antibody, wherein said ligand binds to an epitope comprising the following amino acids: Arg 40, Lys 50, Glu 65, Asp 66, Val 67, Glu 129, Glu 135, Glu 138, Arg 139, Lys 141, Ser 142, Gln 145, and His 149 as set forth in SEQ ID No. 1.
18. A ligand according to any one of embodiments 7-15, wherein said ligand is preferably an antibody, wherein said ligand binds to an epitope comprising 1, 2, 3, 4, 5, 6, 7, 8, 9, 10, 11, 12, 13, 14, or 15 of the following amino acids: Glu 65, Asp 66, Val 67, Glu 68, Thr 69, Asn 70, Glu 72, Trp 73, Lys 117, His 118, Arg 119, leu 143, Lys 146, Met 147, His 149, Gln 150, and His 151.
19. A ligand which binds to IL-21, wherein said ligand is preferably an antibody, wherein said ligand binds to an epitope comprising the following amino acids: Glu 65, Asp 66, Val 67, Glu 68, Thr 69, Asn 70, Glu 72, Trp 73, Lys 117, His 118, Arg 119, leu 143, Lys 146, Met 147, His 149, Gln 150, and His 151, provided that the ligand is not: (i) naturally occurring common γC (SEQ ID No. 8), and not (ii) mAb14, the light and heavy chains of which are set forth in SEQ ID No. 6 and SEQ ID No. 7, respectively.
20. A ligand according to any one of embodiments 7-19, wherein said ligand is preferably an antibody, wherein said ligand comprises one, two, or three of CDR1, CDR2 and CDR3 as set forth in SEQ ID No. 6, and one, two, or three of CDR1, CDR2 and CDR3 as set forth in SEQ ID No. 7, provided that the ligand is not mAb14, the light and heavy chains of which are set forth in SEQ ID NO 6 and SEQ ID NO 7, respectively. The mAb14 antibody is the same antibody which is disclosed in WO2010/055366, designated therein by hybridoma clone number 366.328.10.63.
21. A ligand according to any one of embodiments 7-20, wherein said ligand is preferably an antibody, wherein said ligand interferes with binding of IL-21 to common γC.
22. A ligand according to any one of embodiments 7-21, wherein said ligand is an antibody. The antibody can be an antibody, a monoclonal antibody, an antigen binding fragment of an antibody, a monovalent antibody, a divalent antibody. The antibody may be a human or humanized form of any of these.
23. A ligand according to embodiment 22, wherein said antibody is an IgG1 antibody. The ligand may alternatively be an IgG4 antibody.
24. A ligand according to any one of embodiments 22-23, wherein said antibody comprises an Fc domain, which mediates antibody effector functions.
25. A ligand according to embodiment 24, wherein said ligand comprises an Fc domain having reduced effector functions.
26. A ligand according to embodiment 25, wherein said ligand comprises an IgG1 Fc domain comprising one, two, three, four or all of the following mutations that result in decreased affinity to certain Fc receptors (L234A, L235E, and G237A) and in reduced C1q-mediated complement fixation (A330S and P331S), respectively (residue numbering according to the EU index). Such ligands will retain a relatively long in vivo half life and significantly reduced effector functions.
27. A ligand according to embodiment 20, wherein said ligand is an antibody that is a variant of mAb14, the light and heavy chains thereof which are set forth in SEQ ID No. 6 and SEQ ID No. 7 respectively, wherein said ligand comprises one or more mutations in the CDR sequences, wherein said mutations are selected from one or more from the list consisting of: A61S (SEQ ID NO 7), D62E (SEQ ID NO 7), V64I (SEQ ID NO 7), and K65R (SEQ ID NO 7), R24K (SEQ ID NO 6), S26T (SEQ ID NO 6), Q27N (SEQ ID NO 6), D30E (SEQ ID NO 6), S53T (SEQ ID NO 6), and S56T (SEQ ID NO 6). Each of these mutations thus represents separate embodiments. Any combination thereof also represents separate embodiments.
28. An antibody which binds to an epitope on IL-21, wherein said epitope comprises one or more of the following amino acids: Glu 65, Asp 66, Val 67, Glu 68, Thr 69, Asn 70, Glu 72, Trp 73, one or more of the following amino acids Lys 117, His 118, Arg 119, and one or more of the following amino acids: Leu 143, Lys 146, Met 147, His 149, Gln 150, and His 151 as set forth in SEQ ID No. 1, provided that the antibody is not the monoclonal antibody mAb14, the light and heavy chains of which are set forth in SEQ ID No. 6 and SEQ ID No. 7, respectively. The antibody may alternatively bind to an epitope on IL-21, wherein said epitope comprises one or more of the following amino acids: Glu 65, Asp 66, Val 67, Glu 68, Thr 69, Asn 70, Glu 72, Trp 73, Lys 117, His 118, and Arg 119, and one or more of the following amino acids: Leu 143, Lys 146, Met 147, His 149, Gln 150, and His 151 as set forth in SEQ ID No. 1. The antibody may alternatively bind to an epitope on IL-21, wherein said epitope comprises one or more of the following amino acids: Glu 65, Asp 66, Val 67, Glu 68, Thr 69, Asn 70, Glu 72, and Trp 73, and one or more of the following amino acids: Lys 117, His 118, and Arg 119, Leu 143, Lys 146, Met 147, His 149, Gln 150, and His 151 as set forth in SEQ ID No. 1.
29. An antibody which binds to an epitope on IL-21, wherein said epitope comprises one or more of the following amino acids: Glu 65 to Trp 73, one or more of the following amino acids: Lys 117 to Arg 119, and one or more of the following amino acids: Leu 143 to His 151 as set forth in SEQ ID No. 1, provided that the antibody is not the monoclonal antibody mAb14, the light and heavy chains of which are set forth in SEQ ID No. 6 and SEQ ID No. 7, respectively. The antibody may alternatively bind to an epitope on IL-21, wherein said epitope comprises one or more of the following amino acids: Glu 65 to Trp 73, and one or more of the following amino acids: Leu 143 to His 151 as set forth in SEQ ID No. 1.
30. An antibody which binds to an epitope on IL-21, wherein said epitope comprises one or more of the Arg 40 to Val 67 amino acids as well as one or more of the Glu 129 to His 149 amino acids, as set forth in SEQ ID No. 1, provided that the antibody is not mAb14, the light and heavy chains of which are set forth in SEQ ID No. 6 and SEQ ID No. 7, respectively.
31. An antibody which binds to an epitope on IL-21, wherein said epitope comprises one or more of the Glu 65 to Trp 73 amino acids in IL-21 (SEQ ID NO. 1) provided that the antibody is not mAb14, the light and heavy chains of which are set forth in SEQ ID No. 6 and SEQ ID No. 7, respectively.
32. An antibody which binds to an epitope on IL-21, wherein said epitope comprises one or more of the Glu 65, Asp 66, Val 67, and His 149 amino acids as set forth in SEQ ID NO. 1, provided that the antibody is not mAb14, the light and heavy chains of which are set forth in SEQ ID No. 6 and SEQ ID No. 7, respectively.
33. A pharmaceutical composition comprising a ligand/antibody according to any one of embodiments 7-32 and optionally one or more pharmaceutically acceptable excipients. Such excipients/carriers are well known in the art. Such pharmaceutical compositions are preferably intended for IV administration and/or subcutaneous administration.
34. A kit comprising a ligand/antibody according to any one of embodiments 7-32.
35. Use of a ligand/antibody according to any one of embodiments 7-32 as a medicament.
36. Use of a ligand/antibody according to any one of embodiments 7-32 for treating an immunological disorder.
37. Use of a ligand/antibody according to any one of embodiments 7-32 for treating an autoimmune disease.
38. Use of a ligand/antibody according to any one of embodiments 7-32 for treating SLE.
39. Use of a ligand/antibody according to any one of embodiments 7-32 for treating RA.
40. Use of a ligand/antibody according to any one of embodiments 7-32 for treating IBD.
41. Use of a ligand/antibody according to any one of embodiments 7-32 for treating CD.
42. A method of treating an immunological disorder, wherein said method comprises administering to a person in need thereof an appropriate dosis of a ligand/antibody according to any one of embodiments 7-32.

The provision herein of the detailed 3-dimensional structural knowledge of the complex between the Fab fragment of mAb14 (Fab35) and IL-21, including their binding interface, can form the basis for rationally designing variants of the interacting molecules with desired properties. Properties that might be desirable to improve for antibodies may be chemical or physical properties e.g. solubility, viscosity and stability. Other properties that might be desirable to modulate are the antigenic properties of the antibodies and their ability to be bound by anti-antibodies.

### EXAMPLES

### Example 1

### Crystal Structure of IL-21 in Complex with a Fab Fragment of mAb14 (Fab35)

The 3-dimensional structure of IL-21 in complex with the Fab fragment (Fab35) of the human anti-IL-21 monoclonal antibody mAb14 was solved and refined to 1.64 Å resolution using X-ray crystallography. The results demonstrate that the Fab35 (representing mAb14) epitope on IL-21 is situated on a completely different part of the IL-21 molecule as compared with that of mAb5, and binds with a different binding mode. “mAb5” corresponds to an IgG1 version of the clone 362.78.1.44 antibody disclosed in WO2010055366, the Fc region of mAb5 carrying the L234A, L235E, and G237A (reduced Fc receptor binding) and A330S and P331S mutations (reduced C1q-mediated complement fixation). While mAb5 binds to the surface exposed faces of helix A and C on IL-21 Fab35 (mAb14) binds more towards one end of the four-helix bundle, interacting with the exposed loops but also penetrating in to the IL-21 molecule by inserting the side chain of a Tryptophane residue, W102 of the heavy chain, between helices B and D, and thereby slightly distorting the C-terminal part of helix D. Fab35 (representing mAb14) will, instead of competing with binding of IL-21Rα to IL-21 as mAb5, compete with, and due to its high binding affinity, block the binding of γC to IL-21. Hence, mAb14 will inhibit the biological effects mediated by IL-21 through γC.

The epitope described was characterized using the structure of the complex between Fab35 and IL-21. However, the conclusions regarding the epitope of Fab35 on IL-21 will also apply to the interaction between IL-21 and the corresponding full antibody, mAb14, from which Fab35 was derived.

hIL-21 (expressed in E. coli as the mature peptide; residues 30-162 of SEQ ID NO: 1 with an added N-terminal Methionine residue) in 10 mM histidine buffer, pH 5.3, and anti-IL-21 Fab35 (comprising a light chain corresponding to SEQ ID NO. 9 and a heavy chain fragment corresponding SEQ ID NO. 10), formulated in PBS buffer, pH 7.4 (4 tablets in 2 liter of water, GIBCO Cat. No. 18912-014 Invitrogen Corporation), were mixed in a molar ratio of 1:1. The final concentration of the complex was 10.3 mg/ml. Crystals were grown with the sitting drop technique in 30% w/v PEG1000 and 200 mM magnesium formate mixed in a ratio of 1:1 (precipitant solution volume:protein solution volume). Total drop size was 0.2 μl. A crystal was prepared for cryo-freezing by transferring 3 μl of a cryo-solution containing 75% of the precipitant solution and 25% glycerol to the drop containing the crystal, and soaking was allowed for about half a minute. The crystal was then flash frozen in liquid N₂ and kept at a temperature of 100 K during data collection by a cryogenic N₂ gas stream. Crystallographic data were collected to 1.64 Å resolution at beam-line BL911-2 (1) at MAX-lab, Lund, Sweden. Space group determination, integration and scaling of the data were made by the XDS software package (2). Cell parameters for the data were determined to be 89.4, 65.2, 106.7 Å, 90°, 111.57° and 90°, respectively, and the space group C2. R-sym to 1.64 Å resolution was 6.4% and completeness 98.2%. The molecular replacement technique, using the PHASER software program (3;4) of the CCP4 suite (5) was used for structure determination. The X-ray structure of the anti-IL-21 Fab9 (corresponding to mAb5), in complex with IL-21 (unpublished results), was used as input model for the PHASER software. The IL-21 molecule from the Fab9:IL-21 complex structure was also used, independently from the Fab, as input for the PHASER software. The software ARP/wARP (6) was subsequently used for an initial round of model building and was then followed by crystallographic refinements, using the software programs REFMAC5 (7) of the CCP4 software package and PHENIX.REFINE (8) of the PHENIX software package (9) and by computer graphics inspection of the electron density maps, model corrections and building using the Coot software program (10). The procedure was cycled until no further significant improvements could be made to the model. Final R- and R-free for all data were 0.179 and 0.211, respectively, and the model showed a root-mean-square deviation (RMSD) from ideal bond lengths of 0.022 Å.

### Results

The binding site of Fab35 will compete with, and due to its high binding affinity, block the binding of γC to IL-21. Hence, it will inhibit the biological effects mediated by IL-21 through γC.

Calculation of the areas excluded in pair-wise interactions by the software program Areaimol (11;12) of the CCP4 program suite (5) gave for the IL-21/Fab35 molecular complex in the crystal structure 1082 Å² for IL-21 and 1041 Å² for anti-IL-21, respectively. The average areas excluded in pair-wise interaction between the IL-21 molecule and Fab35 were calculated to be 1061 Å².

The direct contacts between the IL-21 and Fab35 were identified by running the contacts software of the CCP4 program suite (5) using a cut-off distance of 4.0 Å between Fab35 and the IL-21 molecules. The results from the IL-21/Fab35 complex crystal structure are shown in Table 1. The resulting IL-21 epitope for Fab35 (representing mAb14) was found to comprise the following residues of IL-21 (SEQ ID NO. 1): Glu 65, Asp 66, Val 67, Glu 68, Thr 69, Asn 70, Glu 72, Trp 73, Lys 117, His 118, Arg 119, Leu 143, Lys 146, Met 147, His 149, Gln 150 and His 151.

Thus, the Fab35 (mAb14) epitope comprise residues in the N-terminal part of helix B (residues 72-73), and residues in the C-terminal part of helix D (residues 143-151). Additionally, several contact residues were identified in the loop segment proceeding helix B (residues 65-70), and in the loop between helix C and helix D (residues 117-119). This epitope has a partial overlap with the predicted binding site for γC to IL-21.

The Fab35 (representing mAb14) paratope for IL-21 included residues Ser 31, Asp 50, Phe 91, Asn 92 and Tyr 94 of the light (L) chain (SEQ ID NO. 9, Table 2), and residues Ile 28, Ser 30, Ser 31, Tyr 32, Ser 33, Thr 52, Ser 53, Gly 54, Ser 55, Tyr 56, Tyr 57, His 59, Glu 99, Arg 100, Gly 101, Trp 102, Gly 103, Tyr 104 and Tyr 105 of the heavy (H) chain (SEQ ID NO. 10, Table 2). The epitope for the Fab35 fragment/mAb14 antibody is shown in FIG. 2

TABLE 1
Results from the X-ray model refinement to the observed data of the IL-
21/Fab35 complex by the software program Refmac5 (7) of the CCP4 program software
package (5).

| REMARK | 3 | REFINEMENT. | |
|---|---|---|---|
| REMARK | 3 | PROGRAM | : REFMAC 5.6.0085 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | | |
| REMARK | 3 | RESOLUTION RANGE LOW | (ANGSTROMS) | : 27.23 |
| REMARK | 3 | DATA CUTOFF | (SIGMA(F)) | : NONE |
| REMARK | 3 | COMPLETENESS FOR RANGE | (%) | : 98.27 |
| REMARK | 3 | NUMBER OF REFLECTIONS | | : 65231 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.18040 |
| REMARK | 3 | R VALUE | (WORKING SET) | : 0.17877 |

| REMARK | 3 | FREE R VALUE | | : 0.21100 |
|---|---|---|---|---|
| REMARK | 3 | FREE R VALUE TEST SET SIZE | (%) | : 5.1 |
| REMARK | 3 | FREE R VALUE TEST SET COUNT | | : 3487 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 1.640 |
| REMARK | 3 | BIN RESOLUTION RANGE LOW | | : 1.682 |
| REMARK | 3 | REFLECTION IN BIN | (WORKING SET) | : 4786 |
| REMARK | 3 | BIN COMPLETENESS | (WORKING + TEST) (%) | : 97.49 |
| REMARK | 3 | BIN R VALUE | (WORKING SET) | : 0.293 |

| | | | |
|---|---|---|---|
| REMARK | 3 | BIN FREE R VALUE SET COUNT | : 267 |
| REMARK | 3 | BIN FREE R VALUE | : 0.302 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS : 4812 | | |
| REMARK | 3 | | | |
| REMARK | 3 | B VALUES. | | |
| REMARK | 3 | FROM WILSON PLOT | (A**2) | : NULL |
| REMARK | 3 | MEAN B VALUE | (OVERALL, A**2) | : 27.871 |

| REMARK | 3 | OVERALL ANISOTROPIC B VALUE. | |
|---|---|---|---|
| REMARK | 3 | B11 (A**2) : | −0.34 |
| REMARK | 3 | B22 (A**2) : | 0.81 |
| REMARK | 3 | B33 (A**2) : | 0.23 |
| REMARK | 3 | B12 (A**2) : | 0.00 |
| REMARK | 3 | B13 (A**2) : | 0.96 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A) | : 0.097 |
| REMARK | 3 | ESU BASED ON FREE R VALUE | (A) | : 0.096 |
| REMARK | 3 | ESU BASED ON MAXIMUM LIKELIHOOD | (A) | : 0.072 |
| REMARK | 3 | ESU FOR B VALUES BASED ON MAXIMUM LIKELIHOOD | (A**2) | : 4.258 |
| REMARK | 3 | | | |

| REMARK | 3 | CORRELATION COEFFICIENTS. | |
|---|---|---|---|
| REMARK | 3 | CORRELATION COEFFICIENT FO-FC | : 0.967 |
| REMARK | 3 | CORRELATION COEFFICIENT FO-FC FREE | : 0.951 |
| REMARK | 3 | | |

| | | | | | |
|---|---|---|---|---|---|
| REMARK | 3 | RMS DEVIATIONS FROM IDEAL VALUES | COUNT | RMS | WEIGHT |

| | | | | | | |
|---|---|---|---|---|---|---|
| REMARK | 3 | BOND LENGTHS REFINED ATOMS | (A): | 4425 ; | 0.024 ; | 0.022 |
| REMARK | 3 | BOND ANGLES REFINED ATOMS | (DEGREES): | 6019 ; | 2.001 ; | 1.957 |
| REMARK | 3 | TORSION ANGLES, PERIOD 1 | (DEGREES): | 571 ; | 6.320 ; | 5.000 |
| REMARK | 3 | TORSION ANGLES, PERIOD 2 | (DEGREES): | 185 ; | 35.306 ; | 24.000 |
| REMARK | 3 | TORSION ANGLES, PERIOD 3 | (DEGREES): | 760 ; | 14.206 ; | 15.000 |
| REMARK | 3 | TORSION ANGLES, PERIOD 4 | (DEGREES): | 25 ; | 15.286 ; | 15.000 |
| REMARK | 3 | CHIRAL-CENTER RESTRAINTS | (A**3): | 671 ; | 0.156 ; | 0.200 |
| REMARK | 3 | GENERAL PLANES REFINED ATOMS | (A): | 3325 ; | 0.012 ; | 0.021 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |

| REMARK | 3 | | | | |
|---|---|---|---|---|---|
| REMARK | 3 | NCS RESTRAINTS STATISTICS | | | |
| REMARK | 3 | NUMBER OF NCS GROUPS : NULL | | | |
| REMARK | 3 | | | | |
| REMARK | 3 | TWIN DETAILS | | | |
| REMARK | 3 | NUMBER OF TWIN DOMAINS : NULL | | | |
| REMARK | 3 | | | | |
| REMARK | 3 | | | | |
| REMARK | 3 | TLS DETAILS | | | |
| REMARK | 3 | NUMBER OF TLS GROUPS : 3 | | | |
| REMARK | 3 | ATOM RECORD CONTAINS SUM OF TLS AND RESIDUAL B FACTORS | | | |
| REMARK | 3 | | | | |
| REMARK | 3 | TLS GROUP : 1 | | | |
| REMARK | 3 | NUMBER OF COMPONENTS GROUP : 2 | | | |
| REMARK | 3 | COMPONENTS | | | C SSSEQI TO C SSSEQI |
| REMARK | 3 | RESIDUE RANGE: | | | L 1 L 109 |
| REMARK | 3 | RESIDUE RANGE: | | | H 1 H 122 |
| REMARK | 3 | ORIGIN FOR THE GROUP (A): 9.3480 52.1830 33.9230 | | | |
| REMARK | 3 | T TENSOR | | | |
| REMARK | 3 | T11: 0.0431 T22: 0.0196 | | | |
| REMARK | 3 | T33: 0.0276 T12: 0.0114 | | | |
| REMARK | 3 | T13: 0.0100 T23: −0.0020 | | | |
| REMARK | 3 | L TENSOR | | | |
| REMARK | 3 | L11: 1.2847 L22: 0.6769 | | | |
| REMARK | 3 | L33: 2.3566 L12: 0.2152 | | | |
| REMARK | 3 | L13: 0.4752 L23: 0.5847 | | | |
| REMARK | 3 | S TENSOR | | | |
| REMARK | 3 | S11: 0.0830 S12: −0.0041 S13: −0.0198 | | | |
| REMARK | 3 | S21: 0.0073 S22: −0.0057 S23: −0.0459 | | | |
| REMARK | 3 | S31: 0.0390 S32: 0.1660 S33: −0.0773 | | | |
| REMARK | 3 | | | | |
| REMARK | 3 | TLS GROUP : 2 | | | |
| REMARK | 3 | NUMBER OF COMPONENTS GROUP : 2 | | | |
| REMARK | 3 | COMPONENTS | | | C SSSEQI TO C SSSEQI |
| REMARK | 3 | RESIDUE RANGE: | | | L 110 L 250 |
| REMARK | 3 | RESIDUE RANGE: | | | H 123 H 250 |
| REMARK | 3 | ORIGIN FOR THE GROUP (A): 27.2190 42.4690 5.5720 | | | |
| REMARK | 3 | T TENSOR | | | |
| REMARK | 3 | T11: 0.0288 T22: 0.0170 | | | |
| REMARK | 3 | T33: 0.0255 T12: 0.0116 | | | |
| REMARK | 3 | T13: −0.0108 T23: −0.0068 | | | |
| REMARK | 3 | L TENSOR | | | |
| REMARK | 3 | L11: 1.9851 L22: 2.0128 | | | |
| REMARK | 3 | L33: 1.0452 L12: 0.5265 | | | |
| REMARK | 3 | L13: −0.3061 L23: −0.2683 | | | |
| REMARK | 3 | S TENSOR | | | |
| REMARK | 3 | S11: −0.0438 S12: −0.0112 S13: −0.0403 | | | |
| REMARK | 3 | S21: −0.0720 S22: −0.0072 S23: 0.0391 | | | |
| REMARK | 3 | S31: −0.0593 S32: 0.0626 S33: 0.0510 | | | |
| REMARK | 3 | | | | |
| REMARK | 3 | TLS GROUP : 3 | | | |
| REMARK | 3 | NUMBER OF COMPONENTS GROUP: 1 | | | |
| REMARK | 3 | COMPONENTS | | | C SSSEQI TO C SSSEQI |
| REMARK | 3 | RESIDUE RANGE : | | | I 1 I 200 |
| REMARK | 3 | ORIGIN FOR THE GROUP (A): −7.7370 51.3830 61.1860 | | | |
| REMARK | 3 | T TENSOR | | | |
| REMARK | 3 | T11: 0.1110 T22: 0.1464 | | | |
| REMARK | 3 | T33: 0.0970 T12: −0.0398 | | | |
| REMARK | 3 | T13: −0.0116 T23: −0.0399 | | | |
| REMARK | 3 | L TENSOR | | | |
| REMARK | 3 | L11: 2.1367 L22: 1.7294 | | | |
| REMARK | 3 | L33: 3.9727 L12: 0.4565 | | | |
| REMARK | 3 | L13: −2.2072 L23: −1.0335 | | | |
| REMARK | 3 | S TENSOR | | | |
| REMARK | 3 | S11: 0.0766 S12: −0.3405 S13: 0.1642 | | | |
| REMARK | 3 | S21: 0.2556 S22: −0.0443 S23: −0.0496 | | | |
| REMARK | 3 | S31: −0.1361 S32: 0.1334 S33: −0.0323 | | | |
| REMARK | 3 | | | | |
| REMARK | 3 | | | | |
| REMARK | 3 | BULK SOLVENT MODELLING. | | | |
| REMARK | 3 | METHOD USED : MASK | | | |
| REMARK | 3 | PARAMETERS FOR MASK CALCULATION | | | |
| REMARK | 3 | VDW PROBE RADIUS : 1.20 | | | |
| REMARK | 3 | ION PROBE RADIUS : 0.80 | | | |
| REMARK | 3 | SHRINKAGE RADIUS : 0.80 | | | |
| REMARK | 3 | | | | |
| REMARK | 3 | OTHER REFINEMENT REMARKS: | | | |
| REMARK | 3 | U VALUES : WITH TLS ADDED | | | |
| REMARK | 3 | | | | |
| SSBOND | 1 | CYS L | 88 | CYS L | 23 |

| | | | | |
|---|---|---|---|---|
| LINKR | SG ACYS L 194 | SG CYS L | 134 | SS |
| LINKR | SG BCYS L 194 | SG CYS L | 134 | SS |

| | | | | | |
|---|---|---|---|---|---|
| SSBOND | 2 | CYS H | 134 | CYS L | 214 |
| SSBOND | 3 | CYS H | 96 | CYS H | 22 |

| | | | | |
|---|---|---|---|---|
| LINKR | SG ACYS H 203 | SG ACYS H | 147 | SS |
| LINKR | SG BCYS H 203 | SG BCYS H | 147 | SS |

| SSBOND | 4 | CYS I | 71 | CYS I | 122 | |
|---|---|---|---|---|---|---|
| SSBOND | 5 | CYS I | 78 | CYS I | 125 | |
| CISPEP | 1 | SER L | 7 | PRO L | 8 | 0.00 |
| CISPEP | 2 | TYR L | 94 | PRO L | 95 | 0.00 |
| CISPEP | 3 | TYR L | 140 | PRO L | 141 | 0.00 |
| CISPEP | 4 | PHE H | 153 | PRO H | 154 | 0.00 |
| CISPEP | 5 | GLU H | 155 | PRO H | 156 | 0.00 |

| | | | | | |
|---|---|---|---|---|---|
| LINKR | LYS I | 106 | ARG I | 114 | gap |
| LINKR | CYS I | 78 | SER I | 86 | gap |

| | |
|---|---|
| CRYST1 | 89.410 65.160 106.690 90.00 111.57 90.00 C 1 2 1 |

TABLE 2
IL-21, chain I, (SEQ ID NO. 1) interactions with the the heavy chain
(chain H) of Fab35 (SEQ ID NO. 10) and light chain (chain L) of
Fab35 (SEQ ID NO. 9). A distance cut-off of 4.0 Å was used.
The contacts were identified by the CONTACT computer software
program of the CCP4 suite (5). In the last column “***” indicates
a strong possibility for a hydrogen bond at this contact (distance <3.3 Å)
as calculated by CONTACT, “*” indicates a weak possibility
(distance >3.3 Å). Blank indicates that the program considered
there to be no possibility of a hydrogen bond. Hydrogen-bonds are
specific between a donor and an acceptor, are typically strong, and
are easily identifiable.

| IL-21 | | | Fab35 | | | | |
|---|---|---|---|---|---|---|---|
| | Res. # | | | Res. # | | | |
| Res. | and | Atom | Res. | and | Atom | Distance | Possibly |
| Type | Chain | name | Type | Chain | name | [Å] | H-bond |
| Glu | 65 I | OE1 | Tyr | 56 H | OH | 3.69 | * |
| Asp | 66 I | CB | Tyr | 56 H | CB | 3.76 | |
| | | | Tyr | 56 H | CG | 3.81 | |
| | | | Tyr | 56 H | CD2 | 4.00 | |
| Asp | 66 I | CG | Tyr | 56 H | CB | 3.92 | |
| | | | Gly | 54 H | N | 3.31 | |
| | | | Gly | 54 H | CA | 3.33 | |
| | | | Thr | 52 H | CB | 3.96 | |
| | | | Thr | 52 H | OG1 | 3.40 | |
| | | | Gly | 54 H | C | 3.79 | |
| Asp | 66 I | OD1 | Ser | 53 H | OG | 3.56 | * |
| | | | Gly | 54 H | N | 3.07 | *** |
| | | | Gly | 54 H | CA | 3.56 | |
| | | | Thr | 52 H | CB | 3.66 | |
| | | | Thr | 52 H | OG1 | 3.57 | * |
| | | | Ser | 53 H | N | 3.79 | * |
| Asp | 66 I | OD2 | Gly | 54 H | O | 3.71 | * |
| | | | Tyr | 56 H | CB | 3.23 | |
| | | | Tyr | 56 H | CG | 3.93 | |
| | | | Gly | 54 H | N | 3.18 | *** |
| | | | Gly | 54 H | CA | 3.11 | |
| | | | Ser | 55 H | C | 3.89 | |
| | | | Thr | 52 H | CB | 3.57 | |
| | | | Thr | 52 H | OG1 | 2.64 | *** |
| | | | Gly | 54 H | C | 3.08 | |
| | | | Ser | 55 H | N | 3.16 | *** |
| | | | Tyr | 56 H | N | 2.87 | *** |
| | | | Tyr | 56 H | CA | 3.57 | |
| Asp | 66 I | C | Tyr | 57 H | CE2 | 3.76 | |
| Asp | 66 I | O | Tyr | 57 H | OH | 3.96 | * |
| | | | Tyr | 57 H | CE2 | 3.46 | |
| Val | 67 I | N | Tyr | 57 H | CE2 | 3.86 | |
| Val | 67 I | CA | Tyr | 57 H | CE2 | 3.83 | |
| Val | 67 I | C | Tyr | 57 H | CE2 | 3.74 | |
| | | | Tyr | 57 H | CD2 | 3.69 | |
| Val | 67 I | O | Thr | 52 H | CB | 3.86 | |
| | | | Thr | 52 H | CG2 | 3.44 | |
| Glu | 68 I | N | Tyr | 57 H | CE2 | 3.64 | |
| | | | Tyr | 57 H | CD2 | 3.60 | |
| Glu | 68 I | CG | Tyr | 57 H | CG | 3.76 | |
| | | | Tyr | 57 H | CD1 | 3.59 | |
| | | | Tyr | 57 H | CE1 | 3.83 | |
| Glu | 68 I | CD | Tyr | 57 H | CB | 3.91 | |
| | | | Tyr | 57 H | CG | 3.71 | |
| | | | Tyr | 57 H | CD1 | 3.45 | |
| | | | His | 59 H | NE2 | 3.77 | |
| Glu | 68 I | OE1 | Tyr | 57 H | CB | 3.69 | |
| | | | Tyr | 57 H | CG | 3.95 | |
| | | | His | 59 H | NE2 | 3.07 | *** |
| | | | His | 59 H | CD2 | 3.89 | |
| Glu | 68 I | OE2 | Tyr | 57 H | CD1 | 3.36 | |
| | | | Tyr | 57 H | CE1 | 3.92 | |
| | | | His | 59 H | NE2 | 3.69 | * |
| Thr | 69 I | N | Thr | 52 H | CG2 | 3.85 | |
| Thr | 69 I | CB | Ser | 33 H | OG | 3.73 | |
| | | | Tyr | 94 L | OH | 3.61 | |
| Thr | 69 I | OG1 | Ser | 33 H | CB | 3.43 | |
| | | | Ser | 33 H | OG | 2.70 | *** |
| | | | Thr | 52 H | CG2 | 3.66 | |
| | | | Tyr | 94 L | OH | 3.79 | * |
| Thr | 69 I | CG2 | Ser | 33 H | OG | 3.68 | |
| | | | Glu | 99 H | CD | 3.55 | |
| | | | Glu | 99 H | OE1 | 3.82 | |
| | | | Glu | 99 H | OE2 | 3.50 | |
| Asn | 70 I | CB | Tyr | 105 H | CE1 | 3.72 | |
| Asn | 70 I | CG | Gly | 103 H | N | 3.87 | |
| | | | Tyr | 105 H | CD1 | 3.74 | |
| | | | Tyr | 105 H | CE1 | 3.49 | |
| Asn | 70 I | OD1 | Arg | 100 H | O | 3.96 | * |
| | | | Gly | 101 H | CA | 3.25 | |
| | | | Gly | 101 H | C | 3.20 | |
| | | | Gly | 101 H | O | 3.73 | * |
| | | | Trp | 102 H | N | 3.42 | * |
| | | | Trp | 102 H | C | 3.93 | |
| | | | Gly | 103 H | N | 2.83 | *** |
| | | | Tyr | 104 H | N | 3.77 | * |
| | | | Gly | 103 H | CA | 3.40 | |
| | | | Gly | 103 H | C | 3.96 | |
| Asn | 70 I | ND2 | Glu | 99 H | OE2 | 3.87 | * |
| | | | Tyr | 105 H | CD1 | 3.49 | |
| | | | Tyr | 105 H | CE1 | 3.55 | |
| Glu | 72 I | CB | Trp | 102 H | NE1 | 3.82 | |
| | | | Trp | 102 H | CE2 | 3.31 | |
| | | | Trp | 102 H | CD2 | 3.33 | |
| | | | Trp | 102 H | CE3 | 3.61 | |
| | | | Trp | 102 H | CZ3 | 3.86 | |
| | | | Trp | 102 H | CH2 | 3.82 | |
| | | | Trp | 102 H | CZ2 | 3.57 | |
| | | | Trp | 102 H | CG | 3.89 | |
| Glu | 72 I | CG | Trp | 102 H | NE1 | 3.75 | |
| | | | Trp | 102 H | CE2 | 3.73 | |
| Glu | 72 I | CD | Trp | 102 H | N | 3.51 | |
| Glu | 72 I | OE1 | Gly | 101 H | CA | 3.65 | |
| | | | Gly | 101 H | C | 3.61 | |
| | | | Trp | 102 H | N | 2.68 | *** |
| | | | Trp | 102 H | CA | 3.53 | |
| Glu | 72 I | OE2 | Gly | 101 H | CA | 3.89 | |
| Glu | 72 I | C | Trp | 102 H | CZ3 | 3.91 | |
| Glu | 72 I | O | Trp | 102 H | CZ3 | 3.78 | |
| Trp | 73 I | CG | Trp | 102 H | CE3 | 3.94 | |
| Trp | 73 I | CD1 | Trp | 102 H | CE3 | 3.95 | |
| | | | Trp | 102 H | CA | 3.79 | |
| | | | Trp | 102 H | C | 3.63 | |
| | | | Trp | 102 H | O | 3.31 | |
| Trp | 73 I | NE1 | Trp | 102 H | CE3 | 3.72 | |
| | | | Trp | 102 H | CA | 3.98 | |
| | | | Trp | 102 H | CB | 3.75 | |
| | | | Trp | 102 H | C | 3.82 | |
| | | | Trp | 102 H | O | 3.15 | *** |
| Trp | 73 I | CE2 | Trp | 102 H | CE3 | 3.49 | |
| Trp | 73 I | CD2 | Trp | 102 H | CE3 | 3.67 | |
| | | | Trp | 102 H | CZ3 | 3.94 | |
| Trp | 73 I | CE3 | Trp | 102 H | CZ3 | 3.97 | |
| Trp | 73 I | CZ2 | Trp | 102 H | CE3 | 3.95 | |
| Lys | 117 I | CD | Ser | 31 L | OG | 3.81 | |
| | | | Asp | 50 L | OD1 | 3.65 | |
| | | | Asp | 50 L | OD2 | 3.81 | |
| Lys | 117 I | CE | Asp | 50 L | OD1 | 3.92 | |
| | | | Asp | 50 L | OD2 | 3.34 | |
| Lys | 117 I | NZ | Ser | 31 L | CB | 3.94 | |
| | | | Ser | 31 L | OG | 3.22 | *** |
| | | | Asp | 50 L | CG | 3.54 | |
| | | | Asp | 50 L | OD1 | 3.44 | * |
| | | | Asp | 50 L | OD2 | 2.84 | *** |
| Lys | 117 I | O | Trp | 102 H | C | 3.94 | |
| | | | Gly | 103 H | N | 3.73 | * |
| | | | Gly | 103 H | CA | 3.60 | |
| His | 118 I | CA | Tyr | 105 H | OH | 3.82 | |
| His | 118 I | C | Tyr | 105 H | OH | 3.46 | |
| His | 118 I | O | Tyr | 105 H | OH | 3.73 | * |
| Arg | 119 I | N | Tyr | 105 H | OH | 3.56 | * |
| Arg | 119 I | CG | Tyr | 105 H | OH | 3.87 | |
| Arg | 119 I | CD | Phe | 91 L | O | 3.46 | |
| | | | Asn | 92 L | C | 3.96 | |
| | | | Asn | 92 L | O | 3.28 | |
| Arg | 119 I | NH2 | Tyr | 94 L | CE1 | 3.92 | |
| Leu | 143 I | CG | Trp | 102 H | CH2 | 3.62 | |
| | | | Trp | 102 H | CZ2 | 3.79 | |
| Leu | 143 I | CD1 | Trp | 102 H | CZ2 | 3.77 | |
| Leu | 143 I | O | Trp | 102 H | CH2 | 3.71 | |
| | | | Trp | 102 H | CZ2 | 3.33 | |
| Lys | 146 I | CG | Ser | 31 H | OG | 3.69 | |
| Lys | 146 I | CE | Ser | 30 H | O | 3.79 | |
| | | | Ser | 53 H | OG | 3.62 | |
| Met | 147 I | N | Trp | 102 H | NE1 | 3.77 | * |
| | | | Trp | 102 H | CE2 | 3.77 | |
| | | | Trp | 102 H | CZ2 | 3.59 | |
| Met | 147 I | CA | Trp | 102 H | NE1 | 3.75 | |
| | | | Trp | 102 H | CE2 | 3.62 | |
| | | | Trp | 102 H | CZ2 | 3.76 | |
| Met | 147 I | CB | Trp | 102 H | CE2 | 3.66 | |
| | | | Trp | 102 H | CD2 | 3.99 | |
| | | | Trp | 102 H | CZ3 | 3.89 | |
| | | | Trp | 102 H | CH2 | 3.58 | |
| | | | Trp | 102 H | CZ2 | 3.49 | |
| Met | 147 I | CG | Trp | 102 H | CD2 | 3.93 | |
| | | | Trp | 102 H | CE3 | 3.78 | |
| | | | Trp | 102 H | CZ3 | 3.83 | |
| His | 149 I | CB | Ser | 31 H | OG | 3.99 | |
| His | 149 I | CG | Ile | 28 H | CG1 | 4.00 | |
| | | | Ser | 31 H | OG | 3.93 | |
| His | 149 I | ND1 | Ile | 28 H | CB | 3.89 | |
| | | | Ile | 28 H | CG1 | 3.98 | |
| | | | Ile | 28 H | CG2 | 3.71 | |
| | | | Ser | 31 H | OG | 3.07 | *** |
| His | 149 I | CE1 | Ile | 28 H | CG1 | 3.86 | |
| | | | Ile | 28 H | CG2 | 3.81 | |
| His | 149 I | NE2 | Ile | 28 H | CG1 | 3.68 | |
| His | 149 I | CD2 | Ile | 28 H | CG1 | 3.83 | |
| His | 149 I | O | Tyr | 32 H | OH | 3.60 | * |
| Gln | 150 I | CA | Tyr | 32 H | OH | 3.52 | |
| Gln | 150 I | CG | Tyr | 32 H | CE1 | 3.74 | |
| | | | Tyr | 32 H | CZ | 3.81 | |
| Gln | 150 I | CD | Ser | 31 H | O | 3.78 | |
| | | | Tyr | 32 H | CD1 | 3.94 | |
| | | | Tyr | 32 H | CE1 | 3.95 | |
| | | | Gly | 101 H | N | 3.94 | |
| | | | Trp | 102 H | CD1 | 3.90 | |
| Gln | 150 I | OE1 | Arg | 100 H | CB | 3.51 | |
| | | | Arg | 100 H | CG | 3.79 | |
| | | | Arg | 100 H | CA | 3.52 | |
| | | | Arg | 100 H | C | 3.73 | |
| | | | Gly | 101 H | N | 2.99 | *** |
| | | | Gly | 101 H | CA | 3.97 | |
| | | | Trp | 102 H | CD1 | 3.53 | |
| Gln | 150 I | NE2 | Ser | 31 H | C | 3.76 | |
| | | | Tyr | 32 H | CA | 3.82 | |
| | | | Ser | 31 H | O | 2.70 | *** |
| | | | Tyr | 32 H | CG | 3.93 | |
| | | | Tyr | 32 H | CD1 | 3.70 | |
| Gln | 150 I | C | Arg | 100 H | NH2 | 3.84 | |
| Gln | 150 I | O | Arg | 100 H | NE | 3.47 | * |
| | | | Arg | 100 H | CZ | 3.14 | |
| | | | Arg | 100 H | NH1 | 3.57 | * |
| | | | Arg | 100 H | NH2 | 3.19 | *** |
| His | 151 I | CG | Arg | 100 H | NH2 | 3.70 | |
| His | 151 I | CE1 | Trp | 102 H | CB | 3.73 | |
| | | | Trp | 102 H | CG | 3.99 | |
| His | 151 I | NE2 | Arg | 100 H | NH2 | 3.52 | * |
| His | 151 I | CD2 | Arg | 100 H | NH2 | 3.30 | |

### REFERENCES

1. Ursby T et al. The New Macromolecular Crystallography Stations At MAX-lab: The MAD Station. AIP Conference Proceedings 705, 1241-1246. 2004. Ref Type Generic
2. Kabsch W. Automatic processing of rotation diffraction data from crystals of initially unknown symmetry and cell constants. J Appl Crystallogr 26:795-800, 1993.
3. Mccoy A J, Grosse-Kunstleve R W, Storoni L C, Read R J. Likelihood-enhanced fast translation functions. Acta Crystallographica Section D Biological Crystallography 61:458-464, 2005.
4. Mccoy A J, Grosse-Kunstleve R W, Adams P D, Winn M D, Storoni L C, Read R J. Phaser crystallographic software. J Appl Crystallogr 40:658-674, 2007.
5. Bailey S. The ccp4 suite—programs for protein crystallography. Acta Crystallogr Sect D-Biol Crystallogr 50:760-763, 1994.
6. Perrakis A, Morris R, Lamzin V S. Automated protein model building combined with iterative structure refinement. Nat Struct Biol 6:458-463, 1999.
7. Murshudov G N, Vagin A A, Dodson E J. Refinement of macromolecular structures by the maximum-likelihood method. Acta Crystallogr Sect D-Biol Crystallogr 53:240-255, 1997.
8. Afonine P V, Grosse-Kunstleve R W, Adams P D. Contribution 8. CCP4 Newsletter [42]. 2005. Ref Type Generic
9. Adams P D et al. PHENIX: a comprehensive Python-based system for macromolecular structure solution. Acta Cryst D 66:213-221, 2010.
10. Emsley P, Cowtan K. Coot: model-building tools for molecular graphics. Acta Crystallogr Sect D-Biol Crystallogr 60:2126-2132, 2004.
11. Lee B, Richards F M. THE INTERPRETATION OF PROTEIN STRUCTURES ESTIMATION OF STATIC ACCESSIBILITY. J Mol Biol 55:379-400, 1971.
12. Saff E B, Kuijlaars A B J. Distributing many points on a sphere. Math Intel 19:5-11, 1997.

### Example 2

### Description and Comparison of BS1, BS2, mAb14 and mAb5 Epitope

Binding sites and epitopes provided in this example are based on three experimental (crystal/X-ray) structures and one homology model. The three crystal structures are:
(i) the IL-21:IL-21Rα complex,
(ii) the IL-21:Fab35 complex (“Fab35” is the Fab fragment corresponding to mAb14), and
(iii) the IL-21:Fab9 complex (“Fab9” is the Fab fragment corresponding to mAb5 referred to as the 362.78.1.44 antibody disclosed in WO2010/055366).

The crystal structure of IL-21:IL-21Rα (PDB, 3TGX) provided the basis for building a model of the ternary IL-21:IL-21Rα:γC complex. The homology model of the IL-21:IL-21Rα:γC complex was built using the IL-21:IL-21Rα, IL-2:IL-2RA:IL-2RB:γC and IL-4:IL-4R:γC complexes as templates. It should be noted that there may be minor inaccuracies in this model, and that such inaccuracy will affect the accuracy of the prediction of the IL-21 residues belonging to BS2.

Receptor binding sites and epitopes are determined from the experimental and model structures using a 4 Å distance cut-off.

IL-21 BS1 residues (SEQ ID NO. 1) determined from the crystal structure of the IL-21:IL21Rα complex comprises the following residues:

| IL-21 | |
|---|---|
| residues | |
| in BS1 | # |
| ARG | 34 |
| ILE | 37 |
| ARG | 38 |
| ARG | 40 |
| GLN | 41 |
| ASP | 44 |
| ILE | 45 |
| GLN | 48 |
| TYR | 52 |
| ILE | 95 |
| VAL | 98 |
| SER | 99 |
| LYS | 102 |
| ARG | 105 |
| LYS | 106 |
| PRO | 107 |
| PRO | 108 |
| SER | 109 |

IL-21 BS2 residues determined from the homology model structure of the IL-21:IL21Rα:γC complex comprises the following residues:

| IL-21 | |
|---|---|
| residues | |
| in BS2 | # |
| ARG | 40 |
| LYS | 50 |
| GLU | 65 |
| ASP | 66 |
| VAL | 67 |
| GLU | 129 |
| GLU | 135 |
| GLU | 138 |
| ARG | 139 |
| LYS | 141 |
| SER | 142 |
| GLN | 145 |
| HIS | 149 |

IL-21 epitope residues (mAb14) determined from the crystal structure of the IL-21:Fab35 complex (Example 1) comprises the following residues:

| IL-21 | |
|---|---|
| residues in | |
| mAb14 | |
| epitope | # |
| GLU | 65 |
| ASP | 66 |
| VAL | 67 |
| GLU | 68 |
| THR | 69 |
| ASN | 70 |
| GLU | 72 |
| TRP | 73 |
| LYS | 117 |
| HIS | 118 |
| ARG | 119 |
| LEU | 143 |
| LYS | 146 |
| MET | 147 |
| HIS | 149 |
| GLN | 150 |
| HIS | 151 |

IL-21 epitope residues (mAb5) determined from the crystal structure of the IL-21:Fab9 complex (unpublished results) comprises the following residues:

| IL-21 | |
|---|---|
| residues | |
| in mAb5 | |
| epitope | # |
| Ile | 37 |
| Arg | 38 |
| Gln | 41 |
| Asp | 44 |
| Ile | 45 |
| Asp | 47 |
| Gln | 48 |
| Asn | 51 |
| Tyr | 52 |
| Asn | 92 |
| Arg | 94 |
| Ile | 95 |
| Asn | 97 |
| Val | 98 |
| Val | 98 |
| Ser | 99 |
| Lys | 101 |
| Lys | 102 |
| Arg | 105 |
| Lys | 106 |
| Pro | 107 |
| Pro | 108 |

BS1, BS2, mAb14 and mAb5 epitope residues are mapped on to the primary sequence of IL-21 in FIG. 2. Overlap between the predicted BS2 and the mAb14 epitope is observed for amino acid residues E65, D66, V67 and H149.

### Example 3

### Co-Binding Studies of Human IL-21 to Anti-IL-21 mAbs and IL-21Rα/γC Subunits by Surface Plasmon Resonance (SPR)

Binding studies were performed on a Biacore T100 instrument that measures molecular interactions in real time through surface plasmon resonance. Experiments were run at 25° C. The signal (RU, response units) reported by the Biacore is directly correlated to the mass on the individual sensor chip surfaces in four serial flow cells.

Anti-IL-21 monoclonal antibodies mAb6, mAb14 and mAb19 were immobilized directly onto flow cells of a CM5 sensor chip according to the manufacturer's instructions. “mAb6” corresponds to an IgG1 version of the clone 362.78.1.44 antibody disclosed in WO2010055366, the Fc region of mAb6 carrying the L234A, L235E, and G237A for reduced Fc receptor binding and A330S and P331S mutations for reduced C1q-mediated complement fixation), i.e. mAb6 is the same antibody as mAb5. Only difference between the two antibodies is the mammalian expression host used for mAb production. “mAb19” is the antibody produced by the clone “272.21.1.13.4.2”/“272.21.1.3.4.2” disclosed in WO2007111714. The final immobilization level of antibody was approximately 500-800 RU in one experiment. Capture of IL-21 was conducted by diluting the protein to 100 nM into running buffer (10 mM Hepes, 0.15 M NaCl, 3 mM EDTA, 0.05% surfactant P20, pH 7.4) and injected at 30 μl/min for 120 s in flow cell 2, creating a reference surface in flow cell 1 with only respective anti-IL-21 antibody immobilized. This typically resulted in final capture levels of IL-21 of approximately 40 to 140 RU. Binding of the extra cellular domains of hIL-21Rα, hIL21Rα-ECD or γC-ECD was conducted by injecting analyte over all flow cells to allow for comparative analyses of binding to IL-21 captured by different anti-IL21 antibodies relative to binding to the reference flow cell. IL-21Rα-ECD or γC-ECD protein was diluted serially 1:2 to 0.3-10 or 625 nM-10 μM into running buffer, injected at 30 μl/min for 120 s and allowed to dissociate for 300 s. The CM5 surface was regenerated after each injection cycle of analyte via two 8 s injections of 1M Formic acid at 30 μl/min. This regeneration step removed the IL-21 and any bound hIL-21Rα-ECD or γC-ECD chain from the immobilized capture antibody surface, and allowed for the subsequent binding of the next interaction sample pair. The regeneration procedure did not remove the directly immobilized anti-IL-21 capture antibody from the chip surface.

Data analysis was performed using the Biacore T100 evaluation software 2.0.3. No significant non-specific binding to the reference control surface was observed. Binding curves were processed by double referencing (subtraction of reference surface signals as well as blank buffer injections over captured IL-21). This allowed correction for instrument noise, bulk shift and drift during sample injections.

IL-21 captured by immobilized mAb6 was not able to simultaneously interact with hIL-21Rα-ECD, demonstrating that this antibody bind in or close to BS1 on IL-21 and thus compete for binding of the hIL-21Rα receptor subunit to this site. In contrast, IL-21 captured by mAb14 could form a stable complex with IL-21Rα-ECD demonstrating that mAb14 does not compete for binding of the receptor subunit to BS1 and thus bind to a separate epitope on IL-21.

The same competition study was performed with mAb14 and mAb6 together with γC-ECD. IL-21 captured by immobilized mAb14 was not able to simultaneously interact with γC-ECD, demonstrating that this antibody binds in or close to BS2 on IL-21 and thus compete for binding of the γC receptor subunit to this site. In contrast, IL-21 captured by mAb6 could bind weakly to γC-ECD demonstrating that mAb6 does not compete for binding of the receptor subunit to BS2 and thus bind to a separate epitope on IL-21. IL-21 captured by mAb19 was not able to bind simultaneously to neither IL-21Rα-ECD nor γC-ECD but the mechanism for this is not clear.

TABLE 3
Ability of different antibodies to bind simultaneously to (+) or to
compete with (−) binding of different receptor subunits to IL-21.

| IL-21 captured by mAb | hIL21Rα | γC |
|---|---|---|
| mAb6 | − | + |
| mAb14 | + | − |
| mAb19 | − | − |

The SPR binding competition studies clearly demonstrate that mAb6 and mAb14 interfere with the binding of the different receptor subunits of the IL-21 receptor complex to their respective binding sites on IL-21 and that these antibodies thus operate by separate mechanisms. Further mAb/IL-21/IL-21 receptor studies are described in Example 16.

### Example 4

### Study of Interaction Kinetics for Anti-IL-21 Antibody mAb37 to IL-21 By Surface Plasmon Resonance (SPR)

Binding studies were performed on a Biacore T200 instrument that measures molecular interactions in real time through surface plasmon resonance. Experiments were run at 25° C. and the samples were stored at 10° C. in the sample compartment. The signal (RU, response units) reported by the Biacore is directly correlated to the mass on the individual sensor chip surfaces in four serial flow cells.

Anti-human Fc monoclonal antibody from Biacore human Fc capture kit was immobilized onto flow cells of a CM4 sensor chip according to the manufacturer's instructions. The final immobilization level of capture antibody was approximately 2,000 RU in one experiment. Kinetic studies were performed with a variant of mAb14, mAb37 containing a single point mutation, S241P (numbering according to Kabat) in the IgG4 hinge region, which prevents formation of half antibodies, but does not affect binding to the antigen. Capture of the human anti-IL-21 antibody mAb37 was conducted by diluting the antibody to 0.1 μg/ml into running buffer (10 mM Hepes 0.3 M NaCl, 5 mM CaCl2, 0.05% surfactant P20, pH 8.0 containing 1 mg/ml BSA) and injected at 10 μl/min for 180 s in one of flow cells 2-4, creating a reference surface in flow cell 1 with only anti-Fc antibody immobilized. This typically resulted in final capture levels of test antibodies of approximately 30-50 RU and Rmax values of analyte of 6-8 RU. Binding of IL-21 protein was conducted by injecting analyte over all flow cells to allow for comparative analyses of binding to different captured anti-IL-21 antibodies relative to binding to the reference flow cell. IL-21 protein was diluted serially 1:3 to 0.2-54 nM into running buffer, injected at 100 μl/min for 210 s and allowed to dissociate for 600 or 14000 s. The CM4 surface was regenerated after each injection cycle of analyte via two injections of 3M MgCl₂ at 50 μl/min. This regeneration step removed the anti-IL-21 antibody and any bound IL-21 from the immobilized capture antibody surface, and allowed for the subsequent binding of the next interaction sample pair. The regeneration procedure did not remove the directly immobilized anti-Fc capture antibody from the chip surface. In order to obtain kinetic data, such as ka (association rate), kd (dissociation rate) and KD (equilibrium dissociation constant), data analysis was performed using the Biacore T200 evaluation software 1.0, fitting data to 1:1 Langmuir model. No significant non-specific binding to the reference control surface was observed. Binding curves were processed by double referencing (subtraction of reference surface signals as well as blank buffer injections over captured anti-IL-21 antibodies). This allowed correction for instrument noise, bulk shift and drift during sample injections.

Human IL-21 dissociates from mAb37 with an off-rate less than what can be accurately measured by the currently used assay (kd<1E-5 s⁻¹), an average ka 6E+5 (Ms)⁻¹ resulting in a KD of <20 μM. Results are based on triplicate measurements. Individual relative standard errors of parameters ka and kd were <0.6%. These data clearly demonstrates that mAb37 bind to human IL-21 with high affinity.

TABLE 4
Results from triplicate measurements of binding constants ka (association rate), kd (dissociation rate)
and KD (equilibrium dissociation constant) for the interaction of human IL-21 to mAb37 and mAb19.

| Antibody | Ka (1/Ms) | kd (1/s) | KD (M) | RSE ka (%) | RSE kd (%) |
|---|---|---|---|---|---|
| mAb37 | 6.4E+05 | 4.5E−06 | 7.0E−12 | 0.5 | 0.5 |
| mAb37 | 6.0E+05 | 4.5E−06 | 7.5E−12 | 0.4 | 0.4 |
| mAb37 | 6.0E+05 | 6.4E−06 | 1.1E−11 | 0.3 | 0.5 |
| Antibody | ka (1/Ms) | kd (Vs) | KD (M) | RSE ka (%) | RSE kd (%) |
| mAb19 | 2.00E+06 | 1.25E−05 | 6.26E−12 | 0.2 | 0.3 |
| mAb19 | 1.68E+06 | 1.03E−05 | 6.12E−12 | 0.4 | 0.3 |
| mAb19 | 1.93E+06 | 1.01E−05 | 5.22E−12 | 0.4 | 0.3 |

### Example 5

### B Cell Proliferation and Maturation Assays

To test the effect of the anti-IL-21 antibodies in a biologically relevant setting three functional assays were established where relevant IL-21 biology was studied in primary human cells.

Stimulation with a combination of Anti-CD40 antibody and recombinant IL-21 induces proliferation of primary B cells and B cell maturation as measured by the frequency of plasma blasts with a CD19⁺CD27^{high}CD38^{high} phenotype. The Anti-IL-21 antibody(ies) were able to prevent both proliferation and maturation.

The relevance of B cells to chronic inflammatory disease has been described in the literature as well as by the clinical effect of B-cell depletion with Rituximab in e.g. rheumatoid arthritis. In the literature, B cells were shown to play an important role in driving chronic inflammation (Dörner T et al (2009) Arthritis Res. Therapy), both as antigen presenting cells as well as producers of (auto)antibodies. IL-21 induces B cell proliferation (when combined with CD40 co-stimulation), immunoglobulin (Ig) class switching to particular IgG1 and IgG3, and differentiation of activated B cells to Ig-producing plasma cells (Ozaki, K. et al., Science, 2002; Ettinger R. J. et al., J Immunol, 2005; Kuchen, S., et al., J Immunol, 2007; Ettinger, R. et al., Immunol Rev, 2008; Leonard, W. J. et al. Nat. Rev. Immunol. 2005). Neutralization of IL-21 activity is therefore expected to reduce B cell differentiation and thus potentially decrease B cell immune-stimulating properties and autoantibody production in autoimmune patients.

Blood bags were obtained from healthy human volunteers and PBMCs were isolated from 50 ml of heparinised peripheral blood by Ficoll-Paque™ Plus (GE Healthcare) gradient centrifugation. Blood was diluted to 100 ml in phosphate-buffered saline (PBS) at room temperature and 35 ml aliquots were distributed into 50 ml conical tubes carefully overlaying 14 ml of Ficoll-Paque™ Plus (Ge Healthcare) at room temperature. The tubes were spun for 25 minutes at 1680 rpm (600×g) at room temperature without brake. The PBMC interface layer was removed carefully and washed twice with PBS containing 2% FCS. B cells were isolated by negative selection using EasySep human B Cell enrichment Kit (StemCell Technologies SERL, Grenoble, France). A small sample of the purified B cells was tested for purity by FACS analysis and found to be >95-97% pure in all experiments.

B cells were cultured in RPMI-1640 media (InVitrogen) supplemented with heat inactivated foetal calf serum (FCS) (Gibco) or Healthy human serum (HS) (Sigma), and Penicillin/Streptomycin (Gibco). Purified human B cells were plated at 50,000 cells/well in a 96-well U-bottom tissue culture plate (BD Biosciences). The cells were treated with or without 0.1 μg/ml anti-CD40 (goat anti-human CD40 polyclonal; R&D Systems), plus a titration of recombinant human IL-21 (Novo Nordisk A/S) prepared as a 1:3 serial dilution. The plate of cells was then incubated for 3 days at 37° C. and 5% CO₂ in a humidified incubator. After three days, the cells were pulsed with 1 μCi/well of [³H]-Thymidine (Perkin Elmer Life Sciences). After 16 hours, the cells were harvested onto UniFilter-96 GF/C filter plates (Packard, Perkin Elmer) and the amount of [³H]-Thymidine incorporation was quantitated using a TopCount NXT (Perkin Elmer Life Sciences). The effective concentration of IL-21 required for induction of 50% and 90% maximum proliferation (EC₅₀ and EC₉₀, respectively) were calculated using the GraphPad Prism v5.0 software (GraphPad Inc) and the sigmoidal dose-response (variable slope) equation.

The two anti-IL-21 antibodies mAb14 and mAb37 were tested and compared for their ability to neutralise recombinant human IL-21 in the B cell proliferation assay.

Human B cells were isolated from 2 individual donors. The B cells were plated at 50.000 cells per well in a 96-well U-bottom tissue culture plate. The cells were treated with 0.1 μg/ml anti-CD40 (R&D Systems), 50 ng/ml (3.21 nM) recombinant human IL-21. The cells were incubated for 3 days at 37° C. and 5% CO₂ in a humidified incubator. The antibodies were 3-fold titrated and after three days, the cells were pulsed with 1 μCi/well of [³H]-Thymidine (Perkin Elmer Life Sciences) for the last 20 hours. The cells were harvested onto UniFilter-96 GF/C filter plates (Packard Instruments, Perkin Elmer) and the amount of [³H]-thymidine incorporation was quantified using a TopCount NXT (Perkin Elmer). The inhibitive concentration of each antibody required for reducing proliferation by 50% (IC₅₀) was calculated using the GraphPad Prism v5.0 software (GraphPad Inc.) and the sigmoidal dose-response (variable slope, 4-parameters) equation.

The IC₅₀ for both antibodies was determined to be in the low nanomolar range but mAb37 was slightly more efficient in neutralizing IL-21 compared to mAb14, this is most likely due to the increased stability in the mAb37 molecule due the stabilizing S241P hinge mutation.

TABLE 5
IC₅₀ values for mAb14 and mAb37 in B cell proliferation assay

| | Donor 1 | Donor 2 | Donor 1 | Donor 2 |
|---|---|---|---|---|
| | Exp 1 | Exp 1 | Exp 2 | Exp 2 |
| mAb14 | 0.138 | 0.142 | — | — |
| mAb37 | — | — | 0.085 | 0.067 |

### Example 6

### Design of Antibodies According to the Invention

In order to design mutants of mAb14 which bind to the epitope described herein, the Kabat defined CDR-loops for mAb14 were analysed.

CDR-regions in the mAb14 heavy chain and light chain comprise the following residues (CDR-residues) according to SEQ ID NO 7 and 6, respectively:
CDR_H1: S31, Y32, S33, M34, N35
CDR_H2: S50, I51, T52, S53, G54, S55, Y56, Y57, I58, H59 Y60, A61, D62, S63, V64, K65, G66
CDR_H3: E99, R100, G101, W102, G103, Y104, Y105, G106, M107, D108, V109 CDR_L1: R24, A25, S26, Q27, D28, I29, D30, S31, A32, L33, A34
CDR_L2: D50, A51, S52, S53, L54, E55, S56
CDR_L3: Q89, Q90, F91, N92, S93, Y94, P95, Y96, T97

The paratope defined using a 4 Å distance cut-off was determined from the crystal structure of the Fab35:IL-21 complex. Fab35 is the Fab fragment corresponding to mAb14. The paratope is determined to comprise the following residues:
In CDR_H1: I28, S30, S31, Y32, S33
In CDR_H2: T52, S53, G54, S55, Y56, Y57, H59
In CDR_H3: E99, R100, G101, W102, G103, Y104, Y105
In CDR_L1: S31
In CDR_L2: D50
In CDR_L3: F91, N92, Y94

Thus, CDR-residues not included in the paratope are the following (in total 38):
In CDR_H1: M34, N35
In CDR_H2: S50, I51, I58, Y60, A61, D62, S63, V64, K65, G66
In CDR_H3: G106, M107, D108, V109
In CDR_L1: R24, A25, S26, Q27, D28, I29, D30, A32, L33, A34
In CDR_L2: A51, S52, S53, L54, E55, S56
In CDR_L3: Q89, Q90, S93, P95, Y96, T97

Among the 38 non-paratope CDR-residues 10 were selected as potential mutation sites. The selection was based on inspection of the crystal structure. Extensively buried residues and residues for which the side chains appeared to be involved in several important interactions were deselected. The identified potential mutation sites are listed in Table 6. Specific mutations (Table 6) at these sites were chosen such that no or minimal effect on the protein structure would result.

TABLE 6
Selected mutation sites and suggested mutations of the mAb14 antibody. Each of the individual
mutations shown in this table represents different embodiments of the present invention,
i.e. monoclonal antibodies having the ability interfere with binding of γC to IL-21. Antibodies
according to the invention may also comprise two or more of the mutations shown in this table.
It follows that variant antibodies according to the invention can only comprise one mutation
in a specific position.

| Residue | CDR-loop | Mutation |
|---|---|---|
| A61 | H2 | A61S |
| D62 | H2 | D62E |
| V64 | H2 | V64I |
| K65 | H2 | K65R |
| R24 | L1 | R24K |
| S26 | L1 | S26T |
| Q27 | L1 | Q27N |
| D30 | L1 | D30E |
| S53 | L2 | S53T |
| S56 | L2 | S56T |

This example describes one method applicable for designing antibodies according to the invention based on the information contained in the crystal structure of Fab35:IL-21. It follows that several other approaches can be taken in designing ligands according to the invention.

One approach could be e.g. to design a ligand essentially comprising the paratope of mAb14 except that one or more conservative substitutions can be made.

Another approach could be to design an IL-21 ligand based on the structure of the binding interface between IL-21 and γC. This ligand could be in the form of e.g. an antibody or a γC variant/mimic that essentially retains the structure of said γC binding interface.

It follows that one or more of such approaches can be combined.

Autoimmune disorders and other immune related disorders can be treated with e.g. therapeutic human monoclonal antibodies. However, said monoclonal antibodies may be immunogenic and give rise to the formation of anti-antibodies, also referred to as HAHA (human anti-human antibodies). It is conceivable that HAHA bind to areas of the therapeutic antibodies that will affect the binding of the therapeutic antibody to its antigen, i.e. the HAHA is a neutralizing antibody. If such potentially immunogenic sites, leading to development of anti-antibodies against mAb14, are recognized and characterized, the detailed description of the paratope for the antibody mAb14 derived from the 3-dimensional structure of the Fab35:IL-21 complex provides a possibility for rationally designing variants of mAb14 that will retain high-affinity binding to IL-21, but potentially are less immunogenic. Alternatively, variants of mAb14 may be designed in such a way that unwanted binding to specific anti-antibodies is reduced or prevented. It is thus possible to use the crystal structure information to provide improved versions of mAb14.

The provision of the crystal structure of this Fab fragment as well as its paratope also provides the possibility of e.g. replacing residues therein that could potentially result in antibodies improved with respect to stability, solubility or other chemical or physical properties of a molecule comprising this paratope while maintaining its biological functionality including high-affinity binding to IL-21. Stability can e.g. be improved by reducing aggregation, self association, fragmentation, and disulfide formation/exchange. Other properties, such as viscosity, may also be altered by introduction of one or more mutations.

The provision of the Fab35:IL-21 crystal structure furthermore provides a possibility of providing variants of mAb14 having reduced risk of e.g. deamidation, isomerization and/or oxidation and thereby improving the physical/chemical stability of a molecule comprising this paratope while maintaining its biological functionality including high-affinity to IL-21.

One example of potential stability improving mutations in the antibody mAb14 is the elimination of potential oxidation sites by mutation of Methionine residues. One specific example of such a mutation is the change of the Methionine in position 83 in the heavy chain (SEQ ID No. 7) to an amino acid with similar properties, e.g. Isoleucine. A second specific example of such a mutation is the change of the Methionine in position 107 in the heavy chain (SEQ ID No. 7) to an amino acid with similar properties, e.g. Isoleucine.

One example of potential stability improving mutations in the antibody mAb14 is elimination of potential hot-spots (DX-motifs, e.g. DG- and DS-motifs) for isomerisation of Aspartate residues. Such potentially labile DX-motifs can be eliminated by appropriate mutation of one or both of the constituent D or X residues. One specific example of such a mutation is the change of the Aspartate (present in a DS motif) in position 62 in the heavy chain (SEQ ID No. 7) to an amino acid with similar properties, e.g. Glutamate. A second specific example of such a mutation is the change of the Aspartate (present in a DS motif) in position 206 in the heavy chain (SEQ ID No. 7) to an amino acid with similar properties, e.g. Glutamate. A third specific example of such a mutation is the change of the Aspartate (present in a DS motif) in position 167 in the light chain (SEQ ID No. 6) to an amino acid with similar properties, e.g. Glutamate. A fourth specific example of such a mutation is the change of the Aspartate (present in a DS motif) in position 170 in the light chain (SEQ ID No. 6) to an amino acid with similar properties, e.g. Glutamate.

One example of potential stability improving mutations in the antibody mAb14 is elimination of potential hot-spots (NX-motifs, e.g. NG- or NS-motifs) for deamidation of Asparagine residues. Such potentially labile NX-motifs can be eliminated by appropriate mutation of one or both of the constituent N or X residues. One specific example of such a mutation is the change of the Asparagine (present in a NS motif) in position 77 in the heavy chain (SEQ ID No. 7) to an amino acid with similar properties, e.g. Glutamine. A second specific example of such a mutation is the change of the Asparagine (present in a NS motif) in position 84 in the heavy chain (SEQ ID No. 7) to an amino acid with similar properties, e.g. Glutamine. A third specific example of such a mutation is the change of the Asparagine (present in a NS motif) in position 158 in the light chain (SEQ ID No. 6) to an amino acid with similar properties, e.g. Glutamine.

### Example 7

### Epitope Mapping by HX-MS of mAb14 and mAb5

### Introduction to HX-MS

The HX-MS technology exploits that hydrogen exchange (HX) of a protein can readily be followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction. The deuterium labelling information can be sub-localized to regions in the protein by pepsin digestion under quench conditions and following the mass increase of the resulting peptides.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent. Protein-protein complex formation may be detected by HX-MS simply by measuring the total amount of deuterium incorporated in either protein members in the presence and absence of the respective binding partner as a function of time. The HX-MS technique uses the native components, i.e. protein and antibody or Fab fragment, and is performed in solution. Thus HX-MS provides the possibility for mimicking the in vivo conditions (for a recent review on the HX-MS technology, see Wales and Engen, Mass Spectrom. Rev. 25, 158 (2006)).

### Materials

Protein batches used were:

hIL-21: human recombinant IL-21 (expressed in E. coli as the mature peptide; residues 30-162 of SEQ ID NO: 1 with an added N-terminal Methionine residue). Antibodies were mAb5 and mAb14.

All proteins were buffer exchanged into PBS pH 7.4 before experiments.

### Methods: HX-MS Experiments

### Instrumentation and Data Recording

The HX experiments were automated by a Leap robot (H/D-x PAL; Leap Technologies Inc.) operated by the LeapShell software (Leap Technologies Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and HX reactions and maintained at 2° C. for storage of protein and quench solution, respectively. The Leap robot furthermore contained a cooled Trio VS unit (Leap Technologies Inc.) holding the pre- and analytical columns, and the LC tubing and switching valves at 1° C. The switching valves of the Trio VS unit have been upgraded from HPLC to Microbore UHPLC switch valves (Cheminert, VICI AG). For the inline pepsin digestion, 100 μL quenched sample containing 200 pmol hIL-21 was loaded and passed over a Poroszyme® Immobilized Pepsin Cartridge (2.1×30 mm (Applied Biosystems)) placed at 20° C. using a isocratic flow rate of 200 μL/min (0.1% formic acid:CH₃CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (2.1×100 mm (Waters Inc.)), and the peptides separated using a 9 min gradient of 15-35% B delivered at 200 μl/min from an AQUITY UPLC system (Waters Inc.). The mobile phases consisted of A: 0.1% formic acid and B: 0.1% formic acid in CH₃CN. The ESI MS data, and the separate data dependent MS/MS acquisitions (CID) and elevated energy (MS^{E}) experiments were acquired in positive ion mode using a Q-TOF Premier MS (Waters Inc.). Leucine-enkephalin was used as the lock mass ([M+H]⁺ ion at m/z 556.2771) and data was collected in continuum mode (For further description of the set-up, see Andersen and Faber, Int. J. Mass Spec., 302, 139-148 (2011)).

### Data Analysis

Peptic peptides were identified in separate experiments using standard CID MS/MS or MS^{E} methods (Waters Inc.). MS^{E} data were processed using BiopharmaLynx 1.2 (version 017). CID data-dependent MS/MS acquisition was analyzed using the MassLynx software and in-house MASCOT database.

HX-MS raw data files were subjected to continuous lock mass-correction. Data analysis, i.e., centroid determination of deuterated peptides and plotting of in-exchange curves, was performed using prototype custom software (HDX browser, Waters Inc.) and HX-Express ((Version Beta); Weis et al., J. Am. Soc. Mass Spectrom. 17, 1700 (2006)). All data were also visually evaluated to ensure only resolved peptide isotopic envelopes were subjected to analysis.

### Epitope Mapping Experiment

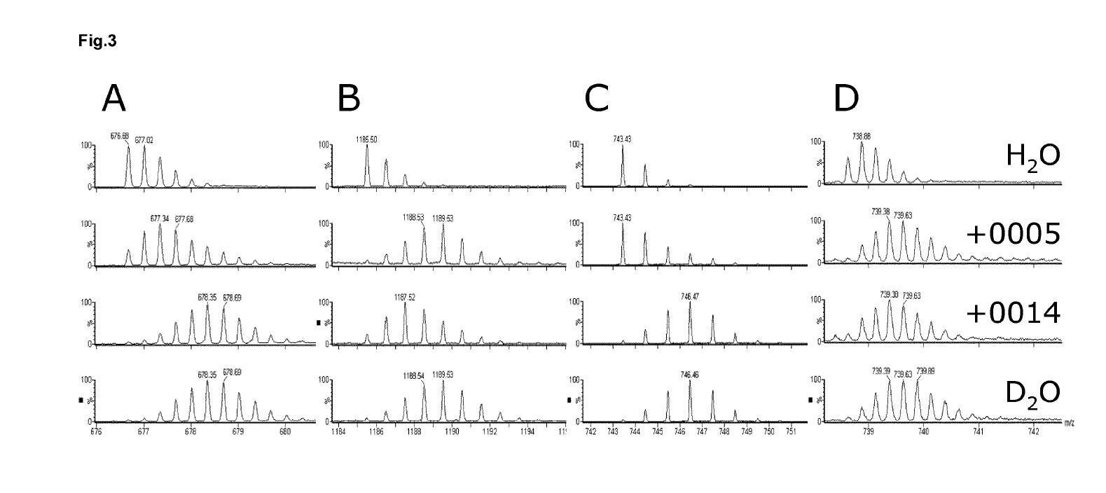

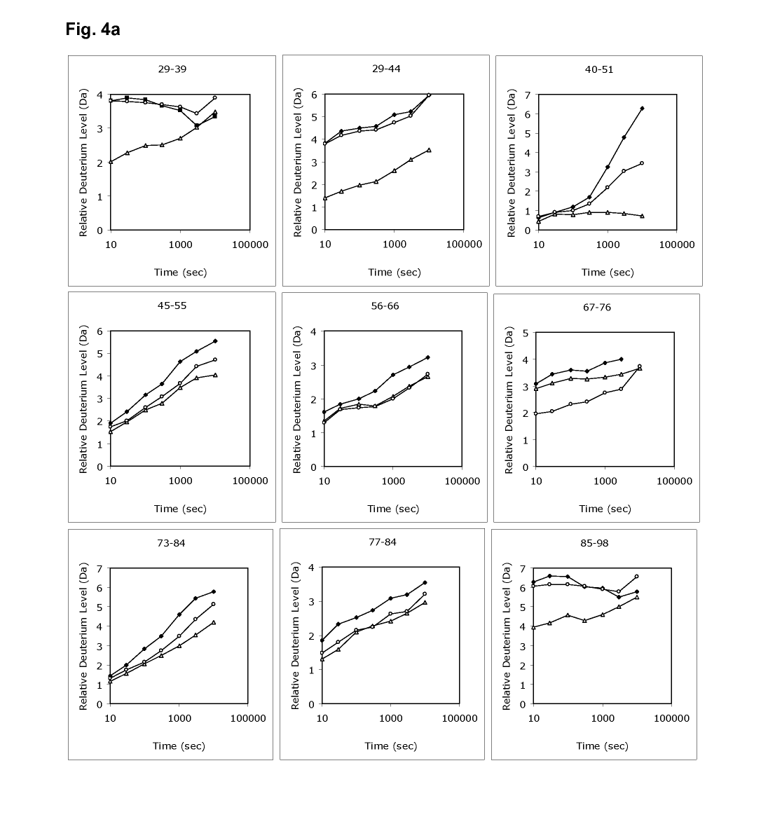

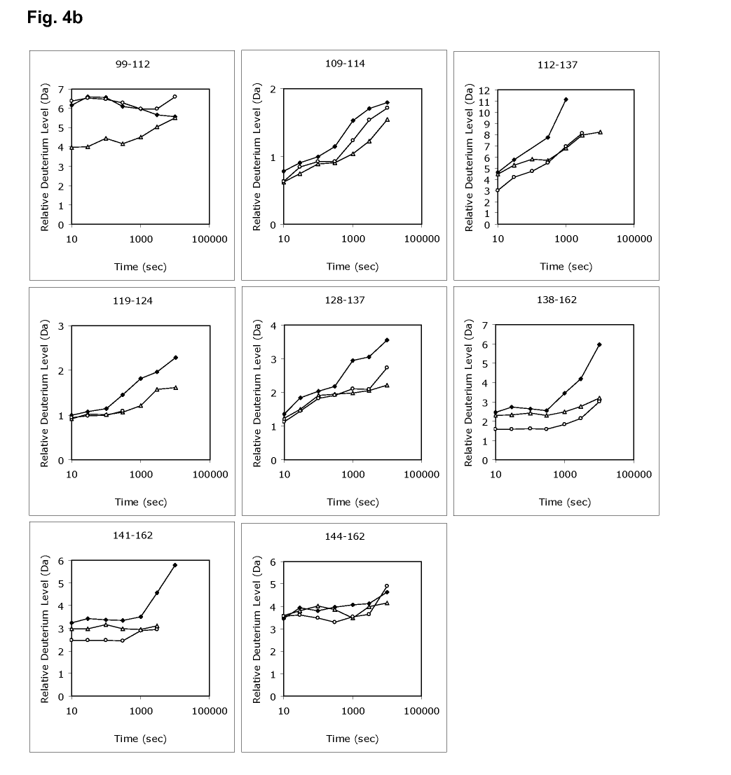

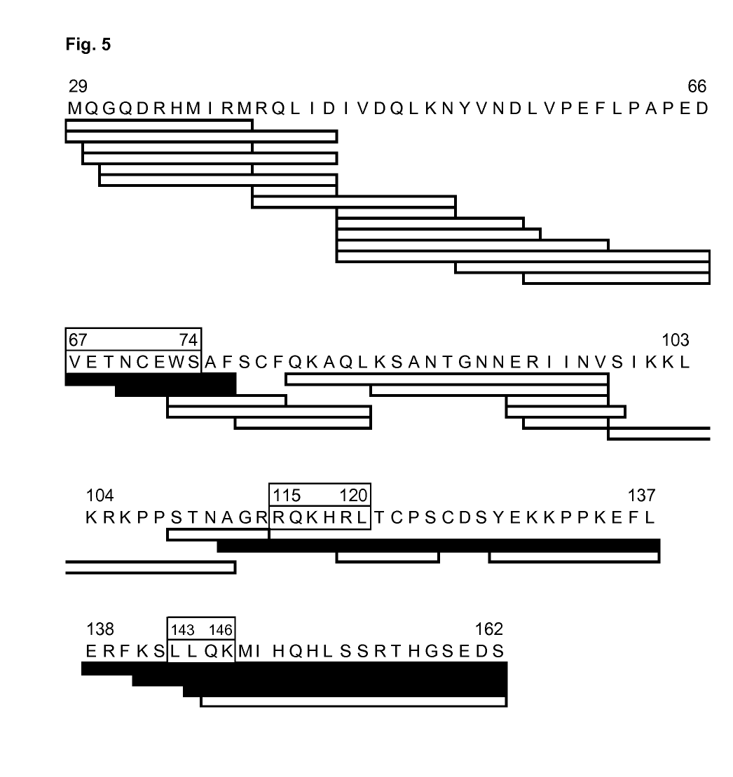

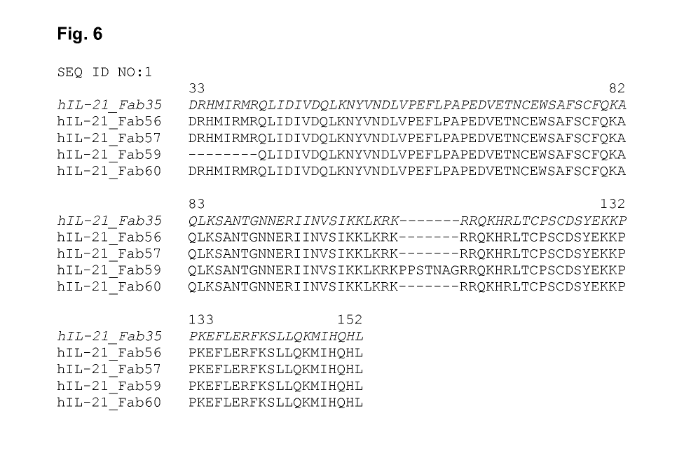

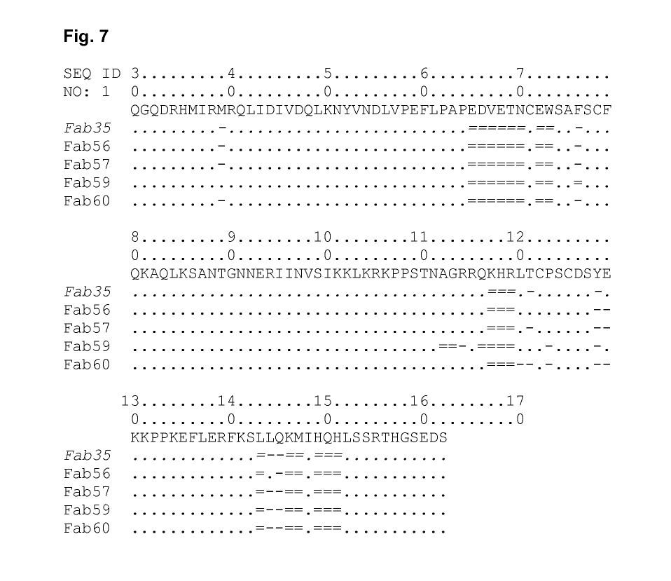

Amide hydrogen/deuterium exchange (HX) was initiated by a 16-fold dilution of hIL-21 in the presence or absence of mAb5 or mAb14 into the corresponding deuterated buffer (i.e. PBS prepared in D₂O, 96% D₂O final, pH 7.4 (uncorrected value)). All HX reactions were carried out at 20° C. and contained 4 μM hIL-21 in the absence or presence of 2.4 μM mAb thus giving a 1.2 fold molar excess of mAb binding sites. At appropriate time intervals ranging from 10 sec to 10000 sec, 50 μl aliquots of the HX reaction were quenched by 50 μl ice-cold quenching buffer (1.35M TCEP) resulting in a final pH of 2.5 (uncorrected value). Examples of raw data identifying the mAb5 and the mAb14 epitopes are shown in FIG. 3.

### Results and Discussion

Epitope Mapping of mAb5 and mAb14

The epitope of mAb5 has previously been mapped (example 2 and FIG. 2).

The HX time-course of 34 peptides, covering 100% of the primary sequence of hIL-21, were monitored in the absence or presence of mAb5 or mAb14 for 10 to 10000 sec (FIGS. 1 and 2). Exchange protection observed in the early time-points, e.g. <300 sec, relate to surface exposed amide protons and thus also relate to protein interfaces. In contrast, effects observed late in the time course are related to slow exchanging amide hydrogens and thus related to the structural core of the protein. Therefore, epitope effects appear in the early time points whereas structural stabilization effects will manifest as exchange reduction in late time points (Garcia, Pantazatos and Villareal, Assay and Drug Dev. Tech. 2, 81 (2004); Mandell, Falick and Komives, Proc. Natl. Acad. Sci. USA, 95, 14705 (1998)).

Epitope Mapping of mAb14

The observed exchange pattern in the early timepoints (<300 sec) in the presence or absence of mAb14 can be divided into two different groups: One group of peptides display an exchange pattern that is unaffected by the binding of mAb14. In contrast, another group of peptides in hIL-21 show protection from exchange upon mAb14 binding (FIGS. 3B, 3D and 4). For example at 30 sec exchange with D₂O, more than 1 amide is protected from exchange in the region V67-F76 upon mAb14 binding (FIGS. 3B, and 4). The regions displaying protection upon mAb14 binding encompass peptides covering residues V67-F76 and A112-S162 (FIGS. 4 and 5). However, by comparing the relative amounts of exchange protection within each peptide upon binding mAb14 and the lack of epitope effects in several other and smaller peptides in these regions, the epitope can be narrowed to residues V67-S74 and L143-K146. Furthermore, the epitope effects in peptide A112-L127 could arise from two different regions within this long peptide. Of these two, only region R115-L120 is in close proximity in the 3D structure of the other two epitope regions and thus the epitope effects are assigned to this region (FIG. 5).

The mAb5 and the mAb14 Epitopes are not Overlapping

As can be seen from the examples in FIG. 5 and the exchange plots in FIG. 4, the epitopes for mAb5 and mAb14 are completely separated and not overlapping.

### Example 8

### Crystal Structures of hIL-21 in Complex with CDR-Loop Mutated Fab Fragments of mAb14

The 3-dimensional structures of hIL-21 in complex with four different Fab fragments, Fab56, Fab57, Fab59 and Fab60 were solved and refined to high resolution using X-ray crystallography. The Fabs are all variants of the Fab35 fragment of anti-IL-21 human monoclonal antibody mAb14 and were designed and generated as described in example 6 and 14, respectively. Fab56, Fab57, Fab59 and Fab60 correspond to Fab fragments of mAb61, mAb62, mAb64 and mAb65, respectively. The results demonstrate that Fab56, Fab57, Fab59 and Fab60 share the epitope on hIL-21 with Fab35. Therefore the binding sites of Fab56, Fab57, Fab59 and Fab60 will, as for Fab35, according to comparative studies/modelling, Example 2, compete with, and due to its high binding affinity, block the binding of the γC receptor chain to hIL-21. Hence, they will inhibit the biological effects mediated by hIL-21 through γC.

Fab59 form a different crystal packing compared to the other mutants, and Fab35, resulting in an epitope including 4 additional residues, when using a 4.0 Å cut-off in the calculation of the epitope, as compared to the other mutants.

The epitopes described were characterized using the 3-dimensional structure of the complexes between Fab56, Fab57, Fab59 or Fab60 and hIL-21, respectively. The conclusions regarding the epitopes of Fab56, Fab57, Fab59 or Fab60 on hIL-21 will, moreover, also apply to the interaction between hIL-21 and the full antibody, mAb14, from which Fab56, Fab57, Fab59 or Fab60, via Fab35, were derived.

### Materials and Methods

IL-21 (expressed in E. coli as the mature peptide; residues 30-162 of SEQ ID NO: 1 with an added N-terminal Methionine residue), in PBS buffer, pH 7.4 (4 tablets in 2 liter of water, GIBCO Cat. No. 18912-014 Invitrogen Corporation), and anti-IL-21 Fabs (comprising light chains and heavy chains corresponding to WT or mutants of SEQ ID No. 9 and 10, respectively, see example 6 and 14) formulated in PBS buffer, pH 7.4, were mixed in a 1:1 molar ratio. The final concentrations of the complexes are shown in Table 7. Crystals were grown with the sitting drop-technique with volumes according to Table 7. Total drop sizes were 0.2 or 0.3 μl, depending on the mixing ratio. Crystals were prepared for cryo-freezing by transferring of 3 μl of a cryo-solution, containing 75% of the precipitant solution and 25% glycerol, to the drop containing the crystal. Soakings were allowed for about one minute. The crystals were then fished into a MiTeGen MicroLoop™, flash frozen in liquid N2 and kept at a temperature of 100 K during data collection by a cryogenic N2 gas stream. Crystallographic data were collected at beam-line BL911-3 (Ursby et al., 2004) at MAX-lab, Lund, Sweden, to resolutions indicated in Table 8. Space group determination, integration and scaling of the data were made with the XDS software package (Kabsch, 2010). A summary of obtained cell parameters, space groups, resolutions, R-sym and completeness are shown in Table 8. For the crystal complexes between hIL-21 and Fab56, Fab57 or Fab60, respectively, the Fab35/hIL-21 crystal structure were used as starting models for rigid body refinements in the Refmac5 software (Murshudov et al., 2011) of the CCP4 crystallography software suite (Bailey, 1994). Rigid body refinements were then followed by restrained crystallographic refinements, using the software programs Refmac5 and by computer graphics inspection of the electron density maps, model corrections and building using the Coot software program (Emsley et al., 2010). The procedure was cycled until no further significant improvements could be made to the model. Table 10, 11 and 13.

TABLE 7
Summary of protein samples and conditions used for crystallizations of the
different mutant-Fab/hIL-21 complexes. Mut: chain name H (heavy chain), L (Light chain)
and amino acid mutation relative to the corresponding WT light or heavy chain reference
(ref) sequence from Fab35.

| | | | Concentration | | | |
|---|---|---|---|---|---|---|
| | | | [mg/ml] | | Crystallization | Protein:precipitant |
| | Ref SEQ | | Before | 1:1 | Precipitant | solution |
| Protein | ID NO | Mut | Mix | Complex | Solution | mix [nL] |
| Soluble | Residues | — | 10.5 | | | |
| hIL-21 | 30-162 | | | | | |
| | of SEQ | | | | | |
| | ID No. 1 | | | | | |
| Fab56 | 10 | H- | 3.5 | 4.4 | 20% PEG4000 | 200:100 |
| | | D62E | | | 200 mM Sodium | |
| | | | | | Choride | |
| Fab57 | 10 | H- | 7.6 | 8.6 | 30% PEG 1000, | 100:100 |
| | | K65R | | | 20 mM | |
| | | | | | Diammonium | |
| | | | | | tartrate | |
| Fab59 | 9 | L- | 9.4 | 9.7 | 30% PEG 1000 | 200:100 |
| | | Q27N | | | 150 mM Sodium | |
| | | | | | Chloride | |
| Fab60 | 9 | L- | 9.0 | 9.3 | 20% PEG 4000, | 200:100 |
| | | D30E | | | 20 mM Calcium | |
| | | | | | Acetate | |

TABLE 8
Some crystallographic data and model statistics for the different mutant-Fab/hIL-
21 complexes. Fab35 data (From Example 1) are added for comparison.

| | | | | | | | | | | | RMSD^{¶} | | |
|---|---|---|---|---|---|---|---|---|---|---|---|---|---|
| Fab | | | | | | | | | | | ideal | | |
| complex | | | | | | | R- | | | R- | bond- | SSM^{$} to Fab35 | |
| with hIL- | a | b | | b | Space | Resol | sym^{†} | Compl* | R^{£} | free [symbol] | lengths | RMSD^{¶} | |
| 21 | [Å] | [Å] | c [Å] | [°] | group | [Å] | [%] | [%] | [%] | [%] | [Å] | [Å] | # Resid^{‡} |
| Fab35 | 89.4 | 65.2 | 106.7 | 111.6 | C2 | 1.64 | 6.4 | 98.2 | 17.9 | 21.1 | 0.024 | — | |
| Fab56 | 89.4 | 65.2 | 106.9 | 111.7 | C2 | 1.65 | 2.8 | 99.2 | 17.7 | 21.4 | 0.022 | 0.144 | 548 |
| Fab57 | 89.7 | 65.1 | 107.1 | 111.6 | C2 | 1.63 | 2.5 | 98.7 | 17.2 | 20.6 | 0.024 | 0.144 | 548 |
| Fab59 | 86.5 | 65.6 | 106.7 | 113.8 | C2 | 1.65 | 3.1 | 97.6 | 16.7 | 20.7 | 0.019 | 0.557 | 533 |
| Fab60 | 89.4 | 65.0 | 106.7 | 111.4 | C2 | 1.75 | 3.7 | 99.3 | 17.1 | 21.5 | 0.020 | 0.120 | 548 |

*To the specified resolution observed diffraction data completeness according to XSCALE (Kabsch, 2010)
^{†}R_{sym} = ΣₕΣᵢ|I(h,i) − [symbol] I(h) [symbol] |/ΣIₕΣᵢ (h,i), where I(h,i) is the intensity of the ith measurement of h and [symbol] I(h) [symbol] is the corresponding average value of all i measurements.
^{£,} [symbol] R = Σₕ||F(h)ₒ| − |F(h)_{c}||/|F(h)ₒ|, where F(h)c is the calculated structure factor of reflection h, R_{free} is equivalent to R_{cryst}, but calculated for randomly chosen 5% of reflections that were omitted from the refinement process.
^{¶}Root-mean-square deviation
^{$}Secondary Structure Matching (Krissinel & Henrick, 2004)
^{‡}Number of amino acid residues used during structure superimpositioning

For the crystal complex between hIL-21 and Fab59 the complex Fab35/hIL-21 crystal structure was used as starting model for structure determination using molecular replacement technique by the Molrep software (Vagin & Teplyakov, 1997) of the CCP4 software suit. It was followed by restrained refinements using the software program Refmac5 and by computer graphics inspection of the model and electron density maps, using the Coot software program (Emsley, Lohkamp, Scott, & Cowtan, 2010). The model needed modifications to the N-terminal part of helix A and to part of the loop-structure between helix C and D. The software ARP/wARP (Perrakis et al., 1999) was used for an initial round if automated model building which was followed by crystallographic refinements, again using the software programs Refmac5 and the Coot software for computer graphic inspections of the electron density maps, model corrections and building. The procedure was cycled until no further significant improvements could be made to the model. The model was then subject to twin-refinement (using the twin-law h,-k, -h-l) in Phenix.Refine (Afonine et al., 2005) of the Phenix software package (Adams et al., 2010). The twin fraction was refined to 0.03 and the resulting R and R-free were 0.166 and 0.201, respectively. Finally the structure was transferred to the CCP4 software system again where a final round of restrained refinements were carried out in Refmac5 followed by structure interpretations, Table 12.

Final R- and R-free, root-mean-square deviation (RMSD) from ideal bond lengths and Secondary Structure Matching (Krissinel & Henrick, 2004) results for the superimpositions of Fab35-hIL-21 onto each of the Fab56-, Fab57-, Fab59- and Fab60-hIL-21 complexes, respectively, are shown in Table 8.

### Results

The results demonstrate that Fab56, Fab57, Fab59 and Fab60 share the epitope on hIL-21 with Fab35. The Fab59/hIL-21 structure show a minor difference in inter-molecular interactions within the crystal (crystal packing) compared to the other Fab variants though. The reason for the difference in crystal packing is that the Fab light chain Gln 27 residue is involved in crystal packing (forming a hydrogen bond to Asp 44 of a symmetry related hIL-21 molecule) in the Fab35, Fab56, Fab57 and Fab60 crystals while that residue is mutated to Asn in Fab59 and cannot form the same inter-molecular contacts (crystal packing interactions) as the other variants, but a slightly different type. The difference result in a closer packing for two symmetry related Fab/hIL21-complex molecules in Fab59 relatively to the equivalent symmetry related packing in Fab35. The distance between the two complexes is reduced about 2.3 Å for Fab59/hIL-21 relative to Fab39/hIL-21 (calculated as the distances between the first axis of the principal moment of inertia for the two systems) and the average areas excluded in pairwise interactions increase from 738 Å2 for the Fab35/hIL-21 crystal to 967 Å2 in the Fab59/hIL-21 crystal, respectively (calculated by the software program Areaimol (Lee & Richards, 1971, Saff & Kuijlaars, 1997)). That, locally, tighter crystals packing of the Fab59/hIL-21 crystals result in that the missing residues of the loop between helices C and D of hIL-21, unobserved in the Fab35/hIL-21 crystal, forms a stable conformation in the Fab59/hIL-21 crystal and are clearly seen in the electron density maps. Moreover the conformation of part of the loop between the hIL-21 helices C and D is, by the symmetry related molecule which is closer in Fab59/hIL21, driven in the direction towards helix A of hIL-21. This force the first part of helix A in hIL-21 to become unstructured and not seen in the electron density maps in the Fab59/hIL-21 complex. Moreover, the ordering and movement of residues 105 to 119 in the loop between helices C and D of hIL-21 make 4 additional residues of hIL-21 (Phe 76, Ala 112, Gly 113, and Gln 116: SEQ ID NO. 1) fall within a 4 Å distance cut-off from the heavy chain of Fab59 as compared to the Fab56, Fab57, Fab60 and Fab35 hIL-21 complexes (See FIG. 6). The hIL-21 binding properties of Fab59 are, however, not different from the other Fab-variants. The binding sites of Fab56, Fab57, Fab59 and Fab60 will all, as for Fab35, instead of competing with the private hIL-21 receptor chain (IL-21Rα), according to comparative studies/modelling, Example 2, compete with, and due to its high binding affinity, block the binding of the γC receptor chain to hIL-21. Hence, it will inhibit the biological effects mediated by hIL-21 through γC.

Table 9 show the calculated (by the software Areaimol (Lee & Richards, 1971, Saff & Kuijlaars, 1997)), average areas excluded in pair-wise interactions for the hIL-21/Fab56, hIL-21/Fab57, hIL-21/Fab59 and hIL-21/Fab60 complexes, respectively. Corresponding calculations for the Fab35/hIL-21 crystal complex show a very similar value (see Example 1), included in the table.

The direct contacts between the hIL-21 and Fab56, Fab57, Fab59 or Fab60, respectively, were identified by running the Contacts software of the CCP4 program suite (Bailey, 1994) using a cut-off distance of 4.0 and 5.0 Å between the anti-IL-21 Fab and the hIL-21 molecules. The results from the hIL-21/Fab56, hIL-21/Fab57, hIL-21/Fab59, hIL-21/Fab60 complex crystal structure are shown in Tables 14, 15, 16 and 17, respectively. The resulting hIL-21 epitopes for Fab56, Fab57, Fab59 and Fab60 were found to comprise the residues of hIL-21 (SEQ ID No. 1) as shown in Table 9 and FIG. 6. Those epitopes agrees very well with the hIL-21 epitope of Fab35, from Example 1, included in Table 9 and FIG. 7.

TABLE 9
Epitopes and paratopes for the different Fab fragments (Fab56, Fab57, Fab59 and Fab60)
using a 4.0 Å distance cut-off between hIL-21 and each of the Fab fragments. The
calculated average areas excluded in pair-wise interactions between hIL-21 and each of
the Fab fragments are also shown. The Seq ID No. for WT light/heavy chain reference
(ref) sequence from Fab35 are listed and mutation (Mut) as also listed in Table 7.

| | Ref. SEQ | | | | |
|---|---|---|---|---|---|
| Antibody | ID No. | Epitope to shIL-21 | Paratope | | Avr |
| fragment | including | (4.0 Å cut-off) | | Light | Area^{#} |
| complex | mutations | SEQ ID No. 1 | Heavy chain | chain | [Å²] |
| Fab35/hIL- | SEQ ID No. | Glu 65, Asp 66, | Ile 28, Ser | Ser 31, | 1061 |
| 21 (From | 9 LC/10 HC | Val 67, Glu 68, | 30, Ser 31, | Asp 50, | |
| Example 1) | | Thr 69, Asn 70, | Tyr 32, Ser | Phe 91, | |
| | | Glu 72, Trp 73, | 33, Thr 52, | Asn 92, | |
| | | Lys 117, His | Ser 53, Gly | Tyr 94 | |
| | | 118, Arg 119, | 54, Ser 55, | | |
| | | Leu 143, Lys | Tyr 56, Tyr | | |
| | | 146, Met 147, | 57, His 59, | | |
| | | His 149, Gln | Glu 99, Arg | | |
| | | 150, His 151 | 100, Gly 101, | | |
| | | | Trp 102, Gly | | |
| | | | 103, Tyr 104, | | |
| | | | Tyr 105 | | |
| Fab56/hIL- | SEQ ID No. | Glu 65, Asp 66, | Ile 28, Ser | Ser 31, | 1068 |
| 21 | 9 LC/10 H | Val 67, Glu 68, | 30, Ser 31, | Asp 50, | |
| | D62E | Thr 69, Asn 70, | Tyr 32, Ser | Phe 91, | |
| | | Glu 72, Trp 73, | 33, Thr 52, | Asn 92, | |
| | | Lys 117, His | Ser 53, Gly | Tyr 94 | |
| | | 118, Arg 119, | 54, Ser 55, | | |
| | | Leu 143, Lys | Tyr 56, Tyr | | |
| | | 146, Met 147, | 57, His 59, | | |
| | | His 149, Gln | Glu 99, Arg | | |
| | | 150, His 151 | 100, Gly 101, | | |
| | | | Trp 102, Gly | | |
| | | | 103, Tyr 104, | | |
| | | | Tyr 105 | | |
| Fab57/hIL- | SEQ ID No. | Glu 65, Asp 66, | Ile 28, Ser | Ser 31, | 1067 |
| 21 | 9 LC/10 H | Val 67, Glu 68, | 30, Ser 31, | Asp 50, | |
| | K65R | Thr 69, Asn 70, | Tyr 32, Ser | Phe 91, | |
| | | Glu 72, Trp 73, | 33, Thr 52, | Asn 92, | |
| | | Lys 117, His | Ser 53, Gly | Tyr 94 | |
| | | 118, Arg 119, | 54, Ser 55, | | |
| | | Leu 143, Lys | Tyr 56, Tyr | | |
| | | 146, Met 147, | 57, His 59, | | |
| | | His 149, Gln | Glu 99, Arg | | |
| | | 150, His 151 | 100, Gly 101, | | |
| | | | Trp 102, Gly | | |
| | | | 103, Tyr 104, | | |
| | | | Tyr 105 | | |
| Fab59/hIL- | SEQ ID No. | Glu 65, Asp 66, | Ile 28, Ser | Asp 30, | 1169 |
| 21 | 9 L Q27N/ | Val 67, Glu 68, | 31, Tyr 32, | Ser 31, | |
| | 10 HC | Thr 69, Asn 70, | Ser 33, Thr | Asp 50, | |
| | | Glu 72, Trp 73, | 52, Ser 53, | Phe 91, | |
| | | Phe 76, Ala | Gly 54, Ser | Asn 92, | |
| | | 112, Gly 113, | 55, Tyr 56, | Tyr 94 | |
| | | Gln 116, Lys | Tyr 57, His | | |
| | | 117, His 118, | 59, Glu 99, | | |
| | | Arg 119, Leu | Arg 100, Gly | | |
| | | 143, Lys 146, | 101, Trp 102, | | |
| | | Met 147, His | Gly 103, Tyr | | |
| | | 149, Gln 150, | 104, Tyr 105 | | |
| | | His 151 | | | |
| Fab60/hIL- | SEQ ID No. | Glu 65, Asp 66, | Ile 28, Ser | Ser 31, | 1103 |
| 21 | 9 L D30E/ | Val 67, Glu 68, | 30, Ser 31, | Asp 50, | |
| | 10 HC | Thr 69, Asn 70, | Tyr 32, Ser | Phe 91, | |
| | | Glu 72, Trp 73, | 33, Thr 52, | Asn 92, | |
| | | Lys 117, His | Ser 53, Gly | Tyr 94 | |
| | | 118, Arg 119, | 54, Ser 55, | | |
| | | Leu 143, Lys | Tyr 56, Tyr | | |
| | | 146, Met 147, | 57, His 59, | | |
| | | His 149, Gln | Glu 99, Arg | | |
| | | 150, His 151 | 100, Gly 101, | | |
| | | | Trp 102, Gly | | |
| | | | 103, Tyr 104, | | |
| | | | Tyr 105 | | |

^{#}Average areas excluded in pairwise interactions

Thus, the Fab56/Fab57/Fab59/Fab60 hIL-21 epitopes comprise residues (SEQ ID No. 1) in the N-terminal part of helix B, residue 72-76, and residues in the C-terminal part of helix D, residues 143-151. Additionally, several contact residues are identified in the loop segment proceeding helix B, residues 65-70, and in the loop between helix C and helix D, residues 112-119, FIG. 7. These contact areas agrees well with what has been determined as the binding site for γC, Example 2.

The Fab56, Fab57, Fab59 and Fab60 paratopes for hIL-21 are shown in Table 9. The hIL-21 paratopes, and the residues involved in hydrogen-binding, are also indicated in Tables 14, 15, 16, and 17.

TABLE 10
Results from the X-ray model refinement to the observed data of the hIL-
21/Fab56 complex by the software program Refmac5 (Murshudov, Skubak, Lebedev,
Pannu, Steiner, Nicholls, Winn, Long, & Vagin, 2011) of the CCP4 program software
package (Bailey, 1994).

| REMARK | 3 REFINEMENT. | |
|---|---|---|
| REMARK | 3 PROGRAM | : REFMAC 5.6.0119 |
| REMARK | 3 AUTHORS | : MURSHUDOV, VAGIN, DODSON |
| REMARK | 3 | |

| REMARK | 3 REFINEMENT TARGET : MAXIMUM LIKELIHOOD | | |
|---|---|---|---|
| REMARK | 3 | | |
| REMARK | 3 DATA USED IN REFINEMENT. | | |
| REMARK | 3 RESOLUTION RANGE HIGH | (ANGSTROMS) : | 1.65 |
| REMARK | 3 RESOLUTION RANGE LOW | (ANGSTROMS) : | 99.39 |
| REMARK | 3 DATA CUTOFF | (SIGMA(F)) : | NONE |
| REMARK | 3 COMPLETENESS FOR RANGE | (%) : | 99.29 |
| REMARK | 3 NUMBER OF REFLECTIONS | : | 64936 |
| REMARK | 3 | | |

| REMARK | 3 FIT TO DATA USED IN REFINEMENT. | | |
|---|---|---|---|
| REMARK | 3 CROSS-VALIDATION METHOD | : | THROUGHOUT |
| REMARK | 3 FREE R VALUE TEST SET SELECTION | : | RANDOM |

| | | | |
|---|---|---|---|
| REMARK | 3 R VALUE | (WORKING + TEST SET) : | 0.17902 |
| REMARK | 3 R VALUE | (WORKING SET) : | 0.17716 |
| REMARK | 3 FREE R VALUE | : | 0.21406 |

| | | | |
|---|---|---|---|
| REMARK | 3 FREE R VALUE TEST SET SIZE | (%) : | 5.1 |
| REMARK | 3 FREE R VALUE TEST SET COUNT | : | 3463 |
| REMARK | 3 | | |

| REMARK | 3 FIT IN THE HIGHEST RESOLUTION BIN. | | |
|---|---|---|---|
| REMARK | 3 TOTAL NUMBER OF BINS USED | : | 20 |
| REMARK | 3 BIN RESOLUTION RANGE HIGH | : | 1.650 |
| REMARK | 3 BIN RESOLUTION RANGE LOW | : | 1.693 |

| | | | |
|---|---|---|---|
| REMARK | 3 REFLECTION IN BIN | (WORKING SET) : | 4541 |
| REMARK | 3 BIN COMPLETENESS | (WORKING + TEST) (%) : | 96.05 |
| REMARK | 3 BIN R VALUE | (WORKING SET) : | 0.290 |

| | | | |
|---|---|---|---|
| REMARK | 3 BIN FREE R VALUE SET COUNT | : | 247 |
| REMARK | 3 BIN FREE R VALUE | : | 0.316 |
| REMARK | 3 | | |

| REMARK | 3 NUMBER OF NON-HYDROGEN ATOMS USED IN REFINEMENT. | | |
|---|---|---|---|
| REMARK | 3 ALL ATOMS | : | 4831 |
| REMARK | 3 | | |
| REMARK | 3 B VALUES. | | |

| | | | |
|---|---|---|---|
| REMARK | 3 FROM WILSON PLOT | (A**2) : | NULL |
| REMARK | 3 MEAN B VALUE | (OVERALL, A**2) : | 28.072 |

| REMARK | 3 OVERALL ANISOTROPIC B VALUE. | |
|---|---|---|
| REMARK | 3 B11 (A**2) : | 0.52 |
| REMARK | 3 B22 (A**2) : | 0.13 |
| REMARK | 3 B33 (A**2) : | −0.11 |
| REMARK | 3 B12 (A**2) : | 0.00 |
| REMARK | 3 B13 (A**2) : | 0.73 |
| REMARK | 3 B23 (A**2) : | 0.00 |
| REMARK | 3 | |

| | | | |
|---|---|---|---|
| REMARK | 3 ESTIMATED OVERALL COORDINATE ERROR. | | |
| REMARK | 3 ESU BASED ON R VALUE | (A): | 0.097 |
| REMARK | 3 ESU BASED ON FREE R VALUE | (A): | 0.098 |
| REMARK | 3 ESU BASED ON MAXIMUM LIKELIHOOD | (A): | 0.070 |
| REMARK | 3 ESU FOR B VALUES BASED ON MAXIMUM LIKELIHOOD | (A**2): | 4.166 |
| REMARK | 3 | | |
| REMARK | 3 CORRELATION COEFFICIENTS. | | |

| REMARK | 3 CORRELATION COEFFICIENT FO-FC : | 0.967 | | | |
|---|---|---|---|---|---|
| REMARK | 3 CORRELATION COEFFICIENT FO-FC FREE : | 0.950 | | | |
| REMARK | 3 | | | | |
| REMARK | 3 RMS DEVIATIONS FROM IDEAL VALUES | | COUNT | RMS | WEIGHT |
| REMARK | 3 BOND LENGTHS REFINED ATOMS | (A): | 4470 | ; 0.022 | ; 0.020 |
| REMARK | 3 BOND ANGLES REFINED ATOMS | (DEGREES): | 6091 | ; 2.181 | ; 1.958 |
| REMARK | 3 TORSION ANGLES, PERIOD 1 | (DEGREES): | 587 | ; 6.416 | ; 5.000 |
| REMARK | 3 TORSION ANGLES, PERIOD 2 | (DEGREES): | 187 | ;37.025 | ;24.064 |
| REMARK | 3 TORSION ANGLES, PERIOD 3 | (DEGREES): | 771 | ;15.116 | ;15.000 |
| REMARK | 3 TORSION ANGLES, PERIOD 4 | (DEGREES): | 25 | ;18.592 | ;15.000 |
| REMARK | 3 CHIRAL-CENTER RESTRAINTS | (A**3): | 681 | ; 0.166 | ; 0.200 |
| REMARK | 3 GENERAL PLANES REFINED ATOMS | (A): | 3365 | ; 0.013 | ; 0.021 |
| REMARK | 3 | | | | |

| | | | | |
|---|---|---|---|---|
| REMARK | 3 ISOTROPIC THERMAL FACTOR RESTRAINTS. | COUNT | RMS | WEIGHT |
| REMARK | 3 | | | |
| REMARK | 3 NCS RESTRAINTS STATISTICS | | | |
| REMARK | 3 NUMBER OF NCS GROUPS : NULL | | | |
| REMARK | 3 | | | |
| REMARK | 3 TWIN DETAILS | | | |
| REMARK | 3 NUMBER OF TWIN DOMAINS : NULL | | | |
| REMARK | 3 | | | |
| REMARK | 3 | | | |
| REMARK | 3 TLS DETAILS | | | |
| REMARK | 3 NUMBER OF TLS GROUPS : 3 | | | |

| REMARK | 3 ATOM RECORD CONTAINS SUM OF TLS AND RESIDUAL B FACTORS | | | | | |
|---|---|---|---|---|---|---|
| REMARK | 3 ANISOU RECORD CONTAINS SUM OF TLS AND RESIDUAL U FACTORS | | | | | |
| REMARK | 3 | | | | | |
| REMARK | 3 TLS GROUP: 1 | | | | | |
| REMARK | 3 NUMBER OF COMPONENTS GROUP : 2 | | | | | |
| REMARK | 3 COMPONENTS | C | SSSEQI | TO | C | SSSEQI |
| REMARK | 3 RESIDUE RANGE : | L | 1 | | L | 109 |
| REMARK | 3 RESIDUE RANGE : | H | 1 | | H | 122 |

| | | | | |
|---|---|---|---|---|
| REMARK | 3 ORIGIN FOR THE GROUP (A): | 9.2840 | 52.2740 | 33.9990 |
| REMARK | 3 T TENSOR | | | |

| REMARK | 3 T11: | 0.0540 T22: | 0.0428 | |
|---|---|---|---|---|
| REMARK | 3 T33: | 0.0291 T12: | 0.0084 | |
| REMARK | 3 T13: | 0.0134 T23: | −0.0009 | |
| REMARK | 3 L TENSOR | | | |
| REMARK | 3 L11: | 1.3802 L22: | 0.7593 | |
| REMARK | 3 L33: | 2.3791 L12: | 0.2416 | |
| REMARK | 3 L13: | 0.4577 L23: | 0.6954 | |
| REMARK | 3 S TENSOR | | | |
| REMARK | 3 S11: | 0.0958 S12: | −0.0684 S13: | −0.0101 |
| REMARK | 3 S21: | 0.0525 S22: | −0.0192 S23: | −0.0359 |
| REMARK | 3 S31: | 0.0395 S32: | 0.1578 S33: | −0.0767 |
| REMARK | 3 | | | |

| REMARK | 3 TLS GROUP : 2 | | | | | |
|---|---|---|---|---|---|---|
| REMARK | 3 NUMBER OF COMPONENTS GROUP : 2 | | | | | |
| REMARK | 3 COMPONENTS | C | SSSEQI | TO | C | SSSEQI |
| REMARK | 3 RESIDUE RANGE : | L | 110 | | L | 214 |
| REMARK | 3 RESIDUE RANGE : | H | 123 | | H | 221 |

| | | | | |
|---|---|---|---|---|
| REMARK | 3 ORIGIN FOR THE GROUP (A): | 27.1960 | 42.5590 | 5.5800 |
| REMARK | 3 T TENSOR | | | |

| REMARK | 3 T11: | 0.0131 T22: | 0.0125 | |
|---|---|---|---|---|
| REMARK | 3 T33: | 0.0096 T12: | 0.0048 | |
| REMARK | 3 T13: | −0.0100 T23: | −0.0061 | |
| REMARK | 3 L TENSOR | | | |
| REMARK | 3 L11: | 1.7376 L22: | 1.9299 | |
| REMARK | 3 L33: | 1.0465 L12: | 0.3656 | |
| REMARK | 3 L13: | −0.2327 L23: | −0.2852 | |
| REMARK | 3 S TENSOR | | | |
| REMARK | 3 S11: | −0.0367 S12: | 0.0026 S13: | −0.0209 |
| REMARK | 3 S21: | −0.0592 S22: | −0.0009 S23: | 0.0273 |
| REMARK | 3 S31: | −0.0484 S32: | 0.0552 S33: | 0.0375 |
| REMARK | 3 | | | |

| REMARK | 3 TLS GROUP : 3 | | | | | |
|---|---|---|---|---|---|---|
| REMARK | 3 NUMBER OF COMPONENTS GROUP : 1 | | | | | |
| REMARK | 3 COMPONENTS | C | SSSEQI | TO | C | SSSEQI |
| REMARK | 3 RESIDUE RANGE: | I | 33 | | I | 152 |

| | | | | |
|---|---|---|---|---|
| REMARK | 3 ORIGIN FOR THE GROUP (A): | −7.8680 | 51.4100 | 61.2480 |
| REMARK | 3 T TENSOR | | | |

| REMARK | 3 T11: | 0.1909 T22: | 0.2025 | |
|---|---|---|---|---|
| REMARK | 3 T33: | 0.1246 T12: | −0.0519 | |
| REMARK | 3 T13: | −0.0005 T23: | −0.0452 | |
| REMARK | 3 L TENSOR | | | |
| REMARK | 3 L11: | 2.1894 L22: | 1.8121 | |
| REMARK | 3 L33: | 4.6135 L12: | 0.6326 | |
| REMARK | 3 L13: | −2.5769 L23: | −1.2482 | |
| REMARK | 3 S TENSOR | | | |
| REMARK | 3 S11: | 0.1031 S12: | −0.3514 S13: | 0.1365 |
| REMARK | 3 S21: | 0.3341 S22: | −0.0740 S23: | −0.1003 |
| REMARK | 3 S31: | −0.1958 S32: | 0.2196 S33: | −0.0292 |
| REMARK | 3 | | | |
| REMARK | 3 | | | |

| REMARK | 3 BULK SOLVENT MODELLING. | | | | |
|---|---|---|---|---|---|
| REMARK | 3 METHOD USED : MASK | | | | |
| REMARK | 3 PARAMETERS FOR MASK CALCULATION | | | | |
| REMARK | 3 VDW PROBE RADIUS : 1.20 | | | | |
| REMARK | 3 ION PROBE RADIUS : 0.80 | | | | |
| REMARK | 3 SHRINKAGE RADIUS : 0.80 | | | | |
| REMARK | 3 | | | | |
| REMARK | 3 OTHER REFINEMENT REMARKS: | | | | |
| REMARK | 3 U VALUES : WITH TLS ADDED | | | | |
| REMARK | 3 HYDROGENS HAVE BEEN USED IF PRESENT IN THE INPUT | | | | |
| REMARK | 3 | | | | |
| SSBOND | 1 CYS L | 88 | CYS | L | 23 |
| SSBOND | 2 CYS H | 134 | CYS | L | 214 |
| SSBOND | 3 CYS H | 96 | CYS | H | 22 |
| SSBOND | 4 CYS I | 71 | CYS | I | 122 |
| SSBOND | 5 CYS I | 78 | CYS | I | 125 |

| | | | | | | |
|---|---|---|---|---|---|---|
| LINKR | SG ACYS L 194 | SG | CYS | L | 134 | SS |
| LINKR | SG BCYS L 194 | SG | CYS | L | 134 | SS |
| LINKR | SG ACYS H 203 | SG | ACYS | H | 147 | SS |
| LINKR | SG BCYS H 203 | SG | BCYS | H | 147 | SS |
| LINKR | LYS I 106 | | ARG | I | 114 | gap |
| LINKR | CYS I 78 | | SER | I | 86 | gap |

| | | | | | |
|---|---|---|---|---|---|
| CISPEP | 1 SER L | 7 | PRO L | 8 | 0.00 |
| CISPEP | 2 TYR L | 94 | PRO L | 95 | 0.00 |
| CISPEP | 3 TYR L | 140 | PRO L | 141 | 0.00 |
| CISPEP | 4 PHE H | 153 | PRO H | 154 | 0.00 |
| CISPEP | 5 GLU H | 155 | PRO H | 156 | 0.00 |

| | | | | | | | |
|---|---|---|---|---|---|---|---|
| CRYST1 | 89.370 | 65.220 | 106.940 | 90.00 | 111.67 | 90.00 | C 1 2 1 |

TABLE 11
Results from the X-ray model refinement to the observed data of the hIL-
21/Fab57 complex by the software program REFMAC5 (Murshudov, Skubak, Lebedev,
Pannu, Steiner, Nicholls, Winn, Long, & Vagin, 2011) of the CCP4 program software
package (Bailey, 1994).

| REMARK | 3 | REFINEMENT. | |
|---|---|---|---|
| REMARK | 3 | PROGRAM | : REFMAC 5.6.0119 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 1.63 |
| REMARK | 3 | RESOLUTION RANGE LOW | (ANGSTROMS) | : 99.59 |
| REMARK | 3 | DATA CUTOFF | (SIGMA(F)) | : NONE |
| REMARK | 3 | COMPLETENESS FOR RANGE | (%) | : 98.79 |
| REMARK | 3 | NUMBER OF REFLECTIONS | | : 67154 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.17343 |
| REMARK | 3 | R VALUE | (WORKING SET) | : 0.17173 |
| REMARK | 3 | FREE R VALUE | | : 0.20563 |

| | | | | |
|---|---|---|---|---|
| REMARK | 3 | FREE R VALUE TEST SET SIZE | (%) | : 5.0 |

| | | | |
|---|---|---|---|
| REMARK | 3 | FREE R VALUE TEST SET COUNT | : 3567 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 1.630 |
| REMARK | 3 | BIN RESOLUTION RANGE LOW | | : 1.672 |
| REMARK | 3 | REFLECTION IN BIN | (WORKING SET) | : 4231 |
| REMARK | 3 | BIN COMPLETENESS | (WORKING + TEST) (%) | : 87.29 |
| REMARK | 3 | BIN R VALUE | (WORKING SET) | : 0.273 |

| | | | |
|---|---|---|---|
| REMARK | 3 | BIN FREE R VALUE SET COUNT | : 232 |
| REMARK | 3 | BIN FREE R VALUE | : 0.289 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS | : | 4888 |
| REMARK | 3 | | | |
| REMARK | 3 | B VALUES. | | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 24.862 |

| REMARK | 3 | OVERALL ANISOTROPIC B VALUE. | |
|---|---|---|---|
| REMARK | 3 | B11 (A**2) : | −0.19 |
| REMARK | 3 | B22 (A**2) : | 0.04 |
| REMARK | 3 | B33 (A**2) : | 0.48 |
| REMARK | 3 | B12 (A**2) : | 0.00 |
| REMARK | 3 | B13 (A**2) : | 0.45 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A) : | 0.092 |
| REMARK | 3 | ESU BASED ON FREE R VALUE | (A) : | 0.092 |
| REMARK | 3 | ESU BASED ON MAXIMUM LIKELIHOOD | (A) : | 0.062 |
| REMARK | 3 | ESU FOR B VALUES BASED ON MAXIMUM LIKELIHOOD | (A**2) : | 3.580 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.967 |
| REMARK | 3 | CORRELATION COEFFICIENT FO-FC FREE | : 0.951 |

| | | |
|---|---|---|
| REMARK | 3 | 0 |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A) : | 4497 | ; 0.024 | ; 0.020 |
| REMARK | 3 | BOND ANGLES REFINED ATOMS | (DEGREES) : | 6140 | ; 2.346 | ; 1.958 |
| REMARK | 3 | TORSION ANGLES, PERIOD 1 | (DEGREES) : | 599 | ; 6.782 | ; 5.000 |
| REMARK | 3 | TORSION ANGLES, PERIOD 2 | (DEGREES) : | 186 | ;35.335 | ;23.925 |
| REMARK | 3 | TORSION ANGLES, PERIOD 3 | (DEGREES) : | 776 | ;14.615 | ;15.000 |
| REMARK | 3 | TORSION ANGLES, PERIOD 4 | (DEGREES) : | 26 | ;17.500 | ;15.000 |
| REMARK | 3 | CHIRAL-CENTER RESTRAINTS | (A**3) : | 692 | ; 0.178 | ; 0.200 |

| | | | | | | |
|---|---|---|---|---|---|---|
| REMARK | 3 | GENERAL PLANES REFINED ATOMS | (A) : | 3386 | ; 0.013 | ; 0.021 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |
| REMARK | 3 | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | TWIN DETAILS | | | | | |
| REMARK | 3 | NUMBER OF TWIN DOMAINS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 3 | | | | | |
| REMARK | 3 | ATOM RECORD CONTAINS SUM OF TLS AND RESIDUAL B FACTORS | | | | | |
| REMARK | 3 | ANISOU RECORD CONTAINS SUM OF TLS AND RESIDUAL U FACTORS | | | | | |
| REMARK | 3 | | | | | | |
| REMARK | 3 | TLS GROUP : 1 | | | | | |
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 1 | | L | 109 |
| REMARK | 3 | RESIDUE RANGE : | H | 1 | | H | 122 |

| | | | |
|---|---|---|---|
| REMARK | 3 | ORIGIN FOR THE GROUP (A) : | 9.3170 52.1750 34.1280 |

| | | | | | | | | |
|---|---|---|---|---|---|---|---|---|
| REMARK | 3 | T | TENSOR | | | | | |
| REMARK | 3 | | T11: | 0.0709 | T22: | 0.0696 | | |
| REMARK | 3 | | T33: | 0.0055 | T12: | 0.0119 | | |
| REMARK | 3 | | T13: | 0.0124 | T23: | −0.0033 | | |
| REMARK | 3 | L | TENSOR | | | | | |
| REMARK | 3 | | L11: | 1.2847 | L22: | 0.7320 | | |
| REMARK | 3 | | L33: | 2.0359 | L12: | 0.2717 | | |
| REMARK | 3 | | L13: | 0.4247 | L23: | 0.5557 | | |
| REMARK | 3 | S | TENSOR | | | | | |
| REMARK | 3 | | S11: | 0.0711 | S12: | −0.0092 | S13: | −0.0205 |
| REMARK | 3 | | S21: | 0.0265 | S22: | −0.0078 | S23: | −0.0337 |
| REMARK | 3 | | S31: | 0.0283 | S32: | 0.1350 | S33: | −0.0633 |
| REMARK | 3 | | | | | | | |

| REMARK | 3 | TLS GROUP : 2 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 110 | | L | 214 |
| REMARK | 3 | RESIDUE RANGE : | H | 123 | | H | 220 |

| | | | |
|---|---|---|---|
| REMARK | 3 | ORIGIN FOR THE GROUP (A): | 27.2680 42.5310 5.6470 |

| | | | | | | | | |
|---|---|---|---|---|---|---|---|---|
| REMARK | 3 | T | TENSOR | | | | | |
| REMARK | 3 | | T11: | 0.0368 | T22: | 0.0396 | | |
| REMARK | 3 | | T33: | 0.0051 | T12: | 0.0215 | | |
| REMARK | 3 | | T13: | −0.0045 | T23: | −0.0093 | | |
| REMARK | 3 | L | TENSOR | | | | | |
| REMARK | 3 | | L11: | 1.5197 | L22: | 1.6538 | | |
| REMARK | 3 | | L33: | 0.8328 | L12: | 0.2355 | | |
| REMARK | 3 | | L13: | −0.2583 | L23: | −0.2416 | | |
| REMARK | 3 | S | TENSOR | | | | | |
| REMARK | 3 | | S11: | −0.0261 | S12: | −0.0122 | S13: | −0.0176 |
| REMARK | 3 | | S21: | −0.0321 | S22: | −0.0010 | S23: | 0.0237 |
| REMARK | 3 | | S31: | −0.0309 | S32: | 0.0316 | S33: | 0.0272 |
| REMARK | 3 | | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE: | I | 33 | | I | 152 |

| | | | |
|---|---|---|---|
| REMARK | 3 | ORIGIN FOR THE GROUP (A): | −7.7920 51.3210 61.3990 |

| | | | | | | | | |
|---|---|---|---|---|---|---|---|---|
| REMARK | 3 | T | TENSOR | | | | | |
| REMARK | 3 | | T11: | 0.1553 | T22: | 0.1892 | | |
| REMARK | 3 | | T33: | 0.0740 | T12: | −0.0298 | | |
| REMARK | 3 | | T13: | −0.0119 | T23: | −0.0378 | | |
| REMARK | 3 | L | TENSOR | | | | | |
| REMARK | 3 | | L11: | 1.8425 | L22: | 1.8733 | | |
| REMARK | 3 | | L33: | 3.8411 | L12: | 0.4705 | | |
| REMARK | 3 | | L13: | −1.9837 | L23: | −1.1584 | | |
| REMARK | 3 | S | TENSOR | | | | | |
| REMARK | 3 | | S11: | 0.0700 | S12: | −0.3050 | S13: | 0.1406 |
| REMARK | 3 | | S21: | 0.2310 | S22: | −0.0397 | S23: | −0.0781 |
| REMARK | 3 | | S31: | −0.1358 | S32: | 0.1565 | S33: | −0.0302 |
| REMARK | 3 | | | | | | | |
| REMARK | 3 | | | | | | | |

| REMARK | 3 | BULK SOLVENT MODELLING. | | | | | |
|---|---|---|---|---|---|---|---|
| REMARK | 3 | METHOD USED : MASK | | | | | |
| REMARK | 3 | PARAMETERS FOR MASK CALCULATION | | | | | |
| REMARK | 3 | VDW PROBE RADIUS | | | | | : 1.20 |
| REMARK | 3 | ION PROBE RADIUS | | | | | : 0.80 |
| REMARK | 3 | SHRINKAGE RADIUS | | | | | : 0.80 |
| REMARK | 3 | | | | | | |
| REMARK | 3 | OTHER REFINEMENT REMARKS: | | | | | |
| REMARK | 3 | U VALUES : WITH TLS ADDED | | | | | |
| REMARK | 3 | HYDROGENS HAVE BEEN USED IF PRESENT IN THE INPUT | | | | | |
| REMARK | 3 | | | | | | |
| SSBOND | 1 | CYS | L | 88 | CYS | L | 23 |
| SSBOND | 2 | CYS | H | 134 | CYS | L | 214 |
| SSBOND | 3 | CYS | H | 96 | CYS | H | 22 |
| SSBOND | 4 | CYS | I | 71 | CYS | I | 122 |
| SSBOND | 5 | CYS | I | 78 | CYS | I | 125 |

| | | | | | | | | | |
|---|---|---|---|---|---|---|---|---|---|
| LINKR | SG | ACYS | L | 194 | SG | CYS | L | 134 | SS |
| LINKR | SG | BCYS | L | 194 | SG | CYS | L | 134 | SS |
| LINKR | SG | ACYS | H | 203 | SG | ACYS | H | 147 | SS |
| LINKR | SG | BCYS | H | 203 | SG | BCYS | H | 147 | SS |
| LINKR | | LYS | I | 106 | | ARG | I | 114 | gap |
| LINKR | | CYS | I | 78 | | SER | I | 86 | gap |

| | | | | | | | | |
|---|---|---|---|---|---|---|---|---|
| CISPEP | 1 | SER | L | 7 | PRO | L | 8 | 0.00 |
| CISPEP | 2 | TYR | L | 94 | PRO | L | 95 | 0.00 |
| CISPEP | 3 | TYR | L | 140 | PRO | L | 141 | 0.00 |
| CISPEP | 4 | PHE | H | 153 | PRO | H | 154 | 0.00 |
| CISPEP | 5 | GLU | H | 155 | PRO | H | 156 | 0.00 |

| | |
|---|---|
| CRYST1 | 89.670 65.120 107.130 90.00 111.62 90.00 C 1 2 1 |

TABLE 12
Results from the X-ray model refinement to the observed data of the hIL-
21/Fab59 complex by the software program REFMAC5 (Murshudov, Skubak, Lebedev,
Pannu, Steiner, Nicholls, Winn, Long, & Vagin, 2011) of the CCP4 program software
package (Bailey, 1994).

| REMARK | 3 | REFINEMENT. | |
|---|---|---|---|
| REMARK | 3 | PROGRAM | : REFMAC 5.6.0119 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 1.65 |
| REMARK | 3 | RESOLUTION RANGE LOW | (ANGSTROMS) | : 97.67 |
| REMARK | 3 | DATA CUTOFF | (SIGMA(F)) | : NONE |
| REMARK | 3 | COMPLETENESS FOR RANGE | (%) | : 97.62 |
| REMARK | 3 | NUMBER OF REFLECTIONS | | : 61004 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.16868 |
| REMARK | 3 | R VALUE | (WORKING SET) | : 0.16667 |
| REMARK | 3 | FREE R VALUE | | : 0.20681 |

| | | | | |
|---|---|---|---|---|
| REMARK | 3 | FREE R VALUE TEST SET SIZE | (%) | : 5.1 |

| | | | |
|---|---|---|---|
| REMARK | 3 | FREE R VALUE TEST SET COUNT | : 3247 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 1.650 |
| REMARK | 3 | BIN RESOLUTION RANGE LOW | | : 1.693 |
| REMARK | 3 | REFLECTION IN BIN | (WORKING SET) | : 3989 |
| REMARK | 3 | BIN COMPLETENESS | (WORKING + TEST) (%) | : 88.41 |
| REMARK | 3 | BIN R VALUE | (WORKING SET) | : 0.279 |

| | | | |
|---|---|---|---|
| REMARK | 3 | BIN FREE R VALUE SET COUNT | : 228 |
| REMARK | 3 | BIN FREE R VALUE | : 0.349 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS | : | 4862 |
| REMARK | 3 | | | |
| REMARK | 3 | B VALUES. | | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 29.931 |

| REMARK | 3 | OVERALL ANISOTROPIC B VALUE. | |
|---|---|---|---|
| REMARK | 3 | B11 (A**2) : | −0.79 |
| REMARK | 3 | B22(A**2) : | 0.32 |
| REMARK | 3 | B33 (A**2) : | −0.20 |
| REMARK | 3 | B12 (A**2) : | 0.00 |
| REMARK | 3 | B13 (A**2) : | −0.83 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A) : | 0.096 |
| REMARK | 3 | ESU BASED ON FREE R VALUE | (A) : | 0.099 |
| REMARK | 3 | ESU BASED ON MAXIMUM LIKELIHOOD | (A) : | 0.072 |
| REMARK | 3 | ESU FOR B VALUES BASED ON MAXIMUM LIKELIHOOD | (A**2) : | 4.204 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.972 |
| REMARK | 3 | CORRELATION COEFFICIENT FO-FC FREE | : 0.955 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A) : | 4455 | ; 0.019 | ; 0.020 |
| REMARK | 3 | BOND ANGLES REFINED ATOMS | (DEGREES) : | 6106 | ; 2.007 | ; 1.957 |
| REMARK | 3 | TORSION ANGLES, PERIOD 1 | (DEGREES) : | 603 | ; 6.728 | ; 5.000 |
| REMARK | 3 | TORSION ANGLES, PERIOD 2 | (DEGREES) : | 181 | ;36.592 | ;24.254 |
| REMARK | 3 | TORSION ANGLES, PERIOD 3 | (DEGREES) : | 760 | ;14.368 | ;15.000 |
| REMARK | 3 | TORSION ANGLES, PERIOD 4 | (DEGREES) : | 22 | ;15.959 | ;15.000 |
| REMARK | 3 | CHIRAL-CENTER RESTRAINTS | (A**3) : | 686 | ; 0.156 | ; 0.200 |

| | | | | | | |
|---|---|---|---|---|---|---|
| REMARK | 3 | GENERAL PLANES REFINED ATOMS | (A) : | 3376 | ; 0.012 | ; 0.021 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |
| REMARK | 3 | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | TWIN DETAILS | | | | | |
| REMARK | 3 | NUMBER OF TWIN DOMAINS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 3 | | | | | |
| REMARK | 3 | ATOM RECORD CONTAINS SUM OF TLS AND RESIDUAL B FACTORS | | | | | |
| REMARK | 3 | ANISOU RECORD CONTAINS SUM OF TLS AND RESIDUAL U FACTORS | | | | | |
| REMARK | 3 | | | | | | |
| REMARK | 3 | TLS GROUP : 1 | | | | | |
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 1 | | L | 109 |
| REMARK | 3 | RESIDUE RANGE : | H | 1 | | H | 122 |

| | | | |
|---|---|---|---|
| REMARK | 3 | ORIGIN FOR THE GROUP (A) : | 29.0754 47.2942 15.5475 |

| | | | | | | | | |
|---|---|---|---|---|---|---|---|---|
| REMARK | 3 | T | TENSOR | | | | | |
| REMARK | 3 | | T11: | 0.0323 | T22: | 0.0800 | | |
| REMARK | 3 | | T33: | 0.0367 | T12: | 0.0301 | | |
| REMARK | 3 | | T13: | −0.0119 | T23: | −0.0453 | | |
| REMARK | 3 | L | TENSOR | | | | | |
| REMARK | 3 | | L11: | 2.8773 | L22: | 0.5802 | | |
| REMARK | 3 | | L33: | 1.2720 | L12: | −0.7786 | | |
| REMARK | 3 | | L13: | −0.4675 | L23: | 0.2500 | | |
| REMARK | 3 | S | TENSOR | | | | | |
| REMARK | 3 | | S11: | −0.1120 | S12: | −0.4390 | S13: | 0.2056 |
| REMARK | 3 | | S21: | 0.1209 | S22: | 0.1658 | S23: | −0.0721 |
| REMARK | 3 | | S31: | −0.0327 | S32: | 0.0715 | S33: | −0.0538 |
| REMARK | 3 | | | | | | | |

| REMARK | 3 | TLS GROUP : 2 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 110 | | L | 214 |
| REMARK | 3 | RESIDUE RANGE : | H | 123 | | H | 220 |

| | | | |
|---|---|---|---|
| REMARK | 3 | ORIGIN FOR THE GROUP (A) : | 47.8498 56.3361 43.1198 |

| | | | | | | | | |
|---|---|---|---|---|---|---|---|---|
| REMARK | 3 | T | TENSOR | | | | | |
| REMARK | 3 | | T11: | 0.1330 | T22: | 0.0092 | | |
| REMARK | 3 | | T33: | 0.0336 | T12: | 0.0037 | | |
| REMARK | 3 | | T13: | −0.0270 | T23: | −0.0126 | | |
| REMARK | 3 | L | TENSOR | | | | | |
| REMARK | 3 | | L11: | 2.6436 | L22: | 1.7178 | | |
| REMARK | 3 | | L33: | 2.2083 | L12: | −1.0715 | | |
| REMARK | 3 | | L13: | 0.2707 | L23: | 0.3686 | | |
| REMARK | 3 | S | TENSOR | | | | | |
| REMARK | 3 | | S11: | −0.0461 | S12: | −0.0631 | S13: | 0.0549 |
| REMARK | 3 | | S21: | 0.1094 | S22: | −0.0586 | S23: | 0.0461 |
| REMARK | 3 | | S31: | −0.1437 | S32: | −0.0273 | S33: | 0.1046 |
| REMARK | 3 | | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE: | I | 41 | | I | 152 |

| | | | |
|---|---|---|---|
| REMARK | 3 | ORIGIN FOR THE GROUP (A): | 10.7692 48.3487 −11.1611 |

| | | | | | | | | |
|---|---|---|---|---|---|---|---|---|
| REMARK | 3 | T | TENSOR | | | | | |
| REMARK | 3 | | T11: | 0.0629 | T22: | 0.1001 | | |
| REMARK | 3 | | T33: | 0.0340 | T12: | 0.0146 | | |
| REMARK | 3 | | T13: | 0.0170 | T23: | −0.0336 | | |
| REMARK | 3 | L | TENSOR | | | | | |
| REMARK | 3 | | L11: | 4.9770 | L22: | 3.1372 | | |
| REMARK | 3 | | L33: | 3.1444 | L12: | −1.0585 | | |
| REMARK | 3 | | L13: | 1.7375 | L23: | −0.9936 | | |
| REMARK | 3 | S | TENSOR | | | | | |
| REMARK | 3 | | S11: | 0.1327 | S12: | 0.6864 | S13: | −0.1705 |
| REMARK | 3 | | S21: | −0.4090 | S22: | −0.1371 | S23: | −0.0276 |
| REMARK | 3 | | S31: | 0.0681 | S32: | 0.1554 | S33: | 0.0044 |
| REMARK | 3 | | | | | | | |
| REMARK | 3 | | | | | | | |

| REMARK | 3 | BULK SOLVENT MODELLING. | | | | | |
|---|---|---|---|---|---|---|---|
| REMARK | 3 | METHOD USED : MASK | | | | | |
| REMARK | 3 | PARAMETERS FOR MASK CALCULATION | | | | | |
| REMARK | 3 | VDW PROBE RADIUS | | | | | : 1.20 |
| REMARK | 3 | ION PROBE RADIUS | | | | | : 0.80 |
| REMARK | 3 | SHRINKAGE RADIUS | | | | | : 0.80 |
| REMARK | 3 | | | | | | |
| REMARK | 3 | OTHER REFINEMENT REMARKS: | | | | | |
| REMARK | 3 | U VALUES : WITH TLS ADDED | | | | | |
| REMARK | 3 | | | | | | |
| SSBOND | 1 | CYS | L | 23 | CYS | L | 88 |
| SSBOND | 2 | CYS | L | 214 | CYS | H | 134 |
| SSBOND | 3 | CYS | H | 22 | CYS | H | 96 |
| SSBOND | 4 | CYS | I | 71 | CYS | I | 122 |
| SSBOND | 5 | CYS | I | 78 | CYS | I | 125 |

| | | | | | | | | | |
|---|---|---|---|---|---|---|---|---|---|
| LINKR | SG | CYS | L | 134 | SG | ACYS | L | 194 | SS |
| LINKR | SG | CYS | L | 134 | SG | BCYS | L | 194 | SS |
| LINKR | SG | ACYS | H | 147 | SG | CYS | H | 203 | SS |
| LINKR | SG | BCYS | H | 147 | SG | CYS | H | 203 | SS |

| | | | | | | | | |
|---|---|---|---|---|---|---|---|---|
| CISPEP | 1 | SER | L | 7 | PRO | L | 8 | 0.00 |
| CISPEP | 2 | TYR | L | 94 | PRO | L | 95 | 0.00 |
| CISPEP | 3 | TYR | L | 140 | PRO | L | 141 | 0.00 |
| CISPEP | 4 | PHE | H | 153 | PRO | H | 154 | 0.00 |
| CISPEP | 5 | GLU | H | 155 | PRO | H | 156 | 0.00 |

| | |
|---|---|
| CRYST1 | 86.510 65.580 106.720 90.00 113.77 90.00 C 1 2 1 |

TABLE 13
Results from the X-ray model refinement to the observed data of the hIL-
21/Fab60 complex by the software program REFMAC5 (Murshudov, Skubak, Lebedev,
Pannu, Steiner, Nicholls, Winn, Long, & Vagin, 2011) of the CCP4 program software
package (Bailey, 1994).

| REMARK | 3 | REFINEMENT. | |
|---|---|---|---|
| REMARK | 3 | PROGRAM | : REFMAC 5.6.0119 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 1.75 |
| REMARK | 3 | RESOLUTION RANGE LOW | (ANGSTROMS) | : 99.36 |
| REMARK | 3 | DATA CUTOFF | (SIGMA(F)) | : NONE |
| REMARK | 3 | COMPLETENESS FOR RANGE | (%) | : 99.32 |
| REMARK | 3 | NUMBER OF REFLECTIONS | | : 54297 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.17370 |
| REMARK | 3 | R VALUE | (WORKING SET) | : 0.17150 |
| REMARK | 3 | FREE R VALUE | | : 0.21523 |

| | | | | |
|---|---|---|---|---|
| REMARK | 3 | FREE R VALUE TEST SET SIZE | (%) | : 5.0 |

| | | | |
|---|---|---|---|
| REMARK | 3 | FREE R VALUE TEST SET COUNT | : 2873 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 1.750 |
| REMARK | 3 | BIN RESOLUTION RANGE LOW | | : 1.795 |
| REMARK | 3 | REFLECTION IN BIN | (WORKING SET) | : 3872 |
| REMARK | 3 | BIN COMPLETENESS | (WORKING + TEST) (%) | : 99.66 |
| REMARK | 3 | BIN R VALUE | (WORKING SET) | : 0.261 |

| | | | |
|---|---|---|---|
| REMARK | 3 | BIN FREE R VALUE SET COUNT | : 218 |
| REMARK | 3 | BIN FREE R VALUE | : 0.326 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS | : | 4853 |
| REMARK | 3 | | | |
| REMARK | 3 | B VALUES. | | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 31.261 |

| REMARK | 3 | OVERALL ANISOTROPIC B VALUE. | |
|---|---|---|---|
| REMARK | 3 | B11 (A**2) : | 0.15 |
| REMARK | 3 | B22 (A**2) : | 0.44 |
| REMARK | 3 | B33 (A**2) : | −0.27 |
| REMARK | 3 | B12 (A**2) : | 0.00 |
| REMARK | 3 | B13 (A**2) : | 0.45 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A) : | 0.114 |
| REMARK | 3 | ESU BASED ON FREE R VALUE | (A) : | 0.115 |
| REMARK | 3 | ESU BASED ON MAXIMUM LIKELIHOOD | (A) : | 0.084 |
| REMARK | 3 | ESU FOR B VALUES BASED ON MAXIMUM LIKELIHOOD | (A**2) : | 5.263 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.969 |
| REMARK | 3 | CORRELATION COEFFICIENT FO-FC FREE | : 0.949 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A) : | 4472 | ; 0.020 | ; 0.020 |
| REMARK | 3 | BOND ANGLES REFINED ATOMS | (DEGREES) : | 6096 | ; 2.047 | ; 1.958 |
| REMARK | 3 | TORSION ANGLES, PERIOD 1 | (DEGREES) : | 587 | ; 6.658 | ; 5.000 |
| REMARK | 3 | TORSION ANGLES, PERIOD 2 | (DEGREES) : | 186 | ;35.861 | ;24.032 |
| REMARK | 3 | TORSION ANGLES, PERIOD 3 | (DEGREES) : | 771 | ;15.436 | ;15.000 |
| REMARK | 3 | TORSION ANGLES, PERIOD 4 | (DEGREES) : | 25 | ;16.721 | ;15.000 |
| REMARK | 3 | CHIRAL-CENTER RESTRAINTS | (A**3) : | 685 | ; 0.156 | ; 0.200 |

| | | | | | | |
|---|---|---|---|---|---|---|
| REMARK | 3 | GENERAL PLANES REFINED ATOMS | (A) : | 3361 | ; 0.012 | ; 0.021 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |
| REMARK | 3 | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | TWIN DETAILS | | | | | |
| REMARK | 3 | NUMBER OF TWIN DOMAINS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 3 | | | | | |
| REMARK | 3 | ATOM RECORD CONTAINS SUM OF TLS AND RESIDUAL B FACTORS | | | | | |
| REMARK | 3 | ANISOU RECORD CONTAINS SUM OF TLS AND RESIDUAL U FACTORS | | | | | |
| REMARK | 3 | | | | | | |
| REMARK | 3 | TLS GROUP : 1 | | | | | |
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 1 | | L | 109 |
| REMARK | 3 | RESIDUE RANGE : | H | 1 | | H | 122 |

| | | | |
|---|---|---|---|
| REMARK | 3 | ORIGIN FOR THE GROUP (A) : | 9.4500 52.1010 34.0400 |

| | | | | | | | | |
|---|---|---|---|---|---|---|---|---|
| REMARK | 3 | T | TENSOR | | | | | |
| REMARK | 3 | | T11: | 0.0551 | T22: | 0.0460 | | |
| REMARK | 3 | | T33: | 0.0379 | T12: | 0.0189 | | |
| REMARK | 3 | | T13: | 0.0116 | T23: | −0.0013 | | |
| REMARK | 3 | L | TENSOR | | | | | |
| REMARK | 3 | | L11: | 1.0795 | L22: | 0.6600 | | |
| REMARK | 3 | | L33: | 2.3184 | L12: | 0.1843 | | |
| REMARK | 3 | | L13: | 0.5068 | L23: | 0.5570 | | |
| REMARK | 3 | S | TENSOR | | | | | |
| REMARK | 3 | | S11: | 0.0616 | S12: | −0.0144 | S13: | −0.0581 |
| REMARK | 3 | | S21: | 0.0275 | S22: | 0.0169 | S23: | −0.0385 |
| REMARK | 3 | | S31: | 0.0405 | S32: | 0.1344 | S33: | −0.0786 |
| REMARK | 3 | | | | | | | |

| REMARK | 3 | TLS GROUP : 2 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | L | 110 | | L | 214 |
| REMARK | 3 | RESIDUE RANGE : | H | 123 | | H | 220 |

| | | | |
|---|---|---|---|
| REMARK | 3 | ORIGIN FOR THE GROUP (A) : | 27.2030 42.4080 5.6610 |

| | | | | | | | | |
|---|---|---|---|---|---|---|---|---|
| REMARK | 3 | T | TENSOR | | | | | |
| REMARK | 3 | | T11: | 0.0234 | T22: | 0.0148 | | |
| REMARK | 3 | | T33: | 0.0247 | T12: | 0.0097 | | |
| REMARK | 3 | | T13: | −0.0073 | T23: | −0.0075 | | |
| REMARK | 3 | L | TENSOR | | | | | |
| REMARK | 3 | | L11: | 1.9247 | L22: | 1.8682 | | |
| REMARK | 3 | | L33: | 0.9227 | L12: | 0.2905 | | |
| REMARK | 3 | | L13: | −0.2738 | L23: | −0.3214 | | |
| REMARK | 3 | S | TENSOR | | | | | |
| REMARK | 3 | | S11: | −0.0453 | S12: | −0.0032 | S13: | −0.0157 |
| REMARK | 3 | | S21: | −0.0520 | S22: | −0.0019 | S23: | 0.0018 |
| REMARK | 3 | | S31: | −0.0496 | S32: | 0.0461 | S33: | 0.0471 |
| REMARK | 3 | | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE: | I | 33 | | I | 152 |

| | | | |
|---|---|---|---|
| REMARK | 3 | ORIGIN FOR THE GROUP (A): | −7.5740 51.3910 61.2800 |

| | | | | | | | | |
|---|---|---|---|---|---|---|---|---|
| REMARK | 3 | T | TENSOR | | | | | |
| REMARK | 3 | | T11: | 0.1810 | T22: | 0.1966 | | |
| REMARK | 3 | | T33: | 0.1218 | T12: | −0.0274 | | |
| REMARK | 3 | | T13: | 0.0045 | T23: | −0.0317 | | |
| REMARK | 3 | L | TENSOR | | | | | |
| REMARK | 3 | | L11: | 2.0882 | L22: | 2.1201 | | |
| REMARK | 3 | | L33: | 4.3804 | L12: | 0.4268 | | |
| REMARK | 3 | | L13: | −2.3533 | L23: | −0.0835 | | |
| REMARK | 3 | S | TENSOR | | | | | |
| REMARK | 3 | | S11: | 0.0647 | S12: | −0.3084 | S13: | 0.1729 |
| REMARK | 3 | | S21: | 0.3921 | S22: | −0.0441 | S23: | −0.0698 |
| REMARK | 3 | | S31: | −0.1554 | S32: | 0.0224 | S33: | −0.0206 |
| REMARK | 3 | | | | | | | |
| REMARK | 3 | | | | | | | |

| REMARK | 3 | BULK SOLVENT MODELLING. | | | | | |
|---|---|---|---|---|---|---|---|
| REMARK | 3 | METHOD USED : MASK | | | | | |
| REMARK | 3 | PARAMETERS FOR MASK CALCULATION | | | | | |
| REMARK | 3 | VDW PROBE RADIUS | | | | | : 1.20 |
| REMARK | 3 | ION PROBE RADIUS | | | | | : 0.80 |
| REMARK | 3 | SHRINKAGE RADIUS | | | | | : 0.80 |
| REMARK | 3 | | | | | | |
| REMARK | 3 | OTHER REFINEMENT REMARKS: | | | | | |
| REMARK | 3 | U VALUES : WITH TLS ADDED | | | | | |
| REMARK | 3 | HYDROGENS HAVE BEEN USED IF PRESENT IN THE INPUT | | | | | |
| REMARK | 3 | | | | | | |
| SSBOND | 1 | CYS | L | 88 | CYS | L | 23 |
| SSBOND | 2 | CYS | L | 134 | CYS | L | 214 |
| SSBOND | 3 | CYS | H | 96 | CYS | H | 22 |
| SSBOND | 4 | CYS | I | 71 | CYS | I | 122 |
| SSBOND | 5 | CYS | I | 78 | CYS | I | 125 |

| | | | | | | | | | |
|---|---|---|---|---|---|---|---|---|---|
| LINKR | SG | ACYS | L | 194 | SG | CYS | L | 134 | SS |
| LINKR | SG | BCYS | L | 194 | SG | CYS | L | 134 | SS |
| LINKR | SG | ACYS | H | 203 | SG | ACYS | H | 147 | SS |
| LINKR | SG | BCYS | H | 203 | SG | BCYS | H | 147 | SS |
| LINKR | | LYS | I | 106 | | ARG | I | 114 | gap |
| LINKR | | CYS | I | 78 | | SER | I | 86 | gap |

| | | | | | | | | |
|---|---|---|---|---|---|---|---|---|
| CISPEP | 1 | SER | L | 7 | PRO | L | 8 | 0.00 |
| CISPEP | 2 | TYR | L | 94 | PRO | L | 95 | 0.00 |
| CISPEP | 3 | TYR | L | 140 | PRO | L | 141 | 0.00 |
| CISPEP | 4 | PHE | H | 153 | PRO | H | 154 | 0.00 |
| CISPEP | 5 | GLU | H | 155 | PRO | H | 156 | 0.00 |

TABLE 14
hIL-21, chain I, (SEQ ID NO: 1) interactions with the the heavy chain
(chain H) of Fab56 (SEQ ID NO: 10, D62E mutation) and light chain
(chain L) of anti-IL-21 Fab56 (SEQ ID NO: 9). A distance cut-off of
5.0 Å was used. The contacts were identified by the CONTACT
computer software program of the CCP4 suite (Bailey, 1994). In the
last column “***” indicates a strong possibility for a hydrogen
bond at this contact (distance <3.3 Å) as calculated by CONTACT,
“*” indicates a weak possibility (distance >3.3 Å). Blank
indicates that the program considered there to be no possibility of a
hydrogen bond. Hydrogen-bonds are specific between a donor and
an acceptor, are typically strong, and are easily identifiable.

| | | | aIL-21 Fab56(Fab35 with | | | | |
|---|---|---|---|---|---|---|---|
| hIL-21 | | | H: D62E mutation) | | | | |
| | Res. # | | | Res. # | | | |
| Res. | and | Atom | Res. | and | Atom | Distance | Possibly |
| Type | Chain | name | Type | Chain | name | [Å] | H-bond |
| Met | 39I | CE | Trp | 102H | CZ3 | 4.46 | |
| | | | Trp | 102H | CH2 | 4.76 | |
| Glu | 65I | CB | Tyr | 56H | CZ | 4.90 | |
| | | | Tyr | 56H | OH | 4.71 | |
| | | | Tyr | 56H | CE2 | 4.59 | |
| Glu | 65I | CG | Tyr | 56H | CZ | 4.91 | |
| | | | Tyr | 56H | OH | 4.64 | |
| | | | Tyr | 56H | CE2 | 4.98 | |
| Glu | 65I | CD | Tyr | 56H | CZ | 4.44 | |
| | | | Tyr | 56H | OH | 3.82 | |
| | | | Tyr | 56H | CE2 | 4.86 | |
| Glu | 65I | OE1 | Tyr | 56H | CZ | 4.38 | |
| | | | Tyr | 56H | OH | 3.46 | * |
| | | | Tyr | 56H | CE2 | 4.79 | |
| Glu | 65I | OE2 | Tyr | 56H | CZ | 4.66 | |
| | | | Tyr | 56H | OH | 4.10 | * |
| Asp | 66I | N | Tyr | 56H | CE2 | 4.44 | |
| | | | Tyr | 56H | CD2 | 4.40 | |
| Asp | 66I | CA | Tyr | 56H | CG | 4.95 | |
| | | | Tyr | 56H | CD2 | 4.58 | |
| | | | Tyr | 57H | CE2 | 4.71 | |
| Asp | 66I | CB | Gly | 54H | O | 4.81 | |
| | | | Tyr | 56H | CG | 3.66 | |
| | | | Tyr | 56H | CD1 | 4.32 | |
| | | | Tyr | 56H | CE1 | 4.92 | |
| | | | Tyr | 56H | CZ | 4.87 | |
| | | | Tyr | 56H | CE2 | 4.31 | |
| | | | Tyr | 56H | CD2 | 3.64 | |
| | | | Gly | 54H | N | 4.56 | |
| | | | Gly | 54H | CA | 4.20 | |
| | | | Thr | 52H | OG1 | 4.74 | |
| | | | Gly | 54H | C | 4.64 | |
| | | | Tyr | 56H | N | 4.69 | |
| | | | Tyr | 56H | CA | 4.87 | |
| | | | Tyr | 56H | CB | 3.75 | |
| | | | Tyr | 57H | CE2 | 4.29 | |
| | | | Tyr | 57H | CD2 | 4.57 | |
| Asp | 66I | CG | Gly | 54H | O | 4.38 | |
| | | | Tyr | 56H | CG | 4.26 | |
| | | | Tyr | 56H | CD1 | 4.82 | |
| | | | Tyr | 56H | CD2 | 4.68 | |
| | | | Ser | 53H | OG | 4.81 | |
| | | | Ser | 53H | C | 4.61 | |
| | | | Gly | 54H | N | 3.35 | |
| | | | Gly | 54H | CA | 3.37 | |
| | | | Thr | 52H | CB | 3.95 | |
| | | | Thr | 52H | OG1 | 3.38 | |
| | | | Thr | 52H | C | 4.73 | |
| | | | Ser | 53H | N | 4.55 | |
| | | | Gly | 54H | C | 3.83 | |
| | | | Ser | 55H | N | 4.21 | |
| | | | Tyr | 56H | N | 4.06 | |
| | | | Tyr | 56H | CA | 4.60 | |
| | | | Tyr | 56H | CB | 3.88 | |
| | | | Tyr | 57H | CE2 | 4.52 | |
| | | | Tyr | 57H | CD2 | 4.32 | |
| | | | Thr | 52H | CA | 4.99 | |
| | | | Thr | 52H | CG2 | 4.99 | |
| Asp | 66I | OD1 | Ser | 53H | CA | 4.41 | |
| | | | Ser | 53H | CB | 4.66 | |
| | | | Ser | 53H | OG | 3.73 | * |
| | | | Ser | 53H | C | 4.19 | |
| | | | Gly | 54H | N | 3.10 | *** |
| | | | Gly | 54H | CA | 3.57 | |
| | | | Thr | 52H | CB | 3.66 | |
| | | | Thr | 52H | OG1 | 3.56 | * |
| | | | Thr | 52H | C | 4.31 | |
| | | | Ser | 53H | N | 3.83 | * |
| | | | Gly | 54H | C | 4.37 | |
| | | | Ser | 55H | N | 4.66 | * |
| | | | Tyr | 57H | CD2 | 4.92 | |
| | | | Thr | 52H | CA | 4.61 | |
| | | | Thr | 52H | CG2 | 4.70 | |
| Asp | 66I | OD2 | Gly | 54H | O | 3.69 | * |
| | | | Tyr | 56H | CG | 3.80 | |
| | | | Tyr | 56H | CD1 | 4.27 | |
| | | | Tyr | 56H | CD2 | 4.57 | |
| | | | Ser | 53H | C | 4.47 | |
| | | | Gly | 54H | N | 3.22 | *** |
| | | | Gly | 54H | CA | 3.14 | |
| | | | Ser | 55H | CA | 4.09 | |
| | | | Thr | 52H | CB | 3.56 | |
| | | | Thr | 52H | OG1 | 2.61 | *** |
| | | | Thr | 52H | C | 4.27 | |
| | | | Thr | 52H | O | 4.55 | * |
| | | | Ser | 53H | N | 4.42 | * |
| | | | Gly | 54H | C | 3.12 | |
| | | | Ser | 55H | N | 3.26 | *** |
| | | | Ser | 55H | C | 3.91 | |
| | | | Tyr | 56H | N | 2.87 | *** |
| | | | Tyr | 56H | CA | 3.56 | |
| | | | Tyr | 56H | CB | 3.18 | |
| | | | Tyr | 57H | N | 4.08 | * |
| | | | Tyr | 56H | C | 4.35 | |
| | | | Tyr | 57H | CE2 | 4.52 | |
| | | | Tyr | 57H | CD2 | 4.06 | |
| | | | Thr | 52H | CA | 4.49 | |
| | | | Thr | 52H | CG2 | 4.69 | |
| Asp | 66I | C | Tyr | 56H | CD2 | 4.96 | |
| | | | Tyr | 57H | CZ | 4.93 | |
| | | | Tyr | 57H | CE2 | 3.86 | |
| | | | Tyr | 57H | CD2 | 4.49 | |
| Asp | 66I | O | Tyr | 56H | CD2 | 4.66 | |
| | | | Tyr | 57H | CZ | 4.33 | |
| | | | Tyr | 57H | OH | 4.19 | * |
| | | | Tyr | 57H | CE2 | 3.57 | |
| | | | Tyr | 57H | CD2 | 4.54 | |
| Val | 67I | N | Tyr | 57H | CE2 | 4.00 | |
| | | | Tyr | 57H | CD2 | 4.32 | |
| Val | 67I | CA | Tyr | 57H | CE2 | 3.98 | |
| | | | Tyr | 57H | CD2 | 4.31 | |
| Val | 67I | C | Thr | 52H | CB | 4.77 | |
| | | | Tyr | 57H | CG | 4.60 | |
| | | | Tyr | 57H | CZ | 4.94 | |
| | | | Tyr | 57H | CE2 | 3.85 | |
| | | | Tyr | 57H | CD2 | 3.70 | |
| | | | Thr | 52H | CG2 | 4.20 | |
| Val | 67I | O | Thr | 52H | CB | 3.80 | |
| | | | Thr | 52H | OG1 | 4.37 | * |
| | | | Tyr | 57H | CG | 4.87 | |
| | | | Tyr | 57H | CE2 | 4.46 | |
| | | | Tyr | 57H | CD2 | 4.00 | |
| | | | Thr | 52H | CG2 | 3.30 | |
| Glu | 68I | N | Tyr | 57H | CB | 4.80 | |
| | | | Tyr | 57H | CG | 4.11 | |
| | | | Tyr | 57H | CD1 | 4.84 | |
| | | | Tyr | 57H | CZ | 4.51 | |
| | | | Tyr | 57H | CE2 | 3.71 | |
| | | | Tyr | 57H | CD2 | 3.55 | |
| | | | Thr | 52H | CG2 | 4.52 | |
| Glu | 68I | CA | Tyr | 57H | CB | 4.43 | |
| | | | Tyr | 57H | CG | 4.16 | |
| | | | Tyr | 57H | CD1 | 4.92 | |
| | | | Tyr | 57H | CE2 | 4.59 | |
| | | | Tyr | 57H | CD2 | 4.06 | |
| | | | Thr | 52H | CG2 | 4.07 | |
| Glu | 68I | CB | Tyr | 57H | CB | 4.90 | |
| | | | Tyr | 57H | CG | 4.56 | |
| | | | Tyr | 57H | CD1 | 4.93 | |
| | | | Tyr | 57H | CD2 | 4.77 | |
| Glu | 68I | CG | Tyr | 57H | CB | 4.31 | |
| | | | Tyr | 57H | CG | 3.78 | |
| | | | Tyr | 57H | CD1 | 3.78 | |
| | | | Tyr | 57H | CE1 | 4.16 | |
| | | | Tyr | 57H | CZ | 4.54 | |
| | | | Tyr | 57H | CE2 | 4.56 | |
| | | | Tyr | 57H | CD2 | 4.26 | |
| Glu | 68I | CD | Tyr | 57H | CB | 3.78 | |
| | | | Tyr | 57H | CG | 3.67 | |
| | | | Tyr | 57H | CD1 | 3.59 | |
| | | | Tyr | 57H | CE1 | 4.36 | |
| | | | Tyr | 57H | CD2 | 4.59 | |
| | | | His | 59H | CE1 | 4.29 | |
| | | | His | 59H | NE2 | 3.64 | |
| | | | His | 59H | CD2 | 4.79 | |
| Glu | 68I | OE1 | Tyr | 57H | CA | 4.92 | |
| | | | Tyr | 57H | CB | 3.61 | |
| | | | Tyr | 57H | CG | 3.98 | |
| | | | Tyr | 57H | CD1 | 4.22 | |
| | | | Tyr | 57H | CD2 | 4.90 | |
| | | | Tyr | 94L | CD1 | 4.89 | |
| | | | Tyr | 94L | CZ | 4.72 | |
| | | | His | 59H | CE1 | 3.85 | |
| | | | His | 59H | NE2 | 2.92 | *** |
| | | | His | 59H | CD2 | 3.86 | |
| | | | Tyr | 94L | CE1 | 4.13 | |
| | | | Tyr | 94L | OH | 4.54 | * |
| Glu | 68I | OE2 | Tyr | 57H | CB | 4.12 | |
| | | | Tyr | 57H | CG | 3.93 | |
| | | | Tyr | 57H | CD1 | 3.41 | |
| | | | Tyr | 57H | CE1 | 4.09 | |
| | | | His | 59H | CE1 | 3.89 | |
| | | | His | 59H | NE2 | 3.60 | * |
| | | | His | 59H | CD2 | 4.91 | |
| Glu | 68I | C | Thr | 52H | CG2 | 4.19 | |
| Thr | 69I | N | Ser | 33H | OG | 4.69 | * |
| | | | Thr | 52H | CG2 | 3.73 | |
| | | | Tyr | 94L | OH | 4.41 | * |
| Thr | 69I | CA | Ser | 33H | OG | 4.25 | |
| | | | Thr | 52H | CG2 | 4.60 | |
| | | | Tyr | 94L | OH | 4.65 | |
| Thr | 69I | CB | Ser | 33H | CB | 4.32 | |
| | | | Ser | 33H | OG | 3.64 | |
| | | | Thr | 52H | CG2 | 4.69 | |
| | | | Tyr | 94L | OH | 3.70 | |
| | | | Glu | 99H | CD | 4.56 | |
| | | | Glu | 99H | OE1 | 4.45 | |
| | | | Glu | 99H | OE2 | 4.43 | |
| | | | Tyr | 96L | OH | 4.29 | |
| Thr | 69I | OG1 | Thr | 52H | CB | 4.73 | |
| | | | Ser | 50H | OG | 4.39 | * |
| | | | Ser | 33H | CA | 4.76 | |
| | | | Ser | 33H | CB | 3.34 | |
| | | | Ser | 33H | OG | 2.63 | *** |
| | | | Thr | 52H | CA | 4.83 | |
| | | | Thr | 52H | CG2 | 3.55 | |
| | | | Tyr | 94L | OH | 3.81 | * |
| | | | Glu | 99H | CD | 4.84 | |
| | | | Glu | 99H | OE1 | 4.39 | * |
| | | | Tyr | 96L | OH | 4.98 | * |
| Thr | 69I | CG2 | Ser | 33H | CB | 4.05 | |
| | | | Ser | 33H | OG | 3.62 | |
| | | | Tyr | 94L | OH | 4.69 | |
| | | | Glu | 99H | CG | 4.21 | |
| | | | Glu | 99H | CD | 3.64 | |
| | | | Glu | 99H | OE1 | 3.88 | |
| | | | Glu | 99H | OE2 | 3.56 | |
| | | | Arg | 100H | C | 4.76 | |
| | | | Arg | 100H | O | 4.46 | |
| | | | Gly | 101H | N | 4.59 | |
| | | | Gly | 101H | CA | 4.12 | |
| | | | Tyr | 96L | OH | 4.23 | |
| Thr | 69I | O | Tyr | 94L | OH | 4.55 | * |
| Asn | 70I | N | Gly | 101H | CA | 4.06 | |
| | | | Gly | 101H | C | 4.65 | |
| | | | Trp | 102H | N | 4.32 | * |
| Asn | 70I | CA | Gly | 101H | CA | 4.46 | |
| | | | Gly | 101H | C | 4.60 | |
| | | | Trp | 102H | N | 4.08 | |
| | | | Trp | 102H | CA | 4.94 | |
| | | | Gly | 103H | N | 4.33 | |
| Asn | 70I | CB | Gly | 101H | CA | 4.90 | |
| | | | Trp | 102H | N | 4.84 | |
| | | | Gly | 103H | N | 4.35 | |
| | | | Gly | 103H | CA | 4.81 | |
| | | | Tyr | 105H | CE1 | 3.70 | |
| | | | Tyr | 105H | CZ | 4.37 | |
| | | | Tyr | 105H | OH | 4.41 | |
| | | | Tyr | 105H | CD1 | 4.40 | |
| Asn | 70I | CG | Arg | 100H | O | 4.53 | |
| | | | Gly | 101H | CA | 4.02 | |
| | | | Gly | 101H | C | 4.27 | |
| | | | Gly | 101H | O | 4.82 | |
| | | | Trp | 102H | N | 4.40 | |
| | | | Gly | 103H | N | 3.85 | |
| | | | Gly | 103H | CA | 4.27 | |
| | | | Tyr | 105H | CE1 | 3.48 | |
| | | | Tyr | 105H | CZ | 4.34 | |
| | | | Tyr | 105H | OH | 4.82 | |
| | | | Gly | 103H | C | 4.89 | |
| | | | Tyr | 104H | N | 4.83 | |
| | | | Tyr | 105H | N | 4.56 | |
| | | | Tyr | 105H | CA | 4.92 | |
| | | | Tyr | 105H | CD1 | 3.69 | |
| | | | Tyr | 105H | CG | 4.70 | |
| Asn | 70I | OD1 | Arg | 100H | C | 4.63 | |
| | | | Arg | 100H | O | 3.98 | * |
| | | | Gly | 101H | N | 4.42 | * |
| | | | Gly | 101H | CA | 3.24 | |
| | | | Gly | 101H | C | 3.21 | |
| | | | Gly | 101H | O | 3.65 | * |
| | | | Trp | 102H | N | 3.41 | * |
| | | | Trp | 102H | CA | 4.25 | |
| | | | Trp | 102H | C | 3.93 | |
| | | | Gly | 103H | N | 2.83 | *** |
| | | | Gly | 103H | CA | 3.43 | |
| | | | Tyr | 105H | CE1 | 4.00 | |
| | | | Tyr | 105H | CZ | 4.65 | |
| | | | Gly | 103H | C | 3.99 | |
| | | | Tyr | 104H | N | 3.80 | * |
| | | | Tyr | 104H | CA | 4.79 | |
| | | | Tyr | 104H | C | 4.95 | |
| | | | Tyr | 105H | N | 4.06 | * |
| | | | Tyr | 105H | CA | 4.77 | |
| | | | Tyr | 105H | CD1 | 4.06 | |
| | | | Tyr | 105H | CG | 4.75 | |
| Asn | 70I | ND2 | Glu | 99H | CD | 4.86 | |
| | | | Glu | 99H | OE2 | 3.88 | * |
| | | | Arg | 100H | O | 4.27 | * |
| | | | Gly | 101H | CA | 4.49 | |
| | | | Phe | 91L | CB | 4.58 | |
| | | | Phe | 91L | CD1 | 4.56 | |
| | | | Phe | 91L | O | 4.91 | * |
| | | | Tyr | 96L | OH | 4.28 | * |
| | | | Tyr | 105H | CE1 | 3.60 | |
| | | | Tyr | 105H | CZ | 4.76 | |
| | | | Tyr | 105H | N | 4.45 | * |
| | | | Phe | 91L | CG | 4.89 | |
| | | | Tyr | 105H | CA | 4.46 | |
| | | | Tyr | 105H | CD1 | 3.49 | |
| | | | Tyr | 105H | CG | 4.62 | |
| Asn | 70I | C | Trp | 102H | N | 4.89 | |
| Asn | 70I | O | Gly | 103H | N | 4.97 | * |
| | | | Tyr | 105H | OH | 4.66 | * |
| Glu | 72I | N | Trp | 102H | N | 4.92 | * |
| Glu | 72I | CA | Trp | 102H | CE2 | 4.68 | |
| | | | Trp | 102H | CZ3 | 4.56 | |
| | | | Trp | 102H | CH2 | 4.62 | |
| | | | Trp | 102H | CZ2 | 4.72 | |
| | | | Trp | 102H | CD2 | 4.59 | |
| | | | Trp | 102H | CE3 | 4.55 | |
| Glu | 72I | CB | Trp | 102H | NE1 | 3.77 | |
| | | | Trp | 102H | CE2 | 3.29 | |
| | | | Trp | 102H | CZ3 | 3.87 | |
| | | | Trp | 102H | CH2 | 3.78 | |
| | | | Trp | 102H | CZ2 | 3.53 | |
| | | | Trp | 102H | N | 4.37 | |
| | | | Trp | 102H | CA | 4.49 | |
| | | | Trp | 102H | CB | 4.83 | |
| | | | Trp | 102H | CG | 3.96 | |
| | | | Trp | 102H | CD1 | 4.11 | |
| | | | Trp | 102H | CD2 | 3.36 | |
| | | | Trp | 102H | CE3 | 3.68 | |
| Glu | 72I | CG | Trp | 102H | NE1 | 3.70 | |
| | | | Trp | 102H | CE2 | 3.73 | |
| | | | Trp | 102H | CH2 | 4.74 | |
| | | | Trp | 102H | CZ2 | 4.08 | |
| | | | Trp | 102H | N | 4.44 | |
| | | | Trp | 102H | CG | 4.43 | |
| | | | Trp | 102H | CD1 | 4.10 | |
| | | | Trp | 102H | CD2 | 4.17 | |
| | | | Trp | 102H | CE3 | 4.86 | |
| Glu | 72I | CD | Trp | 102H | NE1 | 4.23 | |
| | | | Trp | 102H | CE2 | 4.57 | |
| | | | Gly | 101H | CA | 4.08 | |
| | | | Gly | 101H | C | 4.25 | |
| | | | Trp | 102H | N | 3.53 | |
| | | | Trp | 102H | CA | 4.45 | |
| | | | Trp | 102H | CG | 4.51 | |
| | | | Trp | 102H | CD1 | 4.16 | |
| | | | Trp | 102H | CD2 | 4.72 | |
| Glu | 72I | OE1 | Trp | 102H | NE1 | 4.62 | * |
| | | | Trp | 102H | CE2 | 4.84 | |
| | | | Gly | 101H | N | 4.94 | * |
| | | | Gly | 101H | CA | 3.65 | |
| | | | Gly | 101H | C | 3.56 | |
| | | | Gly | 101H | O | 4.75 | * |
| | | | Trp | 102H | N | 2.65 | *** |
| | | | Trp | 102H | CA | 3.53 | |
| | | | Trp | 102H | CB | 4.52 | |
| | | | Trp | 102H | CG | 4.24 | |
| | | | Trp | 102H | CD1 | 4.21 | |
| | | | Trp | 102H | C | 4.63 | |
| | | | Gly | 103H | N | 4.65 | * |
| | | | Trp | 102H | CD2 | 4.60 | |
| Glu | 72I | OE2 | Ser | 033H | OG | 4.96 | * |
| | | | Trp | 102H | NE1 | 4.78 | * |
| | | | Gly | 101H | N | 4.82 | * |
| | | | Gly | 101H | CA | 3.94 | |
| | | | Gly | 101H | C | 4.55 | |
| | | | Trp | 102H | N | 4.15 | * |
| | | | Trp | 102H | CD1 | 4.71 | |
| Glu | 72I | C | Trp | 102H | CE2 | 4.95 | |
| | | | Trp | 102H | CZ3 | 3.92 | |
| | | | Trp | 102H | CH2 | 4.31 | |
| | | | Trp | 102H | CZ2 | 4.84 | |
| | | | Trp | 102H | CD2 | 4.57 | |
| | | | Trp | 102H | CE3 | 4.08 | |
| Glu | 72I | O | Trp | 102H | CZ3 | 3.76 | |
| | | | Trp | 102H | CH2 | 3.99 | |
| | | | Trp | 102H | CZ2 | 4.76 | |
| | | | Trp | 102H | CE3 | 4.35 | |
| Trp | 73I | N | Trp | 102H | CZ3 | 4.22 | |
| | | | Trp | 102H | CH2 | 4.98 | |
| | | | Trp | 102H | CA | 4.93 | |
| | | | Trp | 102H | CD2 | 4.72 | |
| | | | Trp | 102H | CE3 | 4.08 | |
| Trp | 73I | CA | Trp | 102H | CZ3 | 4.41 | |
| | | | Trp | 102H | CE3 | 4.45 | |
| Trp | 73I | CB | Trp | 102H | CE3 | 4.82 | |
| Trp | 73I | CG | Trp | 102H | CZ3 | 4.42 | |
| | | | Trp | 102H | CA | 4.84 | |
| | | | Trp | 102H | C | 4.91 | |
| | | | Trp | 102H | O | 4.65 | |
| | | | Trp | 102H | CD2 | 4.97 | |
| | | | Trp | 102H | CE3 | 3.93 | |
| Trp | 73I | CD1 | Trp | 102H | CZ3 | 4.82 | |
| | | | Trp | 102H | CA | 3.82 | |
| | | | Trp | 102H | CB | 4.12 | |
| | | | Trp | 102H | CG | 4.87 | |
| | | | Trp | 102H | C | 3.64 | |
| | | | Trp | 102H | O | 3.26 | |
| | | | Gly | 103H | N | 4.48 | |
| | | | Trp | 102H | CD2 | 4.70 | |
| | | | Trp | 102H | CE3 | 3.93 | |
| Trp | 73I | NE1 | Trp | 102H | CZ3 | 4.61 | |
| | | | Trp | 102H | CA | 3.99 | |
| | | | Trp | 102H | CB | 3.77 | |
| | | | Trp | 102H | CG | 4.56 | |
| | | | Trp | 102H | C | 3.85 | |
| | | | Trp | 102H | O | 3.15 | *** |
| | | | Gly | 103H | N | 4.95 | * |
| | | | Trp | 102H | CD2 | 4.42 | |
| | | | Trp | 102H | CE3 | 3.64 | |
| Trp | 73I | CE2 | Trp | 102H | CZ3 | 4.04 | |
| | | | Trp | 102H | CB | 4.59 | |
| | | | Trp | 102H | O | 4.50 | |
| | | | Trp | 102H | CD2 | 4.52 | |
| | | | Trp | 102H | CE3 | 3.44 | |
| Trp | 73I | CD2 | Trp | 102H | CZ3 | 3.89 | |
| | | | Trp | 102H | CD2 | 4.88 | |
| | | | Trp | 102H | CE3 | 3.63 | |
| Trp | 73I | CE3 | Trp | 102H | CZ3 | 3.96 | |
| | | | Trp | 102H | CE3 | 4.18 | |
| Trp | 73I | CZ3 | Trp | 102H | CZ3 | 4.14 | |
| | | | Trp | 102H | CE3 | 4.48 | |
| Trp | 73I | CH2 | Trp | 102H | CZ3 | 4.31 | |
| | | | Trp | 102H | CE3 | 4.36 | |
| Trp | 73I | CZ2 | Trp | 102H | CZ3 | 4.26 | |
| | | | Trp | 102H | CD2 | 4.91 | |
| | | | Trp | 102H | CE3 | 3.86 | |
| Phe | 76I | CB | Trp | 102H | CZ3 | 4.37 | |
| | | | Trp | 102H | CH2 | 4.74 | |
| Phe | 76I | CG | Trp | 102H | CZ3 | 4.98 | |
| Lys | 117I | N | Trp | 102H | O | 4.87 | * |
| Lys | 117I | CA | Gly | 103H | CA | 4.79 | |
| Lys | 117I | CB | Gly | 103H | CA | 4.27 | |
| | | | Tyr | 105H | CZ | 4.83 | |
| | | | Tyr | 105H | OH | 4.43 | |
| | | | Gly | 103H | O | 4.83 | |
| | | | Tyr | 105H | CE2 | 4.28 | |
| Lys | 117I | CG | Tyr | 105H | CE2 | 4.97 | |
| Lys | 117I | CD | Ser | 31L | OG | 3.88 | |
| | | | Asp | 50L | CG | 4.18 | |
| | | | Asp | 50L | OD1 | 3.74 | |
| | | | Asp | 50L | OD2 | 3.96 | |
| | | | Gly | 103H | O | 4.45 | |
| | | | Tyr | 105H | CE2 | 4.31 | |
| | | | Tyr | 105H | CD2 | 4.75 | |
| Lys | 117I | CE | Ser | 31L | OG | 4.08 | |
| | | | Asp | 50L | CG | 4.13 | |
| | | | Asp | 50L | OD1 | 3.99 | |
| | | | Asp | 50L | OD2 | 3.51 | |
| Lys | 117I | NZ | Ser | 31L | OG | 3.17 | *** |
| | | | Asp | 50L | CG | 3.58 | |
| | | | Asp | 50L | OD1 | 3.41 | * |
| | | | Asp | 50L | OD2 | 2.96 | *** |
| | | | Ser | 31L | CB | 3.99 | |
| Lys | 117I | C | Gly | 103H | N | 4.70 | |
| | | | Gly | 103H | CA | 4.31 | |
| | | | Tyr | 105H | OH | 4.44 | |
| Lys | 117I | O | Trp | 102H | CA | 4.70 | |
| | | | Trp | 102H | C | 3.91 | |
| | | | Trp | 102H | O | 4.04 | * |
| | | | Gly | 103H | N | 3.67 | * |
| | | | Gly | 103H | CA | 3.58 | |
| | | | Tyr | 105H | OH | 4.75 | * |
| | | | Gly | 103H | C | 4.99 | |
| His | 118I | N | Tyr | 105H | OH | 4.06 | * |
| His | 118I | CA | Tyr | 105H | OH | 3.86 | |
| His | 118I | C | Tyr | 105H | CZ | 4.81 | |
| | | | Tyr | 105H | OH | 3.46 | |
| His | 118I | O | Tyr | 105H | OH | 3.73 | * |
| Arg | 119I | N | Tyr | 105H | CZ | 4.78 | |
| | | | Tyr | 105H | OH | 3.50 | * |
| Arg | 119I | CA | Tyr | 105H | OH | 4.04 | * |
| Arg | 119I | CB | Tyr | 105H | OH | 4.66 | |
| | | | Asn | 92L | O | 4.54 | |
| Arg | 119I | CG | Phe | 91L | O | 4.26 | |
| | | | Tyr | 105H | CE1 | 4.44 | |
| | | | Tyr | 105H | CZ | 4.70 | |
| | | | Tyr | 105H | OH | 3.99 | |
| | | | Asn | 92L | CA | 4.96 | |
| | | | Asn | 92L | C | 4.89 | |
| | | | Asn | 92L | O | 4.15 | |
| Arg | 119I | CD | Phe | 91L | C | 4.58 | |
| | | | Phe | 91L | O | 3.47 | |
| | | | Asn | 92L | N | 4.94 | |
| | | | Asn | 92L | CA | 4.32 | |
| | | | Asn | 92L | C | 3.89 | |
| | | | Asn | 92L | O | 3.15 | |
| | | | Ser | 93L | N | 4.83 | |
| Arg | 119I | NE | Tyr | 94L | CE1 | 4.82 | |
| | | | Phe | 91L | O | 4.07 | * |
| | | | Asn | 92L | C | 4.90 | |
| | | | Asn | 92L | O | 4.21 | * |
| Arg | 119I | CZ | Tyr | 94L | CD1 | 4.94 | |
| | | | Tyr | 94L | CE1 | 4.31 | |
| | | | Asn | 92L | O | 4.69 | |
| Arg | 119I | NH1 | Tyr | 94L | CE1 | 4.79 | |
| | | | Asn | 92L | O | 4.30 | * |
| Arg | 119I | NH2 | Tyr | 94L | CD1 | 4.71 | |
| | | | Tyr | 94L | CZ | 4.61 | |
| | | | Tyr | 94L | CE1 | 3.83 | |
| | | | Tyr | 94L | OH | 4.39 | * |
| Thr | 121I | OG1 | Asn | 92L | O | 4.98 | * |
| Tyr | 128I | CE1 | Tyr | 57H | OH | 4.78 | |
| Glu | 129I | CD | Tyr | 56H | OH | 4.89 | |
| Glu | 129I | OE1 | Tyr | 56H | OH | 4.79 | * |
| Glu | 129I | OE2 | Tyr | 56H | OH | 4.68 | * |
| Leu | 143I | CA | Trp | 102H | CZ2 | 4.82 | |
| Leu | 143I | CB | Trp | 102H | CH2 | 4.85 | |
| | | | Trp | 102H | CZ2 | 4.89 | |
| Leu | 143I | CG | Trp | 102H | CZ3 | 4.82 | |
| | | | Trp | 102H | CH2 | 3.61 | |
| | | | Trp | 102H | CZ2 | 3.72 | |
| Leu | 143I | CD1 | Trp | 102H | CE2 | 4.72 | |
| | | | Trp | 102H | CH2 | 4.01 | |
| | | | Trp | 102H | CZ2 | 3.69 | |
| Leu | 143I | CD2 | Trp | 102H | CZ3 | 4.86 | |
| | | | Trp | 102H | CH2 | 3.96 | |
| | | | Trp | 102H | CZ2 | 4.49 | |
| Leu | 143I | C | Trp | 102H | CH2 | 4.40 | |
| | | | Trp | 102H | CZ2 | 4.19 | |
| Leu | 143I | O | Trp | 102H | NE1 | 4.96 | * |
| | | | Trp | 102H | CE2 | 4.42 | |
| | | | Trp | 102H | CH2 | 3.78 | |
| | | | Trp | 102H | CZ2 | 3.32 | |
| Gln | 145I | O | Ser | 31H | OG | 4.83 | * |
| Lys | 146I | N | Ser | 31H | OG | 4.77 | * |
| Lys | 146I | CA | Ser | 31H | OG | 4.04 | |
| | | | Ser | 31H | CB | 4.40 | |
| | | | Ser | 31H | O | 4.94 | |
| | | | Trp | 102H | NE1 | 4.95 | |
| Lys | 146I | CB | Ser | 31H | OG | 4.34 | |
| | | | Ser | 31H | CA | 4.81 | |
| | | | Ser | 31H | CB | 4.52 | |
| | | | Ser | 31H | O | 4.37 | |
| | | | Trp | 102H | NE1 | 4.44 | |
| | | | Trp | 102H | CE2 | 4.89 | |
| | | | Trp | 102H | CZ2 | 4.61 | |
| Lys | 146I | CG | Ser | 30H | O | 4.77 | |
| | | | Ser | 31H | OG | 3.67 | |
| | | | Ser | 31H | C | 4.66 | |
| | | | Ser | 31H | CA | 4.08 | |
| | | | Ser | 31H | CB | 4.07 | |
| | | | Ser | 31H | O | 4.27 | |
| Lys | 146I | CD | Ser | 30H | O | 4.61 | |
| | | | Ser | 53H | OG | 4.20 | |
| | | | Ser | 31H | OG | 4.83 | |
| | | | Ser | 31H | C | 4.91 | |
| | | | Ser | 31H | CA | 4.58 | |
| | | | Ser | 31H | O | 4.53 | |
| Lys | 146I | CE | Ser | 30H | O | 3.69 | |
| | | | Ser | 53H | CB | 4.39 | |
| | | | Ser | 53H | OG | 3.67 | |
| | | | Ser | 31H | OG | 4.94 | |
| | | | Ser | 30H | C | 4.77 | |
| | | | Ser | 31H | CA | 4.53 | |
| Lys | 146I | NZ | Ser | 30H | O | 4.61 | * |
| Lys | 146I | C | Trp | 102H | NE1 | 4.12 | |
| | | | Trp | 102H | CE2 | 4.53 | |
| | | | Trp | 102H | CZ2 | 4.56 | |
| Lys | 146I | O | Ser | 31H | OG | 4.93 | * |
| | | | Ser | 31H | CB | 4.74 | |
| | | | Ser | 31H | O | 4.93 | * |
| | | | Trp | 102H | NE1 | 4.35 | * |
| | | | Trp | 102H | CE2 | 4.99 | |
| | | | Trp | 102H | CD1 | 4.93 | |
| Met | 147I | N | Trp | 102H | NE1 | 3.83 | * |
| | | | Trp | 102H | CE2 | 3.84 | |
| | | | Trp | 102H | CH2 | 4.55 | |
| | | | Trp | 102H | CZ2 | 3.70 | |
| | | | Trp | 102H | CD1 | 4.77 | |
| | | | Trp | 102H | CD2 | 4.85 | |
| Met | 147I | CA | Trp | 102H | NE1 | 3.81 | |
| | | | Trp | 102H | CE2 | 3.70 | |
| | | | Trp | 102H | CZ3 | 4.92 | |
| | | | Trp | 102H | CH2 | 4.45 | |
| | | | Trp | 102H | CZ2 | 3.84 | |
| | | | Trp | 102H | CG | 4.65 | |
| | | | Trp | 102H | CD1 | 4.40 | |
| | | | Trp | 102H | CD2 | 4.28 | |
| | | | Trp | 102H | CE3 | 4.85 | |
| Met | 147I | CB | Trp | 102H | NE1 | 4.29 | |
| | | | Trp | 102H | CE2 | 3.67 | |
| | | | Trp | 102H | CZ3 | 3.92 | |
| | | | Trp | 102H | CH2 | 3.60 | |
| | | | Trp | 102H | CZ2 | 3.48 | |
| | | | Trp | 102H | CG | 4.77 | |
| | | | Trp | 102H | CD1 | 4.92 | |
| | | | Trp | 102H | CD2 | 4.01 | |
| | | | Trp | 102H | CE3 | 4.13 | |
| Met | 147I | CG | Trp | 102H | NE1 | 4.91 | |
| | | | Trp | 102H | CE2 | 4.18 | |
| | | | Trp | 102H | CZ3 | 3.80 | |
| | | | Trp | 102H | CH2 | 4.04 | |
| | | | Trp | 102H | CZ2 | 4.25 | |
| | | | Trp | 102H | CG | 4.62 | |
| | | | Trp | 102H | CD2 | 3.95 | |
| | | | Trp | 102H | CE3 | 3.75 | |
| His | 149I | CB | Ile | 28H | CB | 4.63 | |
| | | | Ile | 28H | CG1 | 4.77 | |
| | | | Ser | 31H | OG | 3.88 | |
| | | | Ser | 31H | CB | 4.06 | |
| His | 149I | CG | Ile | 28H | CB | 4.09 | |
| | | | Ile | 28H | CG1 | 3.98 | |
| | | | Ile | 28H | CG2 | 4.37 | |
| | | | Ser | 31H | OG | 3.82 | |
| | | | Ser | 31H | CB | 4.44 | |
| His | 149I | ND1 | Ile | 28H | CB | 3.88 | |
| | | | Ile | 28H | CG1 | 4.01 | |
| | | | Ile | 28H | CG2 | 3.68 | |
| | | | Ser | 31H | OG | 2.97 | *** |
| | | | Ser | 31H | CB | 3.96 | |
| His | 149I | CE1 | Ile | 28H | CD1 | 4.64 | |
| | | | Ile | 28H | CB | 4.10 | |
| | | | Ile | 28H | CG1 | 3.89 | |
| | | | Ile | 28H | CG2 | 3.71 | |
| | | | Ser | 31H | OG | 3.93 | |
| His | 149I | NE2 | Ile | 28H | CD1 | 4.45 | |
| | | | Ile | 28H | CB | 4.43 | |
| | | | Ile | 28H | CG1 | 3.76 | |
| | | | Ile | 28H | CG2 | 4.41 | |
| His | 149I | CD2 | Ile | 28H | CD1 | 4.89 | |
| | | | Ile | 28H | CB | 4.48 | |
| | | | Ile | 28H | CG1 | 3.86 | |
| | | | Ile | 28H | CG2 | 4.83 | |
| His | 149I | C | Tyr | 32H | OH | 4.01 | |
| | | | Tyr | 32H | CZ | 4.79 | |
| His | 149I | O | Tyr | 32H | OH | 3.54 | * |
| | | | Tyr | 32H | CZ | 4.60 | |
| Gln | 150I | N | Tyr | 32H | OH | 4.10 | * |
| | | | Tyr | 32H | CZ | 4.66 | |
| Gln | 150I | CA | Tyr | 32H | CE2 | 4.95 | |
| | | | Tyr | 32H | OH | 3.50 | |
| | | | Tyr | 32H | CE1 | 4.46 | |
| | | | Tyr | 32H | CZ | 4.10 | |
| | | | Arg | 100H | NE | 4.69 | |
| | | | Arg | 100H | CZ | 4.90 | |
| | | | Arg | 100H | NH2 | 4.83 | |
| Gln | 150I | CB | Tyr | 32H | OH | 4.22 | |
| | | | Tyr | 32H | CE1 | 4.34 | |
| | | | Tyr | 32H | CZ | 4.40 | |
| | | | Arg | 100H | CG | 4.75 | |
| | | | Trp | 102H | CD1 | 4.81 | |
| | | | Arg | 100H | CD | 4.93 | |
| | | | Arg | 100H | NE | 4.19 | |
| | | | Arg | 100H | CZ | 4.68 | |
| | | | Arg | 100H | NH2 | 4.57 | |
| Gln | 150I | CG | Ser | 31H | C | 4.95 | |
| | | | Tyr | 32H | CD2 | 4.71 | |
| | | | Ser | 31H | CB | 4.97 | |
| | | | Ser | 31H | O | 4.10 | |
| | | | Trp | 102H | NE1 | 4.86 | |
| | | | Tyr | 32H | CE2 | 4.31 | |
| | | | Tyr | 32H | OH | 4.06 | |
| | | | Tyr | 32H | CG | 4.66 | |
| | | | Tyr | 32H | CD1 | 4.11 | |
| | | | Tyr | 32H | CE1 | 3.67 | |
| | | | Tyr | 32H | CZ | 3.77 | |
| | | | Arg | 100H | CG | 4.89 | |
| | | | Trp | 102H | CD1 | 4.64 | |
| Gln | 150I | CD | Ser | 31H | C | 4.86 | |
| | | | Tyr | 32H | CA | 4.97 | |
| | | | Ser | 31H | O | 3.87 | |
| | | | Trp | 102H | NE1 | 4.32 | |
| | | | Tyr | 32H | CE2 | 5.00 | |
| | | | Tyr | 32H | CG | 4.54 | |
| | | | Tyr | 32H | CD1 | 3.92 | |
| | | | Tyr | 32H | CE1 | 3.91 | |
| | | | Tyr | 32H | CZ | 4.47 | |
| | | | Arg | 100H | CB | 4.46 | |
| | | | Arg | 100H | CG | 4.41 | |
| | | | Arg | 100H | CA | 4.25 | |
| | | | Arg | 100H | C | 4.67 | |
| | | | Gly | 101H | N | 3.97 | |
| | | | Gly | 101H | CA | 4.94 | |
| | | | Trp | 102H | CD1 | 3.90 | |
| Gln | 150I | OE1 | Ser | 31H | O | 4.98 | * |
| | | | Trp | 102H | NE1 | 4.34 | * |
| | | | Tyr | 32H | CD1 | 4.50 | |
| | | | Tyr | 32H | CE1 | 4.44 | |
| | | | Glu | 99H | O | 4.97 | * |
| | | | Arg | 100H | CB | 3.55 | |
| | | | Arg | 100H | CG | 3.81 | |
| | | | Arg | 100H | N | 4.88 | * |
| | | | Arg | 100H | CA | 3.50 | |
| | | | Arg | 100H | C | 3.75 | |
| | | | Arg | 100H | O | 4.94 | * |
| | | | Gly | 101H | N | 3.08 | *** |
| | | | Gly | 101H | CA | 4.05 | |
| | | | Gly | 101H | C | 4.11 | |
| | | | Gly | 101H | O | 4.06 | * |
| | | | Trp | 102H | N | 4.92 | * |
| | | | Trp | 102H | CB | 4.87 | |
| | | | Trp | 102H | CG | 4.53 | |
| | | | Trp | 102H | CD1 | 3.54 | |
| | | | Arg | 100H | CD | 4.67 | |
| | | | Arg | 100H | NE | 4.23 | * |
| Gln | 150I | NE2 | Ser | 31H | C | 3.83 | |
| | | | Tyr | 32H | N | 4.34 | * |
| | | | Tyr | 32H | CA | 3.88 | |
| | | | Tyr | 32H | CB | 4.43 | |
| | | | Tyr | 32H | CD2 | 4.60 | |
| | | | Tyr | 32H | C | 4.97 | |
| | | | Ser | 33H | N | 4.94 | * |
| | | | Ser | 31H | O | 2.79 | *** |
| | | | Trp | 102H | NE1 | 4.42 | * |
| | | | Tyr | 32H | CE2 | 4.93 | |
| | | | Tyr | 32H | CG | 4.03 | |
| | | | Tyr | 32H | CD1 | 3.75 | |
| | | | Tyr | 32H | CE1 | 4.15 | |
| | | | Tyr | 32H | CZ | 4.72 | |
| | | | Glu | 99H | O | 4.70 | * |
| | | | Arg | 100H | CA | 4.51 | |
| | | | Arg | 100H | C | 4.83 | |
| | | | Gly | 101H | N | 4.06 | * |
| | | | Gly | 101H | CA | 4.96 | |
| | | | Trp | 102H | CD1 | 4.25 | |
| Gln | 150I | C | Tyr | 32H | OH | 4.30 | |
| | | | Arg | 100H | NE | 4.30 | |
| | | | Arg | 100H | CZ | 4.10 | |
| | | | Arg | 100H | NH1 | 4.71 | |
| | | | Arg | 100H | NH2 | 3.86 | |
| Gln | 150I | O | Tyr | 32H | OH | 4.24 | * |
| | | | Arg | 100H | CD | 4.34 | |
| | | | Arg | 100H | NE | 3.59 | * |
| | | | Arg | 100H | CZ | 3.25 | |
| | | | Arg | 100H | NH1 | 3.64 | * |
| | | | Arg | 100H | NH2 | 3.28 | *** |
| His | 151I | N | Arg | 100H | CZ | 4.89 | |
| | | | Arg | 100H | NH2 | 4.33 | * |
| His | 151I | CA | Arg | 100H | CZ | 4.89 | |
| | | | Arg | 100H | NH2 | 4.12 | |
| His | 151I | CB | Arg | 100H | NH2 | 4.46 | |
| His | 151I | CG | Arg | 100H | CZ | 4.97 | |
| | | | Arg | 100H | NH2 | 3.76 | |
| His | 151I | ND1 | Arg | 100H | NH2 | 4.17 | * |
| His | 151I | CE1 | Trp | 102H | CB | 3.87 | |
| | | | Trp | 102H | CG | 4.10 | |
| | | | Trp | 102H | CD1 | 4.57 | |
| | | | Trp | 102H | CD2 | 4.75 | |
| | | | Arg | 100H | NH2 | 4.11 | |
| His | 151I | NE2 | Trp | 102H | CB | 4.38 | |
| | | | Trp | 102H | CG | 5.00 | |
| | | | Arg | 100H | CZ | 4.83 | |
| | | | Arg | 100H | NH2 | 3.57 | * |
| His | 151I | CD2 | Arg | 100H | CZ | 4.65 | |
| | | | Arg | 100H | NH2 | 3.32 | |

TABLE 15
hIL-21, chain I, (SEQ ID NO: 1) interactions with the the heavy chain
(chain H) of Fab57 (SEQ ID No 10, K65R mutation) and light chain
(chain L) of anti-IL-21 Fab57 (SEQ ID No 9). A distance cut-off of
5.0 Å was used. The contacts were identified by the CONTACT
computer software program of the CCP4 suite (Bailey, 1994).
In the last column “***” indicates a strong possibility for a hydrogen
bond at this contact (distance <3.3 Å) as calculated by CONTACT,
“*” indicates a weak possibility (distance >3.3 Å). Blank indicates
that the program considered there to be no possibility of a hydrogen bond.
Hydrogen-bonds are specific between a donor and an acceptor,
are typically strong, and are easily identifiable.

| | | | aIL-21 Fab57 | | | | |
|---|---|---|---|---|---|---|---|
| hIL-21 | | | (Fab35 with H: K65R) | | | | |
| | Res. # | | | Res. # | | | |
| Res. | and | Atom | Res. | and | Atom | Distance | Possibly |
| Type | Chain | name | T ype | Chain | name | [Å] | H-bond |
| Met | 39I | CE | Trp | 102H | CZ3 | 4.39 | |
| | | | Trp | 102H | CH2 | 4.71 | |
| Glu | 65I | CB | Tyr | 56H | CE2 | 4.83 | |
| Glu | 65I | CD | Tyr | 56H | OH | 4.73 | |
| Glu | 65I | OE1 | Tyr | 56H | CZ | 4.36 | |
| | | | Tyr | 56H | OH | 3.75 | * |
| | | | Tyr | 56H | CE2 | 4.53 | |
| Asp | 66I | N | Tyr | 56H | CE2 | 4.77 | |
| | | | Tyr | 56H | CD2 | 4.49 | |
| Asp | 66I | CA | Tyr | 56H | CD2 | 4.76 | |
| | | | Tyr | 57H | CE2 | 4.75 | |
| Asp | 66I | CB | Gly | 54H | O | 4.76 | |
| | | | Tyr | 56H | CG | 3.82 | |
| | | | Tyr | 56H | CD1 | 4.65 | |
| | | | Tyr | 56H | CE2 | 4.65 | |
| | | | Tyr | 56H | CD2 | 3.84 | |
| | | | Gly | 54H | N | 4.45 | |
| | | | Gly | 54H | CA | 4.10 | |
| | | | Thr | 52H | OG1 | 4.69 | |
| | | | Gly | 54H | C | 4.56 | |
| | | | Tyr | 56H | N | 4.63 | |
| | | | Tyr | 56H | CA | 4.85 | |
| | | | Tyr | 56H | CB | 3.74 | |
| | | | Tyr | 57H | CE2 | 4.44 | |
| | | | Tyr | 57H | CD2 | 4.66 | |
| Asp | 66I | CG | Gly | 54H | O | 4.38 | |
| | | | Tyr | 56H | CG | 4.42 | |
| | | | Tyr | 56H | CD2 | 4.87 | |
| | | | Ser | 53H | OG | 4.67 | |
| | | | Ser | 53H | C | 4.53 | |
| | | | Gly | 54H | N | 3.27 | |
| | | | Gly | 54H | CA | 3.33 | |
| | | | Thr | 52H | CB | 3.90 | |
| | | | Thr | 52H | OG1 | 3.26 | |
| | | | Thr | 52H | C | 4.61 | |
| | | | Ser | 53H | N | 4.46 | |
| | | | Gly | 54H | C | 3.80 | |
| | | | Ser | 55H | N | 4.14 | |
| | | | Tyr | 56H | N | 3.99 | |
| | | | Tyr | 56H | CA | 4.56 | |
| | | | Tyr | 56H | CB | 3.87 | |
| | | | Tyr | 57H | CE2 | 4.57 | |
| | | | Tyr | 57H | CD2 | 4.32 | |
| | | | Thr | 52H | CA | 4.90 | |
| | | | Thr | 52H | CG2 | 4.95 | |
| Asp | 66I | OD1 | Ser | 53H | CA | 4.31 | |
| | | | Ser | 53H | CB | 4.58 | |
| | | | Ser | 53H | OG | 3.59 | * |
| | | | Ser | 53H | C | 4.12 | |
| | | | Gly | 54H | N | 3.06 | *** |
| | | | Gly | 54H | CA | 3.57 | |
| | | | Thr | 52H | CB | 3.64 | |
| | | | Thr | 52H | OG1 | 3.50 | * |
| | | | Thr | 52H | C | 4.22 | |
| | | | Thr | 52H | O | 4.96 | * |
| | | | Ser | 53H | N | 3.76 | * |
| | | | Gly | 54H | C | 4.38 | |
| | | | Ser | 55H | N | 4.65 | * |
| | | | Tyr | 57H | CD2 | 4.93 | |
| | | | Thr | 52H | CA | 4.56 | |
| | | | Thr | 52H | CG2 | 4.71 | |
| Asp | 66I | OD2 | Gly | 54H | O | 3.67 | * |
| | | | Tyr | 56H | CG | 3.88 | |
| | | | Tyr | 56H | CD1 | 4.48 | |
| | | | Tyr | 56H | CD2 | 4.67 | |
| | | | Ser | 53H | C | 4.46 | |
| | | | Gly | 54H | N | 3.21 | *** |
| | | | Gly | 54H | CA | 3.13 | |
| | | | Ser | 55H | O | 4.89 | * |
| | | | Thr | 52H | CB | 3.59 | |
| | | | Thr | 52H | OG1 | 2.56 | *** |
| | | | Thr | 52H | C | 4.23 | |
| | | | Thr | 52H | O | 4.51 | * |
| | | | Ser | 53H | N | 4.43 | * |
| | | | Gly | 54H | C | 3.10 | |
| | | | Ser | 55H | N | 3.22 | *** |
| | | | Ser | 55H | CA | 4.03 | |
| | | | Ser | 55H | C | 3.81 | |
| | | | Tyr | 56H | N | 2.78 | *** |
| | | | Tyr | 56H | CA | 3.48 | |
| | | | Tyr | 56H | CB | 3.12 | |
| | | | Tyr | 56H | C | 4.29 | |
| | | | Tyr | 57H | N | 4.05 | * |
| | | | Tyr | 57H | CE2 | 4.59 | |
| | | | Tyr | 57H | CD2 | 4.10 | |
| | | | Thr | 52H | CA | 4.48 | |
| | | | Thr | 52H | CG2 | 4.73 | |
| Asp | 66I | C | Tyr | 57H | CZ | 4.83 | |
| | | | Tyr | 57H | CE2 | 3.80 | |
| | | | Tyr | 57H | CD2 | 4.45 | |
| | | | Tyr | 57H | OH | 4.82 | |
| Asp | 66I | O | Tyr | 56H | CD2 | 4.84 | |
| | | | Tyr | 57H | CZ | 4.31 | |
| | | | Tyr | 57H | CE2 | 3.58 | |
| | | | Tyr | 57H | CD2 | 4.55 | |
| | | | Tyr | 57H | OH | 3.99 | * |
| Val | 67I | N | Tyr | 57H | CZ | 4.95 | |
| | | | Tyr | 57H | CE2 | 3.81 | |
| | | | Tyr | 57H | CD2 | 4.19 | |
| Val | 67I | CA | Tyr | 57H | CZ | 4.66 | |
| | | | Tyr | 57H | CE2 | 3.73 | |
| | | | Tyr | 57H | CD2 | 4.18 | |
| | | | Tyr | 57H | OH | 4.83 | |
| Val | 67I | C | Thr | 52H | CB | 4.79 | |
| | | | Tyr | 57H | CG | 4.55 | |
| | | | Tyr | 57H | CZ | 4.60 | |
| | | | Tyr | 57H | CE2 | 3.62 | |
| | | | Tyr | 57H | CD2 | 3.59 | |
| | | | Thr | 52H | CG2 | 4.24 | |
| Val | 67I | O | Thr | 52H | CB | 3.78 | |
| | | | Thr | 52H | OG1 | 4.39 | * |
| | | | Tyr | 57H | CG | 4.83 | |
| | | | Tyr | 57H | CE2 | 4.27 | |
| | | | Tyr | 57H | CD2 | 3.91 | |
| | | | Thr | 52H | CG2 | 3.33 | |
| Glu | 68I | N | Tyr | 57H | CB | 4.88 | |
| | | | Tyr | 57H | CG | 4.14 | |
| | | | Tyr | 57H | CD1 | 4.74 | |
| | | | Tyr | 57H | CE1 | 4.77 | |
| | | | Tyr | 57H | CZ | 4.17 | |
| | | | Tyr | 57H | CE2 | 3.52 | |
| | | | Tyr | 57H | CD2 | 3.50 | |
| | | | Tyr | 57H | OH | 4.79 | * |
| | | | Thr | 52H | CG2 | 4.55 | |
| Glu | 68I | CA | Tyr | 57H | CB | 4.54 | |
| | | | Tyr | 57H | CG | 4.19 | |
| | | | Tyr | 57H | CD1 | 4.81 | |
| | | | Tyr | 57H | CZ | 4.97 | |
| | | | Tyr | 57H | CE2 | 4.41 | |
| | | | Tyr | 57H | CD2 | 3.99 | |
| | | | Thr | 52H | CG2 | 4.07 | |
| Glu | 68I | CB | Tyr | 57H | CB | 4.98 | |
| | | | Tyr | 57H | CG | 4.55 | |
| | | | Tyr | 57H | CD1 | 4.76 | |
| | | | Tyr | 57H | CE2 | 4.95 | |
| | | | Tyr | 57H | CD2 | 4.67 | |
| Glu | 68I | CG | Tyr | 57H | CB | 4.34 | |
| | | | Tyr | 57H | CG | 3.71 | |
| | | | Tyr | 57H | CD1 | 3.56 | |
| | | | Tyr | 57H | CE1 | 3.79 | |
| | | | Tyr | 57H | CZ | 4.13 | |
| | | | Tyr | 57H | CE2 | 4.31 | |
| | | | Tyr | 57H | CD2 | 4.11 | |
| | | | Tyr | 57H | OH | 4.93 | |
| Glu | 68I | CD | Tyr | 57H | CB | 3.80 | |
| | | | Tyr | 57H | CG | 3.59 | |
| | | | Tyr | 57H | CD1 | 3.34 | |
| | | | Tyr | 57H | CE1 | 4.01 | |
| | | | Tyr | 57H | CZ | 4.74 | |
| | | | Tyr | 57H | CE2 | 4.96 | |
| | | | Tyr | 57H | CD2 | 4.45 | |
| | | | His | 59H | CE1 | 4.34 | |
| | | | His | 59H | NE2 | 3.64 | |
| | | | His | 59H | CD2 | 4.73 | |
| Glu | 68I | OE1 | Tyr | 57H | CA | 4.91 | |
| | | | Tyr | 57H | CB | 3.65 | |
| | | | Tyr | 57H | CG | 3.92 | |
| | | | Tyr | 57H | CD1 | 4.00 | |
| | | | Tyr | 57H | CE1 | 4.92 | |
| | | | Tyr | 57H | CD2 | 4.80 | |
| | | | Tyr | 94L | CD1 | 4.93 | |
| | | | His | 59H | CG | 4.98 | |
| | | | His | 59H | CE1 | 3.86 | |
| | | | His | 59H | NE2 | 2.89 | *** |
| | | | His | 59H | CD2 | 3.79 | |
| | | | Tyr | 94L | CE1 | 4.13 | |
| | | | Tyr | 94L | CZ | 4.78 | |
| | | | Tyr | 94L | OH | 4.58 | * |
| Glu | 68I | OE2 | Tyr | 57H | CB | 4.15 | |
| | | | Tyr | 57H | CG | 3.85 | |
| | | | Tyr | 57H | CD1 | 3.17 | |
| | | | Tyr | 57H | CE1 | 3.74 | |
| | | | Tyr | 57H | CZ | 4.77 | |
| | | | Tyr | 57H | CD2 | 4.89 | |
| | | | His | 59H | CE1 | 3.98 | |
| | | | His | 59H | NE2 | 3.61 | * |
| | | | His | 59H | CD2 | 4.87 | |
| Glu | 68I | C | Thr | 52H | CG2 | 4.24 | |
| Thr | 69I | N | Ser | 33H | OG | 4.83 | * |
| | | | Thr | 52H | CG2 | 3.83 | |
| | | | Tyr | 94L | OH | 4.38 | * |
| Thr | 69I | CA | Ser | 33H | OG | 4.32 | |
| | | | Thr | 52H | CG2 | 4.64 | |
| | | | Tyr | 94L | OH | 4.65 | |
| Thr | 69I | CB | Ser | 33H | CB | 4.38 | |
| | | | Ser | 33H | OG | 3.76 | |
| | | | Thr | 52H | CG2 | 4.73 | |
| | | | Tyr | 94L | OH | 3.71 | |
| | | | Glu | 99H | CD | 4.54 | |
| | | | Glu | 99H | OE1 | 4.45 | |
| | | | Glu | 99H | OE2 | 4.42 | |
| | | | Tyr | 96L | OH | 4.33 | |
| Thr | 69I | OG1 | Thr | 52H | CB | 4.72 | |
| | | | Ser | 50H | OG | 4.45 | * |
| | | | Ser | 33H | CA | 4.81 | |
| | | | Ser | 33H | CB | 3.41 | |
| | | | Ser | 33H | OG | 2.77 | *** |
| | | | Thr | 52H | CA | 4.86 | |
| | | | Thr | 52H | CG2 | 3.57 | |
| | | | Tyr | 94L | OH | 3.82 | * |
| | | | Glu | 99H | CD | 4.85 | |
| | | | Glu | 99H | OE1 | 4.43 | * |
| Thr | 69I | CG2 | Ser | 33H | CB | 4.10 | |
| | | | Ser | 33H | OG | 3.72 | |
| | | | Tyr | 94L | OH | 4.71 | |
| | | | Glu | 99H | CG | 4.19 | |
| | | | Glu | 99H | CD | 3.61 | |
| | | | Glu | 99H | OE1 | 3.85 | |
| | | | Glu | 99H | OE2 | 3.55 | |
| | | | Arg | 100H | C | 4.73 | |
| | | | Arg | 100H | O | 4.39 | |
| | | | Gly | 101H | N | 4.58 | |
| | | | Gly | 101H | CA | 4.08 | |
| | | | Tyr | 96L | OH | 4.28 | |
| Thr | 69I | C | Tyr | 94L | OH | 4.98 | |
| Thr | 69I | O | Tyr | 94L | OH | 4.46 | * |
| Asn | 70I | N | Gly | 101H | CA | 4.06 | |
| | | | Gly | 101H | C | 4.65 | |
| | | | Trp | 102H | N | 4.36 | * |
| Asn | 70I | CA | Gly | 101H | CA | 4.49 | |
| | | | Gly | 101H | C | 4.63 | |
| | | | Trp | 102H | N | 4.14 | |
| | | | Gly | 103H | N | 4.39 | |
| Asn | 70I | CB | Gly | 101H | CA | 4.90 | |
| | | | Trp | 102H | N | 4.86 | |
| | | | Gly | 103H | N | 4.40 | |
| | | | Gly | 103H | CA | 4.83 | |
| | | | Tyr | 105H | CE1 | 3.67 | |
| | | | Tyr | 105H | CZ | 4.34 | |
| | | | Tyr | 105H | OH | 4.39 | |
| | | | Tyr | 105H | CD1 | 4.41 | |
| Asn | 70I | CG | Arg | 100H | O | 4.51 | |
| | | | Gly | 101H | CA | 3.99 | |
| | | | Gly | 101H | C | 4.25 | |
| | | | Gly | 101H | O | 4.85 | |
| | | | Trp | 102H | N | 4.42 | |
| | | | Gly | 103H | N | 3.92 | |
| | | | Gly | 103H | CA | 4.31 | |
| | | | Tyr | 105H | CE1 | 3.46 | |
| | | | Tyr | 105H | CZ | 4.31 | |
| | | | Tyr | 105H | OH | 4.81 | |
| | | | Tyr | 105H | CA | 4.97 | |
| | | | Gly | 103H | C | 4.89 | |
| | | | Tyr | 104H | N | 4.81 | |
| | | | Tyr | 105H | N | 4.59 | |
| | | | Tyr | 105H | CG | 4.72 | |
| | | | Tyr | 105H | CD1 | 3.72 | |
| Asn | 70I | OD1 | Arg | 100H | C | 4.56 | |
| | | | Arg | 100H | O | 3.92 | * |
| | | | Gly | 101H | N | 4.36 | * |
| | | | Gly | 101H | CA | 3.20 | |
| | | | Gly | 101H | C | 3.17 | |
| | | | Gly | 101H | O | 3.65 | * |
| | | | Trp | 102H | N | 3.43 | * |
| | | | Trp | 102H | CA | 4.30 | |
| | | | Trp | 102H | C | 3.96 | |
| | | | Gly | 103H | N | 2.90 | *** |
| | | | Gly | 103H | CA | 3.45 | |
| | | | Tyr | 105H | CE1 | 3.98 | |
| | | | Tyr | 105H | CZ | 4.59 | |
| | | | Tyr | 105H | CA | 4.77 | |
| | | | Gly | 103H | C | 3.95 | |
| | | | Tyr | 104H | N | 3.73 | * |
| | | | Tyr | 104H | CA | 4.75 | |
| | | | Tyr | 104H | C | 4.91 | |
| | | | Tyr | 105H | N | 4.03 | * |
| | | | Tyr | 105H | CG | 4.72 | |
| | | | Tyr | 105H | CD1 | 4.06 | |
| Asn | 70I | ND2 | Glu | 99H | CD | 4.85 | |
| | | | Glu | 99H | OE2 | 3.85 | * |
| | | | Arg | 100H | O | 4.29 | * |
| | | | Gly | 101H | CA | 4.50 | |
| | | | Phe | 91L | CB | 4.56 | |
| | | | Phe | 91L | CD1 | 4.56 | |
| | | | Phe | 91L | O | 4.91 | * |
| | | | Tyr | 96L | OH | 4.25 | * |
| | | | Tyr | 105H | CE1 | 3.53 | |
| | | | Tyr | 105H | CZ | 4.69 | |
| | | | Tyr | 105H | CA | 4.51 | |
| | | | Tyr | 105H | N | 4.47 | * |
| | | | Phe | 91L | CG | 4.93 | |
| | | | Tyr | 105H | CG | 4.61 | |
| | | | Tyr | 105H | CD1 | 3.47 | |
| Asn | 70I | C | Trp | 102H | N | 4.95 | |
| Asn | 70I | O | Tyr | 105H | OH | 4.64 | * |
| Glu | 72I | N | Trp | 102H | N | 4.98 | * |
| Glu | 72I | CA | Trp | 102H | CE2 | 4.69 | |
| | | | Trp | 102H | CZ3 | 4.56 | |
| | | | Trp | 102H | CH2 | 4.62 | |
| | | | Trp | 102H | CZ2 | 4.74 | |
| | | | Trp | 102H | CD2 | 4.60 | |
| | | | Trp | 102H | CE3 | 4.57 | |
| Glu | 72I | CB | Trp | 102H | NE1 | 3.75 | |
| | | | Trp | 102H | CE2 | 3.29 | |
| | | | Trp | 102H | CZ3 | 3.86 | |
| | | | Trp | 102H | CH2 | 3.77 | |
| | | | Trp | 102H | CZ2 | 3.56 | |
| | | | Trp | 102H | N | 4.37 | |
| | | | Trp | 102H | CA | 4.49 | |
| | | | Trp | 102H | CB | 4.87 | |
| | | | Trp | 102H | CG | 4.00 | |
| | | | Trp | 102H | CD1 | 4.12 | |
| | | | Trp | 102H | CD2 | 3.36 | |
| | | | Trp | 102H | CE3 | 3.68 | |
| Glu | 72I | CG | Trp | 102H | NE1 | 3.67 | |
| | | | Trp | 102H | CE2 | 3.71 | |
| | | | Trp | 102H | CH2 | 4.72 | |
| | | | Trp | 102H | CZ2 | 4.09 | |
| | | | Trp | 102H | N | 4.45 | |
| | | | Trp | 102H | CG | 4.45 | |
| | | | Trp | 102H | CD1 | 4.10 | |
| | | | Trp | 102H | CD2 | 4.15 | |
| | | | Trp | 102H | CE3 | 4.83 | |
| Glu | 72I | CD | Trp | 102H | NE1 | 4.23 | |
| | | | Trp | 102H | CE2 | 4.57 | |
| | | | Gly | 101H | CA | 4.08 | |
| | | | Gly | 101H | C | 4.26 | |
| | | | Trp | 102H | N | 3.55 | |
| | | | Trp | 102H | CA | 4.47 | |
| | | | Trp | 102H | CG | 4.55 | |
| | | | Trp | 102H | CD1 | 4.17 | |
| | | | Trp | 102H | CD2 | 4.71 | |
| Glu | 72I | OE1 | Trp | 102H | NE1 | 4.69 | * |
| | | | Trp | 102H | CE2 | 4.92 | |
| | | | Gly | 101H | N | 4.97 | * |
| | | | Gly | 101H | CA | 3.69 | |
| | | | Gly | 101H | C | 3.63 | |
| | | | Gly | 101H | O | 4.87 | * |
| | | | Trp | 102H | N | 2.74 | *** |
| | | | Trp | 102H | CA | 3.64 | |
| | | | Trp | 102H | CB | 4.66 | |
| | | | Trp | 102H | CG | 4.37 | |
| | | | Trp | 102H | CD1 | 4.31 | |
| | | | Trp | 102H | C | 4.71 | |
| | | | Gly | 103H | N | 4.70 | * |
| | | | Trp | 102H | CD2 | 4.67 | |
| Glu | 72I | OE2 | Ser | 33H | OG | 4.96 | * |
| | | | Trp | 102H | NE1 | 4.69 | * |
| | | | Gly | 101H | N | 4.72 | * |
| | | | Gly | 101H | CA | 3.85 | |
| | | | Gly | 101H | C | 4.46 | |
| | | | Trp | 102H | N | 4.10 | * |
| | | | Trp | 102H | CD1 | 4.63 | |
| Glu | 72I | C | Trp | 102H | CE2 | 4.98 | |
| | | | Trp | 102H | CZ3 | 3.93 | |
| | | | Trp | 102H | CH2 | 4.32 | |
| | | | Trp | 102H | CZ2 | 4.88 | |
| | | | Trp | 102H | CD2 | 4.60 | |
| | | | Trp | 102H | CE3 | 4.12 | |
| Glu | 72I | O | Trp | 102H | CZ3 | 3.80 | |
| | | | Trp | 102H | CH2 | 4.02 | |
| | | | Trp | 102H | CZ2 | 4.80 | |
| | | | Trp | 102H | CE3 | 4.41 | |
| Trp | 73I | N | Trp | 102H | CZ3 | 4.22 | |
| | | | Trp | 102H | CH2 | 4.98 | |
| | | | Trp | 102H | CA | 4.97 | |
| | | | Trp | 102H | CD2 | 4.76 | |
| | | | Trp | 102H | CE3 | 4.12 | |
| Trp | 73I | CA | Trp | 102H | CZ3 | 4.39 | |
| | | | Trp | 102H | CE3 | 4.48 | |
| Trp | 73I | CB | Trp | 102H | CE3 | 4.82 | |
| Trp | 73I | CG | Trp | 102H | CZ3 | 4.38 | |
| | | | Trp | 102H | CA | 4.80 | |
| | | | Trp | 102H | C | 4.91 | |
| | | | Trp | 102H | O | 4.69 | |
| | | | Trp | 102H | CD2 | 4.97 | |
| | | | Trp | 102H | CE3 | 3.93 | |
| Trp | 73I | CD1 | Trp | 102H | CZ3 | 4.78 | |
| | | | Trp | 102H | CA | 3.77 | |
| | | | Trp | 102H | CB | 4.13 | |
| | | | Trp | 102H | CG | 4.87 | |
| | | | Trp | 102H | C | 3.63 | |
| | | | Trp | 102H | O | 3.30 | |
| | | | Gly | 103H | N | 4.42 | |
| | | | Trp | 102H | CD2 | 4.68 | |
| | | | Trp | 102H | CE3 | 3.92 | |
| | | | Gly | 103H | CA | 5.00 | |
| Trp | 73I | NE1 | Trp | 102H | CZ3 | 4.64 | |
| | | | Trp | 102H | CA | 3.97 | |
| | | | Trp | 102H | CB | 3.81 | |
| | | | Trp | 102H | CG | 4.61 | |
| | | | Trp | 102H | C | 3.84 | |
| | | | Trp | 102H | O | 3.16 | *** |
| | | | Gly | 103H | N | 4.88 | * |
| | | | Trp | 102H | CD2 | 4.48 | |
| | | | Trp | 102H | CE3 | 3.70 | |
| Trp | 73I | CE2 | Trp | 102H | CZ3 | 4.02 | |
| | | | Trp | 102H | CA | 4.98 | |
| | | | Trp | 102H | CB | 4.57 | |
| | | | Trp | 102H | O | 4.48 | |
| | | | Trp | 102H | CD2 | 4.51 | |
| | | | Trp | 102H | CE3 | 3.44 | |
| Trp | 73I | CD2 | Trp | 102H | CZ3 | 3.85 | |
| | | | Trp | 102H | CD2 | 4.86 | |
| | | | Trp | 102H | CE3 | 3.62 | |
| Trp | 73I | CE3 | Trp | 102H | CZ3 | 3.89 | |
| | | | Trp | 102H | CE3 | 4.15 | |
| Trp | 73I | CZ3 | Trp | 102H | CZ3 | 4.14 | |
| | | | Trp | 102H | CE3 | 4.48 | |
| Trp | 73I | CH2 | Trp | 102H | CZ3 | 4.34 | |
| | | | Trp | 102H | CE3 | 4.38 | |
| Trp | 73I | CZ2 | Trp | 102H | CZ3 | 4.32 | |
| | | | Trp | 102H | CD2 | 4.96 | |
| | | | Trp | 102H | CE3 | 3.92 | |
| Phe | 76I | CB | Trp | 102H | CZ3 | 4.38 | |
| | | | Trp | 102H | CH2 | 4.71 | |
| Phe | 76I | CG | Trp | 102H | CZ3 | 4.99 | |
| Lys | 117I | N | Trp | 102H | O | 4.83 | * |
| | | | Gly | 103H | CA | 4.98 | |
| Lys | 117I | CA | Gly | 103H | CA | 4.74 | |
| Lys | 117I | CB | Gly | 103H | CA | 4.16 | |
| | | | Tyr | 105H | CZ | 4.93 | |
| | | | Tyr | 105H | OH | 4.52 | |
| | | | Gly | 103H | C | 4.97 | |
| | | | Gly | 103H | O | 4.72 | |
| | | | Tyr | 105H | CE2 | 4.33 | |
| Lys | 117I | CD | Gly | 103H | O | 4.47 | |
| | | | Ser | 31L | OG | 3.85 | |
| | | | Asp | 50L | CG | 4.15 | |
| | | | Asp | 50L | OD1 | 3.73 | |
| | | | Asp | 50L | OD2 | 3.91 | |
| | | | Tyr | 105H | CE2 | 4.40 | |
| | | | Tyr | 105H | CD2 | 4.86 | |
| Lys | 117I | CE | Ser | 31L | OG | 4.04 | |
| | | | Asp | 50L | CG | 4.16 | |
| | | | Asp | 50L | OD1 | 4.02 | |
| | | | Asp | 50L | OD2 | 3.52 | |
| Lys | 117I | NZ | Ser | 31L | OG | 3.25 | *** |
| | | | Asp | 50L | CB | 4.96 | |
| | | | Asp | 50L | CG | 3.48 | |
| | | | Asp | 50L | OD1 | 3.38 | * |
| | | | Asp | 50L | OD2 | 2.80 | *** |
| | | | Ser | 31L | CB | 4.00 | |
| | | | Asp | 50L | O | 4.84 | * |
| Lys | 117I | C | Gly | 103H | N | 4.64 | |
| | | | Gly | 103H | CA | 4.25 | |
| | | | Tyr | 105H | OH | 4.40 | |
| Lys | 117I | O | Trp | 102H | CA | 4.71 | |
| | | | Trp | 102H | C | 3.92 | |
| | | | Trp | 102H | O | 4.05 | * |
| | | | Gly | 103H | N | 3.64 | * |
| | | | Gly | 103H | CA | 3.55 | |
| | | | Tyr | 105H | OH | 4.71 | * |
| | | | Gly | 103H | C | 4.97 | |
| His | 118I | N | Tyr | 105H | OH | 3.96 | * |
| His | 118I | CA | Tyr | 105H | CZ | 5.00 | |
| | | | Tyr | 105H | OH | 3.81 | |
| His | 118I | C | Tyr | 105H | CZ | 4.82 | |
| | | | Tyr | 105H | OH | 3.45 | |
| His | 118I | O | Tyr | 105H | OH | 3.70 | * |
| Arg | 119I | N | Tyr | 105H | CZ | 4.82 | |
| | | | Tyr | 105H | OH | 3.55 | * |
| Arg | 119I | CA | Tyr | 105H | OH | 4.10 | |
| Arg | 119I | CB | Tyr | 105H | OH | 4.72 | |
| | | | Asn | 92L | O | 4.67 | |
| Arg | 119I | CG | Phe | 91L | O | 4.23 | |
| | | | Tyr | 105H | CE1 | 4.41 | |
| | | | Tyr | 105H | CZ | 4.71 | |
| | | | Tyr | 105H | OH | 4.04 | |
| | | | Asn | 92L | CA | 4.97 | |
| | | | Asn | 92L | C | 4.90 | |
| | | | Asn | 92L | O | 4.14 | |
| Arg | 119I | CD | Phe | 91L | C | 4.63 | |
| | | | Phe | 91L | O | 3.51 | |
| | | | Asn | 92L | CA | 4.33 | |
| | | | Asn | 92L | C | 3.90 | |
| | | | Asn | 92L | O | 3.12 | |
| | | | Ser | 93L | N | 4.81 | |
| Arg | 119I | NE | Tyr | 94L | CE1 | 4.87 | |
| | | | Phe | 91L | O | 4.09 | * |
| | | | Asn | 92L | C | 4.92 | |
| | | | Asn | 92L | O | 4.21 | * |
| Arg | 119I | CZ | Tyr | 94L | CD1 | 5.00 | |
| | | | Tyr | 94L | CE1 | 4.33 | |
| | | | Asn | 92L | O | 4.73 | |
| Arg | 119I | NH1 | Tyr | 94L | CE1 | 4.84 | |
| | | | Asn | 92L | O | 4.41 | * |
| Arg | 119I | NH2 | Tyr | 94L | CD1 | 4.76 | |
| | | | Tyr | 94L | CE1 | 3.83 | |
| | | | Tyr | 94L | CZ | 4.62 | |
| | | | Tyr | 94L | OH | 4.33 | * |
| Thr | 121I | OG1 | Asn | 92L | O | 4.93 | * |
| Tyr | 128I | CD1 | Tyr | 57H | OH | 4.88 | |
| Tyr | 128I | CE1 | Tyr | 57H | OH | 4.33 | |
| Glu | 129I | OE2 | Tyr | 56H | OH | 4.71 | * |
| Leu | 143I | CA | Trp | 102H | CZ2 | 4.82 | |
| Leu | 143I | CB | Trp | 102H | CH2 | 4.85 | |
| | | | Trp | 102H | CZ2 | 4.89 | |
| Leu | 143I | CG | Trp | 102H | CZ3 | 4.83 | |
| | | | Trp | 102H | CH2 | 3.61 | |
| | | | Trp | 102H | CZ2 | 3.75 | |
| Leu | 143I | CD1 | Trp | 102H | CE2 | 4.77 | |
| | | | Trp | 102H | CH2 | 3.98 | |
| | | | Trp | 102H | CZ2 | 3.70 | |
| Leu | 143I | CD2 | Trp | 102H | CZ3 | 4.88 | |
| | | | Trp | 102H | CH2 | 3.97 | |
| | | | Trp | 102H | CZ2 | 4.51 | |
| Leu | 143I | C | Trp | 102H | CH2 | 4.39 | |
| | | | Trp | 102H | CZ2 | 4.17 | |
| Leu | 143I | O | Trp | 102H | CE2 | 4.44 | |
| | | | Trp | 102H | CH2 | 3.74 | |
| | | | Trp | 102H | CZ2 | 3.27 | |
| Leu | 144I | N | Trp | 102H | CH2 | 4.98 | |
| Leu | 144I | CA | Trp | 102H | CH2 | 4.97 | |
| Gln | 145I | O | Ser | 31H | OG | 4.91 | * |
| Lys | 146I | N | Ser | 31H | OG | 4.81 | * |
| Lys | 146I | CA | Ser | 31H | OG | 4.04 | |
| | | | Ser | 31H | CB | 4.39 | |
| | | | Ser | 31H | O | 4.96 | |
| | | | Trp | 102H | NE1 | 4.95 | |
| Lys | 146I | CB | Ser | 31H | OG | 4.33 | |
| | | | Ser | 31H | CA | 4.82 | |
| | | | Ser | 31H | CB | 4.49 | |
| | | | Ser | 31H | O | 4.37 | |
| | | | Trp | 102H | NE1 | 4.42 | |
| | | | Trp | 102H | CE2 | 4.85 | |
| | | | Trp | 102H | CZ2 | 4.56 | |
| Lys | 146I | CG | Ser | 30H | O | 4.78 | |
| | | | Ser | 31H | OG | 3.68 | |
| | | | Ser | 31H | CA | 4.13 | |
| | | | Ser | 31H | CB | 4.08 | |
| | | | Ser | 31H | C | 4.73 | |
| | | | Ser | 31H | O | 4.32 | |
| Lys | 146I | CD | Ser | 30H | O | 4.53 | |
| | | | Ser | 53H | CB | 4.97 | |
| | | | Ser | 53H | OG | 4.16 | |
| | | | Ser | 31H | OG | 4.76 | |
| | | | Ser | 31H | CA | 4.50 | |
| | | | Ser | 31H | CB | 4.93 | |
| | | | Ser | 31H | C | 4.84 | |
| | | | Ser | 31H | O | 4.45 | |
| Lys | 146I | CE | Ser | 30H | O | 3.72 | |
| | | | Ser | 53H | CB | 4.49 | |
| | | | Ser | 53H | OG | 3.75 | |
| | | | Ser | 31H | OG | 4.87 | |
| | | | Ser | 30H | C | 4.79 | |
| | | | Ser | 31H | CA | 4.57 | |
| Lys | 146I | NZ | Ser | 30H | O | 4.76 | * |
| Lys | 146I | C | Trp | 102H | NE1 | 4.11 | |
| | | | Trp | 102H | CE2 | 4.49 | |
| | | | Trp | 102H | CZ2 | 4.52 | |
| | | | Trp | 102H | CD1 | 4.99 | |
| Lys | 146I | O | Ser | 31H | OG | 4.95 | * |
| | | | Ser | 31H | CB | 4.71 | |
| | | | Ser | 31H | O | 4.86 | * |
| | | | Trp | 102H | NE1 | 4.28 | * |
| | | | Trp | 102H | CE2 | 4.91 | |
| | | | Trp | 102H | CD1 | 4.86 | |
| Met | 147I | N | Trp | 102H | NE1 | 3.87 | * |
| | | | Trp | 102H | CE2 | 3.86 | |
| | | | Trp | 102H | CH2 | 4.58 | |
| | | | Trp | 102H | CZ2 | 3.71 | |
| | | | Trp | 102H | CD1 | 4.79 | |
| | | | Trp | 102H | CD2 | 4.88 | |
| Met | 147I | CA | Trp | 102H | NE1 | 3.79 | |
| | | | Trp | 102H | CE2 | 3.68 | |
| | | | Trp | 102H | CZ3 | 4.93 | |
| | | | Trp | 102H | CH2 | 4.48 | |
| | | | Trp | 102H | CZ2 | 3.84 | |
| | | | Trp | 102H | CG | 4.59 | |
| | | | Trp | 102H | CD1 | 4.36 | |
| | | | Trp | 102H | CD2 | 4.27 | |
| | | | Trp | 102H | CE3 | 4.83 | |
| Met | 147I | CA | Trp | 102H | NE1 | 3.73 | |
| | | | Trp | 102H | CE2 | 3.65 | |
| | | | Trp | 102H | CZ3 | 4.97 | |
| | | | Trp | 102H | CH2 | 4.52 | |
| | | | Trp | 102H | CZ2 | 3.84 | |
| | | | Trp | 102H | CG | 4.55 | |
| | | | Trp | 102H | CD1 | 4.30 | |
| | | | Trp | 102H | CD2 | 4.26 | |
| | | | Trp | 102H | CE3 | 4.85 | |
| Met | 147I | CB | Trp | 102H | NE1 | 4.33 | |
| | | | Trp | 102H | CE2 | 3.72 | |
| | | | Trp | 102H | CZ3 | 4.02 | |
| | | | Trp | 102H | CH2 | 3.71 | |
| | | | Trp | 102H | CZ2 | 3.54 | |
| | | | Trp | 102H | CG | 4.80 | |
| | | | Trp | 102H | CD1 | 4.94 | |
| | | | Trp | 102H | CD2 | 4.09 | |
| | | | Trp | 102H | CE3 | 4.21 | |
| Met | 147I | CB | Trp | 102H | NE1 | 4.01 | |
| | | | Trp | 102H | CE2 | 3.42 | |
| | | | Trp | 102H | CZ3 | 3.85 | |
| | | | Trp | 102H | CH2 | 3.58 | |
| | | | Trp | 102H | CZ2 | 3.36 | |
| | | | Trp | 102H | CG | 4.41 | |
| | | | Trp | 102H | CD1 | 4.57 | |
| | | | Trp | 102H | CD2 | 3.75 | |
| | | | Trp | 102H | CE3 | 3.93 | |
| Met | 147I | CG | Trp | 102H | NE1 | 4.84 | |
| | | | Trp | 102H | CE2 | 4.15 | |
| | | | Trp | 102H | CZ3 | 3.89 | |
| | | | Trp | 102H | CH2 | 4.12 | |
| | | | Trp | 102H | CZ2 | 4.26 | |
| | | | Trp | 102H | CG | 4.55 | |
| | | | Trp | 102H | CD2 | 3.95 | |
| | | | Trp | 102H | CE3 | 3.81 | |
| Met | 147I | CG | Trp | 102H | NE1 | 4.25 | |
| | | | Trp | 102H | CE2 | 3.64 | |
| | | | Trp | 102H | CZ3 | 3.67 | |
| | | | Trp | 102H | CH2 | 3.96 | |
| | | | Trp | 102H | CZ2 | 3.97 | |
| | | | Trp | 102H | CB | 4.49 | |
| | | | Trp | 102H | CG | 3.81 | |
| | | | Trp | 102H | CD1 | 4.36 | |
| | | | Trp | 102H | CD2 | 3.33 | |
| | | | Trp | 102H | CE3 | 3.34 | |
| Met | 147I | SD | Trp | 102H | CZ3 | 4.15 | |
| | | | Trp | 102H | CH2 | 4.66 | |
| | | | Trp | 102H | CD2 | 4.64 | |
| | | | Trp | 102H | CE3 | 4.15 | |
| His | 149I | CB | Ile | 28H | CB | 4.70 | |
| | | | Ile | 28H | CG1 | 4.85 | |
| | | | Ser | 31H | OG | 3.93 | |
| | | | Ser | 31H | CB | 4.05 | |
| His | 149I | CG | Ile | 28H | CB | 4.14 | |
| | | | Ile | 28H | CG1 | 4.03 | |
| | | | Ile | 28H | CG2 | 4.47 | |
| | | | Ser | 31H | OG | 3.83 | |
| | | | Ser | 31H | CB | 4.42 | |
| His | 149I | ND1 | Ile | 28H | CB | 3.91 | |
| | | | Ile | 28H | CG1 | 4.03 | |
| | | | Ile | 28H | CG2 | 3.77 | |
| | | | Ser | 31H | OG | 2.95 | *** |
| | | | Ser | 31H | CB | 3.91 | |
| His | 149I | CE1 | Ile | 28H | CD1 | 4.79 | |
| | | | Ile | 28H | CB | 4.07 | |
| | | | Ile | 28H | CG1 | 3.83 | |
| | | | Ile | 28H | CG2 | 3.75 | |
| | | | Ser | 31H | OG | 3.90 | |
| His | 149I | NE2 | Ile | 28H | CD1 | 4.60 | |
| | | | Ile | 28H | CB | 4.38 | |
| | | | Ile | 28H | CG1 | 3.69 | |
| | | | Ile | 28H | CG2 | 4.44 | |
| His | 149I | CD2 | Ile | 28H | CD1 | 5.00 | |
| | | | Ile | 28H | CB | 4.42 | |
| | | | Ile | 28H | CG1 | 3.81 | |
| | | | Ile | 28H | CG2 | 4.84 | |
| His | 149I | C | Tyr | 32H | OH | 4.03 | |
| | | | Tyr | 32H | CZ | 4.82 | |
| His | 149I | O | Tyr | 32H | OH | 3.58 | * |
| | | | Tyr | 32H | CZ | 4.66 | |
| Gln | 150I | N | Tyr | 32H | OH | 4.14 | * |
| | | | Tyr | 32H | CZ | 4.70 | |
| Gln | 150I | CA | Tyr | 32H | OH | 3.57 | |
| | | | Tyr | 32H | CE1 | 4.46 | |
| | | | Tyr | 32H | CZ | 4.16 | |
| | | | Arg | 100H | NE | 4.70 | |
| | | | Arg | 100H | CZ | 4.91 | |
| | | | Arg | 100H | NH2 | 4.85 | |
| Gln | 150I | CB | Tyr | 32H | OH | 4.29 | |
| | | | Tyr | 32H | CE1 | 4.35 | |
| | | | Tyr | 32H | CZ | 4.46 | |
| | | | Arg | 100H | CG | 4.64 | |
| | | | Trp | 102H | CD1 | 4.73 | |
| | | | Arg | 100H | CD | 4.89 | |
| | | | Arg | 100H | NE | 4.18 | |
| | | | Arg | 100H | CZ | 4.67 | |
| | | | Arg | 100H | NH2 | 4.59 | |
| Gln | 150I | CG | Tyr | 32H | CD2 | 4.78 | |
| | | | Ser | 31H | CB | 4.99 | |
| | | | Ser | 31H | C | 4.97 | |
| | | | Ser | 31H | O | 4.06 | |
| | | | Trp | 102H | NE1 | 4.79 | |
| | | | Tyr | 32H | CE2 | 4.43 | |
| | | | Tyr | 32H | OH | 4.13 | |
| | | | Tyr | 32H | CG | 4.70 | |
| | | | Tyr | 32H | CD1 | 4.15 | |
| | | | Tyr | 32H | CE1 | 3.72 | |
| | | | Tyr | 32H | CZ | 3.85 | |
| | | | Arg | 100H | CG | 4.82 | |
| | | | Trp | 102H | CD1 | 4.55 | |
| Gln | 150I | CD | Tyr | 32H | CA | 4.97 | |
| | | | Ser | 31H | C | 4.86 | |
| | | | Ser | 31H | O | 3.82 | |
| | | | Trp | 12H | NE1 | 4.28 | |
| | | | Tyr | 32H | CG | 4.57 | |
| | | | Tyr | 32H | CD1 | 3.94 | |
| | | | Tyr | 32H | CE1 | 3.95 | |
| | | | Tyr | 32H | CZ | 4.51 | |
| | | | Arg | 100H | CB | 4.55 | |
| | | | Arg | 100H | CG | 4.37 | |
| | | | Arg | 100H | CA | 4.24 | |
| | | | Arg | 100H | C | 4.65 | |
| | | | Gly | 101H | N | 3.93 | |
| | | | Gly | 101H | CA | 4.90 | |
| | | | Trp | 102H | CD1 | 3.85 | |
| Gln | 150I | OE1 | Ser | 31H | O | 4.94 | * |
| | | | Trp | 102H | NE1 | 4.33 | * |
| | | | Tyr | 32H | CD1 | 4.49 | |
| | | | Tyr | 32H | CE1 | 4.47 | |
| | | | Glu | 99H | O | 4.93 | * |
| | | | Arg | 100H | CB | 3.63 | |
| | | | Arg | 100H | CG | 3.74 | |
| | | | Arg | 100H | N | 4.81 | * |
| | | | Arg | 100H | CA | 3.46 | |
| | | | Arg | 100H | C | 3.71 | |
| | | | Arg | 100H | O | 4.89 | * |
| | | | Gly | 101H | N | 3.01 | *** |
| | | | Gly | 101H | CA | 3.99 | |
| | | | Gly | 101H | C | 4.07 | |
| | | | Gly | 101H | O | 4.01 | * |
| | | | Trp | 102H | N | 4.89 | * |
| | | | Trp | 102H | CB | 4.86 | |
| | | | Trp | 102H | CG | 4.50 | |
| | | | Trp | 102H | CD1 | 3.51 | |
| | | | Arg | 100H | CD | 4.70 | |
| | | | Arg | 100H | NE | 4.28 | * |
| Gln | 150I | NE2 | Tyr | 32H | N | 4.30 | * |
| | | | Tyr | 32H | CA | 3.82 | |
| | | | Tyr | 32H | CB | 4.39 | |
| | | | Tyr | 32H | CD2 | 4.59 | |
| | | | Tyr | 32H | C | 4.87 | |
| | | | Ser | 33H | N | 4.84 | * |
| | | | Ser | 31H | CA | 4.95 | |
| | | | Ser | 31H | CB | 4.98 | |
| | | | Ser | 31H | C | 3.78 | |
| | | | Ser | 31H | O | 2.71 | *** |
| | | | Trp | 102H | NE1 | 4.44 | * |
| | | | Tyr | 32H | CE2 | 4.96 | |
| | | | Tyr | 32H | CG | 4.01 | |
| | | | Tyr | 32H | CD1 | 3.73 | |
| | | | Tyr | 32H | CE1 | 4.17 | |
| | | | Glu | 99H | O | 4.64 | * |
| | | | Tyr | 32H | CZ | 4.72 | |
| | | | Arg | 100H | CA | 4.54 | |
| | | | Arg | 100H | C | 4.86 | |
| | | | Gly | 101H | N | 4.08 | * |
| | | | Gly | 101H | CA | 4.97 | |
| | | | Trp | 102H | CD1 | 4.28 | |
| Gln | 150I | C | Tyr | 32H | OH | 4.36 | |
| | | | Arg | 100H | NE | 4.31 | |
| | | | Arg | 100H | CZ | 4.10 | |
| | | | Arg | 100H | NH1 | 4.75 | |
| | | | Arg | 100H | NH2 | 3.88 | |
| Gln | 150I | O | Tyr | 32H | OH | 4.33 | * |
| | | | Arg | 100H | CG | 4.99 | |
| | | | Arg | 100H | CD | 4.26 | |
| | | | Arg | 100H | NE | 3.53 | * |
| | | | Arg | 100H | CZ | 3.18 | |
| | | | Arg | 100H | NH1 | 3.62 | * |
| | | | Arg | 100H | NH2 | 3.22 | *** |
| His | 151I | N | Arg | 100H | CZ | 4.88 | |
| | | | Arg | 100H | NH2 | 4.33 | * |
| His | 151I | CA | Arg | 100H | CZ | 4.82 | |
| | | | Arg | 100H | NH2 | 4.07 | |
| His | 151I | CB | Arg | 100H | NH2 | 4.39 | |
| His | 151I | CG | Arg | 100H | CZ | 4.91 | |
| | | | Arg | 100H | NH2 | 3.71 | |
| His | 151I | ND1 | Trp | 102H | CB | 4.95 | |
| | | | Trp | 102H | CG | 4.94 | |
| | | | Arg | 100H | NH2 | 4.13 | * |
| His | 151I | CE1 | Trp | 102H | CB | 3.73 | |
| | | | Trp | 102H | CG | 3.99 | |
| | | | Trp | 102H | CD1 | 4.50 | |
| | | | Trp | 102H | CD2 | 4.70 | |
| | | | Arg | 100H | NH2 | 4.09 | |
| His | 151I | NE2 | Trp | 102H | CB | 4.23 | |
| | | | Trp | 102H | CG | 4.89 | |
| | | | Trp | 102H | O | 4.90 | * |
| | | | Arg | 100H | CZ | 4.82 | |
| | | | Arg | 100H | NH2 | 3.58 | * |
| His | 151I | CD2 | Arg | 100H | CZ | 4.64 | |
| | | | Arg | 100H | NH2 | 3.31 | |

TABLE 16
hIL-21, chain I, (SEQ ID No 1) interactions with the the heavy chain
(chain H) of Fab59 (SEQ ID No 10) and light chain (chain L) of
anti-IL-21 Fab59 (SEQ ID No 9, mutation Q27N). A distance cut-off
of 5.0 Å was used. The contacts were identified by the CONTACT
computer software program of the CCP4 suite (Bailey, 1994). In the last
column “***” indicates a strong possibility for a hydrogen bond
at this contact (distance <3.3 Å) as calculated by CONTACT,
“*” indicates a weak possibility (distance >3.3 Å).
Blank indicates that the program considered there to be no possibility
of a hydrogen bond. Hydrogen-bonds are specific between a donor and
an acceptor, are typically strong, and are easily identifiable.

| | | | aIL-21 Fab59 (Fab35 | | | | |
|---|---|---|---|---|---|---|---|
| hIL-21 | | | with L, Q27N mutation) | | | | |
| | Res. # | | | Res. # | | | |
| Res. | and | Atom | Res. | and | Atom | Distance | Possibly |
| Type | Chain | name | Type | Chain | name | [Å] | H-bond |
| Glu | 65I | N | Tyr | 56H | CE2 | 4.80 | |
| Glu | 65I | CA | Tyr | 56H | OH | 4.99 | |
| | | | Tyr | 56H | CE2 | 4.73 | |
| Glu | 65I | CB | Tyr | 56H | CD2 | 4.93 | |
| | | | Tyr | 56H | CZ | 4.04 | |
| | | | Tyr | 56H | OH | 3.62 | |
| | | | Tyr | 56H | CE2 | 3.94 | |
| Glu | 65I | CG | Tyr | 56H | CZ | 4.72 | |
| | | | Tyr | 56H | OH | 3.90 | |
| | | | Tyr | 56H | CE2 | 4.78 | |
| Glu | 65I | CD | Tyr | 56H | CZ | 4.31 | |
| | | | Tyr | 56H | OH | 3.18 | |
| | | | Tyr | 56H | CE2 | 4.77 | |
| Glu | 65I | OE1 | Tyr | 56H | CZ | 4.59 | |
| | | | Tyr | 56H | OH | 3.50 | * |
| Glu | 65I | OE2 | Tyr | 56H | CZ | 4.20 | |
| | | | Tyr | 56H | OH | 2.93 | *** |
| | | | Tyr | 56H | CE2 | 4.70 | |
| Glu | 65I | C | Tyr | 56H | CE2 | 4.78 | |
| Asp | 66I | N | Tyr | 56H | CD2 | 4.39 | |
| | | | Tyr | 56H | CE2 | 4.35 | |
| Asp | 66I | CA | Tyr | 56H | CD2 | 4.55 | |
| | | | Tyr | 56H | CE2 | 4.91 | |
| Asp | 66I | CB | Tyr | 56H | CD2 | 3.61 | |
| | | | Tyr | 56H | CG | 3.74 | |
| | | | Tyr | 56H | CE1 | 4.82 | |
| | | | Tyr | 56H | CZ | 4.71 | |
| | | | Tyr | 56H | CE2 | 4.15 | |
| | | | Tyr | 56H | CD1 | 4.31 | |
| | | | Tyr | 57H | CE2 | 4.71 | |
| | | | Tyr | 57H | CD2 | 4.82 | |
| | | | Tyr | 56H | CA | 4.98 | |
| | | | Tyr | 56H | CB | 3.97 | |
| | | | Thr | 52H | OG1 | 4.85 | |
| | | | Gly | 54H | N | 4.62 | |
| | | | Gly | 54H | CA | 4.31 | |
| | | | Gly | 54H | C | 4.75 | |
| | | | Tyr | 56H | N | 4.75 | |
| Asp | 66I | CG | Tyr | 56H | CD2 | 4.62 | |
| | | | Tyr | 56H | CG | 4.30 | |
| | | | Gly | 54H | O | 4.52 | |
| | | | Tyr | 56H | CD1 | 4.73 | |
| | | | Tyr | 57H | CE2 | 4.93 | |
| | | | Tyr | 57H | CD2 | 4.59 | |
| | | | Tyr | 56H | CA | 4.72 | |
| | | | Tyr | 56H | CB | 4.12 | |
| | | | Thr | 52H | CB | 4.03 | |
| | | | Thr | 52H | OG1 | 3.51 | |
| | | | Thr | 52H | C | 4.86 | |
| | | | Ser | 53H | N | 4.65 | |
| | | | Gly | 54H | N | 3.37 | |
| | | | Gly | 54H | CA | 3.44 | |
| | | | Gly | 54H | C | 3.91 | |
| | | | Ser | 55H | N | 4.25 | |
| | | | Tyr | 56H | N | 4.12 | |
| | | | Ser | 53H | OG | 4.67 | |
| | | | Ser | 53H | C | 4.62 | |
| | | | Thr | 52H | CG2 | 5.00 | |
| Asp | 66I | OD1 | Thr | 52H | CB | 3.80 | |
| | | | Thr | 52H | OG1 | 3.73 | * |
| | | | Thr | 52H | C | 4.40 | |
| | | | Ser | 53H | N | 3.89 | * |
| | | | Gly | 54H | N | 3.07 | *** |
| | | | Gly | 54H | CA | 3.55 | |
| | | | Gly | 54H | C | 4.38 | |
| | | | Ser | 55H | N | 4.63 | * |
| | | | Ser | 53H | CB | 4.51 | |
| | | | Ser | 53H | OG | 3.53 | * |
| | | | Ser | 53H | CA | 4.33 | |
| | | | Ser | 53H | C | 4.11 | |
| | | | Thr | 52H | CA | 4.77 | |
| | | | Thr | 52H | CG2 | 4.81 | |
| Asp | 66I | OD2 | Tyr | 56H | CD2 | 4.42 | |
| | | | Tyr | 56H | CG | 3.76 | |
| | | | Gly | 54H | O | 3.91 | * |
| | | | Tyr | 56H | CD1 | 4.16 | |
| | | | Tyr | 57H | CE2 | 4.70 | |
| | | | Tyr | 57H | CD2 | 4.13 | |
| | | | Tyr | 56H | CA | 3.66 | |
| | | | Tyr | 56H | CB | 3.32 | |
| | | | Tyr | 56H | C | 4.41 | |
| | | | Tyr | 57H | N | 4.14 | * |
| | | | Thr | 52H | CB | 3.57 | |
| | | | Thr | 52H | OG1 | 2.68 | *** |
| | | | Thr | 52H | C | 4.44 | |
| | | | Thr | 52H | O | 4.76 | * |
| | | | Ser | 53H | N | 4.57 | * |
| | | | Gly | 54H | N | 3.31 | * |
| | | | Gly | 54H | CA | 3.33 | |
| | | | Gly | 54H | C | 3.32 | |
| | | | Ser | 55H | N | 3.42 | * |
| | | | Ser | 55H | CA | 4.24 | |
| | | | Ser | 55H | C | 4.02 | |
| | | | Tyr | 56H | N | 2.96 | *** |
| | | | Ser | 53H | C | 4.57 | |
| | | | Thr | 52H | CA | 4.61 | |
| | | | Thr | 52H | CG2 | 4.59 | |
| Asp | 66I | C | Tyr | 56H | CD2 | 4.77 | |
| | | | Tyr | 57H | CE2 | 3.95 | |
| | | | Tyr | 57H | CD2 | 4.49 | |
| Asp | 66I | O | Tyr | 56H | CD2 | 4.32 | |
| | | | Tyr | 56H | CE2 | 4.91 | |
| | | | Tyr | 57H | CZ | 4.40 | |
| | | | Tyr | 57H | OH | 4.25 | * |
| | | | Tyr | 57H | CE2 | 3.56 | |
| | | | Tyr | 57H | CD2 | 4.46 | |
| Val | 67I | N | Tyr | 57H | CE2 | 3.90 | |
| | | | Tyr | 57H | CD2 | 4.19 | |
| Val | 67I | CA | Tyr | 57H | CZ | 4.47 | |
| | | | Tyr | 57H | OH | 4.76 | |
| | | | Tyr | 57H | CE2 | 3.57 | |
| | | | Tyr | 57H | CD2 | 4.00 | |
| Val | 67I | CA | Tyr | 57H | CZ | 4.71 | |
| | | | Tyr | 57H | OH | 4.99 | |
| | | | Tyr | 57H | CE2 | 3.81 | |
| | | | Tyr | 57H | CD2 | 4.21 | |
| Val | 67I | CB | Tyr | 57H | CE2 | 4.91 | |
| Val | 67I | C | Tyr | 57H | CZ | 4.58 | |
| | | | Tyr | 57H | CE2 | 3.67 | |
| | | | Tyr | 57H | CD2 | 3.61 | |
| | | | Tyr | 57H | CG | 4.49 | |
| | | | Thr | 52H | CB | 4.97 | |
| | | | Thr | 52H | CG2 | 4.36 | |
| Val | 67I | O | Tyr | 57H | CE2 | 4.26 | |
| | | | Tyr | 57H | CD2 | 3.83 | |
| | | | Tyr | 57H | CB | 5.00 | |
| | | | Tyr | 57H | CG | 4.67 | |
| | | | Thr | 52H | CB | 3.92 | |
| | | | Thr | 52H | OG1 | 4.43 | * |
| | | | Thr | 52H | CG2 | 3.40 | |
| Glu | 68I | N | Tyr | 57H | CE1 | 4.68 | |
| | | | Tyr | 57H | CZ | 4.20 | |
| | | | Tyr | 57H | OH | 4.87 | * |
| | | | Tyr | 57H | CE2 | 3.63 | |
| | | | Tyr | 57H | CD2 | 3.60 | |
| | | | Tyr | 57H | CB | 4.86 | |
| | | | Tyr | 57H | CG | 4.13 | |
| | | | Tyr | 57H | CD1 | 4.64 | |
| | | | Thr | 52H | CG2 | 4.68 | |
| Glu | 68I | CA | Tyr | 57H | CE1 | 4.96 | |
| | | | Tyr | 57H | CZ | 4.89 | |
| | | | Tyr | 57H | CE2 | 4.45 | |
| | | | Tyr | 57H | CD2 | 4.02 | |
| | | | Tyr | 57H | CB | 4.43 | |
| | | | Tyr | 57H | CG | 4.09 | |
| | | | Tyr | 57H | CD1 | 4.58 | |
| | | | Thr | 52H | CG2 | 4.18 | |
| Glu | 68I | CB | Tyr | 57H | CE1 | 4.80 | |
| | | | Tyr | 57H | CD2 | 4.76 | |
| | | | Tyr | 57H | CB | 4.92 | |
| | | | Tyr | 57H | CG | 4.49 | |
| | | | Tyr | 57H | CD1 | 4.53 | |
| Glu | 68I | CG | Tyr | 57H | CE1 | 3.51 | |
| | | | Tyr | 57H | CZ | 4.03 | |
| | | | Tyr | 57H | OH | 4.83 | |
| | | | Tyr | 57H | CE2 | 4.34 | |
| | | | Tyr | 57H | CD2 | 4.18 | |
| | | | Tyr | 57H | CB | 4.29 | |
| | | | Tyr | 57H | CG | 3.65 | |
| | | | Tyr | 57H | CD1 | 3.30 | |
| Glu | 68I | CD | Tyr | 57H | CE1 | 3.87 | |
| | | | Tyr | 57H | CZ | 4.77 | |
| | | | Tyr | 57H | CD2 | 4.66 | |
| | | | Tyr | 57H | CB | 3.92 | |
| | | | Tyr | 57H | CG | 3.71 | |
| | | | Tyr | 57H | CD1 | 3.24 | |
| | | | His | 59H | CE1 | 4.25 | |
| | | | Tyr | 94L | CE2 | 4.87 | |
| | | | His | 59H | NE2 | 3.54 | |
| | | | His | 59H | CD2 | 4.63 | |
| Glu | 68I | OE1 | Tyr | 57H | CE1 | 4.67 | |
| | | | Tyr | 57H | CD2 | 4.86 | |
| | | | Tyr | 57H | CA | 4.79 | |
| | | | Tyr | 57H | CB | 3.56 | |
| | | | Tyr | 57H | CG | 3.83 | |
| | | | Tyr | 57H | CD1 | 3.74 | |
| | | | Tyr | 94L | CD2 | 4.69 | |
| | | | His | 59H | ND1 | 4.96 | * |
| | | | His | 59H | CE1 | 3.83 | |
| | | | Tyr | 94L | CZ | 4.61 | |
| | | | Tyr | 94L | OH | 4.58 | * |
| | | | Tyr | 94L | CE2 | 3.97 | |
| | | | His | 59H | CG | 4.90 | |
| | | | His | 59H | NE2 | 2.81 | *** |
| | | | His | 59H | CD2 | 3.67 | |
| Glu | 68I | OE2 | Tyr | 57H | CE1 | 3.86 | |
| | | | Tyr | 57H | CZ | 4.99 | |
| | | | Tyr | 57H | CB | 4.52 | |
| | | | Tyr | 57H | CG | 4.23 | |
| | | | Tyr | 57H | CD1 | 3.38 | |
| | | | His | 59H | CE1 | 3.79 | |
| | | | His | 59H | NE2 | 3.47 | * |
| | | | His | 59H | CD2 | 4.76 | |
| Glu | 68I | C | Thr | 52H | CG2 | 4.29 | |
| Thr | 69I | N | Tyr | 94L | OH | 4.53 | * |
| | | | Ser | 33H | OG | 4.78 | * |
| | | | Thr | 52H | CG2 | 3.80 | |
| Thr | 69I | CA | Tyr | 94L | OH | 4.69 | |
| | | | Ser | 33H | OG | 4.30 | |
| | | | Thr | 52H | CG2 | 4.67 | |
| Thr | 69I | CB | Tyr | 94L | OH | 3.73 | |
| | | | Ser | 33H | CB | 4.38 | |
| | | | Ser | 33H | OG | 3.68 | |
| | | | Thr | 52H | CG2 | 4.73 | |
| | | | Tyr | 96L | OH | 4.41 | |
| | | | Glu | 99H | CD | 4.63 | |
| | | | Glu | 99H | OE1 | 4.57 | |
| | | | Glu | 99H | OE2 | 4.55 | |
| Thr | 69I | OG1 | Thr | 52H | CB | 4.69 | |
| | | | Tyr | 94L | OH | 3.93 | * |
| | | | Ser | 50H | OG | 4.64 | * |
| | | | Ser | 33H | CA | 4.76 | |
| | | | Ser | 33H | CB | 3.37 | |
| | | | Ser | 33H | OG | 2.64 | *** |
| | | | Thr | 52H | CA | 4.77 | |
| | | | Thr | 52H | CG2 | 3.59 | |
| | | | Glu | 99H | CD | 4.91 | |
| | | | Glu | 99H | OE1 | 4.55 | * |
| Thr | 69I | CG2 | Tyr | 94L | OH | 4.69 | |
| | | | Ser | 33H | CB | 4.12 | |
| | | | Ser | 33H | OG | 3.66 | |
| | | | Tyr | 96L | OH | 4.28 | |
| | | | Arg | 100H | C | 4.70 | |
| | | | Arg | 100H | O | 4.37 | |
| | | | Gly | 101H | N | 4.53 | |
| | | | Gly | 101H | CA | 4.09 | |
| | | | Glu | 99H | CG | 4.21 | |
| | | | Glu | 99H | CD | 3.63 | |
| | | | Glu | 99H | OE1 | 3.93 | |
| | | | Glu | 99H | OE2 | 3.57 | |
| Thr | 69I | O | Tyr | 94L | OH | 4.54 | * |
| Asn | 70I | N | Trp | 102H | N | 4.38 | * |
| | | | Gly | 101H | CA | 4.14 | |
| | | | Gly | 101H | C | 4.75 | |
| Asn | 70I | CA | Trp | 102H | N | 4.20 | |
| | | | Gly | 103H | N | 4.46 | |
| | | | Gly | 101H | CA | 4.56 | |
| | | | Gly | 101H | C | 4.74 | |
| Asn | 70I | CB | Gly | 103H | CA | 4.91 | |
| | | | Tyr | 105H | CE1 | 3.59 | |
| | | | Tyr | 105H | CZ | 4.07 | |
| | | | Tyr | 105H | OH | 4.04 | |
| | | | Trp | 102H | N | 4.95 | |
| | | | Gly | 103H | N | 4.56 | |
| | | | Tyr | 105H | CD1 | 4.33 | |
| Asn | 70I | CG | Gly | 103H | CA | 4.36 | |
| | | | Tyr | 105H | CE1 | 3.37 | |
| | | | Tyr | 105H | CZ | 4.07 | |
| | | | Tyr | 105H | OH | 4.54 | |
| | | | Trp | 102H | N | 4.48 | |
| | | | Gly | 103H | N | 4.05 | |
| | | | Arg | 100H | O | 4.62 | |
| | | | Gly | 101H | CA | 4.10 | |
| | | | Gly | 101H | C | 4.37 | |
| | | | Gly | 101H | O | 4.98 | |
| | | | Tyr | 105H | CA | 4.92 | |
| | | | Tyr | 105H | CG | 4.55 | |
| | | | Tyr | 105H | CD1 | 3.61 | |
| | | | Tyr | 105H | CE2 | 4.95 | |
| | | | Gly | 103H | C | 4.97 | |
| | | | Tyr | 104H | N | 4.91 | |
| | | | Tyr | 105H | N | 4.64 | |
| Asn | 70I | OD1 | Gly | 103H | CA | 3.39 | |
| | | | Tyr | 105H | CE1 | 3.90 | |
| | | | Tyr | 105H | CZ | 4.36 | |
| | | | Tyr | 105H | OH | 4.95 | * |
| | | | Trp | 102H | N | 3.47 | * |
| | | | Trp | 102H | CA | 4.34 | |
| | | | Trp | 102H | C | 4.02 | |
| | | | Gly | 103H | N | 2.93 | *** |
| | | | Arg | 100H | C | 4.69 | |
| | | | Arg | 100H | O | 4.08 | * |
| | | | Gly | 101H | N | 4.50 | * |
| | | | Gly | 101H | CA | 3.34 | |
| | | | Gly | 101H | C | 3.29 | |
| | | | Gly | 101H | O | 3.78 | * |
| | | | Tyr | 105H | CA | 4.70 | |
| | | | Tyr | 105H | CG | 4.54 | |
| | | | Tyr | 105H | CD1 | 3.94 | |
| | | | Tyr | 105H | CE2 | 4.93 | |
| | | | Tyr | 105H | CD2 | 4.98 | |
| | | | Gly | 103H | C | 3.95 | |
| | | | Tyr | 104H | N | 3.78 | * |
| | | | Tyr | 104H | CA | 4.86 | |
| | | | Tyr | 104H | C | 4.95 | |
| | | | Tyr | 105H | N | 4.05 | * |
| Asn | 70I | ND2 | Tyr | 105H | CE1 | 3.46 | |
| | | | Tyr | 105H | CZ | 4.49 | |
| | | | Phe | 91L | CB | 4.55 | |
| | | | Phe | 91L | CD2 | 4.42 | |
| | | | Tyr | 96L | OH | 4.15 | * |
| | | | Arg | 100H | O | 4.35 | * |
| | | | Gly | 101H | CA | 4.55 | |
| | | | Glu | 99H | CD | 4.80 | |
| | | | Glu | 99H | OE2 | 3.81 | * |
| | | | Tyr | 105H | CA | 4.49 | |
| | | | Tyr | 105H | CG | 4.49 | |
| | | | Tyr | 105H | CD1 | 3.39 | |
| | | | Phe | 91L | CG | 4.84 | |
| | | | Tyr | 105H | N | 4.53 | * |
| Asn | 70I | O | Tyr | 105H | OH | 4.32 | * |
| Glu | 72I | CA | Trp | 102H | CE2 | 4.68 | |
| | | | Trp | 102H | CD2 | 4.57 | |
| | | | Trp | 102H | CE3 | 4.51 | |
| | | | Trp | 102H | CZ3 | 4.52 | |
| | | | Trp | 102H | CH2 | 4.59 | |
| | | | Trp | 102H | CZ2 | 4.72 | |
| Glu | 72I | CB | Trp | 102H | NE1 | 3.78 | |
| | | | Trp | 102H | CE2 | 3.36 | |
| | | | Trp | 102H | CD2 | 3.35 | |
| | | | Trp | 102H | CE3 | 3.66 | |
| | | | Trp | 102H | CZ3 | 3.93 | |
| | | | Trp | 102H | CH2 | 3.90 | |
| | | | Trp | 102H | CZ2 | 3.68 | |
| | | | Trp | 102H | N | 4.30 | |
| | | | Trp | 102H | CA | 4.36 | |
| | | | Trp | 102H | CB | 4.71 | |
| | | | Trp | 102H | CG | 3.87 | |
| | | | Trp | 102H | CD1 | 4.10 | |
| Glu | 72I | CG | Trp | 102H | NE1 | 3.76 | |
| | | | Trp | 102H | CE2 | 3.82 | |
| | | | Trp | 102H | CD2 | 4.18 | |
| | | | Trp | 102H | CE3 | 4.86 | |
| | | | Trp | 102H | CH2 | 4.83 | |
| | | | Trp | 102H | CZ2 | 4.22 | |
| | | | Trp | 102H | N | 4.50 | |
| | | | Trp | 102H | CG | 4.40 | |
| | | | Trp | 102H | CD1 | 4.17 | |
| Glu | 72I | CD | Trp | 102H | NE1 | 4.30 | |
| | | | Trp | 102H | CE2 | 4.68 | |
| | | | Trp | 102H | CD2 | 4.79 | |
| | | | Trp | 102H | N | 3.68 | |
| | | | Trp | 102H | CA | 4.59 | |
| | | | Trp | 102H | CG | 4.54 | |
| | | | Trp | 102H | CD1 | 4.25 | |
| | | | Gly | 101H | CA | 4.13 | |
| | | | Gly | 101H | C | 4.42 | |
| Glu | 72I | OE1 | Trp | 102H | NE1 | 4.76 | * |
| | | | Trp | 102H | CD2 | 4.77 | |
| | | | Trp | 102H | N | 2.85 | *** |
| | | | Trp | 102H | CA | 3.77 | |
| | | | Trp | 102H | CB | 4.68 | |
| | | | Trp | 102H | CG | 4.38 | |
| | | | Trp | 102H | CD1 | 4.38 | |
| | | | Trp | 102H | C | 4.87 | |
| | | | Gly | 103H | N | 4.82 | * |
| | | | Gly | 101H | N | 4.90 | * |
| | | | Gly | 101H | CA | 3.62 | |
| | | | Gly | 101H | C | 3.72 | |
| | | | Gly | 101H | O | 4.94 | * |
| Glu | 72I | OE2 | Trp | 102H | NE1 | 4.74 | * |
| | | | Ser | 33H | OG | 4.98 | * |
| | | | Trp | 102H | N | 4.29 | * |
| | | | Trp | 102H | CD1 | 4.69 | |
| | | | Gly | 101H | N | 4.83 | * |
| | | | Gly | 101H | CA | 4.01 | |
| | | | Gly | 101H | C | 4.70 | |
| Glu | 72I | C | Trp | 102H | CE2 | 4.90 | |
| | | | Trp | 102H | CD2 | 4.55 | |
| | | | Trp | 102H | CE3 | 4.02 | |
| | | | Trp | 102H | CZ3 | 3.82 | |
| | | | Trp | 102H | CH2 | 4.20 | |
| | | | Trp | 102H | CZ2 | 4.76 | |
| Glu | 72I | O | Trp | 102H | CD2 | 4.94 | |
| | | | Trp | 102H | CE3 | 4.27 | |
| | | | Trp | 102H | CZ3 | 3.64 | |
| | | | Trp | 102H | CH2 | 3.82 | |
| | | | Trp | 102H | CZ2 | 4.59 | |
| Trp | 73I | N | Trp | 102H | CD2 | 4.69 | |
| | | | Trp | 102H | CE3 | 3.99 | |
| | | | Trp | 102H | CZ3 | 4.07 | |
| | | | Trp | 102H | CH2 | 4.84 | |
| | | | Trp | 102H | CA | 4.92 | |
| Trp | 73I | CA | Trp | 102H | CE3 | 4.35 | |
| | | | Trp | 102H | CZ3 | 4.23 | |
| Trp | 73I | CB | Trp | 102H | CE3 | 4.73 | |
| | | | Trp | 102H | CZ3 | 4.93 | |
| Trp | 73I | CG | Trp | 102H | CE3 | 3.91 | |
| | | | Trp | 102H | CZ3 | 4.35 | |
| | | | Trp | 102H | CA | 4.76 | |
| | | | Trp | 102H | C | 4.85 | |
| | | | Trp | 102H | O | 4.73 | |
| Trp | 73I | CD1 | Trp | 102H | CD2 | 4.81 | |
| | | | Trp | 102H | CE3 | 3.97 | |
| | | | Trp | 102H | CZ3 | 4.80 | |
| | | | Gly | 103H | CA | 4.80 | |
| | | | Trp | 102H | CA | 3.80 | |
| | | | Trp | 102H | CB | 4.27 | |
| | | | Trp | 102H | C | 3.61 | |
| | | | Trp | 102H | O | 3.38 | |
| | | | Gly | 103H | N | 4.31 | |
| Trp | 73I | NE1 | Trp | 102H | CD2 | 4.59 | |
| | | | Trp | 102H | CE3 | 3.76 | |
| | | | Trp | 102H | CZ3 | 4.68 | |
| | | | Trp | 102H | CA | 3.94 | |
| | | | Trp | 102H | CB | 3.93 | |
| | | | Trp | 102H | CG | 4.74 | |
| | | | Trp | 102H | C | 3.73 | |
| | | | Trp | 102H | O | 3.11 | *** |
| | | | Gly | 103H | N | 4.71 | * |
| Trp | 73I | CE2 | Trp | 102H | CD2 | 4.62 | |
| | | | Trp | 102H | CE3 | 3.51 | |
| | | | Trp | 102H | CZ3 | 4.09 | |
| | | | Trp | 102H | CA | 4.92 | |
| | | | Trp | 102H | CB | 4.64 | |
| | | | Trp | 102H | C | 4.96 | |
| | | | Trp | 102H | O | 4.38 | |
| Trp | 73I | CD2 | Trp | 102H | CD2 | 4.94 | |
| | | | Trp | 102H | CE3 | 3.64 | |
| | | | Trp | 102H | CZ3 | 3.85 | |
| Trp | 73I | CE3 | Trp | 102H | CE3 | 4.12 | |
| | | | Trp | 102H | CZ3 | 3.84 | |
| Trp | 73I | CZ3 | Trp | 102H | CE3 | 4.47 | |
| | | | Trp | 102H | CZ3 | 4.10 | |
| Trp | 73I | CH2 | Trp | 102H | CE3 | 4.44 | |
| | | | Trp | 102H | CZ3 | 4.40 | |
| Trp | 73I | CZ2 | Trp | 102H | CE3 | 3.98 | |
| | | | Trp | 102H | CZ3 | 4.39 | |
| Phe | 76I | CB | Trp | 102H | CZ3 | 3.95 | |
| | | | Trp | 102H | CH2 | 4.19 | |
| Phe | 76I | CG | Trp | 102H | CZ3 | 4.48 | |
| | | | Trp | 102H | CH2 | 4.78 | |
| Phe | 76I | CD1 | Trp | 102H | CZ3 | 4.59 | |
| | | | Trp | 102H | CH2 | 4.62 | |
| Ala | 112I | C | Trp | 102H | O | 4.12 | |
| Ala | 112I | O | Trp | 102H | CB | 4.62 | |
| | | | Trp | 102H | C | 4.57 | |
| | | | Trp | 102H | O | 3.39 | * |
| Gly | 113I | N | Trp | 102H | O | 4.18 | * |
| Gly | 113I | CA | Gly | 103H | CA | 4.35 | |
| | | | Trp | 102H | C | 4.50 | |
| | | | Trp | 102H | O | 3.38 | |
| | | | Gly | 103H | N | 4.93 | |
| | | | Asp | 50L | OD2 | 4.85 | |
| | | | Gly | 103H | O | 4.10 | |
| | | | Gly | 103H | C | 4.30 | |
| Gly | 113I | C | Gly | 103H | CA | 4.66 | |
| | | | Trp | 102H | C | 4.76 | |
| | | | Trp | 102H | O | 3.81 | |
| | | | Gly | 103H | O | 4.98 | |
| Gly | 113I | O | Gly | 103H | CA | 4.14 | |
| | | | Trp | 102H | C | 4.62 | |
| | | | Trp | 102H | O | 3.94 | * |
| | | | Gly | 103H | N | 4.76 | * |
| | | | Gly | 103H | O | 4.76 | * |
| | | | Gly | 103H | C | 4.81 | |
| Arg | 114I | N | Trp | 102H | O | 4.66 | * |
| Gln | 116I | CG | Asp | 50L | OD1 | 4.81 | |
| | | | Tyr | 105H | CE2 | 4.89 | |
| Gln | 116I | CD | Gly | 103H | CA | 4.60 | |
| | | | Ser | 31L | CB | 4.98 | |
| | | | Asp | 50L | CG | 3.93 | |
| | | | Asp | 50L | OD1 | 3.39 | |
| | | | Asp | 50L | OD2 | 3.74 | |
| | | | Gly | 103H | O | 4.02 | |
| | | | Tyr | 105H | CE2 | 3.87 | |
| | | | Tyr | 105H | CD2 | 4.25 | |
| | | | Gly | 103H | C | 4.77 | |
| Gln | 116I | OE1 | Ser | 31L | N | 4.89 | * |
| | | | Ser | 31L | CA | 4.73 | |
| | | | Ser | 31L | CB | 3.74 | |
| | | | Asp | 50L | CG | 3.31 | |
| | | | Asp | 50L | OD1 | 2.49 | *** |
| | | | Asp | 50L | OD2 | 3.40 | * |
| | | | Gly | 103H | O | 4.30 | * |
| | | | Ser | 31L | C | 4.80 | |
| | | | Ser | 31L | O | 4.75 | * |
| | | | Asp | 50L | CB | 4.75 | |
| | | | Tyr | 105H | CE2 | 3.72 | |
| | | | Tyr | 105H | CD2 | 3.89 | |
| | | | Ser | 31L | OG | 4.34 | * |
| Gln | 116I | NE2 | Gly | 103H | CA | 3.44 | |
| | | | Tyr | 105H | CZ | 4.94 | |
| | | | Trp | 102H | O | 4.87 | * |
| | | | Gly | 103H | N | 4.76 | * |
| | | | Asp | 50L | CG | 3.83 | |
| | | | Asp | 50L | OD1 | 3.59 | * |
| | | | Asp | 50L | OD2 | 3.54 | * |
| | | | Gly | 103H | O | 2.94 | *** |
| | | | Tyr | 105H | CE2 | 3.83 | |
| | | | Tyr | 105H | CD2 | 4.05 | |
| | | | Gly | 103H | C | 3.58 | |
| | | | Tyr | 104H | N | 4.86 | * |
| Lys | 117I | CA | Asp | 30L | CG | 4.74 | |
| | | | Asp | 30L | OD2 | 4.33 | |
| Lys | 117I | CB | Asp | 30L | OD1 | 3.90 | |
| | | | Asp | 30L | CB | 4.08 | |
| | | | Asp | 30L | CG | 3.56 | |
| | | | Asp | 30L | OD2 | 3.38 | |
| Lys | 117I | CG | Asp | 30L | OD1 | 4.25 | |
| | | | Asp | 30L | CB | 4.79 | |
| | | | Asp | 30L | CG | 4.31 | |
| | | | Asp | 30L | OD2 | 4.47 | |
| Lys | 117I | CD | Asp | 30L | OD1 | 3.48 | |
| | | | Asp | 30L | CB | 4.68 | |
| | | | Asp | 30L | CG | 3.98 | |
| | | | Asp | 30L | OD2 | 4.38 | |
| Lys | 117I | CE | Asp | 30L | OD1 | 4.56 | |
| Lys | 117I | NZ | Asp | 30L | OD1 | 4.47 | * |
| | | | Ser | 67L | OG | 4.88 | * |
| Lys | 117I | C | Asp | 30L | CB | 4.88 | |
| | | | Asp | 30L | CG | 4.71 | |
| | | | Asp | 30L | OD2 | 4.04 | |
| Lys | 117I | O | Tyr | 105H | OH | 4.71 | * |
| | | | Asp | 30L | CB | 4.39 | |
| | | | Asp | 30L | CG | 4.60 | |
| | | | Asp | 30L | OD2 | 4.07 | * |
| | | | Tyr | 105H | CE2 | 4.96 | |
| His | 118I | N | Asp | 30L | OD2 | 4.47 | * |
| His | 118I | CA | Tyr | 105H | OH | 4.54 | |
| | | | Asp | 30L | OD2 | 4.88 | |
| His | 118I | ND1 | Gly | 103H | CA | 4.99 | |
| | | | Tyr | 105H | OH | 4.33 | * |
| His | 118I | CE1 | Gly | 103H | CA | 4.46 | |
| | | | Gly | 103H | N | 4.81 | |
| His | 118I | C | Tyr | 105H | OH | 4.74 | |
| | | | Asp | 30L | OD2 | 4.49 | |
| His | 118I | O | Asp | 30L | OD2 | 3.90 | * |
| Arg | 119I | N | Tyr | 105H | OH | 4.16 | * |
| Arg | 119I | CA | Tyr | 105H | OH | 4.92 | |
| Arg | 119I | CB | Asn | 92L | O | 4.54 | |
| | | | Tyr | 105H | OH | 4.55 | |
| Arg | 119I | CG | Asn | 92L | CA | 4.83 | |
| | | | Asn | 92L | ND2 | 4.87 | |
| | | | Asn | 92L | O | 4.24 | |
| | | | Phe | 91L | O | 4.82 | |
| | | | Asn | 92L | C | 4.98 | |
| | | | Tyr | 105H | CE1 | 4.63 | |
| | | | Tyr | 105H | CZ | 4.39 | |
| | | | Tyr | 105H | OH | 3.20 | |
| Arg | 119I | CD | Asn | 92L | CA | 4.04 | |
| | | | Asn | 92L | O | 3.46 | |
| | | | Phe | 91L | C | 4.43 | |
| | | | Phe | 91L | O | 3.47 | |
| | | | Asn | 92L | N | 4.73 | |
| | | | Asn | 92L | C | 4.06 | |
| | | | Tyr | 105H | CE1 | 4.13 | |
| | | | Tyr | 105H | CZ | 4.34 | |
| | | | Tyr | 105H | OH | 3.53 | |
| Arg | 119I | NE | Asn | 92L | O | 4.22 | * |
| | | | Phe | 91L | O | 3.94 | * |
| | | | Asn | 92L | C | 4.88 | |
| | | | Tyr | 105H | CE1 | 4.56 | |
| | | | Tyr | 105H | CZ | 4.93 | |
| | | | Tyr | 105H | OH | 4.18 | * |
| Arg | 119I | CZ | Tyr | 94L | CE2 | 4.85 | |
| | | | Asn | 92L | O | 4.03 | |
| | | | Phe | 91L | O | 4.18 | |
| | | | Asn | 92L | C | 4.78 | |
| Arg | 119I | NH1 | Tyr | 94L | CD2 | 4.96 | |
| | | | Tyr | 94L | CE2 | 4.69 | |
| | | | Asn | 92L | CA | 4.71 | |
| | | | Asn | 92L | O | 2.95 | *** |
| | | | Phe | 91L | O | 4.00 | * |
| | | | Asn | 92L | C | 3.81 | |
| | | | Ser | 93L | N | 4.48 | * |
| | | | Ser | 93L | CA | 4.59 | |
| | | | Ser | 93L | CA | 4.42 | |
| Arg | 119I | NH2 | Tyr | 94L | OH | 4.96 | * |
| | | | Tyr | 94L | CE2 | 4.33 | |
| Pro | 123I | CG | Tyr | 57H | OH | 4.85 | |
| Tyr | 128I | CD1 | Tyr | 57H | OH | 4.87 | |
| Tyr | 128I | CE1 | Tyr | 57H | OH | 4.32 | |
| Leu | 143I | CA | Trp | 102H | CZ2 | 4.90 | |
| Leu | 143I | CB | Trp | 102H | CZ2 | 4.87 | |
| Leu | 143I | CG | Trp | 102H | CE2 | 4.93 | |
| | | | Trp | 102H | CH2 | 3.72 | |
| | | | Trp | 102H | CZ2 | 3.64 | |
| Leu | 143I | CD1 | Trp | 102H | CE2 | 4.85 | |
| | | | Trp | 102H | CH2 | 4.25 | |
| | | | Trp | 102H | CZ2 | 3.86 | |
| Leu | 143I | CD2 | Trp | 102H | CZ3 | 4.97 | |
| | | | Trp | 102H | CH2 | 3.91 | |
| | | | Trp | 102H | CZ2 | 4.32 | |
| Leu | 143I | C | Trp | 102H | CH2 | 4.73 | |
| | | | Trp | 102H | CZ2 | 4.21 | |
| Leu | 143I | O | Trp | 102H | NE1 | 4.65 | * |
| | | | Trp | 102H | CE2 | 4.35 | |
| | | | Trp | 102H | CH2 | 4.16 | |
| | | | Trp | 102H | CZ2 | 3.38 | |
| Leu | 144I | CD2 | Trp | 102H | CH2 | 4.96 | |
| Gln | 145I | C | Ser | 31H | OG | 4.98 | |
| Gln | 145I | O | Ser | 31H | OG | 4.67 | * |
| Lys | 146I | N | Ser | 31H | OG | 4.68 | * |
| Lys | 146I | CA | Ser | 31H | CA | 4.90 | |
| | | | Ser | 31H | CB | 4.13 | |
| | | | Ser | 31H | OG | 3.91 | |
| | | | Ser | 31H | O | 4.74 | |
| Lys | 146I | CB | Trp | 102H | NE1 | 4.78 | |
| | | | Ser | 31H | CA | 4.55 | |
| | | | Ser | 31H | CB | 4.26 | |
| | | | Ser | 31H | OG | 4.21 | |
| | | | Ser | 31H | C | 4.83 | |
| | | | Ser | 31H | O | 4.15 | |
| Lys | 146I | CG | Ser | 30H | O | 4.72 | |
| | | | Ser | 53H | OG | 4.92 | |
| | | | Ser | 31H | CA | 3.95 | |
| | | | Ser | 31H | CB | 3.98 | |
| | | | Ser | 31H | OG | 3.65 | |
| | | | Ser | 31H | C | 4.56 | |
| | | | Ser | 31H | O | 4.24 | |
| Lys | 146I | CD | Ser | 30H | O | 4.76 | |
| | | | Ser | 53H | CB | 4.79 | |
| | | | Ser | 53H | OG | 3.86 | |
| | | | Ser | 31H | CA | 4.58 | |
| | | | Ser | 31H | OG | 4.92 | |
| | | | Ser | 31H | C | 4.89 | |
| | | | Ser | 31H | O | 4.50 | |
| Lys | 146I | CE | Ser | 30H | O | 4.19 | |
| | | | Ser | 53H | CB | 4.29 | |
| | | | Ser | 53H | OG | 3.31 | |
| | | | Ser | 31H | CA | 4.91 | |
| Lys | 146I | NZ | Ser | 53H | OG | 4.68 | * |
| Lys | 146I | C | Trp | 102H | NE1 | 4.31 | |
| | | | Trp | 102H | CE2 | 4.94 | |
| | | | Ser | 31H | CB | 4.75 | |
| | | | Ser | 31H | OG | 4.86 | |
| | | | Ser | 31H | O | 4.98 | |
| Lys | 146I | O | Trp | 102H | NE1 | 4.67 | * |
| | | | Ser | 31H | CB | 4.33 | |
| | | | Ser | 31H | OG | 4.68 | * |
| | | | Ser | 31H | O | 4.62 | * |
| Met | 147I | N | Trp | 102H | NE1 | 3.83 | * |
| | | | Trp | 102H | CE2 | 4.14 | |
| | | | Trp | 102H | CZ2 | 4.15 | |
| | | | Trp | 102H | CD1 | 4.77 | |
| Met | 147I | CA | Trp | 102H | NE1 | 3.62 | |
| | | | Trp | 102H | CE2 | 3.78 | |
| | | | Trp | 102H | CD2 | 4.55 | |
| | | | Trp | 102H | CH2 | 4.94 | |
| | | | Trp | 102H | CZ2 | 4.05 | |
| | | | Trp | 102H | CG | 4.77 | |
| | | | Trp | 102H | CD1 | 4.25 | |
| Met | 147I | CA | Trp | 102H | NE1 | 3.62 | |
| | | | Trp | 102H | CE2 | 3.76 | |
| | | | Trp | 102H | CD2 | 4.53 | |
| | | | Trp | 102H | CH2 | 4.88 | |
| | | | Trp | 102H | CZ2 | 4.00 | |
| | | | Trp | 102H | CG | 4.78 | |
| | | | Trp | 102H | CD1 | 4.27 | |
| Met | 147I | CB | Trp | 102H | NE1 | 3.84 | |
| | | | Trp | 102H | CE2 | 3.45 | |
| | | | Trp | 102H | CD2 | 4.08 | |
| | | | Trp | 102H | CE3 | 4.51 | |
| | | | Trp | 102H | CZ3 | 4.43 | |
| | | | Trp | 102H | CH2 | 3.91 | |
| | | | Trp | 102H | CZ2 | 3.38 | |
| | | | Trp | 102H | CG | 4.70 | |
| | | | Trp | 102H | CD1 | 4.55 | |
| Met | 147I | CB | Trp | 102H | NE1 | 3.98 | |
| | | | Trp | 102H | CE2 | 3.56 | |
| | | | Trp | 102H | CD2 | 4.23 | |
| | | | Trp | 102H | CE3 | 4.63 | |
| | | | Trp | 102H | CZ3 | 4.48 | |
| | | | Trp | 102H | CH2 | 3.90 | |
| | | | Trp | 102H | CZ2 | 3.39 | |
| | | | Trp | 102H | CG | 4.90 | |
| | | | Trp | 102H | CD1 | 4.74 | |
| Met | 147I | CG | Trp | 102H | NE1 | 4.22 | |
| | | | Trp | 102H | CE2 | 3.69 | |
| | | | Trp | 102H | CD2 | 3.73 | |
| | | | Trp | 102H | CE3 | 3.92 | |
| | | | Trp | 102H | CZ3 | 4.10 | |
| | | | Trp | 102H | CH2 | 4.11 | |
| | | | Trp | 102H | CZ2 | 3.92 | |
| | | | Trp | 102H | CG | 4.26 | |
| | | | Trp | 102H | CD1 | 4.50 | |
| Met | 147I | CG | Trp | 102H | NE1 | 4.16 | |
| | | | Trp | 102H | CE2 | 3.47 | |
| | | | Trp | 102H | CD2 | 3.58 | |
| | | | Trp | 102H | CE3 | 3.67 | |
| | | | Trp | 102H | CZ3 | 3.65 | |
| | | | Trp | 102H | CH2 | 3.58 | |
| | | | Trp | 102H | CZ2 | 3.50 | |
| | | | Trp | 102H | CG | 4.32 | |
| | | | Trp | 102H | CD1 | 4.59 | |
| Met | 147I | SD | Trp | 102H | CE2 | 4.84 | |
| | | | Trp | 102H | CD2 | 4.70 | |
| | | | Trp | 102H | CE3 | 4.34 | |
| | | | Trp | 102H | CZ3 | 4.13 | |
| | | | Trp | 102H | CH2 | 4.33 | |
| | | | Trp | 102H | CZ2 | 4.69 | |
| Met | 147I | SD | Trp | 102H | CE2 | 4.48 | |
| | | | Trp | 102H | CD2 | 4.50 | |
| | | | Trp | 102H | CE3 | 4.01 | |
| | | | Trp | 102H | CZ3 | 3.46 | |
| | | | Trp | 102H | CH2 | 3.49 | |
| | | | Trp | 102H | CZ2 | 4.02 | |
| Met | 147I | C | Trp | 102H | NE1 | 4.97 | |
| His | 149I | CB | Ser | 31H | CB | 3.80 | |
| | | | Ser | 31H | OG | 3.77 | |
| | | | Ile | 28H | CB | 4.57 | |
| | | | Ile | 28H | CG1 | 4.68 | |
| His | 149I | CG | Ser | 31H | CB | 4.25 | |
| | | | Ser | 31H | OG | 3.71 | |
| | | | Ile | 28H | CB | 4. | |
| | | | Ile | 28H | CG1 | 3.94 | |
| | | | Ile | 28H | CG2 | 4.41 | |
| His | 149I | ND1 | Ser | 31H | CB | 3.82 | |
| | | | Ser | 31H | OG | 2.88 | *** |
| | | | Ile | 28H | CB | 3.88 | |
| | | | Ile | 28H | CG1 | 3.96 | |
| | | | Ile | 28H | CG2 | 3.70 | |
| His | 149I | CE1 | Ser | 31H | OG | 3.92 | |
| | | | Ile | 28H | CB | 4.15 | |
| | | | Ile | 28H | CG1 | 3.87 | |
| | | | Ile | 28H | CD1 | 4.82 | |
| | | | Ile | 28H | CG2 | 3.80 | |
| His | 149I | NE2 | Ile | 28H | CB | 4.55 | |
| | | | Ile | 28H | CG1 | 3.84 | |
| | | | Ile | 28H | CD1 | 4.72 | |
| | | | Ile | 28H | CG2 | 4.57 | |
| His | 149I | CD2 | Ser | 31H | OG | 4.93 | |
| | | | Ile | 28H | CB | 4.52 | |
| | | | Ile | 28H | CG1 | 3.86 | |
| | | | Ile | 28H | CG2 | 4.90 | |
| His | 149I | C | Tyr | 32H | CE1 | 4.99 | |
| | | | Tyr | 32H | CZ | 4.67 | |
| | | | Tyr | 32H | OH | 3.97 | |
| His | 149I | O | Tyr | 32H | CE1 | 4.96 | |
| | | | Tyr | 32H | CZ | 4.48 | |
| | | | Tyr | 32H | OH | 3.48 | * |
| Gln | 150I | N | Tyr | 32H | CZ | 4.58 | |
| | | | Tyr | 32H | OH | 4.13 | * |
| Gln | 150I | CA | Tyr | 32H | CE1 | 4.87 | |
| | | | Tyr | 32H | CZ | 4.03 | |
| | | | Tyr | 32H | CE2 | 4.32 | |
| | | | Arg | 100H | NE | 4.67 | |
| | | | Tyr | 32H | OH | 3.57 | |
| Gln | 150I | CB | Trp | 102H | CD1 | 4.90 | |
| | | | Tyr | 32H | CZ | 4.48 | |
| | | | Tyr | 32H | CE2 | 4.37 | |
| | | | Arg | 100H | CG | 4.58 | |
| | | | Arg | 100H | CD | 4.86 | |
| | | | Arg | 100H | NE | 4.22 | |
| | | | Tyr | 32H | OH | 4.44 | |
| | | | Arg | 100H | NH2 | 4.88 | |
| | | | Arg | 100H | CZ | 4.86 | |
| Gln | 150I | CG | Ser | 31H | CB | 4.93 | |
| | | | Ser | 31H | C | 4.81 | |
| | | | Ser | 31H | O | 3.93 | |
| | | | Tyr | 32H | CD1 | 4.77 | |
| | | | Trp | 102H | CD1 | 4.82 | |
| | | | Tyr | 32H | CG | 4.62 | |
| | | | Tyr | 32H | CE1 | 4.46 | |
| | | | Tyr | 32H | CZ | 3.94 | |
| | | | Tyr | 32H | CE2 | 3.79 | |
| | | | Tyr | 32H | CD2 | 4.13 | |
| | | | Arg | 100H | CG | 4.74 | |
| | | | Tyr | 32H | OH | 4.34 | |
| Gln | 150I | CD | Trp | 102H | NE1 | 4.57 | |
| | | | Ser | 31H | C | 4.85 | |
| | | | Ser | 31H | O | 3.79 | |
| | | | Trp | 102H | CD1 | 4.02 | |
| | | | Arg | 100H | CA | 4.16 | |
| | | | Arg | 100H | CB | 4.42 | |
| | | | Arg | 100H | C | 4.56 | |
| | | | Gly | 101H | N | 3.86 | |
| | | | Gly | 101H | CA | 4.81 | |
| | | | Tyr | 32H | CG | 4.73 | |
| | | | Tyr | 32H | CZ | 4.79 | |
| | | | Tyr | 32H | CE2 | 4.26 | |
| | | | Tyr | 32H | CD2 | 4.20 | |
| | | | Arg | 100H | CG | 4.44 | |
| Gln | 150I | OE1 | Trp | 102H | NE1 | 4.52 | * |
| | | | Ser | 31H | O | 4.95 | * |
| | | | Trp | 102H | N | 4.84 | * |
| | | | Trp | 102H | CB | 4.81 | |
| | | | Trp | 102H | CG | 4.56 | |
| | | | Trp | 102H | CD1 | 3.60 | |
| | | | Arg | 100H | N | 4.84 | * |
| | | | Arg | 100H | CA | 3.48 | |
| | | | Arg | 100H | CB | 3.57 | |
| | | | Arg | 100H | C | 3.68 | |
| | | | Arg | 100H | O | 4.86 | * |
| | | | Gly | 101H | N | 2.98 | *** |
| | | | Gly | 101H | CA | 3.92 | |
| | | | Gly | 101H | C | 4.05 | |
| | | | Gly | 101H | O | 4.00 | * |
| | | | Tyr | 32H | CE2 | 4.81 | |
| | | | Tyr | 32H | CD2 | 4.81 | |
| | | | Arg | 100H | CG | 3.91 | |
| | | | Arg | 100H | CD | 4.81 | |
| | | | Arg | 100H | NE | 4.42 | * |
| Gln | 150I | NE2 | Trp | 102H | NE1 | 4.55 | * |
| | | | Ser | 33H | N | 4.95 | * |
| | | | Ser | 31H | C | 3.92 | |
| | | | Ser | 31H | O | 2.78 | *** |
| | | | Tyr | 32H | N | 4.50 | * |
| | | | Tyr | 32H | CA | 4.08 | |
| | | | Tyr | 32H | CB | 4.72 | |
| | | | Trp | 102H | CD1 | 4.27 | |
| | | | Arg | 100H | CA | 4.54 | |
| | | | Arg | 100H | C | 4.80 | |
| | | | Gly | 101H | N | 3.98 | * |
| | | | Gly | 101H | CA | 4.82 | |
| | | | Glu | 99H | O | 4.79 | * |
| | | | Tyr | 32H | CG | 4.45 | |
| | | | Tyr | 32H | CE2 | 4.67 | |
| | | | Tyr | 32H | CD2 | 4.25 | |
| Gln | 150I | C | Arg | 100H | NE | 4.26 | |
| | | | Tyr | 32H | OH | 4.29 | |
| | | | Arg | 100H | NH2 | 4.15 | |
| | | | Arg | 100H | CZ | 4.24 | |
| | | | Arg | 100H | NH1 | 4.92 | |
| Gln | 150I | O | Arg | 100H | CG | 4.80 | |
| | | | Arg | 100H | CD | 4.11 | |
| | | | Arg | 100H | NE | 3.37 | * |
| | | | Tyr | 32H | OH | 4.31 | * |
| | | | Arg | 100H | NH2 | 3.33 | * |
| | | | Arg | 100H | CZ | 3.21 | |
| | | | Arg | 100H | NH1 | 3.73 | * |
| His | 151I | N | Arg | 100H | NH2 | 4.64 | * |
| His | 151I | CA | Arg | 100H | NH2 | 4.29 | |
| | | | Arg | 100H | CZ | 4.94 | |
| His | 151I | CB | Arg | 100H | NH2 | 4.60 | |
| His | 151I | CG | Arg | 100H | NH2 | 3.85 | |
| | | | Arg | 100H | CZ | 4.95 | |
| His | 151I | ND1 | Trp | 102H | CB | 4.97 | |
| | | | Trp | 102H | CG | 4.92 | |
| | | | Trp | 102H | CD1 | 4.97 | |
| | | | Arg | 100H | NH2 | 4.43 | * |
| His | 151I | CE1 | Trp | 102H | CD2 | 4.70 | |
| | | | Trp | 102H | CB | 3.75 | |
| | | | Trp | 102H | CG | 3.98 | |
| | | | Trp | 102H | CD1 | 4.26 | |
| | | | Arg | 100H | NH2 | 4.25 | |
| His | 151I | NE2 | Trp | 102H | CB | 4.27 | |
| | | | Trp | 102H | CG | 4.89 | |
| | | | Trp | 102H | O | 4.92 | * |
| | | | Arg | 100H | NE | 4.92 | * |
| | | | Arg | 100H | NH2 | 3.45 | * |
| | | | Arg | 100H | CZ | 4.61 | |
| His | 151I | CD2 | Arg | 100H | NE | 4.97 | |
| | | | Arg | 100H | NH2 | 3.09 | |
| | | | Arg | 100H | CZ | 4.35 | |

TABLE 17
hIL-21, chain I, (SEQ ID NO: 1) interactions with the the heavy
chain (chain H) of Fab60 (SEQ ID No 10) and light chain (chain L)
of anti-IL-21 Fab60 (SEQ ID No 9, mutation D30E). A distance cut-
off of 5.0 Å was used. The contacts were identified by the CONTACT
computer software program of the CCP4 suite (Bailey, 1994).

| | | | aIL-21 Fab60(Fab35 | | | | |
|---|---|---|---|---|---|---|---|
| hIL-21 | | | with L, D30E mutation) | | | | |
| Res. | Res. # | Atom | Res. | Res. # | Atom | Distance | Possibly |
| Type | and Chain | name | Type | and Chain | name | [Å] | H-bond |
| Met | 39I | CE | Trp | 102H | CZ3 | 4.48 | |
| | | | Trp | 102H | CH2 | 4.79 | |
| Glu | 65I | CB | Tyr | 56H | CE2 | 4.78 | |
| Glu | 65I | CD | Tyr | 56H | CZ | 4.81 | |
| | | | Tyr | 56H | OH | 4.36 | |
| | | | Tyr | 56H | CE2 | 4.95 | |
| Glu | 65I | OE1 | Tyr | 56H | CE1 | 4.70 | |
| | | | Tyr | 56H | CZ | 3.74 | |
| | | | Tyr | 56H | OH | 3.16 | *** |
| | | | Tyr | 56H | CE2 | 3.94 | |
| Asp | 66I | N | Tyr | 56H | CE2 | 4.78 | |
| | | | Tyr | 56H | CD2 | 4.53 | |
| Asp | 66I | CA | Tyr | 56H | CD2 | 4.78 | |
| | | | Tyr | 57H | CE2 | 4.70 | |
| Asp | 66I | CB | Gly | 54H | O | 4.87 | |
| | | | Tyr | 56H | CA | 4.78 | |
| | | | Tyr | 56H | CB | 3.71 | |
| | | | Tyr | 56H | CG | 3.78 | |
| | | | Tyr | 56H | CD1 | 4.58 | |
| | | | Tyr | 56H | CE2 | 4.61 | |
| | | | Tyr | 56H | CD2 | 3.82 | |
| | | | Gly | 54H | N | 4.51 | |
| | | | Gly | 54H | CA | 4.14 | |
| | | | Thr | 52H | OG1 | 4.69 | |
| | | | Gly | 54H | C | 4.59 | |
| | | | Tyr | 56H | N | 4.60 | |
| | | | Tyr | 57H | CE2 | 4.33 | |
| | | | Tyr | 57H | CD2 | 4.56 | |
| Asp | 66I | CG | Gly | 54H | O | 4.38 | |
| | | | Tyr | 56H | CA | 4.52 | |
| | | | Tyr | 56H | CB | 3.90 | |
| | | | Tyr | 56H | CG | 4.38 | |
| | | | Tyr | 56H | CD2 | 4.86 | |
| | | | Ser | 53H | OG | 4.68 | |
| | | | Ser | 53H | C | 4.54 | |
| | | | Gly | 54H | N | 3.27 | |
| | | | Gly | 54H | CA | 3.29 | |
| | | | Thr | 52H | CA | 4.98 | |
| | | | Thr | 52H | CB | 3.92 | |
| | | | Thr | 52H | OG1 | 3.32 | |
| | | | Thr | 52H | C | 4.70 | |
| | | | Ser | 53H | N | 4.55 | |
| | | | Gly | 54H | C | 3.75 | |
| | | | Ser | 55H | N | 4.05 | |
| | | | Ser | 55H | C | 4.98 | |
| | | | Tyr | 56H | N | 3.96 | |
| | | | Tyr | 57H | N | 4.99 | |
| | | | Tyr | 57H | CE2 | 4.58 | |
| | | | Tyr | 57H | CD2 | 4.34 | |
| | | | Thr | 52H | CG2 | 4.96 | |
| Asp | 66I | OD1 | Ser | 53H | CA | 4.36 | |
| | | | Ser | 53H | CB | 4.63 | |
| | | | Ser | 53H | OG | 3.61 | * |
| | | | Ser | 53H | C | 4.08 | |
| | | | Gly | 54H | N | 2.99 | *** |
| | | | Gly | 54H | CA | 3.46 | |
| | | | Thr | 52H | CA | 4.57 | |
| | | | Thr | 52H | CB | 3.59 | |
| | | | Thr | 52H | OG1 | 3.45 | * |
| | | | Thr | 52H | C | 4.24 | |
| | | | Thr | 52H | O | 4.88 | * |
| | | | Ser | 53H | N | 3.80 | * |
| | | | Gly | 54H | C | 4.25 | |
| | | | Ser | 55H | N | 4.46 | * |
| | | | Tyr | 56H | N | 4.92 | * |
| | | | Tyr | 57H | CD2 | 4.90 | |
| | | | Thr | 52H | CG2 | 4.68 | |
| Asp | 66I | OD2 | Ser | 55H | O | 4.93 | * |
| | | | Gly | 54H | O | 3.68 | * |
| | | | Tyr | 56H | CA | 3.51 | |
| | | | Tyr | 56H | CB | 3.23 | |
| | | | Tyr | 56H | CG | 3.92 | |
| | | | Tyr | 56H | CD1 | 4.42 | |
| | | | Tyr | 56H | CD2 | 4.72 | |
| | | | Ser | 53H | C | 4.41 | |
| | | | Gly | 54H | N | 3.16 | *** |
| | | | Gly | 54H | CA | 3.10 | |
| | | | Thr | 52H | CA | 4.51 | |
| | | | Thr | 52H | CB | 3.58 | |
| | | | Thr | 52H | OG1 | 2.61 | *** |
| | | | Thr | 52H | C | 4.26 | |
| | | | Thr | 52H | O | 4.47 | * |
| | | | Ser | 53H | N | 4.44 | * |
| | | | Gly | 54H | C | 3.05 | |
| | | | Ser | 55H | N | 3.12 | *** |
| | | | Ser | 55H | CA | 3.95 | |
| | | | Ser | 55H | C | 3.78 | |
| | | | Tyr | 56H | N | 2.78 | *** |
| | | | Tyr | 56H | C | 4.25 | |
| | | | Tyr | 57H | N | 4.01 | * |
| | | | Tyr | 57H | CE2 | 4.66 | |
| | | | Tyr | 57H | CD2 | 4.15 | |
| | | | Thr | 52H | CG2 | 4.71 | |
| Asp | 66I | C | Tyr | 57H | CZ | 4.81 | |
| | | | Tyr | 57H | CE2 | 3.79 | |
| | | | Tyr | 57H | CD2 | 4.46 | |
| | | | Tyr | 57H | OH | 4.78 | |
| Asp | 66I | O | Tyr | 57H | CZ | 4.25 | |
| | | | Tyr | 57H | CE2 | 3.54 | |
| | | | Tyr | 57H | CD2 | 4.55 | |
| | | | Tyr | 57H | OH | 3.91 | * |
| Val | 67I | N | Tyr | 57H | CZ | 4.97 | |
| | | | Tyr | 57H | CE2 | 3.85 | |
| | | | Tyr | 57H | CD2 | 4.26 | |
| Val | 67I | CA | Tyr | 57H | CZ | 4.64 | |
| | | | Tyr | 57H | CE2 | 3.74 | |
| | | | Tyr | 57H | CD2 | 4.24 | |
| | | | Tyr | 57H | OH | 4.77 | |
| Val | 67I | C | Thr | 52H | CB | 4.81 | |
| | | | Tyr | 57H | CG | 4.60 | |
| | | | Tyr | 57H | CZ | 4.54 | |
| | | | Tyr | 57H | CE2 | 3.59 | |
| | | | Tyr | 57H | CD2 | 3.64 | |
| | | | Thr | 52H | CG2 | 4.22 | |
| Val | 67I | O | Thr | 52H | CB | 3.83 | |
| | | | Thr | 52H | OG1 | 4.45 | * |
| | | | Tyr | 57H | CG | 4.86 | |
| | | | Tyr | 57H | CE2 | 4.24 | |
| | | | Tyr | 57H | CD2 | 3.94 | |
| | | | Thr | 52H | CG2 | 3.35 | |
| Glu | 68I | N | Tyr | 57H | CB | 4.89 | |
| | | | Tyr | 57H | CG | 4.11 | |
| | | | Tyr | 57H | CD1 | 4.68 | |
| | | | Tyr | 57H | CE1 | 4.62 | |
| | | | Tyr | 57H | CZ | 4.02 | |
| | | | Tyr | 57H | CE2 | 3.40 | |
| | | | Tyr | 57H | CD2 | 3.46 | |
| | | | Tyr | 57H | OH | 4.59 | * |
| | | | Thr | 52H | CG2 | 4.51 | |
| Glu | 68I | CA | Tyr | 57H | CB | 4.55 | |
| | | | Tyr | 57H | CG | 4.15 | |
| | | | Tyr | 57H | CD1 | 4.71 | |
| | | | Tyr | 57H | CE1 | 4.98 | |
| | | | Tyr | 57H | CZ | 4.78 | |
| | | | Tyr | 57H | CE2 | 4.28 | |
| | | | Tyr | 57H | CD2 | 3.94 | |
| | | | Thr | 52H | CG2 | 4.07 | |
| Glu | 68I | CB | Tyr | 57H | CG | 4.52 | |
| | | | Tyr | 57H | CD1 | 4.66 | |
| | | | Tyr | 57H | CE1 | 4.81 | |
| | | | Tyr | 57H | CZ | 4.90 | |
| | | | Tyr | 57H | CE2 | 4.83 | |
| | | | Tyr | 57H | CD2 | 4.64 | |
| Glu | 68I | CG | Tyr | 57H | CB | 4.36 | |
| | | | Tyr | 57H | CG | 3.69 | |
| | | | Tyr | 57H | CD1 | 3.46 | |
| | | | Tyr | 57H | CE1 | 3.58 | |
| | | | Tyr | 57H | CZ | 4.00 | |
| | | | Tyr | 57H | CE2 | 4.25 | |
| | | | Tyr | 57H | CD2 | 4.11 | |
| | | | Tyr | 57H | OH | 4.77 | |
| Glu | 68I | CD | Tyr | 57H | CB | 3.98 | |
| | | | Tyr | 57H | CG | 3.75 | |
| | | | Tyr | 57H | CD1 | 3.44 | |
| | | | Tyr | 57H | CE1 | 4.01 | |
| | | | Tyr | 57H | CZ | 4.81 | |
| | | | Tyr | 57H | CD2 | 4.60 | |
| | | | His | 59H | CE1 | 4.40 | |
| | | | His | 59H | NE2 | 3.62 | |
| | | | His | 59H | CD2 | 4.67 | |
| Glu | 68I | OE1 | Tyr | 57H | CA | 4.94 | |
| | | | Tyr | 57H | CB | 3.66 | |
| | | | Tyr | 57H | CG | 3.88 | |
| | | | Tyr | 57H | CD1 | 3.94 | |
| | | | Tyr | 57H | CE1 | 4.79 | |
| | | | Tyr | 57H | CD2 | 4.76 | |
| | | | Tyr | 94L | CD1 | 4.92 | |
| | | | Tyr | 94L | CZ | 4.75 | |
| | | | His | 59H | CE1 | 4.02 | |
| | | | His | 59H | NE2 | 2.96 | *** |
| | | | His | 59H | CD2 | 3.77 | |
| | | | Tyr | 94L | CE1 | 4.13 | |
| | | | Tyr | 94L | OH | 4.55 | * |
| Glu | 68I | OE2 | Tyr | 57H | CB | 4.55 | |
| | | | Tyr | 57H | CG | 4.27 | |
| | | | Tyr | 57H | CD1 | 3.58 | |
| | | | Tyr | 57H | CE1 | 4.05 | |
| | | | His | 59H | CE1 | 3.90 | |
| | | | His | 59H | NE2 | 3.49 | * |
| | | | His | 59H | CD2 | 4.75 | |
| Glu | 68I | C | Thr | 52H | CG2 | 4.22 | |
| Thr | 69I | N | Ser | 33H | OG | 4.82 | * |
| | | | Thr | 52H | CG2 | 3.80 | |
| | | | Tyr | 94L | OH | 4.48 | * |
| Thr | 69I | CA | Ser | 33H | OG | 4.33 | |
| | | | Thr | 52H | CG2 | 4.62 | |
| | | | Tyr | 94L | OH | 4.70 | |
| Thr | 69I | CB | Ser | 33H | CB | 4.44 | |
| | | | Ser | 33H | OG | 3.80 | |
| | | | Thr | 52H | CG2 | 4.71 | |
| | | | Tyr | 94L | OH | 3.68 | |
| | | | Glu | 99H | CD | 4.60 | |
| | | | Glu | 99H | OE1 | 4.44 | |
| | | | Glu | 99H | OE2 | 4.47 | |
| | | | Tyr | 96L | OH | 4.32 | |
| Thr | 69I | OG1 | Thr | 52H | CA | 4.86 | |
| | | | Thr | 52H | CB | 4.74 | |
| | | | Ser | 50H | OG | 4.40 | * |
| | | | Ser | 33H | CA | 4.82 | |
| | | | Ser | 33H | CB | 3.45 | |
| | | | Ser | 33H | OG | 2.82 | *** |
| | | | Thr | 52H | CG2 | 3.60 | |
| | | | Tyr | 94L | OH | 3.76 | * |
| | | | Glu | 99H | CD | 4.85 | |
| | | | Glu | 99H | OE1 | 4.35 | * |
| | | | Tyr | 96L | OH | 4.97 | * |
| Thr | 69I | CG2 | Ser | 33H | CB | 4.11 | |
| | | | Ser | 33H | OG | 3.72 | |
| | | | Tyr | 94L | OH | 4.69 | |
| | | | Glu | 99H | CG | 4.24 | |
| | | | Glu | 99H | CD | 3.63 | |
| | | | Glu | 99H | OE1 | 3.80 | |
| | | | Glu | 99H | OE2 | 3.54 | |
| | | | Arg | 100H | C | 4.73 | |
| | | | Arg | 100H | O | 4.37 | |
| | | | Gly | 101H | N | 4.55 | |
| | | | Gly | 101H | CA | 4.17 | |
| | | | Tyr | 96L | OH | 4.27 | |
| Thr | 69I | O | Tyr | 94L | OH | 4.61 | * |
| Asn | 70I | N | Gly | 101H | CA | 3.98 | |
| | | | Gly | 101H | C | 4.63 | |
| | | | Trp | 102H | N | 4.32 | * |
| Asn | 70I | CA | Gly | 101H | CA | 4.39 | |
| | | | Gly | 101H | C | 4.62 | |
| | | | Trp | 102H | N | 4.11 | |
| | | | Trp | 102H | CA | 4.97 | |
| | | | Gly | 103H | N | 4.35 | |
| Asn | 70I | CB | Gly | 101H | CA | 4.86 | |
| | | | Trp | 102H | N | 4.85 | |
| | | | Gly | 103H | N | 4.37 | |
| | | | Gly | 103H | CA | 4.77 | |
| | | | Tyr | 105H | CD1 | 4.32 | |
| | | | Tyr | 105H | CE1 | 3.65 | |
| | | | Tyr | 105H | CZ | 4.35 | |
| | | | Tyr | 105H | OH | 4.45 | |
| Asn | 70I | CG | Arg | 100H | O | 4.46 | |
| | | | Gly | 101H | CA | 3.97 | |
| | | | Gly | 101H | C | 4.27 | |
| | | | Gly | 101H | O | 4.86 | |
| | | | Trp | 102H | N | 4.43 | |
| | | | Gly | 103H | N | 3.90 | |
| | | | Gly | 103H | C | 4.83 | |
| | | | Tyr | 104H | N | 4.79 | |
| | | | Gly | 103H | CA | 4.26 | |
| | | | Tyr | 105H | CD1 | 3.65 | |
| | | | Tyr | 105H | CE1 | 3.49 | |
| | | | Tyr | 105H | CZ | 4.37 | |
| | | | Tyr | 105H | OH | 4.90 | |
| | | | Tyr | 105H | N | 4.50 | |
| | | | Tyr | 105H | CA | 4.90 | |
| | | | Tyr | 105H | CG | 4.65 | |
| Asn | 70I | OD1 | Arg | 100H | C | 4.50 | |
| | | | Arg | 100H | O | 3.86 | * |
| | | | Gly | 101H | N | 4.30 | * |
| | | | Gly | 101H | CA | 3.13 | |
| | | | Gly | 101H | C | 3.15 | |
| | | | Gly | 101H | O | 3.65 | * |
| | | | Trp | 102H | N | 3.41 | * |
| | | | Trp | 102H | CA | 4.29 | |
| | | | Trp | 102H | C | 3.97 | |
| | | | Gly | 103H | N | 2.89 | *** |
| | | | Gly | 103H | C | 3.94 | |
| | | | Tyr | 104H | N | 3.73 | * |
| | | | Gly | 103H | CA | 3.44 | |
| | | | Tyr | 105H | CD1 | 4.05 | |
| | | | Tyr | 105H | CE1 | 4.05 | |
| | | | Tyr | 105H | CZ | 4.71 | |
| | | | Tyr | 104H | CA | 4.72 | |
| | | | Tyr | 104H | C | 4.86 | |
| | | | Tyr | 105H | N | 3.99 | * |
| | | | Tyr | 105H | CA | 4.75 | |
| | | | Tyr | 105H | CG | 4.73 | |
| Asn | 70I | ND2 | Glu | 99H | CD | 4.88 | |
| | | | Glu | 99H | OE2 | 3.87 | * |
| | | | Arg | 10H | O | 4.26 | * |
| | | | Gly | 11H | CA | 4.53 | |
| | | | Phe | 91L | CB | 4.53 | |
| | | | Phe | 91L | CD1 | 4.48 | |
| | | | Phe | 91L | O | 4.88 | * |
| | | | Tyr | 96L | OH | 4.34 | * |
| | | | Tyr | 105H | CD1 | 3.42 | |
| | | | Tyr | 105H | CE1 | 3.56 | |
| | | | Tyr | 105H | CZ | 4.73 | |
| | | | Tyr | 105H | N | 4.36 | * |
| | | | Phe | 91L | CG | 4.83 | |
| | | | Tyr | 105H | CA | 4.42 | |
| | | | Tyr | 105H | CG | 4.52 | |
| Asn | 70I | C | Trp | 102H | N | 4.93 | |
| Asn | 70I | O | Gly | 103H | N | 4.98 | * |
| | | | Tyr | 105H | OH | 4.65 | * |
| Glu | 72I | N | Trp | 102H | N | 4.97 | * |
| Glu | 72I | CA | Trp | 102H | CE2 | 4.68 | |
| | | | Trp | 102H | CD2 | 4.58 | |
| | | | Trp | 102H | CE3 | 4.49 | |
| | | | Trp | 102H | CZ3 | 4.52 | |
| | | | Trp | 102H | CH2 | 4.62 | |
| | | | Trp | 102H | CZ2 | 4.71 | |
| Glu | 72I | CB | Trp | 102H | NE1 | 3.78 | |
| | | | Trp | 102H | CE2 | 3.27 | |
| | | | Trp | 102H | CD2 | 3.34 | |
| | | | Trp | 102H | CE3 | 3.61 | |
| | | | Trp | 102H | CZ3 | 3.82 | |
| | | | Trp | 102H | CH2 | 3.76 | |
| | | | Trp | 102H | CZ2 | 3.51 | |
| | | | Trp | 102H | N | 4.39 | |
| | | | Trp | 102H | CA | 4.47 | |
| | | | Trp | 102H | CB | 4.82 | |
| | | | Trp | 102H | CG | 3.96 | |
| | | | Trp | 102H | CD1 | 4.13 | |
| Glu | 72I | CG | Trp | 102H | NE1 | 3.70 | |
| | | | Trp | 102H | CE2 | 3.68 | |
| | | | Trp | 102H | CD2 | 4.13 | |
| | | | Trp | 102H | CE3 | 4.77 | |
| | | | Trp | 102H | CH2 | 4.69 | |
| | | | Trp | 102H | CZ2 | 4.02 | |
| | | | Trp | 102H | N | 4.50 | |
| | | | Trp | 102H | CG | 4.41 | |
| | | | Trp | 102H | CD1 | 4.11 | |
| Glu | 72I | CD | Trp | 102H | NE1 | 4.20 | |
| | | | Trp | 102H | CE2 | 4.52 | |
| | | | Trp | 102H | CD2 | 4.67 | |
| | | | Gly | 101H | CA | 4.04 | |
| | | | Gly | 101H | C | 4.31 | |
| | | | Trp | 102H | N | 3.58 | |
| | | | Trp | 102H | CA | 4.46 | |
| | | | Trp | 102H | CG | 4.47 | |
| | | | Trp | 102H | CD1 | 4.13 | |
| Glu | 72I | OE1 | Trp | 102H | NE1 | 4.70 | * |
| | | | Trp | 102H | CE2 | 4.90 | |
| | | | Trp | 102H | CD2 | 4.67 | |
| | | | Gly | 101H | N | 4.92 | * |
| | | | Gly | 101H | CA | 3.65 | |
| | | | Gly | 101H | C | 3.69 | |
| | | | Gly | 101H | O | 4.92 | * |
| | | | Trp | 102H | N | 2.79 | *** |
| | | | Trp | 102H | CA | 3.65 | |
| | | | Trp | 102H | CB | 4.61 | |
| | | | Trp | 102H | CG | 4.33 | |
| | | | Trp | 102H | CD1 | 4.32 | |
| | | | Trp | 102H | C | 4.73 | |
| | | | Gly | 103H | N | 4.73 | * |
| Glu | 72I | OE2 | Ser | 1033H | OG | 4.93 | * |
| | | | Trp | 102H | NE1 | 4.64 | * |
| | | | Gly | 101H | N | 4.64 | * |
| | | | Gly | 101H | CA | 3.86 | |
| | | | Gly | 101H | C | 4.54 | |
| | | | Trp | 102H | N | 4.15 | * |
| | | | Trp | 102H | CD1 | 4.57 | |
| Glu | 72I | C | Trp | 102H | CE2 | 4.97 | |
| | | | Trp | 102H | CD2 | 4.60 | |
| | | | Trp | 102H | CE3 | 4.05 | |
| | | | Trp | 102H | CZ3 | 3.90 | |
| | | | Trp | 102H | CH2 | 4.31 | |
| | | | Trp | 102H | CZ2 | 4.86 | |
| Glu | 72I | O | Trp | 102H | CE3 | 4.33 | |
| | | | Trp | 102H | CZ3 | 3.77 | |
| | | | Trp | 102H | CH2 | 4.01 | |
| | | | Trp | 102H | CZ2 | 4.79 | |
| Trp | 73I | N | Trp | 102H | CD2 | 4.70 | |
| | | | Trp | 102H | CE3 | 4.02 | |
| | | | Trp | 102H | CZ3 | 4.16 | |
| | | | Trp | 102H | CH2 | 4.94 | |
| | | | Trp | 102H | CA | 4.93 | |
| Trp | 73I | CA | Trp | 102H | CE3 | 4.42 | |
| | | | Trp | 102H | CZ3 | 4.38 | |
| Trp | 73I | CB | Trp | 102H | CE3 | 4.79 | |
| Trp | 73I | CG | Trp | 102H | CD2 | 4.96 | |
| | | | Trp | 102H | CE3 | 3.93 | |
| | | | Trp | 102H | CZ3 | 4.43 | |
| | | | Trp | 102H | CA | 4.73 | |
| | | | Trp | 102H | C | 4.82 | |
| | | | Trp | 102H | O | 4.54 | |
| Trp | 73I | CD1 | Trp | 102H | CD2 | 4.74 | |
| | | | Trp | 102H | CE3 | 3.98 | |
| | | | Trp | 102H | CZ3 | 4.86 | |
| | | | Trp | 102H | CA | 3.75 | |
| | | | Trp | 102H | CB | 4.17 | |
| | | | Trp | 102H | CG | 4.90 | |
| | | | Trp | 102H | C | 3.56 | |
| | | | Trp | 102H | O | 3.15 | |
| | | | Gly | 103H | N | 4.37 | |
| | | | Gly | 103H | CA | 4.92 | |
| Trp | 73I | NE1 | Trp | 102H | CD2 | 4.47 | |
| | | | Trp | 102H | CE3 | 3.72 | |
| | | | Trp | 102H | CZ3 | 4.67 | |
| | | | Trp | 102H | CA | 3.91 | |
| | | | Trp | 102H | CB | 3.82 | |
| | | | Trp | 102H | CG | 4.59 | |
| | | | Trp | 102H | C | 3.76 | |
| | | | Trp | 102H | O | 3.03 | *** |
| | | | Gly | 103H | N | 4.83 | * |
| Trp | 73I | CE2 | Trp | 102H | CD2 | 4.55 | |
| | | | Trp | 102H | CE3 | 3.50 | |
| | | | Trp | 102H | CZ3 | 4.09 | |
| | | | Trp | 102H | CA | 4.96 | |
| | | | Trp | 102H | CB | 4.63 | |
| | | | Trp | 102H | O | 4.38 | |
| Trp | 73I | CD2 | Trp | 102H | CD2 | 4.86 | |
| | | | Trp | 102H | CE3 | 3.63 | |
| | | | Trp | 102H | CZ3 | 3.88 | |
| Trp | 73I | CE3 | Trp | 102H | CE3 | 4.12 | |
| | | | Trp | 102H | CZ3 | 3.89 | |
| Trp | 73I | CZ3 | Trp | 102H | CE3 | 4.45 | |
| | | | Trp | 102H | CZ3 | 4.08 | |
| Trp | 73I | CH2 | Trp | 102H | CE3 | 4.34 | |
| | | | Trp | 102H | CZ3 | 4.27 | |
| Trp | 73I | CZ2 | Trp | 102H | CD2 | 4.92 | |
| | | | Trp | 102H | CE3 | 3.92 | |
| | | | Trp | 102H | CZ3 | 4.30 | |
| Phe | 76I | CB | Trp | 102H | CZ3 | 4.38 | |
| | | | Trp | 102H | CH2 | 4.73 | |
| Phe | 76I | CG | Trp | 102H | CZ3 | 4.96 | |
| Lys | 117I | N | Trp | 102H | O | 4.82 | * |
| Lys | 117I | CA | Gly | 103H | CA | 4.80 | |
| Lys | 117I | CB | Gly | 103H | CA | 4.30 | |
| | | | Tyr | 105H | CZ | 4.86 | |
| | | | Tyr | 105H | OH | 4.50 | |
| | | | Gly | 103H | O | 4.83 | |
| | | | Tyr | 105H | CE2 | 4.32 | |
| Lys | 117I | CD | Ser | 31L | OG | 3.88 | |
| | | | Asp | 50L | CG | 4.01 | |
| | | | Asp | 50L | OD1 | 3.64 | |
| | | | Asp | 50L | OD2 | 3.69 | |
| | | | Gly | 103H | O | 4.55 | |
| | | | Tyr | 105H | CE2 | 4.53 | |
| | | | Tyr | 105H | CD2 | 4.92 | |
| Lys | 117I | CE | Ser | 31L | OG | 4.06 | |
| | | | Asp | 50L | CG | 4.16 | |
| | | | Asp | 50L | OD1 | 4.04 | |
| | | | Asp | 50L | OD2 | 3.45 | |
| Lys | 117I | NZ | Ser | 31L | CB | 4.05 | |
| | | | Ser | 31L | OG | 3.21 | *** |
| | | | Asp | 50L | CB | 4.94 | |
| | | | Asp | 50L | CG | 3.46 | |
| | | | Asp | 50L | OD1 | 3.37 | * |
| | | | Asp | 50L | OD2 | 2.77 | *** |
| | | | Asp | 50L | O | 4.85 | * |
| Lys | 117I | C | Gly | 103H | N | 4.71 | |
| | | | Gly | 103H | CA | 4.29 | |
| | | | Tyr | 105H | OH | 4.45 | |
| Lys | 117I | O | Trp | 102H | CA | 4.72 | |
| | | | Trp | 102H | C | 3.95 | |
| | | | Trp | 102H | O | 4.00 | * |
| | | | Gly | 103H | N | 3.72 | * |
| | | | Gly | 103H | CA | 3.61 | |
| | | | Tyr | 105H | OH | 4.80 | * |
| His | 118I | N | Tyr | 105H | OH | 4.01 | * |
| His | 118I | CA | Tyr | 105H | CZ | 4.98 | |
| | | | Tyr | 105H | OH | 3.85 | |
| His | 118I | C | Tyr | 105H | CZ | 4.72 | |
| | | | Tyr | 105H | OH | 3.39 | |
| His | 118I | O | Tyr | 105H | CZ | 4.95 | |
| | | | Tyr | 105H | OH | 3.58 | * |
| Arg | 119I | N | Tyr | 105H | CZ | 4.79 | |
| | | | Tyr | 105H | OH | 3.55 | * |
| Arg | 119I | CA | Tyr | 105H | OH | 4.03 | |
| Arg | 119I | CB | Tyr | 105H | OH | 4.64 | |
| | | | Asn | 92L | O | 4.73 | |
| Arg | 119I | CG | Phe | 91L | O | 4.25 | |
| | | | Tyr | 105H | CE1 | 4.34 | |
| | | | Tyr | 105H | CZ | 4.63 | |
| | | | Tyr | 105H | OH | 3.94 | |
| | | | Asn | 92L | C | 5.00 | |
| | | | Asn | 92L | O | 4.25 | |
| Arg | 119I | CD | Phe | 91L | C | 4.52 | |
| | | | Phe | 91L | O | 3.41 | |
| | | | Tyr | 105H | CE1 | 4.96 | |
| | | | Asn | 92L | N | 4.95 | |
| | | | Asn | 92L | CA | 4.38 | |
| | | | Asn | 92L | C | 3.96 | |
| | | | Asn | 92L | O | 3.24 | |
| | | | Ser | 93L | N | 4.87 | |
| Arg | 119I | NE | Tyr | 94L | CE1 | 4.94 | |
| | | | Phe | 91L | O | 4.05 | * |
| | | | Asn | 92L | C | 4.98 | |
| | | | Asn | 92L | O | 4.30 | * |
| Arg | 119I | CZ | Tyr | 94L | CD1 | 4.89 | |
| | | | Tyr | 94L | CE1 | 4.32 | |
| | | | Asn | 92L | O | 4.67 | |
| Arg | 119I | NH1 | Tyr | 94L | CD1 | 4.88 | |
| | | | Tyr | 94L | CE1 | 4.66 | |
| | | | Asn | 92L | O | 4.15 | * |
| Arg | 119I | NH2 | Tyr | 94L | CD1 | 4.70 | |
| | | | Tyr | 94L | CZ | 4.72 | |
| | | | Tyr | 94L | CE1 | 3.87 | |
| | | | Tyr | 94L | OH | 4.48 | * |
| Leu | 120I | CG | Glu | 30L | OE2 | 4.89 | |
| Leu | 120I | CD1 | Glu | 30L | OE2 | 4.27 | |
| Leu | 120I | CD2 | Glu | 30L | OE2 | 4.49 | |
| Thr | 121I | OG1 | Asn | 92L | O | 4.82 | * |
| Pro | 123I | CG | Tyr | 57H | OH | 4.92 | |
| Tyr | 128I | CD1 | Tyr | 57H | OH | 4.80 | |
| Tyr | 128I | CE1 | Tyr | 57H | OH | 4.22 | |
| Glu | 129I | OE2 | Tyr | 56H | OH | 4.96 | * |
| Leu | 143I | CA | Trp | 102H | CZ2 | 4.82 | |
| Leu | 143I | CB | Trp | 102H | CH2 | 4.81 | |
| | | | Trp | 102H | CZ2 | 4.88 | |
| Leu | 143I | CG | Trp | 102H | CZ3 | 4.78 | |
| | | | Trp | 102H | CH2 | 3.56 | |
| | | | Trp | 102H | CZ2 | 3.71 | |
| Leu | 143I | CD1 | Trp | 102H | CE2 | 4.73 | |
| | | | Trp | 102H | CH2 | 3.96 | |
| | | | Trp | 102H | CZ2 | 3.68 | |
| Leu | 143I | CD2 | Trp | 102H | CZ3 | 4.87 | |
| | | | Trp | 102H | CH2 | 3.96 | |
| | | | Trp | 102H | CZ2 | 4.52 | |
| Leu | 143I | C | Trp | 102H | CH2 | 4.37 | |
| | | | Trp | 102H | CZ2 | 4.16 | |
| Leu | 143I | O | Trp | 102H | NE1 | 4.93 | * |
| | | | Trp | 102H | CE2 | 4.40 | |
| | | | Trp | 102H | CH2 | 3.75 | |
| | | | Trp | 102H | CZ2 | 3.28 | |
| Leu | 144I | N | Trp | 102H | CH2 | 4.96 | |
| Leu | 144I | CA | Trp | 102H | CH2 | 4.98 | |
| Gln | 145I | O | Ser | 31H | OG | 4.76 | * |
| Lys | 146I | N | Ser | 31H | OG | 4.74 | * |
| Lys | 146I | CA | Ser | 31H | OG | 4.01 | |
| | | | Ser | 31H | CB | 4.33 | |
| | | | Ser | 31H | O | 4.87 | |
| | | | Trp | 102H | NE1 | 4.91 | |
| Lys | 146I | CB | Ser | 31H | OG | 4.33 | |
| | | | Ser | 31H | CA | 4.84 | |
| | | | Ser | 31H | CB | 4.49 | |
| | | | Ser | 31H | O | 4.36 | |
| | | | Trp | 102H | NE1 | 4.44 | |
| | | | Trp | 102H | CE2 | 4.91 | |
| | | | Trp | 102H | CZ2 | 4.63 | |
| Lys | 146I | CG | Ser | 30H | O | 4.92 | |
| | | | Ser | 31H | OG | 3.78 | |
| | | | Ser | 31H | CA | 4.27 | |
| | | | Ser | 31H | CB | 4.19 | |
| | | | Ser | 31H | C | 4.84 | |
| | | | Ser | 31H | O | 4.44 | |
| Lys | 146I | CD | Ser | 30H | O | 4.75 | |
| | | | Ser | 53H | OG | 4.25 | |
| | | | Ser | 31H | OG | 4.86 | |
| | | | Ser | 31H | CA | 4.67 | |
| | | | Ser | 31H | C | 4.97 | |
| | | | Ser | 31H | O | 4.58 | |
| Lys | 146I | CE | Ser | 30H | C | 5.00 | |
| | | | Ser | 30H | O | 3.92 | |
| | | | Ser | 53H | CB | 4.44 | |
| | | | Ser | 53H | OG | 3.63 | |
| | | | Ser | 31H | CA | 4.69 | |
| Lys | 146I | NZ | Ser | 30H | O | 4.78 | * |
| | | | Ser | 53H | OG | 4.97 | * |
| Lys | 146I | C | Trp | 102H | NE1 | 4.06 | |
| | | | Trp | 102H | CE2 | 4.52 | |
| | | | Trp | 102H | CZ2 | 4.57 | |
| | | | Trp | 102H | CD1 | 4.94 | |
| Lys | 146I | O | Ser | 31H | OG | 4.94 | * |
| | | | Ser | 31H | CB | 4.68 | |
| | | | Ser | 31H | O | 4.78 | * |
| | | | Trp | 102H | NE1 | 4.27 | * |
| | | | Trp | 102H | CE2 | 4.98 | |
| | | | Trp | 102H | CD1 | 4.84 | |
| Met | 147I | N | Trp | 102H | NE1 | 3.79 | * |
| | | | Trp | 102H | CE2 | 3.87 | |
| | | | Trp | 102H | CD2 | 4.88 | |
| | | | Trp | 102H | CH2 | 4.64 | |
| | | | Trp | 102H | CZ2 | 3.75 | |
| | | | Trp | 102H | CD1 | 4.73 | |
| Met | 147I | CA | Trp | 102H | NE1 | 3.70 | |
| | | | Trp | 102H | CE2 | 3.67 | |
| | | | Trp | 102H | CD2 | 4.27 | |
| | | | Trp | 102H | CE3 | 4.90 | |
| | | | Trp | 102H | CZ3 | 4.99 | |
| | | | Trp | 102H | CH2 | 4.53 | |
| | | | Trp | 102H | CZ2 | 3.86 | |
| | | | Trp | 102H | CG | 4.60 | |
| | | | Trp | 102H | CD1 | 4.29 | |
| Met | 147I | CB | Trp | 102H | NE1 | 4.04 | |
| | | | Trp | 102H | CE2 | 3.48 | |
| | | | Trp | 102H | CD2 | 3.84 | |
| | | | Trp | 102H | CE3 | 4.05 | |
| | | | Trp | 102H | CZ3 | 3.90 | |
| | | | Trp | 102H | CH2 | 3.58 | |
| | | | Trp | 102H | CZ2 | 3.38 | |
| | | | Trp | 102H | CG | 4.56 | |
| | | | Trp | 102H | CD1 | 4.65 | |
| Met | 147I | CG | Trp | 102H | NE1 | 4.66 | |
| | | | Trp | 102H | CE2 | 4.03 | |
| | | | Trp | 102H | CD2 | 3.79 | |
| | | | Trp | 102H | CE3 | 3.72 | |
| | | | Trp | 102H | CZ3 | 3.84 | |
| | | | Trp | 102H | CH2 | 4.08 | |
| | | | Trp | 102H | CZ2 | 4.20 | |
| | | | Trp | 102H | CG | 4.40 | |
| | | | Trp | 102H | CD1 | 4.89 | |
| His | 149I | CB | Ile | 28H | CB | 4.66 | |
| | | | Ile | 28H | CG1 | 4.89 | |
| | | | Ser | 31H | OG | 3.98 | |
| | | | Ser | 31H | CB | 4.04 | |
| His | 149I | CG | Ile | 28H | CB | 4.10 | |
| | | | Ile | 28H | CG1 | 4.06 | |
| | | | Ile | 28H | CG2 | 4.39 | |
| | | | Ser | 31H | OG | 3.87 | |
| | | | Ser | 31H | CB | 4.40 | |
| His | 149I | ND1 | Ile | 28H | CB | 3.96 | |
| | | | Ile | 28H | CG1 | 4.13 | |
| | | | Ile | 28H | CG2 | 3.77 | |
| | | | Ser | 31H | OG | 3.01 | *** |
| | | | Ser | 31H | CB | 3.92 | |
| His | 149I | CE1 | Ile | 28H | CD1 | 4.90 | |
| | | | Ile | 28H | CB | 4.17 | |
| | | | Ile | 28H | CG1 | 3.98 | |
| | | | Ile | 28H | CG2 | 3.81 | |
| | | | Ser | 31H | OG | 3.95 | |
| His | 149I | NE2 | Ile | 28H | CD1 | 4.68 | |
| | | | Ile | 28H | CB | 4.42 | |
| | | | Ile | 28H | CG1 | 3.79 | |
| | | | Ile | 28H | CG2 | 4.44 | |
| His | 149I | CD2 | Ile | 28H | CB | 4.45 | |
| | | | Ile | 28H | CG1 | 3.90 | |
| | | | Ile | 28H | CG2 | 4.83 | |
| His | 149I | C | Tyr | 32H | OH | 4.03 | |
| | | | Tyr | 32H | CZ | 4.82 | |
| His | 149I | O | Tyr | 32H | OH | 3.59 | * |
| | | | Tyr | 32H | CZ | 4.66 | |
| Gln | 150I | N | Tyr | 32H | OH | 4.13 | * |
| | | | Tyr | 32H | CZ | 4.70 | |
| Gln | 150I | CA | Tyr | 32H | CE2 | 4.96 | |
| | | | Tyr | 32H | OH | 3.57 | |
| | | | Tyr | 32H | CE1 | 4.46 | |
| | | | Tyr | 32H | CZ | 4.16 | |
| | | | Arg | 100H | NE | 4.61 | |
| | | | Arg | 100H | CZ | 4.84 | |
| | | | Arg | 100H | NH2 | 4.80 | |
| Gln | 150I | CB | Tyr | 32H | OH | 4.33 | |
| | | | Tyr | 32H | CE1 | 4.41 | |
| | | | Tyr | 32H | CZ | 4.49 | |
| | | | Arg | 100H | CG | 4.62 | |
| | | | Arg | 100H | CD | 4.88 | |
| | | | Arg | 100H | NE | 4.14 | |
| | | | Trp | 102H | CD1 | 4.72 | |
| | | | Arg | 100H | CZ | 4.64 | |
| | | | Arg | 100H | NH2 | 4.56 | |
| Gln | 150I | CG | Tyr | 32H | CG | 4.67 | |
| | | | Tyr | 32H | CD2 | 4.73 | |
| | | | Ser | 31H | C | 4.91 | |
| | | | Ser | 31H | O | 4.00 | |
| | | | Trp | 102H | NE1 | 4.83 | |
| | | | Tyr | 32H | CE2 | 4.39 | |
| | | | Tyr | 32H | OH | 4.18 | |
| | | | Tyr | 32H | CD1 | 4.19 | |
| | | | Tyr | 32H | CE1 | 3.77 | |
| | | | Tyr | 32H | CZ | 3.90 | |
| | | | Arg | 100H | CG | 4.76 | |
| | | | Trp | 102H | CD1 | 4.56 | |
| Gln | 150I | CD | Tyr | 32H | CA | 4.99 | |
| | | | Tyr | 32H | CG | 4.59 | |
| | | | Ser | 31H | C | 4.90 | |
| | | | Ser | 31H | O | 3.86 | |
| | | | Trp | 102H | NE1 | 4.36 | |
| | | | Tyr | 32H | CD1 | 4.02 | |
| | | | Tyr | 32H | CE1 | 4.02 | |
| | | | Tyr | 32H | CZ | 4.57 | |
| | | | Arg | 100H | CB | 4.33 | |
| | | | Arg | 100H | CG | 4.31 | |
| | | | Arg | 100H | NE | 4.98 | |
| | | | Arg | 100H | CA | 4.12 | |
| | | | Arg | 100H | C | 4.52 | |
| | | | Gly | 101H | N | 3.85 | |
| | | | Gly | 101H | CA | 4.83 | |
| | | | Trp | 102H | CD1 | 3.88 | |
| Gln | 150I | OE1 | Ser | 31H | O | 4.98 | * |
| | | | Trp | 102H | NE1 | 4.38 | * |
| | | | Tyr | 32H | CD1 | 4.61 | |
| | | | Tyr | 32H | CE1 | 4.57 | |
| | | | Glu | 99H | O | 4.95 | * |
| | | | Arg | 100H | CB | 3.48 | |
| | | | Arg | 100H | CG | 3.76 | |
| | | | Arg | 100H | CD | 4.67 | |
| | | | Arg | 100H | NE | 4.24 | * |
| | | | Arg | 100H | N | 4.82 | * |
| | | | Arg | 100H | CA | 3.43 | |
| | | | Arg | 100H | C | 3.65 | |
| | | | Arg | 100H | O | 4.85 | * |
| | | | Gly | 101H | N | 3.00 | *** |
| | | | Gly | 101H | CA | 3.96 | |
| | | | Gly | 101H | C | 4.04 | |
| | | | Gly | 101H | O | 3.95 | * |
| | | | Trp | 102H | N | 4.85 | * |
| | | | Trp | 102H | CB | 4.79 | |
| | | | Trp | 102H | CG | 4.51 | |
| | | | Trp | 102H | CD1 | 3.53 | |
| Gln | 150I | NE2 | Tyr | 32H | N | 4.32 | * |
| | | | Tyr | 32H | CA | 3.86 | |
| | | | Tyr | 32H | CB | 4.42 | |
| | | | Tyr | 32H | CG | 4.04 | |
| | | | Tyr | 32H | CD2 | 4.61 | |
| | | | Tyr | 32H | C | 4.94 | |
| | | | Ser | 33H | N | 4.85 | * |
| | | | Ser | 31H | C | 3.87 | |
| | | | Ser | 31H | O | 2.82 | *** |
| | | | Trp | 102H | NE1 | 4.54 | * |
| | | | Tyr | 32H | CE2 | 4.98 | |
| | | | Tyr | 32H | CD1 | 3.79 | |
| | | | Tyr | 32H | CE1 | 4.22 | |
| | | | Glu | 99H | O | 4.54 | * |
| | | | Tyr | 32H | CZ | 4.78 | |
| | | | Arg | 100H | CB | 4.98 | |
| | | | Arg | 100H | CA | 4.37 | |
| | | | Arg | 100H | C | 4.68 | |
| | | | Gly | 101H | N | 3.93 | * |
| | | | Gly | 101H | CA | 4.88 | |
| | | | Trp | 102H | CD1 | 4.30 | |
| Gln | 150I | C | Tyr | 32H | OH | 4.38 | |
| | | | Arg | 100H | NE | 4.20 | |
| | | | Arg | 100H | CZ | 4.00 | |
| | | | Arg | 100H | NH1 | 4.63 | |
| | | | Arg | 100H | NH2 | 3.80 | |
| Gln | 150I | O | Tyr | 32H | OH | 4.32 | * |
| | | | Arg | 100H | CG | 4.93 | |
| | | | Arg | 100H | CD | 4.22 | |
| | | | Arg | 100H | NE | 3.46 | * |
| | | | Arg | 100H | CZ | 3.12 | |
| | | | Arg | 100H | NH1 | 3.53 | * |
| | | | Arg | 100H | NH2 | 3.21 | *** |
| His | 151I | N | Arg | 100H | CZ | 4.75 | |
| | | | Arg | 100H | NH2 | 4.21 | * |
| His | 151I | CA | Arg | 100H | CZ | 4.69 | |
| | | | Arg | 100H | NH2 | 3.95 | |
| His | 151I | CB | Arg | 100H | NH2 | 4.31 | |
| His | 151I | CG | Arg | 100H | CZ | 4.84 | |
| | | | Arg | 100H | NH2 | 3.63 | |
| His | 151I | ND1 | Trp | 102H | CB | 4.98 | |
| | | | Arg | 100H | NH2 | 3.97 | * |
| His | 151I | CE1 | Trp | 102H | CD2 | 4.84 | |
| | | | Trp | 102H | CB | 3.82 | |
| | | | Trp | 102H | CG | 4.13 | |
| | | | Trp | 102H | CD1 | 4.58 | |
| | | | Arg | 100H | NH2 | 3.91 | |
| His | 151I | NE2 | Trp | 102H | CB | 4.41 | |
| | | | Arg | 100H | CZ | 4.72 | |
| | | | Arg | 100H | NH2 | 3.45 | * |
| His | 151I | CD2 | Arg | 100H | CZ | 4.56 | |
| | | | Arg | 100H | NH2 | 3.23 | |

In the last column
“***” indicates a strong possibility for a hydrogen bond at this contact (distance < 3.3 Å) as calculated by CONTACT,
“*” indicates a weak possibility (distance > 3.3 Å).
Blank indicates that the program considered there to be no possibility of a hydrogen bond.
Hydrogen-bonds are specific between a donor and an acceptor, are typically strong, and are easily identifiable.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., & Zwart, P. H. (2010). PHENIX: a comprehensive Python-based system for macromolecular structure solution. Acta Cryst. D 66, 213-221.
Afonine, P. V., Grosse-Kunstleve, R. W., & Adams, P. D. (2005). Contribution 8.
Bailey, S. (1994). The ccp4 suite—programs for protein crystallography. Acta Crystallogr. Sect. D-Biol. Crystallogr. 50, 760-763.
Emsley, P., Lohkamp, B., Scott, W. G., & Cowtan, K. (2010). Features and development of Coot. Acta Crystallogr. Sect. D-Biol. Crystallogr. 66, 486-501.
Kabsch, W. (2010). Integration, scaling, space-group assignment and post-refinement. Acta Crystallographica Section D Biological Crystallography 66, 133-144.
Krissinel, E. & Henrick, K. (2004). Secondary-structure matching (SSM), a new tool for fast protein structure alignment in three dimensions. Acta Crystallographica Section D Biological Crystallography 60, 2256-2268.
Lee, B. & Richards, F. M. (1971). THE INTERPRETATION OF PROTEIN STRUCTURES ESTIMATION OF STATIC ACCESSIBILITY. J Mol Biol 55, 379-400.
Murshudov, G. N., Skubak, P., Lebedev, A. A., Pannu, N. S., Steiner, R. A., Nicholls, R. A., Winn, M. D., Long, F., & Vagin, A. A. (2011). REFMAC5 for the refinement of macromolecular crystal structures. Acta Crystallographica Section D Biological Crystallography 67, 355-367.
Perrakis, A., Morris, R., & Lamzin, V. S. (1999). Automated protein model building combined with iterative structure refinement. Nat Struct Biol 6, 458-463.
Saff, E. B. & Kuijlaars, A. B. J. (1997). Distributing many points on a sphere. Math Intell 19, 5-11.
Ursby, T., Mammen, C. B., Cerenius, Y., Svensson, C., Sommarin, B., Fodje, M. N., Kvick, Å., Logan, D. T., Als-Nielsen, J., Thunnissen, M. M. G. M., Larsen, S., & Liljas, A. The New Macromolecular Crystallography Stations At MAX-lab: The MAD Station, pp. 1241-1246.
Vagin, A. & Teplyakov, A. (1997). Molrep—an automated program for molecular replacement. J. Appl. Crystallogr. 30, 1022-1025.

### Example 9

### Comparison of Interaction Kinetics for Anti-hIL-21 mAb37, mAb61, mAb62 and mAb65 to hIL-21 by Surface Plasmon Resonance (SPR)

Binding studies were performed on a Biacore T200 instrument that measures molecular interactions in real time through surface plasmon resonance. Experiments were run at 25° C. and the samples were stored at 10° C. in the sample compartment. The signal (RU, response units) reported by the Biacore is directly correlated to the mass on the individual sensor chip surfaces in four serial flow cells.

Anti-human Fc monoclonal antibodies from Biacore human Fc capture kit was immobilized onto flow cells of a CM4 sensor chip according to the manufacturer's instructions. The final immobilization level of capture antibody was approximately 2,500 RU in one experiment. Capture of the human anti-hIL21 antibodies mAb37, mAb61, mAb62, mAb65 was conducted by diluting the antibody to 0.125 μg/ml into running buffer (10 mM Hepes 0,3 M NaCl, 5 mM CaCl2, 0.05% surfactant P20, pH 8.0 containing 1 mg/ml BSA) and injected at 10 μl/min for 180 s in one of flow cells 2-4, creating a reference surface in flow cell 1 with only anti-Fc antibody immobilized. This typically resulted in final capture levels of test antibodies of approximately 50-85 RU and Rmax values of analyte of 10-16 RU. Binding of hIL-21 protein was conducted by injecting analyte over all flow cells to allow for comparative analyses of binding to different captured anti-IL-21 antibodies relative to binding to the reference flow cell. hIL-21 protein was diluted serially 1:3 to 2-162 nM into running buffer, injected at 100 μl/min for 210 s and allowed to dissociate for 600 or 14000 s. The CM4 surface was regenerated after each injection cycle of analyte via two injections of 3M MgCl₂ at 50 μl/min. This regeneration step removed the anti-IL-21 antibody and any bound IL-21 from the immobilized capture antibody surface, and allowed for the subsequent binding of the next interaction sample pair. The regeneration procedure did not remove the directly immobilized anti-Fc capture antibody from the chip surface.

In order to obtain kinetic data, such as ka (association rate), kd (dissociation rate) and KD (equilibrium dissociation constant), data analysis was performed using the Biacore T200 evaluation software 1.0, fitting data to 1:1 Langmuir model. No significant non-specific binding to the reference control surface was observed. Binding curves were processed by double referencing (subtraction of reference surface signals as well as blank buffer injections over captured anti-IL-21 antibodies). This allowed correction for instrument noise, bulk shift and drift during sample injections.

Human IL-21 dissociates from mAb37, mAb61, mAb62 and mAb65 with off-rates less than what can be accurately measured by the currently used assay (kd<1E-5 s⁻¹) and average ka values of 5-7 E+5 (Ms)⁻¹ resulting in KD of <20 μM. Results are based on two different experiments. Individual relative standard errors (RSE) of parameter ka were <1.1%. Results are shown in Table 18.

These data clearly demonstrates that the four different antibodies tested share similar binding properties to human IL-21.

TABLE 18
Results from individual experiments of binding constants ka (association rate), kd
(dissociation rate) and calculated KD (equilibrium dissociation constant) for the
interaction of human IL21 to monoclonal antibodies mAb37, mAb61, mAb62
and mAb65.

| Antibody | Exp no | ka (1/Ms) | RSE ka (%) | kd (1/s) | KD calc. (M) |
|---|---|---|---|---|---|
| mAb37 | 1 | 5.7E+05 | 0.1 | <1E−5 | <1.8E−11 |
| mAb37 | 1 | 5.9E+05 | 0.3 | <1E−5 | <1.7E−11 |
| mAb37 | 1 | 5.8E+05 | 0.1 | <1E−5 | <1.7E−11 |
| mAb37 | 2 | 6.6E+05 | 0.4 | <1E−5 | <1.5E−11 |
| mAb37 | 2 | 6.2E+05 | 0.2 | <1E−5 | <1.6E−11 |
| mAb61 | 2 | 6.6E+05 | 0.4 | <1E−5 | <1.5E−11 |
| mAb61 | 2 | 6.7E+05 | 0.7 | <1E−5 | <1.5E−11 |
| mAb62 | 1 | 5.6E+05 | 0.2 | <1E−5 | <1.8E−11 |
| mAb62 | 1 | 5.8E+05 | 0.3 | <1E−5 | <1.7E−11 |
| mAb62 | 1 | 6.0E+05 | 0.9 | <1E−5 | <1.7E−11 |
| mAb65 | 1 | 5.2E+05 | 0.1 | <1E−5 | <1.9E−11 |
| mAb65 | 1 | 5.3E+05 | 0.7 | <1E−5 | <1.9E−11 |
| mAb65 | 1 | 5.4E+05 | 0.2 | <1E−5 | <1.9E−11 |

### Example 10

### Inhibitory Effect of Anti-IL-21 mAb37 Variants on Human B Cell Proliferation

The neutralizing potential of 6 anti-IL-21 antibodies was compared in a B cell proliferation assay. The 6 antibodies include mAb37 and the 5 variants, mAb61, mAb62, mAb63, mAb64 and mAb65 described in example 12. The antibodies were tested for their ability to neutralise the recombinant human IL-21 in the B cell proliferation assay.

Blood bags were obtained from healthy human volunteers and PBMCs were isolated from 50 ml of heparinised peripheral blood by Ficoll-Paque™ Plus (GE Healthcare) gradient centrifugation. Blood was diluted to 100 ml in phosphate-buffered saline (PBS) at room temperature and 35 ml aliquots were distributed into 50 ml conical tubes carefully overlaying 14 ml of Ficoll-Paque™ Plus (Ge Healthcare) at room temperature. The tubes were spun for 25 minutes at 1680 rpm (600×g) at room temperature without brake. The PBMC interface layer was removed carefully and washed twice with PBS containing 2% FCS. B cells were isolated by negative selection using EasySep human B Cell enrichment Kit (StemCell Technologies SERL, Grenoble, France). A small sample of the purified B cells was tested for purity by FACS analysis and found to be >95-97% pure in all experiments.

B cells were cultured in RPMI-1640 media (Invitrogen) supplemented with heat inactivated foetal calf serum (FCS) (Gibco) or Healthy human serum (HS) (Sigma), and Penicillin/Streptomycin (Gibco). To test the inhibitory effect of mAb37 variants, human B cells were isolated from 2 individual donors, donor 1 and 2.

The B cells were plated at 50.000 cells per well in a 96-well U-bottom tissue culture plate. Cells were treated with 0.1 μg/ml anti-CD40 (R&D Systems), 50 ng/ml (3.21 nM) recombinant human IL-21. The cells were incubated for 3 days at 37° C. and 5% CO₂ in a humidified incubator. The antibodies were titrated and after three days, the cells were pulsed with 1 μCi/well of [³H]-Thymidine (Perkin Elmer Life Sciences) for the last 20 hours. The cells were harvested onto UniFilter-96 GF/C filter plates (Packard Instruments, Perkin Elmer) and the amount of [³H]-thymidine incorporation was quantified using a TopCount NXT (Perkin Elmer). The concentration of anti-IL-21 mAb required for reducing proliferation by 50% (IC₅₀) was calculated using the GraphPad Prism v5.0 software (GraphPad Inc.) and the sigmoidal dose-response (variable slope, 4-parameters) equation.

The IC₅₀ for the WT mAb37 and the 5 variants were all found to be very similar, with IC₅₀ values in the sub-nanomolar range. All antibodies were tested on B-cells from both donors and the data is listed in table 19 below. Due to technical issues a full data set for mAb62 was only obtained for donor 2.

TABLE 19
IC₅₀ values for mAb37, mAb61, mAb62, mAb63, mAb64 and
mAb65 in B cell proliferation assay

| | IC₅₀ (nM) | IC₅₀ (nM) |
|---|---|---|
| | Donor 1 | Donor 2 |
| mAb37 | 0.14 | 0.18 |
| mAb61 | 0.22 | 0.21 |
| mAb62 | N/A | 0.23 |
| mAb63 | 0.16 | 0.25 |
| mAb64 | 0.80 | 0.74 |
| mAb65 | 0.49 | 0.19 |

### Example 11

Bioactivity of Anti-IL-21 Antibodies in NK-92 Assay.

The antibodies were tested for their ability to neutralise the recombinant human IL-21 in the NK-cell based bioassay. The anti-IL-21 mAb37 was included as reference material.

The NK-cell based bioassay was used for in vitro determination of the bioactivity of anti-IL-21 antibodies. The NK-92 cell line (ATCC/LGC Promochem) is a human suspension lymphoblast derived from peripheral blood mononuclear cells. Cells express the IL-21 receptor endogenously and are dependent on IL-2 or IL-21 for cell proliferation. The neutralization of IL-21 by anti-IL-21 is measured by growth inhibition via addition of alamarBlue® (a cell viability indicator).

During maintenance the NK-92 cells were kept proliferating by addition of IL-2. For assay, NK-92 cells were washed and plated out in 96 well plates (Matrix Technology) at a density of 1.6×10⁵ cells/ml (equal to 12,800 cells per well). The cells were stimulated with recombinant human IL-21 at a fixed concentration of 5431 pg/ml. Serial dilutions of Anti-IL-21 antibodies prepared in assay media, ranging from 0-12,800 pg/ml, was added in triplicates in three different positions in the 96-well plate. The cells were incubated for 3 days at 37° C. and 5% CO₂ in a humidified incubator. On day three 10 μl alamarBlue® (Biosource) was added and fluorescence was measured after 5 hours of incubation on a Synergy instrument (Bio Tek).

Data was analyzed in BioCalc (MicroLex) in a four-parameter logistic curve model. Results are given as percentage (%) of reference material mAb37, based on single determinations.

The bioactivity measured for the 5 mutated antibodies (table 20) were all found to be very similar when compared relative to the bioactivity of the reference material mAb37.

TABLE 20
Bioactivity for mAb61, mAb62, mAb63, mAb64
and mAb65 NK-92 assay relative to mAb37

| | Bioactivity as % of RM |
|---|---|
| Antibody | (mAb37) |
| mAb63 | 92.1 |
| mAb64 | 116.4 |
| mAb61 | 86.2 |
| mAb62 | 73.2 |
| mAb65 | 96.8 |

### Example 12

### Cloning and Sequencing of Anti-IL-21 mAb14

This example describes cloning and sequencing of the human heavy chain and light chain sequences of anti-IL-21 mAb14 from hybridoma 366.328.10.63

Total RNA was extracted from hybridoma cells using the RNeasy-Mini Kit from Qiagen and used as template for cDNA synthesis. cDNA was synthesized in a 5′-RACE reaction using the SMARTer™ RACE cDNA amplification kit from Clontech. Subsequent target amplification of HC and LC sequences was performed by PCR using Phusion Hot Start polymerase (Finnzymes) and the universal primer mix (UPM) included in the SMARTer™ RACE kit as forward primer. Reverse primers specific for human IgG constant regions or the human Kappa constant region were used for PCR amplification of the HC and LC sequences, respectively. The PCR products were separated by gel electrophoresis, extracted using the GFX PCR DNA & Gel Band Purification Kit from GE Healthcare Bio-Sciences and cloned for sequencing using a Zero Blunt TOPO PCR Cloning Kit and chemically competent TOP10 E. coli (Invitrogen). Colony PCR was performed on selected colonies using an AmpliTaq Gold® FAST Master Mix from Applied Biosystems and M13uni/M13rev primers. Colony PCR clean-up was performed using the ExoSAP-IT enzyme mix (USB). Sequencing was performed at MWG Biotech, Martinsried Germany using either M13uni(−21)/M13rev(−29) or T3/T7 sequencing primers. Sequences were analyzed and annotated using the Vector NTI program. All kits and reagents were used according to the manufacturer's instructions.

A single unique human kappa type LC and a single unique human HC, subclass IgG4 were identified.

### Example 13

### Generation of Expression Vectors for Transient Expression of Anti-IL-21 mAb14 Antibody and Fab Fragment Variants

To enable epitope mapping and binding analyses, a series of CMV promotor-based expression vectors (pTT vectors) were generated for transient expression of mAb14 variants in the HEK293-6E EBNA-based expression system developed by Yves Durocher (Durocher et al. Nucleic Acid Research, 2002). In addition to the CMV promotor, the vectors contain a pMB1 origin, an EBV origin and the Amp resistance gene.

The region corresponding to the anti-IL-21 mAb14 VH domain was cloned into a linearized pTT-based vector containing the sequence of an engineered human IgG4 CH domain using standard PCR and restriction-based cloning methods. As part of the PCR amplification, the sequence for the native IgG signal peptide was exchanged by standard overlapping PCR with the signal peptide sequences derived from human CD33. The PCR template used was a topo-vector generated as described in Example 12. The engineered human IgG4 CH domain contains a single amino acid substitution: S241P in the hinge region. The proline mutation at position 241 (S241P residue numbering according to Kabat, S228P residue numbering according to the EU numbering system (Edelman G. M. et AL., Proc. Natl. Acad. USA 63, 78-85 (1969) and S228P numbering in SEQ ID No. 7) was introduced in the IgG4 hinge region to eliminated formation of monomeric antibody fragments, i.e. “half-antibodies” comprising of one LC and one HC.

Vector constructs were transformed into E. coli for selection. The sequence of the final construct was verified by DNA sequencing. The stabilizing S241P mutation in the human IgG4 hinge region constitutes the only difference between mAb14 and mAb37, i.e. mAb37 is the hinge stabilized version of mAb14. The amino acid of HC mAb37 corresponds to SEQ ID No 7 with an S228P substitution at residue 228. The mAb14 and mAb37 nomenclature is used interchangeably, but for all recombinantly produced mAb variants the IgG4 constant region contains the stabilizing S241P mutation.

A pTT-based vector was also generated for transient expression of the mAb37 Fab fragment; Fab35. The region corresponding to the VH domain was cloned into a linearized pTT-based vector containing the sequence of a truncated human IgG4 constant domain. The IgG4 CH domain is terminated in the hinge region—generating a truncated HC, constituting amino acid residues 1-221 of the full HC listed as SEQ ID No. 7. The VH domain was swapped into the Fab expression vector by restriction-based cloning and transformed into E. coli for selection. The sequence of the final construct was verified by DNA sequencing. The Fab35 HC amino acid sequence is listed as SEQ ID No. 10. The Fab35 LC corresponds to the mAb37 LC, the amino acid sequence is listed as SEQ ID No. 9 (and SEQ ID No. 6).

The region corresponding to the mAb37 VL domain was cloned into a linearized pTT-based vector containing the sequence for a human kappa CL domain using the standard PCR methods for amplification and signal peptide exchange described for mAb37 HC above and standard restriction-based cloning methods. The PCR template used was a topo-vector generated as described in Example 12. Vector constructs were transformed into E. coli for selection. The sequence of the final construct was verified by DNA sequencing. The mAb37 LC amino acid sequence corresponds to mAb14 LC and is listed as SEQ ID No 6 (and SEQ ID No. 9).

Recombinant expression of mAb37 and Fab35 were performed as described in Example 14.

### Example 14

### Site-Directed Mutagenesis of Anti-IL-21 mAb37

Site-directed mutagenesis was performed to generate the variants of anti-IL-21 mAb37/Fab35 listed in table 21. The mutations are listed according to numbering on reference sequences corresponding to mAb14 LC SEQ ID 6, mAb14 HC SEQ ID No. 7, Fab35 LC SEQ ID 9, Fab35 HC SEQ ID No. 10. Mutations were introduced in the HC or LC by standard site directed mutagenesis using the QuikChange™ Site-Directed mutagenesis kit from Stratagene and specific mutagenic primers were used to introduce point mutations. The kit was used according to the manufacturer's protocol. The pTT-based expression plasmid for WT mAb37/Fab35 LC described in Example 13 was used as template for the LC mutagenesis. The HC mutants were generated using the truncated HC expression plasmid for WT Fab35 described in Example 13 as template. Subsequently the plasmid for expression of full length HC mutants were generated by swapping the mutated VH domains into the linearized pTT-based vector containing the sequence of the human IgG4(S241P)CH domain. Domain swapping was done by standard restriction-based cloning methods. Vector constructs were transformed into E. coli for selection. The sequences of all final constructs were verified by DNA sequencing.

TABLE 21
Variants of mAb37/Fab35

| | | | | | LC Reference | HC reference |
|---|---|---|---|---|---|---|
| | | muta- | | | SEQ ID No. | SEQ ID No. |
| mAb | Fab | tion ID | CDR | Chain | mAb/Fab | mAb/Fab |
| mAb37 | Fab35 | WT | | | 6/9 | 7/10 |
| mAb61 | Fab56 | D62E | H2 | H | 6/9 | 7/10 |
| mAb62 | Fab57 | K65R | H2 | H | 6/9 | 7/10 |
| mAb63 | Fab58 | R24K | L1 | L | 6/9 | 7/10 |
| mAb64 | Fab59 | Q27N | L1 | L | 6/9 | 7/10 |
| mAb65 | Fab60 | D30E | L1 | L | 6/9 | 7/10 |

To express mAb37 mutants, HEK293-6E cells were co-transfected with LC plasmids (WT or mutants) and HC plasmids (WT or mutant) as described below. To express mAb37 Fab fragment, HEK293-6E cells were co-transfected with LC plasmids (WT or mutants) and truncated HC plasmids (WT or mutant).

### Recombinant Expression of mAb Variants

Variants of mAb37 including variants of Fab35 were expressed by co-transfection of HEK293-6E cells with pTT-based HC and LC vectors according to the generic antibody expression protocol listed below.

Cell Maintenance:

HEK293-6E cells were grown in suspension in FreeStyle™ 293 expression medium (Gibco) supplemented with 25 μg/ml Geneticin (Gibco), 0.1% v/v of the surfactant Pluronic F-68 (Gibco) & 1% v/v Penicillin-Streptomycin (Gibco). Cells were cultured in Erlenmeyer shaker flasks in shaker incubators at 37° C., 8% CO₂ and 125 rpm and maintained at cell densities between 0.1−1.5×10⁶ cells/ml.

DNA Transfection:
The cell density of cultures used for transfection was 0.9−2.0×10⁶ cells/ml.
A mix of 0.5 μg LC vector DNA+0.5 μg HC vector DNA was used per ml cell culture.
The DNA was diluted in Opti-MEM media (Gibco) 30 μl media/μg DNA, mixed and incubated at room temperature (23-25° C.) for 5 min.
293Fectin™ (Invitrogen) was used as transfection reagent at a concentration of 1 μl per μg DNA.
The 293Fectin™ was diluted 30× in Opti-MEM media (Gibco), mixed and incubated at room temperature (23-25° C.) for 5 min.
The DNA and 293Fectin solutions were mixed and left to incubate at room temperature (23-25° C.) for 25 min.
The DNA-293Fectin mix was then added directly to the cell culture.
The transfected cell culture was transferred to a shaker incubator at 37° C., 8% CO₂ and 125 rpm.
5 days post transfection, cell culture supernatants were harvested by centrifugation, followed by filtration through a 0.22 μm PES filter (Corning).
Quantitative analysis of antibody production was performed by BioLayer Interferometry directly on clarified cell culture supernatants using the FortéBio Octet system or by SDS-PAGE analysis.

### Purification of mAb and Fab Fragment Variants

mAb37 variants were purified by standard affinity chromatography using MabSelectSuRe resin from GE Healthcare. The purified antibodies were buffer exchanged to PBS buffer pH7.2.

Fab fragments were purified by standard affinity chromatography using KappaSelect resin from GE Healthcare. The purified Fab fragments were buffer exchanged to PBS buffer pH7.2.

Quality assessment and concentration determination was done by SEC-HPLC, endotoxin levels were measured by the standard Kinetic Turbidimetric LAL method.

### Abbreviations

Aa: amino acid
mAb: monoclonal antibody
HC: heavy chain
LC: light chain
VH: variable domain—heavy chain
VL: variable domain—light chain
CH: constant region—heavy chain
CL: constant region—light chain
PCR: polymerase chain reaction
WT: wild type

### Example 15

### Epitope Mapping by HX-MS of mAb37 and Variants mAb61, mAb62 and mAb65 on hIL-21 (See Also Example 7)

### Materials

Protein Batches Used were:

hIL-21: human recombinant IL-21 (expressed in E. coli as the mature peptide; residues 30-162 of SEQ ID NO: 1 with an added N-terminal Methionine residue), mAb37 and variants mAb61, mAb62 and mAb65, sequences as described in example 14

All proteins were buffer exchanged into PBS pH 7.4 before experiments.

### Methods: HX-MS Experiments

### Instrumentation and Data Recording

The HX experiments were performed on a nanoACQUITY UPLC System with HDX Technology (Waters Inc.) coupled to a Synapt G2 mass spectrometer (Waters Inc.). The Waters HDX system contained a Leap robot (H/D-x PAL; Waters Inc.) operated by the LeapShell software (Leap Technologies Inc/Waters Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and HX reactions and maintained at 2° C. for storage of protein and quench solution, respectively. The Waters HDX system furthermore contained a temperature controlled chamber holding the pre- and analytical columns, and the LC tubing and switching valves at 1° C. A separately temperature controlled chamber holds the pepsin column at 25° C. For the inline pepsin digestion, 100 μL quenched sample containing 100 pmol hIL-21 was loaded and passed over a Poroszyme® Immobilized Pepsin Cartridge (2.1×30 mm (Applied Biosystems)) placed at 25° C. using a isocratic flow rate of 100 μL/min (0.1% formic acid:CH₃CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (1×100 mm (Waters Inc.)), and the peptides separated using a 9 min gradient of 10-40% B delivered at 200 μl/min from the nanoAQUITY UPLC system (Waters Inc.). The mobile phases consisted of A: 0.1% formic acid and B: 0.1% formic acid in CH₃CN. The ESI MS data, and the separate elevated energy (MS^{E}) experiments were acquired in positive ion mode using a Synapt G2 mass spectrometer (Waters Inc.). Leucine-enkephalin was used as the lock mass ([M+]⁺ ion at m/z 556.2771) and data was collected in continuum mode (For further description, see Andersen and Faber, Int. J. Mass Spec., 302, 139-148 (2011)).

### Data Analysis

Peptic peptides were identified in separate experiments using standard MS^{E} methods where the peptides and fragments are further aligned utilizing the ion mobility properties of the Synapt G2 (Waters Inc.). MS^{E} data were processed using ProteinLynx Global Server version 2.5 (Waters Inc.). The HX-MS raw data files were processed in the DynamX software (Waters Inc.). DynamX automatically performs the lock mass-correction and deuterium incorporation determination, i.e., centroid determination of deuterated peptides. Furthermore, all peptides were inspected manually to ensure correct peak and deuteration assignment by the software.

### Epitope Mapping Experiment

Amide hydrogen/deuterium exchange (HX) was initiated by a 10-fold dilution of hIL-21 in the presence or absence of mAb37, mAb61, mAb62 or mAb65 into the corresponding deuterated buffer (i.e. PBS prepared in D₂O, 96% D₂O final, pH 7.4 (uncorrected value)). All HX reactions were carried out at 20° C. and contained 2 μM hIL-21 in the absence or presence of 1.2 μM mAb thus giving a 1.2 fold molar excess of mAb binding sites. At appropriate time intervals ranging from 10 sec to 3000 sec, 50 μl aliquots of the HX reaction were quenched by 50 μl ice-cold quenching buffer (1.35M TCEP) resulting in a final pH of 2.5 (uncorrected value).

### Results and Discussion

Epitope Mapping mAb37, mAb61, mAb62 and mAb65

The epitope mapping of mAb14 on hIL-21 is described in example 7. However, mAb14, in the form of mAb37 (see example 12-13), was also included in these experiments for reference.

The HX time-course of 29 peptides, covering 97% of the primary sequence of hIL-21 were monitored in the absence or presence of mAb37, mAb61, mAb62 or mAb65 for 10 to 3000 sec (table 22).

### Epitope Mapping

The observed exchange pattern in the early timepoints (<300 sec) in the presence or absence of mAb37, mAb61, mAb62 or mAb65 can be divided into different groups: One group of peptides display an exchange pattern that is unaffected by the binding of these mAbs in the early timepoints. In contrast, another group of peptides in hIL-21 show protection from exchange upon mAb37, mAb61, mAb62 or mAb65 binding in the very early timepoints (Table 22, fx peptide F76-L84 at less than 1 min exchange). Interestingly, the same group of hIL-21 derived peptides were affected by binding of these mAbs thus the epitopes for mAb37, mAb61, mAb62 or mAb65 appear identical and thus identical to the epitope for mAb14 as determined in example 7. A group of peptides showed weak protection at slightly longer timelines. These could be secondary effects of mAb binding, e.g. stabilization effects (Table 22, e.g. peptide 145-D55).

### CONCLUSION

Upon binding of either mAb37, mAb61, mAb62 or mAb65 all regions of hIL-21 showed similar responses. The same group of peptides were affected by mAb binding in the early time-points thus the epitopes for mAb37, mAb61, mAb62 or mAb65 appear identical to the epitope for mAb14 determined in example 7.

TABLE 22
HXMS analysis of hIL-21 yielding epitope information for mAb molecules.
After the deuterium exchange reaction, IL-21 was digested with pepsin
yielding the following peptic peptide regions that were analyzed.

| | Compound | | | |
|---|---|---|---|---|
| Sequence | mAb37 | mAb61 | mAb62 | mAb65 |
| M29-M39 | N | N | N | N |
| M29-D44 | N | N | N | N |
| Q30-M39 | N | N | N | N |
| G31-M39 | N | N | N | N |
| R40-D44 | N | N | N | N |
| I45-N51 | W | W | W | W |
| I45-D55 | W | W | W | W |
| L56-D66 | W | W | W | W |
| P58-D66 | W | W | W | W |
| L61-D66 | N | N | N | N |
| N70-F76 | EX | na | na | na |
| F76-L84 | EX | EX | EX | EX |
| S77-L84 | EX | EX | EX | EX |
| Q80-V98 | N | N | N | N |
| K85-V98 | N | N | N | N |
| E93-V98 | N | N | N | N |
| E93-S127 | N | N | N | N |
| R94-V98 | N | N | N | N |
| S127-S162 | EX | EX | EX | EX |
| F136-L143 | W | W | W | W |
| F136-L144 | W | W | W | W |
| F136-S162 | EX | EX | EX | EX |
| L137-L143 | W | W | W | W |
| L137-L144 | W | W | W | W |
| E138-L144 | W | W | W | W |
| E138-S162 | EX | EX | EX | EX |
| K141-S162 | W | W | W | W |
| L144-S162 | N | N | N | N |
| Q145-S162 | N | N | N | N |

EX: exchange protection upon mAb binding indicating epitope region (>0.6 Da at both two timepoints below 1 min exchange time).
W: Weak exchange protection upon mAb binding (>0.6 Da at more than two timepoints below 10 min exchange time).
N: No exchange protection upon mAb binding (<0.2 Da).
na: Not analyzable in respective experiment.

### Example 16

### Co-Binding Studies of Human IL-21 to Anti IL-21 and IL-21Rα/γC Subunits by Surface Plasmon Resonance (SPR) with mAb6, mAb37 and mAb24

Binding studies were performed on a Biacore T200 as described in Example 3 but in the current example, anti-human IL-21 monoclonal antibodies mAb6, mAb37 and mAb24 (binding to IL-21 but not competing with mAb6 or mAb37), were immobilized directly onto flow cells of a CM5 sensor chip. mAb24 is the antibody produced by the hybridoma clone 338.28.6.3/338.28.6 disclosed in WO2010055366. Another difference from Example 3 was that individual IL-21 receptor chains IL-21Rα-ECD and common γC-ECD protein were injected in series, creating a stepwise binding of (mAb)/IL-21/IL-21Rα/γC. In this setup, any lack of common γC protein binding was not dependent on absence of IL-21Rα but on competing antibody used to capture IL-21.

Data analysis was performed as described in Example 3, but using the Biacore T200 evaluation software 1.0.

In the current example it was shown that binding of IL-21Rα to captured IL-21 is a prerequisite for binding of common γC. It was also concluded that mAb37 prevents interaction of γC to IL-21/IL-21Rα complex. Hence, mAb37 will inhibit the biological effects mediated by IL-21 through γC and form ligand:IL-21 complexes having the ability to bind specifically to IL-21Rα present on cell surfaces.

When IL-21 was captured by a control antibody, binding to a separate site on IL-21 compared to both mAb6 and mAb37, sequential binding of both individual IL-21 receptor chains IL-21Rα and common γC protein was observed.

These results also explain why IL-21 captured by mAb19, as described in Example 3, was not able to bind simultaneously to neither IL-21Rα-ECD nor γC-ECD.

TABLE 23
Ability of different antibodies to bind simultaneously to (+) or to compete with (−) binding
of different receptor subunits to IL-21. Injection number indicate sequence of injections.
Y/N indicates whether receptor subunits were injected or not.

| | | Injection 2 | | Injection 3 | |
|---|---|---|---|---|---|
| Immobilized | Injection 1 | IL-21Rα | IL-21Rα | γC | γC |
| mAb | Capture | injected | binding | injected | binding |
| mAb6 | 100 nM hIL-21 | N | n/a | Y | (+) |
| mAb6 | 100 nM hIL-21 | Y (50 nM) | − | Y | (+) |
| mAb24 | 100 nM hIL-21 | N | n/a | Y | − |
| mAb24 | 100 nM hIL-21 | Y (50 nM) | + | Y | + |
| mAb37 | 100 nM hIL-21 | N | n/a | Y | − |
| mAb37 | 100 nM hIL-21 | Y (50 nM) | + | Y | − |

1. An antibody which binds to an epitope on IL-21, wherein said epitope comprises one or more of the following amino acids: Glu 65, Asp 66, Val 67, Glu 68, Thr 69, Asn 70, Glu 72, Trp 73, one or more of the following amino acids Lys 117, His 118, Arg 119, and one or more of the following amino acids: Leu 143, Lys 146, Met 147, His 149, Gln 150, and His 151 as set forth in SEQ ID No. 1, provided that the antibody is not the monoclonal antibody mAb14, the light and heavy chains of which are set forth in SEQ ID No. 6 and SEQ ID No. 7, respectively.

2. An antibody which binds to an epitope on IL-21, wherein said epitope comprises one or more of the following amino acids: Glu 65 to Trp 73, one or more of the following amino acids: Lys 117 to Arg 119, and one or more of the following amino acids: Leu 143 to His 151 as set forth in SEQ ID No. 1, provided that the antibody is not the monoclonal antibody mAb14, the light and heavy chains of which are set forth in SEQ ID No. 6 and SEQ ID No. 7, respectively.

3. An antibody which binds to an epitope on IL-21, wherein said epitope comprises one or more of the Arg 40 to Val 67 amino acids as well as one or more of the Glu 129 to His 149 amino acids, as set forth in SEQ ID No. 1, provided that the antibody is not mAb14, the light and heavy chains of which are set forth in SEQ ID No. 6 and SEQ ID No. 7, respectively.

4. An antibody which binds to an epitope on IL-21, wherein said epitope comprises one or more of the Glu 65 to Trp 73 amino acids in IL-21 (SEQ ID NO. 1) provided that the antibody is not mAb14, the light and heavy chains of which are set forth in SEQ ID No. 6 and SEQ ID No. 7, respectively.

5. An antibody according to claim 2, wherein said antibody binds one or more of Glu 65, Asp 66, and Val 67 as set forth in SEQ ID NO. 1.

6. An antibody according to claim 2, wherein said antibody binds His 149 as set forth in SEQ ID NO. 1.

7. An antibody according to claim 4, wherein said epitope comprises one or more of the Glu 65, Asp 66, Val 67, and His 149 amino acids as set forth in SEQ ID NO. 1.

8. An antibody according to claim 3, wherein said epitope comprises one or more of the following amino acids: Arg 40, Lys 50, Glu 65, Asp 66, Val 67, Glu 129, Glu 135, Glu 138, Arg 139, Lys 141, Ser 142, Gln 145, and His 149 as set forth in SEQ ID No. 1.

9. An antibody according to claim 1, wherein said epitope comprises one or more of the following amino acids: Glu 65, Asp 66, Val 67, Glu 68, Thr 69, Asn 70, Glu 72, Trp 73, Lys 117, His 118, Arg 119, leu 143, Lys 146, Met 147, His 149, Gln 150, and His 151.

10. An antibody according to claim 9, wherein said antibody comprises a light chain comprising at least one of CDR1, CDR2, and CDR3 as set forth in SEQ ID No. 6, and a heavy chain comprising at least one of CDR1, CDR2, and CDR3 as set forth in SEQ ID No. 7.

11. An antibody according to claim 2, wherein said antibody interferes with the binding of common γC chain to IL-21.

12. An antibody according to claim 10, wherein said antibody is a variant of mAb14, the light and heavy chains thereof which are set forth in SEQ ID No. 6 and SEQ ID No. 7 respectively, wherein said antibody comprises one or more mutations in the CDR sequences, wherein said mutations are selected from one or more from the list consisting of: A61S (SEQ ID NO 7), D62E (SEQ ID NO 7), V64I (SEQ ID NO 7), and K65R (SEQ ID NO 7), R24K (SEQ ID NO 6), S26T (SEQ ID NO 6), Q27N (SEQ ID NO 6), D30E (SEQ ID NO 6), S53T (SEQ ID NO 6), and S56T (SEQ ID NO 6).

13. A pharmaceutical composition comprising an antibody according to claim 1, and optionally one or more pharmaceutically acceptable excipients.

14. A method of treating an immunological disorder administering an antibody according to claim 1 to a patient in need thereof in an amount effective for treating the disorder.

15. A method for selecting a ligand which binds to IL-21, wherein said method comprises screening one or more libraries of ligands with an IL-21 mimic, wherein said IL-21 mimic comprises an epitope comprising the following amino acids: Glu 65, Asp 66, Val 67, and His 149 as set forth in SEQ ID No. 1, and isolating one or more ligands which bind to said epitope.
